(19) United States
(12) Patent Application Publication
(10) Pub. No.: US 2004/0014040 A1
(43) Pub. Date: Jan. 22, 2004

(54) Cardiotoxin molecular toxicology modeling

(76) Inventors: Donna Mendrick, Gaithersburg, MD (US); Mark Porter, Gaithersburg, MD (US); Kory Johnson, Gaithersburg, MD (US); Brandon Higgs, Gaithersburg, MD (US); Arthur Castle, Gaithersburg, MD (US); Michael Elashoff, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/191,803

(22) Filed: Jul. 10, 2002

Related U.S. Application Data
(60) Provisional application No. 60/303,819, filed on Jul. 10, 2001.
(60) Provisional application No. 60/305,623, filed on Jul. 17, 2001.
(60) Provisional application No. 60/369,351, filed on Apr. 3, 2002.
(60) Provisional application No. 60/377,611, filed on May 6, 2002.

(51) Int. Cl. C12Q 1/68; G06F 19/00; G01N 33/48; G01N 33/50

(52) U.S. Cl. 435/6; 702/20

(57) ABSTRACT

The present invention is based on the elucidation of the global changes in gene expression and the identification of toxicity markers in tissues or cells exposed to a known cardiotoxin. The genes may be used as toxicity markers in drug screening and toxicity assays. The invention includes a database of genes characterized by toxin-induced differential expression that is designed for use with microarrays and other solid-phase probes.

### RELATED APPLICATIONS

This application claims priority to U.S. Provisional Applications 60/303,819; 60/305,623; 60/369,351; and 60/377,611, all of which are herein incorporated by reference in their entirety. This application is also related to U.S. application Ser. Nos. 09/917,800; 10/060,087; and 10/152,319, all of which are also herein incorporated by reference in their entirety.

### SEQUENCE LISTING SUBMISSION ON COMPACT DISC

The Sequence Listing submitted concurrently herewith on compact disc is herein incorporated by reference in its entirety. Three copies of the Sequence Listing, one on each of three compact discs are provided. Copy 1 and Copy 2 are identical. Copies 1 and 2 are also identical to the CRF. Each electronic copy of the Sequence Listing was created on Jun. 19, 2002 with a file size of 1523 KB. The file names are as follows: Copy 1-g15090us.txt; Copy 2-g15090us.txt; and CRF-g15090us.txt.

### BACKGROUND OF THE INVENTION

The need for methods of assessing the toxic impact of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are the most easily maintained and manipulated. In addition, unicellular screening systems often use easily detectable changes in phenotype to monitor the effect of test compounds on the cell. Unicellular organisms, however, are inadequate models for estimating the potential effects of many compounds on complex multicellular animals, as they do not have the ability to carry out biotransformations.

The biotransformation of chemical compounds by multicellular organisms is a significant factor in determining the overall toxicity of agents to which they are exposed. Accordingly, multicellular screening systems may be preferred or required to detect the toxic effects of compounds. The use of multicellular organisms as toxicology screening tools has been significantly hampered, however, by the lack of convenient screening mechanisms or endpoints, such as those available in yeast or bacterial systems.

### SUMMARY OF THE INVENTION

The present invention is based, in part, on the elucidation of the global changes in gene expression in tissues or cells exposed to known toxins, in particular cardiotoxins, as compared to unexposed tissues or cells as well as the identification of individual genes that are differentially expressed upon toxin exposure.

In various aspects, the invention includes methods of predicting at least one toxic effect of a compound, predicting the progression of a toxic effect of a compound, and predicting the cardiotoxicity of a compound. The invention also includes methods of identifying agents that modulate the onset or progression of a toxic response. Also provided are methods of predicting the cellular pathways that a compound modulates in a cell. The invention also includes methods of identifying agents that modulate protein activities.

In a further aspect, the invention includes probes comprising sequences that specifically hybridize to genes in Tables 1-5I. Also included are solid supports comprising at least two of the previously mentioned probes. The invention also includes a computer system that has a database containing information identifying the expression level in a tissue or cell sample exposed to a cardiotoxin of a set of genes in Tables 1-5I.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death, are often characterized by the variations in the expression levels of groups of genes.

Changes in gene expression are also associated with the effects of various chemicals, drugs, toxins, pharmaceutical agents and pollutants on an organism or cell. Thus, changes in the expression levels of particular genes (e.g. oncogenes or tumor suppressors) may serve as signposts for the presence and progression of toxicity or other cellular responses to exposure to a particular compound.

Monitoring changes in gene expression may also provide certain advantages during drug screening and development. Often drugs are screened for the ability to interact with a major target without regard to other effects the drugs have on cells. These cellular effects may cause toxicity in the whole animal, which prevents the development and clinical use of the potential drug.

The present inventors have examined tissue from animals exposed to known cardiotoxins which induce detrimental heart effects, to identify global changes in gene expression and individual changes in gene expression induced by these compounds. These global changes in gene expression, which can be detected by the production of expression profiles (an expression level of one or more genes), provide useful toxicity markers that can be used to monitor toxicity and/or toxicity progression by a test compound. Some of these markers may also be used to monitor or detect various disease or physiological states, disease progression, drug efficacy and drug metabolism.

Identification of Toxicity Markers

To evaluate and identify gene expression changes that are predictive of toxicity, studies using selected compounds with well characterized toxicity have been conducted by the present inventors to catalogue altered gene expression during exposure in vivo and in vitro. In the present study, cyclophosphamide, ifosfamide, minoxidil, hydralazine, BI-QT, clenbuterol, isoproterenol, norepinephrine, and epinephrine were selected as known cardiotoxins.

Cyclophosphamide, an alkylating agent, is highly toxic to dividing cells and is commonly used in chemotherapy to treat non-Hodgkin's lymphomas, Burkitt's lymphoma and carcinomas of the lung, breast, and ovary (Goodman & Gilman's The Pharmacological Basis of Therapeutics 9^{th} ed., p. 1234, 1237-1239, J. G. Hardman et al., Eds., McGraw Hill, New York, 1996). Additionally, cyclophosphamide is used as an immunosuppressive agent in bone marrow transplantation and following organ transplantation. Though cyclophosphamide is therapeutically useful, it is also associated with cardiotoxicity, nephrotoxicity, and hemorrhagic cystitis. Once in the liver, cyclophosphamide is hydroxylated by the cytochrome P450 mixed function oxidase system. The active metabolites, phosphoramide mustard and acrolein, cross-link DNA and cause growth arrest and cell death. Acrolein has been shown to decrease cellular glutathione levels (Dorr and Lagel (1994), Chem Biol Interact 93: 117-128).

The cardiotoxic effects of cyclophosphamide have been partially elucidated. One study analyzed plasma levels in 19 women with metastatic breast carcinoma who had been treated with cyclophosphamide, thiotepa, and carboplatin (Ayash et al. (1992), J Clin Oncol 10: 995-1000). Of the 19 women in the study, six developed moderate congestive heart failure. In another case study, a 10-year old boy, who had been treated with high-dose cyclophosphamide, developed cardiac arrhythmias and intractable hypotension (Tsai et al. (1990), Am J Pediatr Hematol Oncol 12: 472-476). The boy died 23 days after the transplantation.

Another clinical study examined the relationship between the amount of cyclophosphamide administered and the development of cardiotoxicity (Goldberg et al. (1986), Blood 68: 1114-1118). When the cyclophosphamide dosage was ≦1.55 g/m²/d, only 1 out of 32 patients had symptoms consistent with cyclophosphamide cardiotoxicity. Yet when the dosage was greater than 1.55 g/m²/d, 13 out of 52 patients were symptomatic. Six of the high-dose patients died of congestive heart failure.

In a related study, Braverman et al. compared the effects of once daily low-dose administration of cyclophosphamide (87+/−11 mg/kg) and twice-daily high-dose treatment (174+/−34 mg/kg) on bone marrow transplantation patients (Braverman et al. (1991), J Clin Oncol 9: 1215-1223). Within a week, the high-dose patients had an increase in left ventricular mass index. Out of five patients who developed clinical cardiotoxicity, four were in the high-dose group.

Ifosfamide, an oxazaphosphorine, is an analog of cyclophosphamide. Whereas cyclophosphamide has two chloroethyl groups on the exocyclic nitrogen, ifosfamide contains one chloroethyl group on the ring nitrogen and the other on the exocyclic nitrogen. Ifosfamide is a nitrogen mustard and alkylating agent, commonly used in chemotherapy to treat testicular, cervical, and lung cancer, as well as sarcomas and lymphomas. Like cyclophosphamide, it is activated in the liver by hydroxylation, but it reacts more slowly and produces more dechlorinated metabolites and chloroacetaldehyde. Comparatively higher doses of ifosfamide are required to match the efficacy of cyclophosphamide.

Alkylating agents can cross-link DNA, resulting in growth arrest and cell death. Despite its therapeutic value, ifosfamide is associated with nephrotoxicity (affecting the proximal and distal renal tubules), urotoxicity, venooclusive disease, myclosuppression, pulmonary fibrosis and central neurotoxicity (Goodman & Gilman's The Pharmacological Basis of Therapeutics 9^{th} ed., p.1234-1240, J. G. Hardman et al., Eds., McGraw Hill, New York, 1996). Ifosfamide can also cause acute severe heart failure and malignant ventricular arrhythmia, which may be reversible. Death from cardiogenic shock has also been reported (Cecil Textbook of Medicine 20^{th} ed., Bennett et al. eds., p. 331, W. B. Saunders Co., Philadelphia, 1996).

Studies of patients with advanced or resistant lymphomas or carcinomas showed that high-dose ifosfamide treatment produced various symptoms of cardiac disease, including dyspnea, tachycardia, decreased left ventricular contractility and malignant ventricular arrhythmia (Quezado et al. (1993), Ann Intern Med 118: 31-36; Wilson et al. (1992), J Clin Oncol 19: 1712-1722). Other patient studies have noted that ifosfamide-induced cardiac toxicity may be asymptomatic, although it can be detected by electrocardiogram and should be monitored (Pai et al.(2000), Drug Saf 22: 263-302).

Minoxidil is an antihypertensive medicinal agent used in the treatment of high blood pressure. It works by relaxing blood vessels so that blood may pass through them more easily, thereby lowering blood pressure. By applying minoxidil to the scalp, it has recently been shown to be effective at combating hair loss by stimulating hair growth. Once minoxidil is metabolized by hepatic sulfotransferase, it is converted to the active molecule minoxidil N—O sulfate (Goodman & Gilman's The Pharmacological Basis of Therapeutics 9^{th} ed., pp. 796-797, J. G. Hardman et al., Eds., McGraw Hill, New York, 1996). The active minoxidil sulfate stimulates the ATP-modulated potassium channel consequently causing hyperpolarization and relaxation of smooth muscle. Early studies on minoxidil demonstrated that following a single dose of the drug, patients suffering from left ventricular failure exhibited a slightly increased heart rate, a fall in the mean arterial pressure, a fall in the systemic vascular resistance, and a slight increase in cardiac index (Franciosa and Cohn (1981) Circulation 63: 652-657).

Some common side effects associated with minoxidil treatment are an increase in hair growth, weight gain, and a fast or irregular heartbeat. More serious side effects are numbness of the hands, feet, or face, chest pain, shortness of breath, and swelling of the feet or lower legs. Because of the risks of fluid retention and reflex cardiovascular effects, minoxidil is often given concomitantly with a diuretic and a sympatholytic drug.

While minoxidil is effective at lowering blood pressure, it does not lead to a regression of cardiac hypertrophy. To the contrary, minoxidil has been shown to cause cardiac enlargement when administered to normotensive animals (Moravec et al. (1994) J Pharmacol Exp Ther 269: 290-296). Moravec et al. examined normotensive rats that had developed myocardial hypertrophy following treatment with minoxidil. The authors found that minoxidil treatment led to enlargement of the left ventricle, right ventricle, and interventricular septum.

Another rat study investigated the age- and dose-dependency of minoxidil- induced cardiotoxicity (Herman et al. (1996) Toxicology 110: 71-83). Rats ranging in age from 3 months to 2 years were given varying amounts of minoxidil over the period of two days. The investigators observed interstitial hemorrhages at all dose levels, however the hemorrhages were more frequent and severe in the older animals. The 2 year old rats had vascular lesions composed of arteriolar damage and calcification.

Hydralazine, an antihypertensive drug, causes relaxation of arteriolar smooth muscle. Such vasodilation is linked to vigorous stimulation of the sympathetic nervous system, which in turn leads to increased heart rate and contractility, increased plasma renin activity, and fluid retention (Goodman & Gilman's The Pharmacological Basis of Therapeutics 9^{th} ed., p. 794, J. G. Hardman et al., Eds., McGraw Hill, New York, 1996). The increased renin activity leads to an increase in angiotensin II, which in turn causes stimulation of aldosterone and sodium reabsorption.

Hydralazine is used for the treatment of high blood pressure (hypertension) and for the treatment of pregnant women suffering from high blood pressure (pre-eclampsia or eclampsia). Some common side effects associated with hydralazine use are diarrhea, rapid heartbeat, headache, decreased appetite, and nausea. Hydralazine is often used concomitantly with drugs that inhibit sympathetic activity to combat the mild pulmonary hypertension that can be associated with hydralazine usage.

In one hydralazine study, rats were given one of five cardiotoxic compounds (isoproterenol, hydralazine, caffeine, cyclophosphamide, or adriamycin) by intravenous injection (Kemi et al. (1996), J Vet Med Sci 58: 699-702). At one hour and four hours post-dose, early focal myocardial lesions were observed histopathologically. Lesions were observed in the rats treated with hydralazine four hours post-dose. The lesions were found in the inner one third of the left ventricular walls including the papillary muscles.

Another study compared the effects of isoproterenol, hydralazine and minoxidil on young and mature rats (Hanton et al. (1991), Res Commun Chem Pathol Pharmacol 71: 231-234). Myocardial necrosis was observed in both age groups, but it was more severe in the mature rats. Hypotension and reflex tachycardia were also seen in the hydralazine-treated rats.

BI-QT, has been shown to induce QC prolongation in dogs and liver alterations in rats. Over a four week period, dogs treated with BI-QT exhibited sedation, decreased body weight, increased liver weight, and slightly increased levels of AST, ALP, and BUN. After three months of treatment, the dogs exhibited signs of cardiovascular effects.

Clenbuterol, a β2 adrenergic agonist, can be used therapeutically as a bronchial dilator for asthmatics. It also has powerful muscle anabolic and lipolytic effects. It has been banned in the United States but continues to be used illegally by athletes to increase muscle growth. In a number of studies, rats treated with clenbuterol developed hypertrophy of the heart and latissimus dorsi muscle (Doheny et al. (1998), Amino Acids 15: 13-25; Murphy et al. (1999), Proc Soc Exp Biol Med 221: 184-187; Petrou et al. (1995), Circulation 92: II483-II489).

In one study, mares treated with therapeutic levels of clenbuterol were compared to mares that were exercised and mares in a control group (Sleeper et al. (2002), Med Sci Sports Exerc 34: 643-650). The clenbuterol-treated mares demonstrated significantly higher left ventricular internal dimension and interventricular septal wall thickness at end diastole. In addition, the clenbuterol-treated mares had significantly increased aortic root dimensions, which could lead to an increased chance of aortic rupture.

In another study, investigators reported a case of acute clenbuterol toxicity in a human (Hoffman et al. (2001), J Toxicol 39: 339-344). A 28-year old woman had ingested a small quantity of clenbuterol, and the patient developed sustained sinus tachycardia, hypokalemia, hypophosphatemia, and hypomagnesemia.

Catecholamines are neurotransmitters that are synthesized in the adrenal medulla and in the sympathetic nervous system. Epinephrine, norepinephrine, and isoproterenol are members of the catecholamine sympathomimetic amine family (Casarett & Doull's Toxicology, The Basic Science of Poisons 6^{th} ed., p. 618-619, C. D. Klaassen, Ed., McGraw Hill, New York, 2001). They are chemically similar by having an aromatic portion (catechol) to which is attached an amine, or nitrogen-containing group.

Isoproterenol, an antiarrhythmic agent, is used therapeutically as a bronchodilator for the treatment of asthma, chronic bronchitis, emphysema, and other lung diseases. Some side effects of usage are myocardial ischemia, arrhythmias, angina, hypertension, and tachycardia. As a β receptor agonist, isoproterenol exerts direct positive inotropic and chronotropic effects. Peripheral vascular resistance is decreased along with the pulse pressure and mean arterial pressure. However, the heart rate increases due to the decrease in the mean arterial pressure.

Norepinephrine, an α and β receptor agonist, is also known as noradrenaline. It is involved in behaviors such as attention and general arousal, stress, and mood states. By acting on β-1 receptors, it causes increased peripheral vascular resistance, pulse pressure and mean arterial pressure. Reflex bradycardia occurs due to the increase in mean arterial pressure. Some contraindications associated with norepinephrine usage are myocardial ischemia, premature ventricular contractions (PVCs), and ventricular tachycardia.

Epinephrine, a potent α and β adrenergic agonist, is used for treating bronchoconstriction and hypotension resulting from anaphylaxis as well as all forms of cardiac arrest. Injection of epinephrine leads to an increase in systolic pressure, ventricular contractility, and heart rate. Some side effects associated with epinephrine usage are cardiac arrhythmias, particularly PVCs, ventricular tachycardia, renal vascular ischemia, increased myocardial oxygen requirements, and hypokalemia.

Toxicity Prediction and Modeling

The genes and gene expression information, gene expression profiles, as well as the portfolios and subsets of the genes provided in Tables 1-5I, may be used to predict at least one toxic effect, including the cardiotoxicity of a test or unknown compound. As used, herein, at least one toxic effect includes, but is not limited to, a detrimental change in the physiological status of a cell or organism. The response may be, but is not required to be, associated with a particular pathology, such as tissue necrosis, myocarditis, arrhythmias, tachycardia, myocardial ischemia, angina, hypertension, hypotension, dyspnea, and cardiogenic shock. Accordingly, the toxic effect includes effects at the molecular and cellular level. Cardiotoxicity is an effect as used herein and includes but is not limited to the pathologies of tissue necrosis, myocarditis, arrhythmias, tachycardia, myocardial ischemia, angina, hypertension, hypotension, dyspnea, and cardiogenic shock. As used herein, a gene expression profile comprises any representation, quantitative or not, of the expression of at least one mRNA species in a cell sample or population and includes profiles made by various methods such as differential display, PCR, hybridization analysis, etc.

In general, assays to predict the toxicity or cardiotoxicity of a test agent (or compound or multi-component composition) comprise the steps of exposing a cell population to the test compound, assaying or measuring the level of relative or absolute gene expression of one or more of the genes in Tables 1-5I and comparing the identified expression level(s) to the expression levels disclosed in the Tables and database(s) disclosed herein. Assays may include the measurement of the expression levels of about 2, 3, 4, 5, 6, 7, 8, 9, 10, 15, 20, 25, 30, 50, 75, 100 or more genes from Tables 1-5I.

In the methods of the invention, the gene expression level for a gene or genes induced by the test agent, compound or compositions may be comparable to the levels found in the Tables or databases disclosed herein if the expression level varies within a factor of about 2, about 1.5 or about 1.0 fold. In some cases, the expression levels are comparable if the agent induces a change in the expression of a gene in the same direction (e.g., up or down) as a reference toxin.

The cell population that is exposed to the test agent, compound or composition may be exposed in vitro or in vivo. For instance, cultured or freshly isolated heart cells, in particular rat heart cells, may be exposed to the agent under standard laboratory and cell culture conditions. In another assay format, in vivo exposure may be accomplished by administration of the agent to a living animal, for instance a laboratory rat.

Procedures for designing and conducting toxicity tests in in vitro and in vivo systems are well known, and are described in many texts on the subject, such as Loomis et al., Loomis's Essentials of Toxicology, 4th Ed., Academic Press, New York, 1996; Echobichon, The Basics of Toxicity Testing, CRC Press, Boca Raton, 1992; Frazier, editor, In Vitro Toxicity Testing, Marcel Dekker, New York, 1992; and the like.

In in vitro toxicity testing, two groups of test organisms are usually employed: One group serves as a control and the other group receives the test compound in a single dose (for acute toxicity tests) or a regimen of doses (for prolonged or chronic toxicity tests). Because, in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. When rats or mice are used, the volume administered by the oral route generally should not exceed about 0.005 ml per gram of animal. Even when aqueous or physiological saline solutions are used for parenteral injection the volumes that are tolerated are limited, although such solutions are ordinarily thought of as being innocuous. The intravenous LD₅₀ of distilled water in the mouse is approximately 0.044 ml per gram and that of isotonic saline is 0.068 ml per gram of mouse. In some instances, the route of administration to the test animal should be the same as, or as similar as possible to, the route of administration of the compound to man for therapeutic purposes.

When a compound is to be administered by inhalation, special techniques for generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparatuses and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some preferred embodiments of the methods of the invention, the cells to be exposed to the agent are derived from heart tissue. For instance, cultured or freshly isolated rat heart cells may be used.

The methods of the invention may be used generally to predict at least one toxic response, and, as described in the Examples, may be used to predict the likelihood that a compound or test agent will induce various specific heart pathologies, such as tissue necrosis, myocarditis, arrhythmias, tachycardia, myocardial ischemia, angina, hypertension, hypotension, dyspnea, cardiogenic shock, or other pathologies associated with at least one of the toxins herein described. The methods of the invention may also be used to determine the similarity of a toxic response to one or more individual compounds. In addition, the methods of the invention may be used to predict or elucidate the potential cellular pathways influenced, induced or modulated by the compound or test agent due to the similarity of the expression profile compared to the profile induced by a known toxin (see Tables 5-5I).

Diagnostic Uses for the Toxicity Markers

As described above, the genes and gene expression information or portfolios of the genes with their expression information as provided in Tables 1-5I may be used as diagnostic markers for the prediction or identification of the physiological state of a tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or genes from Tables 5-5I may be compared to the expression levels found in tissues or cells exposed to the toxins described herein. These methods may result in the diagnosis of a physiological state in the cell, may be used to diagnose toxin exposure or may be used to identify the potential toxicity of a compound, for instance a new or unknown compound or agent that the subject has been exposed to. The comparison of expression data, as well as available sequence or other information may be done by researcher or diagnostician or may be done with the aid of a computer and databases as described below.

In another format, the levels of a gene(s) of Tables 5-5I, its encoded protein(s), or any metabolite produced by the encoded protein may be monitored or detected in a sample, such as a bodily tissue or fluid sample to identify or diagnose a physiological state of an organism. Such samples may include any tissue or fluid sample, including urine, blood and easily obtainable cells such as peripheral lymphocytes.

Use of the Markers for Monitoring Toxicity Progression

As described above, the genes and gene expression information provided in Tables 5-5I may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or genes from Tables 5-5I may be compared to the expression levels found in tissue or cells exposed to the cardiotoxins described herein. The comparison of the expression data, as well as available sequence or other information may be done by a researcher or diagnostician or may be done with the aid of a computer and databases.

Use of the Toxicity Markers for Drug Screening

According to the present invention, the genes identified in Tables 1-5I may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to stimulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Tables 1-5I may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, two or more genes from Tables 1-5I may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes of Tables 1-5I are compared to the expression levels of those same genes exposed to a known toxin alone. Compounds that modulate the expression patterns of the known toxin(s) would be expected to modulate potential toxic physiological effects in vivo. The genes in Tables 1-5I are particularly appropriate markers in these assays as they are differentially expressed in cells upon exposure to a known cardiotoxin. Tables 1 and 2 disclose those genes that are differentially expressed upon exposure to the named toxins and their corresponding GenBank Accession numbers. Table 3 discloses the human homologues and the corresponding GenBank Accession numbers of the differentially expressed genes of Tables 1 and 2.

In another format, cell lines that contain reporter gene fusions between the open reading frame and/or the transcriptional regulatory regions of a gene in Tables 1-5I and any assayable fusion partner may be prepared. Numerous assayable fusion partners are known and readily available including the firefly luciferase gene and the gene encoding chloramphenicol acetyltransferase (Alam et al. (1990), Anal Biochem 188: 245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene identified in Tables 5-5I. For instance, as described above, mRNA expression may be monitored directly by hybridization of probes to the nucleic acids of the invention. Cell lines are exposed to the agent to be tested under appropriate conditions and time, and total RNA or mRNA is isolated by standard procedures such those disclosed in Sambrook et al. (Molecular Cloning: A Laboratory Manual, 2nd Ed., Cold Spring Harbor Laboratory Press, Cold Spring Harbor, N.Y., 1989).

In another assay format, cells or cell lines are first identified which express the gene products of the invention physiologically. Cells and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines may be transduced or transfected with an expression vehicle (e.g., a plasmid or viral vector) construct comprising an operable non-translated 5′-promoter containing end of the structural gene encoding the gene products of Tables 1-5I fused to one or more antigenic fragments or other detectable markers, which are peculiar to the instant gene products, wherein said fragments are under the transcriptional control of said promoter and are expressed as polypeptides whose molecular weight can be distinguished from the naturally occurring polypeptides or may further comprise an immunologically distinct or other detectable tag. Such a process is well known in the art (see Sambrook et al., supra).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells are disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the agent-contacted sample is then compared with the control samples (no exposure and exposure to a known toxin) where only the excipient is contacted with the cells and an increase or decrease in the immunologically generated signal from the agent-contacted sample compared to the control is used to distinguish the effectiveness and/or toxic effects of the agent.

Use of Toxicity Markers to Identify Agents that Modulate Protein Activity or Levels

Another embodiment of the present invention provides methods for identifying agents that modulate at least one activity of a protein(s) encoded by the genes in Tables 1-5I. Such methods or assays may utilize any means of monitoring or detecting the desired activity.

In one format, the relative amounts of a protein (Tables 1-5I) between a cell population that has been exposed to the agent to be tested compared to an unexposed control cell population and a cell population exposed to a known toxin may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see G. A. Grant in: Molecular Biology and Biotechnology, Meyers, ed., pp. 659-664, VCH Publishers, New York, 1995). A skilled artisan can readily recognize that there is no limit as to the structural nature of the agents of the present invention.

Nucleic Acid Assay Formats

As previously discussed, the genes identified as being differentially expressed upon exposure to a known cardiotoxin (Tables 1-5I) may be used in a variety of nucleic acid detection assays to detect or quantify the expression level of a gene or multiple genes in a given sample. The genes described in Tables 1-5I may also be used in combination with one or more additional genes whose differential expression is associate with toxicity in a cell or tissue. In preferred embodiments, the genes in Tables 5-5I may be combined with one or more of the genes described in prior and related applications 60/303,819; 60/305,623; 60/369,351; 60/377,611; 09/917,800; 10/060,087; and 10/152,319, all of which are incorporated by reference on page 1 of this application.

Any assay format to detect gene expression may be used. For example, traditional Northern blotting, dot or slot blot, nuclease protection, primer directed amplification, RT- PCR, semi- or quantitative PCR, branched-chain DNA and differential display methods may be used for detecting gene expression levels. Those methods are useful for some embodiments of the invention. In cases where smaller numbers of genes are detected, amplification based assays may be most efficient. Methods and assays of the invention, however, may be most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000, 400,000 or 1,000,000 or more of such features on a single solid support. The solid support, or the area within which the probes are attached may be on the order of about a square centimeter. Probes corresponding to the genes of Tables 5-5I or from the related applications described above may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al. (1996), Nat Biotechnol 14: 1675-1680; McGall et al. (1996), Proc Nat Acad Sci USA 93: 13555-13460). Such probe arrays may contain at least two or more oligonucleotides that are complementary to or hybridize to two or more of the genes described in Tables 5-5I. For instance, such arrays may contain oligonucleotides that are complementary to or hybridize to at least 2, 3, 4, 5, 6, 7, 8, 9, 10, 20, 30, 50, 70, 100 or more of the genes described herein. Preferred arrays contain all or nearly all of the genes listed in Tables 1-5I, or individually, the gene sets of Tables 5-5I. In a preferred embodiment, arrays are constructed that contain oligonucleotides to detect all or nearly all of the genes in any one of or all of Tables 1-5I on a single solid support substrate, such as a chip.

The sequences of the expression marker genes of Tables 1-5I are in the public databases. Table 1 provides the GenBank Accession Number or NCBI RefSeq ID for each of the sequences (see www.ncbi.nlm.nih.gov/) as well as a corresponding SEQ ID NO. in the sequence listing filed with this application. Table 3 provides the LocusLink and Unigene names and descriptions for the human homologues of the genes described in Tables 1 and 2. The sequences of the genes in GenBank and/or RefSeq are expressly herein incorporated by reference in their entirety as of the filing date of this application, as are related sequences, for instance, sequences from the same gene of different lengths, variant sequences, polymorphic sequences, genomic sequences of the genes and related sequences from different species, including the human counterparts, where appropriate. These sequences may be used in the methods of the invention or may be used to produce the probes and arrays of the invention. In some embodiments, the genes in Tables 1-5I that correspond to the genes or fragments previously associated with a toxic response may be excluded from the Tables.

As described above, in addition to the sequences of the GenBank Accession Numbers or NCBI RefSeq ID's disclosed in the Tables 1-5I, sequences such as naturally occurring variants or polymorphic sequences may be used in the methods and compositions of the invention. For instance, expression levels of various allelic or homologous forms of a gene disclosed in Tables 1-5I may be assayed. Any and all nucleotide variations that do not significantly alter the functional activity of a gene listed in the Tables 1-5I, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes described in Tables 1-5I refer to oligonucleotides that are capable of hybridizing under stringent conditions to at least part of the nucleotide sequences of said genes, their encoded RNA or mRNA, or amplified versions of the RNA such as cRNA. Such hybridizable oligonucleotides will typically exhibit at least about 75% sequence identity at the nucleotide level to said genes, preferably about 80% or 85% sequence identity or more preferably about 90% or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 different nucleic acid hybridizations.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(es) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical submit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Probe Design

One of skill in the art will appreciate that an enormous number of array designs are suitable for the practice of this invention. The high density array will typically include a number of test probes that specifically hybridize to the sequences of interest. Probes may be produced from any region of the genes identified in the Tables and the attached representative sequence listing. In instances where the gene reference in the Tables is an EST, probes may be designed from that sequence or from other regions of the corresponding full-length transcript that may be available in any of the sequence databases, such as those herein described. See WO 99/32660 for methods of producing probes for a given gene or genes. In addition, any available software may be used to produce specific probe sequences, including, for instance, software available from Molecular Biology Insights, Olympus Optical Co. and Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are 20 or 25 nucleotides in length. In another preferred embodiment, test probes are double or single strand DNA sequences such as cDNA fragments. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene in the accompanying Tables 1-5I. The difference in intensity between the perfect match and the mismatch probe provides a good measure of the concentration of the hybridized material.

Nucleic Acid Samples

Cell or tissue samples may be exposed to the test agent in vitro or in vivo. When cultured cells or tissues are used, appropriate mammalian cell extracts, such as liver cell extracts, may also be added with the test agent to evaluate agents that may require biotransformation to exhibit toxicity.

The genes which are assayed according to the present invention are typically in the form of mRNA or reverse transcribed mRNA. The genes may or may not be cloned. The genes may or may not be amplified and cRNA produced. The cloning and/or amplification do not appear to bias the representation of genes within a population. In some assays, it may be preferable, however, to use polyA+ RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology, Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or tissue of interest. Such samples also include DNA amplified from the cDNA, and RNA transcribed from the amplified DNA (cRNA). One of skill in the art would appreciate that it is desirable to inhibit or destroy RNase present in homogenates before homogenates are used.

Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

Forming High Density Arrays

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6×SSPET at 37° C. (0.005% Triton X-100), to ensure hybridization and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art. See WO 99/32660.

Databases

The present invention includes relational databases containing sequence information, for instance, for the genes of Tables 1-5I, as well as gene expression information from tissue or cells exposed to various standard toxins, such as those herein described (see Tables 5-5I). Databases may also contain information associated with a given sequence or tissue sample such as descriptive information about the gene associated with the sequence information (see Tables 1 and 2), or descriptive information concerning the clinical status of the tissue sample, or the animal from which the sample was derived. The database may be designed to include different parts, for instance a sequence database and a gene expression database. Methods for the configuration and construction of such databases and computer-readable media to which such databases are saved are widely available, for instance, see U.S. Pat. No. 5,953,727, which is herein incorporated by reference in its entirety.

The databases of the invention may be linked to an outside or external database such as GenBank (www.ncbi.nlm.nih.gov/entrez.index.html); KEGG (www.genome.ad.jp/kegg); SPAD (www.grt.kyushu-u.acjp/spad/index.html); HUGO (www.gene.ucl.ac.uk/hugo); Swiss-Prot (www.expasy.ch.sprot); Prosite (www.expasy.ch/tools/scnpsitl.html); OMIM (www.ncbi.nlm.nih.gov/omim); and GDB (www.gdb.org). In a preferred embodiment, as described in Tables 1-5I, the external database is GenBank and the associated databases maintained by the National Center for Biotechnology Information (NCBI) (www.ncbi.nlm.nih.gov).

Any appropriate computer platform, user interface, etc. may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, electronic Northerns that allow the user to determine the cell type or tissue in which a given gene is expressed and to allow determination of the abundance or expression level of a given gene in a particular tissue or cell.

The databases of the invention may also be used to present information identifying the expression level in a tissue or cell of a set of genes comprising one or more of the genes in Tables 5-5I, comprising the step of comparing the expression level of at least one gene in Tables 5-5I in a cell or tissue exposed to a test agent to the level of expression of the gene in the database. Such methods may be used to predict the toxic potential of a given compound by comparing the level of expression of a gene or genes in Tables 5-5I from a tissue or cell sample exposed to the test agent to the expression levels found in a control tissue or cell samples exposed to a standard toxin or cardiotoxin such as those herein described. Such methods may also be used in the drug or agent screening assays as described herein.

Kits

The invention further includes kits combining, in different combinations, high- density oligonucleotide arrays, reagents for use with the arrays, protein reagents encoded by the genes of the Tables, signal detection and array-processing instruments, gene expression databases and analysis and database management software described above. The kits may be used, for example, to predict or model the toxic response of a test compound, to monitor the progression of heart disease states, to identify genes that show promise as new drug targets and to screen known and newly designed drugs as discussed above.

The databases packaged with the kits are a compilation of expression patterns from human or laboratory animal genes and gene fragments (corresponding to the genes of Tables 1-5I). In particular, the database software and packaged information that may contain the databases saved to a computer-readable medium include the expression results of Tables 1-5I that can be used to predict toxicity of a test agent by comparing the expression levels of the genes of Tables 1-5I induced by the test agent to the expression levels presented in Tables 5-5I. In another format, database and software information may be provided in a remote electronic format, such as a website, the address of which may be packaged in the kit.

Databases and software designed for use with microarrays is discussed in PCT/US99/20449, filed Sep. 8, 1999, Genomic Knowledge Discovery, PCT/IB00/00863, filed Jun. 28, 2000, Biological Data Processing, and in Balaban et al., U.S. Pat. No. 6,229,911, a computer-implemented method for managing information, stored as indexed tables, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Identification of Toxicity Markers

The cardiotoxins cyclophosphamide, ifosfamide, minoxidil, hydralazine, BI-QT, clenbuterol, isoproterenol, norepinephrine, and epinephrine and control compositions were administered to male Sprague-Dawley rats at various timepoints using administration diluents, protocols and dosing regimes as previously described in the art and previously described in the priority applications discussed above. The low and high dose level for each compound are provided in the chart below.

| Heart Toxin | Low Dose (mg/kg) | High Dose (mg/kg) |
|---|---|---|
| cyclophosphamide | 20 | 200 |
| ifosfamide | 5 | 100 |
| minoxidil | 12 mg/L | 120 mg/L |
| hydralazine | 2.5 | 25 |
| BI-QT | 10 | 50 |
| clenbuterol | 0.4 | 4 |
| isoproterenol | 0.05 | 0.5 |
| norepinephrine | 0.05 | 0.5 |
| epinephrine | 0.1 | 1 |

After administration, the dosed animals were observed and tissues were collected as described below:

Observation of Animals

1. Clinical Observations—Twice daily: mortality and moribundity check. Cage Side Observations—skin and fur, eyes and mucous membrane, respiratory system, circulatory system, autonomic and central nervous system, somatomotor pattern, and behavior pattern.

Potential signs of toxicity, including tremors, convulsions, salivation, diarrhea, lethargy, coma or other a typical behavior or appearance, were recorded as they occurred and included a time of onset, degree, and duration.

2. Physical Examinations—Prior to randomization, prior to initial treatment, and prior to sacrifice.

3. Body Weights—Prior to randomization, prior to initial treatment, and prior to sacrifice.

Clinical Pathology

1. Frequency Prior to necropsy.

2. Number of animals All surviving animals.

3. Bleeding Procedure Blood was obtained by puncture of the orbital sinus while under 70% CO₂/30% O₂ anesthesia.

4. Collection of Blood Samples Approximately 0.5 mL of blood was collected into EDTA tubes for evaluation of hematology parameters. Approximately 1 mL of blood was collected into serum separator tubes for clinical chemistry analysis. Approximately 200 uL of plasma was obtained and frozen at ˜−80° C. for test compound/metabolite estimation. An additional ˜2 mL of blood was collected into a 15 mL conical polypropylene vial to which ˜3 mL of Trizol was immediately added. The contents were immediately mixed with a vortex and by repeated inversion. The tubes were frozen in liquid nitrogen and stored at ˜−80° C.

Termination Procedures

Terminal Sacrifice

Approximately 3, 6, 24, 48, 144, 168, 192, 336, and/or 360 hours after the initial dose, rats were weighed, physically examined, sacrificed by decapitation, and exsanguinated. The animals were necropsied within approximately five minutes of sacrifice. Separate sterile, disposable instruments were used for each animal, with the exception of bone cutters, which were used to open the skull cap. The bone cutters were dipped in disinfectant solution between animals.

Necropsies were conducted on each animal following procedures approved by board-certified pathologists.

Animals not surviving until terminal sacrifice were discarded without necropsy (following euthanasia by carbon dioxide asphyxiation, if moribund). The approximate time of death for moribund or found dead animals was recorded.

Postmortem Procedures

Fresh and sterile disposable instruments were used to collect tissues. Gloves were worn at all times when handling tissues or vials. All tissues were collected and frozen within approximately 5 minutes of the animal's death. The liver sections and kidneys were frozen within approximately 3-5 minutes of the animal's death. The time of euthanasia, an interim time point at freezing of liver sections and kidneys, and time at completion of necropsy were recorded. Tissues were stored at approximately −80° C. or preserved in 10% neutral buffered formalin.

Tissue Collection and Processing

Liver

1. Right medial lobe—snap frozen in liquid nitrogen and stored at ˜−80° C.

2. Left medial lobe—Preserved in 10% neutral-buffered formalin (NBF) and evaluated for gross and microscopic pathology.

3. Left lateral lobe—snap frozen in liquid nitrogen and stored at ˜−80° C.

Heart

A sagittal cross-section containing portions of the two atria and of the two ventricles was preserved in 10% NBF. The remaining heart was frozen in liquid nitrogen and stored at ˜−80° C.

Kidneys (Both)

1. Left—Hemi-dissected; half was preserved in 10% NBF and the remaining half was frozen in liquid nitrogen and stored at ˜−80° C.

2. Right—Hemi-dissected; half was preserved in 10% NBF and the remaining half was frozen in liquid nitrogen and stored at ˜−80° C.

Testes (Both)

A sagittal cross-section of each testis was preserved in 10% NBF. The remaining testes were frozen together in liquid nitrogen and stored at ˜−80° C.

Brain (Whole)

A cross-section of the cerebral hemispheres and of the diencephalon was preserved in 10% NBF, and the rest of the brain was frozen in liquid nitrogen and stored at ˜−80° C.

Microarray sample preparation was conducted with minor modifications, following the protocols set forth in the Affymetrix GeneChip Expression Analysis Manual. Frozen tissue was ground to a powder using a Spex Certiprep 6800 Freezer Mill. Total RNA was extracted with Trizol (GibcoBRL) utilizing the manufacturer's protocol. The total RNA yield for each sample was 200-500 μg per 300 mg tissue weight. mRNA was isolated using the Oligotex mRNA Midi kit (Qiagen) followed by ethanol precipitation. Double stranded cDNA was generated from mRNA using the SuperScript Choice system (GibcoBRL). First strand cDNA synthesis was primed with a T7-(dT24) oligonucleotide. The cDNA was phenol-chloroform extracted and ethanol precipitated to a final concentration of 1μg/ml. From 2 μg of cDNA, cRNA was synthesized using Ambion's T7 MegaScript in vitro Transcription Kit.

To biotin label the cRNA, nucleotides Bio-11-CTP and Bio-16-UTP (Enzo Diagnostics) were added to the reaction. Following a 37° C. incubation for six hours, impurities were removed from the labeled cRNA following the RNeasy Mini kit protocol (Qiagen). cRNA was fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA was hybridized on the Affymetrix rat array set for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Data was analyzed using Affymetrix GeneChip® version 2.0 and Expression Data Mining (EDMT) software (version 1.0), Gene Logic's GeneExpress® 2000 software and S-Plus™ software.

Tables 1 and 2 disclose those genes that are differentially expressed upon exposure to the named toxins and their corresponding GenBank Accession and Sequence Identification numbers, the identities of the metabolic pathways in which the genes function, the gene names if known, and the unigene cluster titles. The model code represents the various toxicity state that each gene is able to discriminate as well as the individual toxin type associated with each gene. The codes are defined in Table 4. The GLGC ID is the internal Gene Logic identification number.

Table 3 discloses those genes that are the human homologues of those genes in Tables 1 and 2 that are differentially expressed upon exposure to the named toxins. The corresponding GenBank Accession and Sequence Identification numbers, the gene names if known, and the unigene cluster titles of the human homologues are listed.

Table 4 defines the comparison codes used in Tables 1, 2, 3, and 5.

Tables 5-5I disclose the summary statistics for each of the comparisons performed. Each of these tables contains a set of predictive genes and creates a model for predicting the cardiotoxicity of an unknown, i.e., untested compound. Each gene is identified by its Gene Logic identification number and can be cross-referenced to a gene name and representative SEQ ID NO. in Tables 1 and 2. For each comparison of gene expression levels between samples in the toxicity group (samples affected by exposure to a specific toxin) and samples in the non-toxicity group (samples not affected by exposure to that same specific toxin), the tox mean (for toxicity group samples) is the mean signal intensity, as normalized for the various chip parameters that are being assayed. The non-tox mean represents the mean signal intensity, as normalized for the various chip parameters that are being assayed, in samples from animals other than those treated with the high dose of the specific toxin. These animals were treated with a low dose of the specific toxin, or with vehicle alone, or with a different toxin. Samples in the toxicity groups were obtained from animals sacrificed at the timepoint(s) indicated in the Table 5-5I headings, while samples in the non-toxicity groups were obtained from animals sacrificed at all time points in the experiments. For individual genes, an increase in the tox mean compared to the non-tox mean indicates up-regulation upon exposure to a toxin. Conversely, a decrease in the tox mean compared to the non-tox mean indicates down-regulation.

The mean values are derived from Average Difference (AveDiff) values for a particular gene, averaged across the corresponding samples. Each individual Average Difference value is calculated by integrating the intensity information from multiple probe pairs that are tiled for a particular fragment. The normalization multiplies each expression intensity for a given experiment (chip) by a global scaling factor. The intent of this normalization is to make comparisons of individual genes between chips possible. The scaling factor is calculated as follows:

1. From all the unnormalized expression values in the experiment, delete the largest 2% and smallest 2% of the values. That is, if the experiment yields 10,000 expression values, order the values and delete the smallest 200 and largest 200.

2. Compute the trimmed mean, which is equal to the mean of the remaining values.

3. Compute the scale factor SF=100/(trimmed mean)

The value of 100 used here is the standard target valued used. Some AveDiff values may be negative due to the general noise involved in nucleic acid hybridization experiments. Although many conclusions can be made corresponding to a negative value on the GeneChip platform, it is difficult to assess the meaning behind the negative value for individual fragments. Our observations show that, although negative values are observed at times within the predictive gene set, these values reflect a real biological phenomenon that is highly reproducible across all the samples from which the measurement was taken. For this reason, those genes that exhibit a negative value are included in the predictive set. It should be noted that other platforms of gene expression measurement may be able to resolve the negative numbers for the corresponding genes. The predictive ability of each of those genes should extend across platforms, however. Each mean value is accompanied by the standard deviation for the mean. The linear discriminant analysis score (discriminant score), as disclosed in the tables, measures the ability of each gene to predict whether or not a sample is toxic. The discriminant score is calculated by the following steps:

Calculation of a discriminant score

Let Xᵢ represent the AveDiff values for a given gene across the non-tox samples, i=1 . . . n.

Let Yᵢ represent the AveDiff values for a given gene across the tox samples, i=1 . . . t.

The calculations proceed as follows:

1. Calculate mean and standard deviation for Xᵢ's and Yᵢ's, and denote these by m_{X}, m_{Y}, s_{X},s_{Y}.

2. For all Xᵢ's and Yᵢ's, evaluate the function f(z)=((1/s_{Y})*exp(−0.5*((z−m_{Y})/s_{Y})²))/(((1/s_{Y})*exp(−0.5*((z−m_{Y})/s_{Y})²))+((1/s_{X})*exp(−0.5*((z−m_{X})/s_{X})²))).

3. The number of correct predictions, say P, is then the number of Yᵢ's such that f(Yᵢ)>0.5 plus the number of Xᵢ's such that f(Xᵢ )<0.5.

4. The discriminant score is then P/(n+t).

Linear discriminant analysis uses both the individual measurements of each gene and the calculated measurements of all combinations of genes to classify samples. For each gene a weight is derived from the mean and standard deviation of the toxic and nontox groups. Every gene is multiplied by a weight and the sum of these values results in a collective discriminate score. This discriminant score is then compared against collective centroids of the tox and nontox groups. These centroids are the average of all tox and nontox samples respectively. Therefore, each gene contributes to the overall prediction. This contribution is dependent on weights that are large positive or negative numbers if the relative distances between the tox and nontox samples for that gene are large and small numbers if the relative distances are small. The discriminant score for each unknown sample and centroid values can be used to calculate a probability between zero and one as to the group in which the unknown sample belongs.

### Example 2

### General Toxicity Modeling

Samples were selected for grouping into tox-responding and non-tox-responding groups by examining each study individually with Principal Components Analysis (PCA) to determine which treatments had an observable response. Only groups where confidence of their tox-responding and non-tox-responding status was established were included in building a general tox model (Tables 5-5I).

Linear discriminant models were generated to describe toxic and non-toxic samples. The top discriminant genes and/or EST's were used to determine toxicity by calculating each gene's contribution with homo and heteroscedastic treatment of variance and inclusion or exclusion of mutual information between genes. Prediction of samples within the database exceeded 80% true positives with a false positive rate of less than 5%. It was determined that combinations of genes and/or EST's generally provided a better predictive ability than individual genes and that the more genes and/or EST used the better predictive ability. Although the preferred embodiment includes fifty or more genes, many pairings or greater combinations of genes and/or EST can work better than individual genes. All combinations of two or more genes from the selected list (Tables 5-5I) could be used to predict toxicity. These combinations could be selected by pairing in an agglomerate, divisive, or random approach. Further, as yet undetermined genes and/or EST's could be combined with individual or combination of genes and/or EST's described here to increase predictive ability. However, the genes and/or EST's described here would contribute most of the predictive ability of any such undetermined combinations.

Other variations on the above method can provide adequate predictive ability. These include selective inclusion of components via agglomerate, divisive, or random approaches or extraction of loading and combining them in agglomerate, divisive, or random approaches. Also the use of composite variables in logistic regression to determine classification of samples can also be accomplished with linear discriminate analysis, neural or Bayesian networks, or other forms of regression and classification based on categorical or continual dependent and independent variables.

### Example 3

### Modeling Methods

The above modeling methods provide broad approaches of combining the expression of genes to predict sample toxicity. One could also provide no weight in a simple voting method or determine weights in a supervised or unsupervised method using agglomerate, divisive, or random approaches. All or selected combinations of genes may be combined in ordered, agglomerate, or divisive, supervised or unsupervised clustering algorithms with unknown samples for classification. Any form of correlation matrix may also be used to classify unknown samples. The spread of the group distribution and discriminate score alone provide enough information to enable a skilled person to generate all of the above types of models with accuracy that can exceed discriminate ability of individual genes. Some examples of methods that could be used individually or in combination after transformation of data types include but are not limited to: Discriminant Analysis, Multiple Discriminant Analysis, logistic regression, multiple regression analysis, linear regression analysis, conjoint analysis, canonical correlation, hierarchical cluster analysis, k-means cluster analysis, self-organizing maps, multidimensional scaling, structural equation modeling, support vector machine determined boundaries, factor analysis, neural networks, bayesian classifications, and resampling methods.

### Example 4

### Individual Compound Markers

The mechanism of action of a particular compound's induced toxicity, as exhibited by gene expression, may differ from all other compounds' mechanisms of induced toxicity. Therefore, markers of toxicity were identified that separated a specific compound's mode of toxicity from all other modes of toxicity exhibited by all other compounds in the database. These markers were identified for each of the cardiotoxins. The top 10, 25, 50, 100 genes based on individual discriminate scores were used in a model to ensure that combination of genes provided a better prediction than individual genes. As described above, all combinations of two or more genes from this list could potentially provide better prediction than individual genes when selected in any order or by ordered, agglomerate, divisive, or random approaches. In addition, combining these genes with other genes could provide better predictive ability, but most of this predictive ability would come from the genes listed herein.

Samples may be considered toxic if they score positive in any individual compound represented here or in any modeling method mentioned under general toxicology models based on combination of individual time and dose grouping of individual toxic compounds obtainable from the data. Most logical groupings with one or more genes and one or more sample dose and time points should produce better predictions of general toxicity or similarity to known toxicant than individual genes.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

TABLE 1
Attorney Docket No. 44921-5090US Document No. 1828250.1

| Seq. | | GenBank | | | Model |
|---|---|---|---|---|---|
| ID No. | Identifier | Acc. No. | Gene Name | Unigene Cluster Title | Code |
| 1 | 16130 | J01435 | unknown Glu-Pro dipeptide | unknown Glu-Pro dipeptide repeat | a, c, f, |
| | | | repeat protein | protein | General |
| 1 | 25319 | J01435 | unknown Glu-Pro dipeptide | unknown Glu-Pro dipeptide repeat | a, c, f |
| | | | repeat protein | protein | |
| 1 | 25050 | J01435 | unknown Glu-Pro dipeptide | unknown Glu-Pro dipeptide repeat | c, f, g |
| | | | repeat protein | protein | |
| 2 | 25051 | J01436 | | | a, c |
| 3 | 25439 | M35826 | | | a, c, f, g |
| 4 | 16132 | AA850885 | unknown Glu-Pro dipeptide | unknown Glu-Pro dipeptide repeat | a, c |
| | | | repeat protein | protein | |
| 5 | 6153 | AA875531 | procollagen, type I, alpha 2 | Rattus norvegicus CDK110 mRNA, | e |
| | | | | procollagen, type I, alpha 2 | |
| 6 | 15666 | AA892248 | | | a, f |
| 7 | 19421 | AA945152 | NG, NG dimethylarginine | NG, NG dimethylarginine | a, c |
| | | | dimethylaminohydrolase | dimethylaminohydrolase | |
| 8 | 14981 | AI103396 | | Rattus norvegicus CDK110 mRNA | a, c, f |
| 9 | 14982 | AI171355 | | Rattus norvegicus CDK110 mRNA | a, f |
| 10 | 14983 | AI179150 | | Rattus norvegicus CDK110 mRNA | a, c, f |
| 11 | 21152 | X14848 | golgi SNAP receptor complex | golgi SNAP receptor complex | a, b, c, f, g |
| | | | member 1 | member 1 | |
| 12 | 15511 | NM_012498 | Rattus norvegicus Aldehyde | Aldehyde reductase 1 (low Km | h |
| | | | reductase 1 (low Km aldose | aldose reductase) (5.8 kb Pstl | |
| | | | reductase) (5.8 kb Pstl fragment, | fragment, probably the functional | |
| | | | probably the functional gene) | gene) | |
| | | | (Akr1b1), mRNA. Length = 1339 | | |
| 13 | 7063 | NM_012495 | Rattus norvegicus Aldolase A, | Aldolase A, fructose-bisphosphate | c |
| | | | fructose-bisphosphate (Aldoa), | | |
| | | | mRNA. Length = 1442 | | |
| 14 | 7427 | NM_012515 | Rattus norvegicus | Benzodiazepin receptor (peripheral) | h |
| | | | Benzodiazepin receptor | | |
| | | | (peripheral) (Bzrp), mRNA. | | |
| | | | Length = 781 | | |
| 15 | 15741 | NM_012520 | Rattus norvegicus Catalase | Catalase | c, g |
| | | | (Cat), mRNA. Length = 2495 | | |
| 16 | 4468 | NM_012529 | Rattus norvegicus Creatine | Creatine kinase, brain | e |
| | | | kinase, brain (Ckb), mRNA. | | |
| | | | Length = 1146 | | |
| 17 | 23869 | NM_012551 | Rattus norvegicus Early growth | Early growth response 1 | General |
| | | | response 1 (Egr1), mRNA. | | |
| | | | Length = 3112 | | |
| 18 | 19408 | NM_012554 | Rattus norvegicus Enolase 1, | Enolase 1, alpha | h |
| | | | alpha (Eno1), mRNA. Length = | | |
| | | | 1725 | | |
| 19 | 6477 | NM_012559 | Rattus norvegicus Fibrinogen, | Fibrinogen, gamma polypeptide | g |
| | | | gamma polypeptide (Fgg), | | |
| | | | mRNA. Length = 1358 | | |
| 20 | 16080 | NM_012580 | Rattus norvegicus Heme | Heme oxygenase | General |
| | | | oxygenase (Hmox1), mRNA. | | |
| | | | Length = 870 | | |
| 21 | 20313 | NM_012585 | Rattus norvegicus 5- | 5-Hydroxytryptamine (serotonin) | General |
| | | | Hydroxytryptamine (serotonin) | receptor 1A | |
| | | | receptor 1A (Htr1a), mRNA. | | |
| | | | Length = 1269 | | |
| 22 | 15098 | NM_012588 | Rattus norvegicus Insulin-like | Insulin-like growth factor-binding | c |
| | | | growth factor-binding protein | protein (IGF-BP3) | |
| | | | (IGF-BP3) (Igfbp3), mRNA. | | |
| | | | Length = 2352 | | |
| 23 | 24716 | NM_012589 | Rattus norvegicus Interleukin 6 | Interleukin 6 (interferon, beta 2) | General |
| | | | (interferon, beta 2) (II6), mRNA. | | |
| | | | Length = 1046 | | |
| 24 | 2629 | NM_012603 | Rattus norvegicus Avian | Avian myelocytomatosis viral (v- | General |
| | | | myelocytomatosis viral (v-myc) | myc) oncogene homolog | |
| | | | oncogene homolog (Myc), | | |
| | | | mRNA. Length = 2168 | | |
| 25 | 23523 | NM_012615 | Rattus norvegicus Ornitine | Ornitine decarboxylase | General |
| | | | decarboxylase (Odc1), mRNA. | | |
| | | | Length = 2442 | | |
| 26 | 20589 | NM_012618 | Rattus norvegicus S100 calcium- | Protein 9 Ka homologous to | General |
| | | | binding protein A4 (S100a4), | calcium-binding protein | |
| | | | mRNA. Length = 487 | | |
| 27 | 15540 | NM_012620 | Rattus norvegicus serine (or | Plasminogen activator inhibitor | General |
| | | | cysteine) proteinase inhibitor, | | |
| | | | clade E (nexin, plasminogen | | |
| | | | activator inhibitor type 1), | | |
| | | | member 1 (Serpine1), mRNA. | | |
| | | | Length = 3053 | | |
| 28 | 18725 | NM_012645 | Rattus norvegicus RT1 class Ib | Rattus norvegicus MHC class Ib | c |
| | | | gene (RT1Aw2), mRNA. Length = | RT1.S3 (RT1.S3) mRNA, partial | |
| | | | 1540 | cds | |
| 28 | 18726 | NM_012645 | Rattus norvegicus RT1 class Ib | Rattus norvegicus MHC class Ib | General |
| | | | gene (RT1Aw2), mRNA. Length = | RT1.S3 (RT1.S3) mRNA, partial | |
| | | | 1540 | cds | |
| 29 | 9423 | NM_012649 | Rattus norvegicus | Ryudocan/syndecan 4 | General |
| | | | Ryudocan/syndecan 4 (Sdc4), | | |
| | | | mRNA. Length = 2462 | | |
| 29 | 9424 | NM_012649 | Rattus norvegicus | Ryudocan/syndecan 4 | General |
| | | | Ryudocan/syndecan 4 (Sdc4), | | |
| | | | mRNA. Length = 2462 | | |
| 30 | 16224 | NM_012656 | Rattus norvegicus Secreted | Secreted acidic cystein-rich | c |
| | | | acidic cystein-rich glycoprotein | glycoprotein (osteonectin) | |
| | | | (osteonectin) (Sparc), mRNA. | | |
| | | | Length = 2025 | | |
| 31 | 1514 | NM_012678 | Rattus norvegicus Tropomyosin | Tropomycin 4 | c, h, |
| | | | 4 (Tpm4), mRNA. Length = 900 | | General |
| 32 | 20776 | NM_012680 | Rattus norvegicus Tuberous | Tuberous sclerosis 2, (renal | General |
| | | | sclerosis 2, (renal carcinoma) | carcinoma) | |
| | | | (Tsc2), mRNA. Length = 5573 | | |
| 33 | 25650 | NM_012736 | Rattus norvegicus Glycerol-3- | | f |
| | | | phosophate dehydrogenase 2 | | |
| | | | (mitochondrial) (Gpd2), mRNA. | | |
| | | | Length = 2400 | | |
| 34 | 344 | NM_012747 | Rattus norvegicus Signal | Signal transducer and activator of | b |
| | | | transducer and activator of | transcription 3 | |
| | | | transcription 3 (Stat3), mRNA. | | |
| | | | Length = 2924 | | |
| 35 | 8829 | NM_012749 | Rattus norvegicus Nucleolin | Nucleolin | h, General |
| | | | (Ncl), mRNA. Length = 2142 | | |
| 36 | 3601 | NM_012751 | Rattus norvegicus solute carrier | Glucose transporter 4, insuline- | d |
| | | | family 2 (facilitated glucose | responsive | |
| | | | transporter), member 4 (Glut4), | | |
| | | | mRNA. Length = 2506 | | |
| 37 | 15174 | NM_012756 | Rattus norvegicus Insulin-like | Insulin-like growth factor 2 receptor | b |
| | | | growth factor 2 receptor (Igf2r), | | |
| | | | mRNA. Length = 8810 | | |
| 38 | 1349 | NM_012776 | Rattus norvegicus adrenergic | G-protein-linked receptor kinase | General |
| | | | receptor kinase, beta 1 (Adrbk1), | (beta adrenergic receptor kinase 1) | |
| | | | mRNA. Length = 2683 | | |
| 39 | 449 | NM_012786 | Rattus norvegicus Cytochrome c | Cytochrom c oxidase subunit VIII-H | a, b |
| | | | oxidase subunit VIII-H | (heart/muscle) | |
| | | | (heart/muscle) (Cox8h), mRNA. | | |
| | | | Length = 330 | | |
| 40 | 961 | NM_012796 | Rattus norvegicus glutathione S- | glutathione S-transferase, theta 2 | e |
| | | | transferase, theta 2 (Gstt2), | | |
| | | | mRNA. Length = 1258 | | |
| 41 | 15032 | NM_012816 | Rattus norvegicus alpha- | Methylacyl-CoA racemase alpha | g, i |
| | | | methylacyl-CoA racemase | | |
| | | | (Amacr), mRNA. Length = 1504 | | |
| 42 | 18109 | NM_012823 | Rattus norvegicus Annexin A3 | ESTs, Weakly similar to LURT3 | General |
| | | | (Anx3), mRNA. Length = 1454 | annexin III - rat [R. norvegicus] | |
| 43 | 11137 | NM_012839 | Rattus norvegicus Cytochrome | Cytochrome C, expressed in | g |
| | | | C, expressed in somatic tissues | somatic tissues | |
| | | | (Cycs), mRNA. Length = 318 | | |
| 44 | 17541 | NM_012844 | Rattus norvegicus Epoxide | Epoxide hydrolase 1 (microsomal | a, c, |
| | | | hydrolase 1 (microsomal | xenobiotic hydrolase) | General |
| | | | xenobiotic hydrolase) (Ephx1), | | |
| | | | mRNA. Length = 1242 | | |
| 45 | 17305 | NM_012876 | Rattus norvegicus Ribosomal | Ribosomal protein S29 | b, e, g |
| | | | protein S29 (Rps29), mRNA. | | |
| | | | Length = 318 | | |
| 46 | 1748 | NM_012889 | Rattus norvegicus Vascular cell | Vascular cell adhesion molecule 1 | a, General |
| | | | adhesion molecule 1 (Vcam1), | | |
| | | | mRNA. Length = 3007 | | |
| 47 | 16721 | NM_012891 | Rattus norvegicus Acyl-Coa | Acyl-Coa dehydrogenase, Very long | General |
| | | | dehydrogenase, Very long chain | chain | |
| | | | (Acadvl), mRNA. Length = 2117 | | |
| 48 | 16274 | NM_012898 | Rattus norvegicus alpha 2 HS- | alpha 2 HS-glycoprotein alpha 2 | a |
| | | | glycoprotein alpha 2 (fetuin) | (fetuin) | |
| | | | (Ahsg), mRNA. Length = 1464 | | |
| 49 | 24783 | NM_012914 | Rattus norvegicus ATPase, | ATPase, Ca++ transporting, | h, General |
| | | | Ca++ transporting, ubiquitous | ubiquitous | |
| | | | (Atp2a3), mRNA. Length = 4472 | | |
| 50 | 18695 | NM_012931 | Rattus norvegicus v-crk- | v-crk-associated tyrosine kinase | b, General |
| | | | associated tyrosine kinase | substrate | |
| | | | substrate (Crkas), mRNA. | | |
| | | | Length = 3335 | | |
| 51 | 2555 | NM_012967 | Rattus norvegicus Intercellular | Intercellular adhesion molecule 1 | General |
| | | | adhesion molecule 1 (Icam1), | | |
| | | | mRNA. Length = 2602 | | |
| 52 | 24528 | NM_012973 | Rattus norvegicus Potassium | Potassium (K+) channel protein, | h |
| | | | (K+) channel protein, slowly | slowly activating (Isk) | |
| | | | activating (Isk) (Kcne1), mRNA. | | |
| | | | Length = 585 | | |
| 53 | 956 | NM_012976 | Rattus norvegicus Lectin, | Lectin, galactose binding, soluble 9 | a, General |
| | | | galactose binding, soluble 5 | (Galectin-9) | |
| | | | (Galectin-5) (Lgals5), mRNA. | | |
| | | | Length = 872 | | |
| 54 | 958 | NM_012977 | Rattus norvegicus Lectin, | Lectin, galactose binding, soluble 9 | General |
| | | | galactose binding, soluble 9 | (Galectin-9) | |
| | | | (Galectin-9) (Lgals9), mRNA. | | |
| | | | Length = 1545 | | |
| 55 | 17394 | NM_012992 | Rattus norvegicus | Nucleoplasmin-related protein | General |
| | | | Nucleoplasmin-related protein | (Nuclear protein B23 | |
| | | | (Nuclear protein B23 (Npm1), | | |
| | | | mRNA. Length = 1232 | | |
| 56 | 19392 | NM_012998 | Rattus norvegicus Protein | Protein disulfide isomerase (Prolyl | i |
| | | | disulfide isomerase(Prolyl 4- | 4-hydroxylase, beta polypeptide) | |
| | | | hydroxylase, beta polypeptide) | | |
| | | | (P4hb), mRNA. Length = 2460 | | |
| 57 | 24264 | NM_012999 | Rattus norvegicus Subtilisin - like | Subtilisin - like endoprotease | e |
| | | | endoprotease (Pace4), mRNA. | | |
| | | | Length = 4153 | | |
| 58 | 3405 | NM_013011 | Rattus norvegicus Tyrosine 3- | Tyrosine 3- | i, General |
| | | | monooxygenase/tryptophan 5- | monooxygenase/tryptophan 5- | |
| | | | monooxygenase activation | monooxygenase activation protein, | |
| | | | protein, zeta polypeptide | zeta polypeptide | |
| | | | (Ywhaz), mRNA. Length = 1687 | | |
| 59 | 20229 | NM_013018 | Rattus norvegicus Ras-related | Ras-related small GTP binding | General |
| | | | small GTP binding protein 3A | protein 3A | |
| | | | (Rab3a), mRNA. Length = 743 | | |
| 60 | 1606 | NM_013039 | Rattus norvegicus ATP-binding | Sulfonylurea receptor | g |
| | | | cassette, sub-family C | | |
| | | | (CFTR/MRP), member 8 | | |
| | | | (Abcc8), mRNA. Length = 5099 | | |
| 61 | 730 | NM_013040 | Rattus norvegicus ATP-binding | Sulfonylurea receptor 2 | h |
| | | | cassette, sub-family C | | |
| | | | (CFTR/MRP), member 9 | | |
| | | | (Abcc9), mRNA. Length = 5000 | | |
| 62 | 17401 | NM_013043 | Rattus norvegicus Transforming | Transforming growth factor beta | g, General |
| | | | growth factor beta stimulated | stimulated clone 22 | |
| | | | clone 22 (Tgfb1i4), mRNA. | | |
| | | | Length = 1666 | | |
| 63 | 11114 | NM_013046 | Rattus norvegicus Thyrotropin | Thyrotropin releasing hormone | f, General |
| | | | releasing hormone (Trh), mRNA. | | |
| | | | Length = 768 | | |
| 63 | 11113 | NM_013046 | Rattus norvegicus Thyrotropin | Thyrotropin releasing hormone | f |
| | | | releasing hormone (Trh), mRNA. | | |
| | | | Length = 768 | | |
| 64 | 16924 | NM_013069 | Rattus norvegicus CD74 antigen | CD74 antigen (invariant | b |
| | | | (invariant polpypeptide of major | polpypeptide of major | |
| | | | histocompatibility class II antigen- | histocompatibility class II antigen- | |
| | | | associated) (Cd74), mRNA. | associated) | |
| | | | Length = 1150 | | |
| 64 | 25676 | NM_013069 | Rattus norvegicus CD74 antigen | | b |
| | | | (invariant polpypeptide of major | | |
| | | | histocompatibility class II antigen- | | |
| | | | associated) (Cd74), mRNA. | | |
| | | | Length = 1150 | | |
| 64 | 16925 | NM_013069 | Rattus norvegicus CD74 antigen | CD74 antigen (invariant | b |
| | | | (invariant polpypeptide of major | polpypeptide of major | |
| | | | histocompatibility class II antigen- | histocompatibility class II antigen- | |
| | | | associated) (Cd74), mRNA | associated) | |
| | | | Length = 1150 | | |
| 65 | 357 | NM_013086 | Rattus norvegicus CAMP | CAMP responsive element | General |
| | | | responsive element modulator | modulator | |
| | | | (Crem), mRNA. Length = 607 | | |
| 66 | 1521 | NM_013091 | Rattus norvegicus Tumor | Tumor necrosis factor receptor | i |
| | | | necrosis factor receptor | | |
| | | | superfamily, member 1a (Tnfr1), | | |
| | | | mRNA. Length = 2130 | | |
| 67 | 1684 | NM_013096 | Rattus norvegicus Hemoglobin, | Hemoglobin, alpha 1 | a, b |
| | | | alpha 1 (Hba1), mRNA. Length = | | |
| | | | 556 | | |
| 67 | 26150 | NM_013096 | Rattus norvegicus Hemoglobin, | | a, b |
| | | | alpha 1 (Hba1), mRNA. Length = | | |
| | | | 556 | | |
| 67 | 1689 | NM_013096 | Rattus norvegicus Hemoglobin, | Hemoglobin, alpha 1 | a, b, |
| | | | alpha 1 (Hba1), mRNA. Length = | | General |
| | | | 556 | | |
| 67 | 1685 | NM_013096 | Rattus norvegicus Hemoglobin, | Hemoglobin, alpha 1 | a |
| | | | alpha 1 (Hba1), mRNA. Length = | | |
| | | | 556 | | |
| 67 | 1688 | NM_013096 | Rattus norvegicus Hemoglobin, | Hemoglobin, alpha 1 | a, b |
| | | | alpha 1 (Hba1), mRNA. Length = | | |
| | | | 556 | | |
| 68 | 1321 | NM_013098 | Rattus norvegicus Glucose-6- | Glucose-6-phosphatase | a |
| | | | phosphatase (G6pc), mRNA. | | |
| | | | Length = 2237 | | |
| 69 | 19949 | NM_013106 | Rattus norvegicus Guanine | Guanine nucleotide binding, protein, | General |
| | | | nucleotide binding, protein, alpha | alpha inhibiting polypeptide 3 | |
| | | | inhibiting polypeptide 3 (Gnai3), | | |
| | | | mRNA. Length = 3072 | | |
| 70 | 23709 | NM_013113 | Rattus norvegicus ATPase | ATPase Na+/K+ transporting beta 1 | g |
| | | | Na+/K+ transporting beta 1 | polypeptide | |
| | | | polypeptide (Atp1b1), mRNA. | | |
| | | | Length = 2528 | | |
| 71 | 2005 | NM_013127 | Rattus norvegicus CD38 antigen | CD38 antigen (ADP-ribosyl cyclase/ | c |
| | | | (ADP-ribosyl cyclase/cyclic | cyclic ADP-ribose hydrolase) | |
| | | | ADP-ribose hydrolase) (Cd38), | | |
| | | | mRNA. Length = 2248 | | |
| 72 | 14299 | NM_013129 | Rattus norvegicus Interleukin 15 | Interleukin 15 | General |
| | | | (II15), mRNA. Length = 1313 | | |
| 73 | 16982 | NM_013144 | Rattus norvegicus Insulin-like | Insulin-like growth factor binding | General |
| | | | growth factor binding protein 1 | protein 1 | |
| | | | (Igfbp1), mRNA. Length = 1500 | | |
| 74 | 21683 | NM_013154 | Rattus norvegicus | CCAAT/enhancerbinding, protein | General |
| | | | CCAAT/enhancerbinding, protein | (C/EBP) delta | |
| | | | (C/EBP) delta (Cebpd), mRNA. | | |
| | | | Length = 1200 | | |
| 74 | 21682 | NM_013154 | Rattus norvegicus | CCAAT/enhancerbinding, protein | General |
| | | | CCAAT/enhancerbinding, protein | (C/EBP) delta | |
| | | | (C/EBP) delta (Cebpd), mRNA. | | |
| | | | Length = 1200 | | |
| 75 | 24867 | NM_013155 | Rattus norvegicus Very low | Very low density lipoprotein | General |
| | | | density lipoprotein receptor | receptor | |
| | | | (Vldlr), mRNA. Length = 2952 | | |
| 76 | 25567 | NM_013156 | Rattus norvegicus Cathepsin L | | d, General |
| | | | (Ctsl), mRNA. Length = 1386 | | |
| 76 | 3431 | NM_013156 | Rattus norvegicus Cathepsin L | Cathepsin L | General |
| | | | (Ctsl), mRNA. Length = 1386 | | |
| 76 | 3430 | NM_013156 | Rattus norvegicus Cathepsin L | Cathepsin L | d, General |
| | | | (Ctsl), mRNA. Length = 1386 | | |
| 77 | 1714 | NM_013187 | Rattus norvegicus | Phospholipase C, gamma 1 | g, i, |
| | | | Phospholipase C, gamma 1 | | General |
| | | | (Plcg1), mRNA. Length = 5106 | | |
| 78 | 16448 | NM_013197 | Rattus norvegicus | Aminolevulinate synthase 2, delta | a, b, |
| | | | Aminolevulinate synthase 2, | | General |
| | | | delta (Alas2), mRNA. Length = | | |
| | | | 1899 | | |
| 79 | 20826 | NM_013218 | Rattus norvegicus adenylate | adenylate kinase 3 | h |
| | | | kinase 3 (Ak3), mRNA. Length = | | |
| | | | 1061 | | |
| 80 | 18313 | NM_013220 | Rattus norvegicus cardiac | cardiac ankyrin repeat protein | f, g, i, |
| | | | ankyrin repeat protein (AIrp), | | General |
| | | | mRNA. Length = 1749 | | |
| 81 | 815 | NM_013224 | Rattus norvegicus ribosomal | ribosomal protein S26 | e, General |
| | | | protein S26 (Rps26), mRNA. | | |
| | | | Length = 435 | | |
| 82 | 15239 | NM_016989 | Rattus norvegicus adenylate | R. norvegicus (Sprague Dawley) | h |
| | | | cyclase activating polypeptide 1 | ribosomal protein L15 mRNA | |
| | | | (Adcyap1), mRNA. Length = | | |
| | | | 2681 | | |
| 83 | 3202 | NM_017039 | Rattus norvegicus Protein | Protein phosphatase 2 (formerly | h |
| | | | phosphatase 2 (formerly 2A), | 2A), catalytic subunit, alpha isoform | |
| | | | catalytic subunit, alpha isoform | | |
| | | | (Ppp2ca), mRNA. Length = 1804 | | |
| 83 | 3203 | NM_017039 | Rattus norvegicus Protein | Protein phosphatase 2 (formerly | h |
| | | | phosphatase 2 (formerly 2A), | 2A), catalytic subunit, alpha isoform | |
| | | | catalytic subunit, alpha isoform | | |
| | | | (Ppp2ca), mRNA. Length = 1804 | | |
| 84 | 24596 | NM_017040 | Rattus norvegicus Protein | Protein phosphatase 2 (formerly | h |
| | | | phosphatase 2 (formerly 2A), | 2A), catalytic subunit, beta isoform | |
| | | | catalytic subunit, beta isoform | | |
| | | | (Ppp2cb), mRNA. Length = 1843 | | |
| 85 | 6654 | NM_017068 | Rattus norvegicus Lysosomal- | Lysosomal-associated membrane | a |
| | | | associated membrane protein 2 | protein 2 | |
| | | | (Lamp2), mRNA. Length = 1548 | | |
| 86 | 11152 | NM_017073 | Rattus norvegicus Glutamine | Glutamine synthetase (glutamate- | General |
| | | | synthetase (glutamate-ammonia | ammonia ligase) | |
| | | | ligase) (Glul), mRNA. Length = | | |
| | | | 2793 | | |
| 86 | 11153 | NM_017073 | Rattus norvegicus Glutamine | Glutamine synthetase (glutamate- | General |
| | | | synthetase (glutamate-ammonia | ammonia ligase) | |
| | | | ligase) (Glul), mRNA. Length = | | |
| | | | 2793 | | |
| 87 | 22552 | NM_017087 | Rattus norvegicus Small | Small proteoglycan I (biglycan), | General |
| | | | proteoglycan I (biglycan), bone | bone (BSPG1) (bone/cartilage | |
| | | | (BSPG1) (bone/cartilage | proteclycan 1 precursor) | |
| | | | proteclycan 1 precursor) (Bgn), | | |
| | | | mRNA. Length = 2446 | | |
| 88 | 8888 | NM_017090 | Rattus norvegicus guanylate | Guanylate cyclase, soluble, alpha 1 | General |
| | | | cyclase 1, soluble, alpha 3 | (GTP pyrophosphate - lyase) | |
| | | | (Gucy1a3), mRNA. Length = | | |
| | | | 4775 | | |
| 89 | 1435 | NM_017125 | Rattus norvegicus Cd63 antigen | Cd63 antigen | g, General |
| | | | (Cd63), mRNA. Length = 860 | | |
| 90 | 21663 | NM_017126 | Rattus norvegicus ferredoxin 1 | ferredoxin 1 | General |
| | | | (Fdx1), mRNA. Length = 838 | | |
| 91 | 24886 | NM_017138 | Rattus norvegicus laminin | laminin receptor 1 | General |
| | | | receptor 1 (Lamr1), mRNA. | | |
| | | | Length = 1018 | | |
| 92 | 24106 | NM_017141 | Rattus norvegicus DNA | DNA polymerase beta | c |
| | | | polymerase beta (Polb), mRNA. | | |
| | | | Length = 3298 | | |
| 92 | 24107 | NM_017141 | Rattus norvegicus DNA | DNA polymerase beta | General |
| | | | polymerase beta (Polb), mRNA. | | |
| | | | Length = 3298 | | |
| 93 | 20859 | NM_017144 | Rattus norvegicus troponin I, | troponin I, cardiac | a, b, f |
| | | | cardiac (Tnni3), mRNA. Length = | | |
| | | | 830 | | |
| 94 | 15365 | NM_017147 | Rattus norvegicus cofilin 1, non- | cofilin 1, non-muscle | g, h, |
| | | | muscle (Cfl1), mRNA. Length = | | General |
| | | | 1039 | | |
| 95 | 5351 | NM_017150 | Rattus norvegicus ribosomal | ribosomal protein L29 | General |
| | | | protein L29 (Rpl29), mRNA. | | |
| | | | Length = 630 | | |
| 96 | 16953 | NM_017151 | Rattus norvegicus ribosomal | ribosomal protein S15 | e, h |
| | | | protein S15 (Rps15), mRNA. | | |
| | | | Length = 487 | | |
| 97 | 21975 | NM_017154 | Rattus norvegicus xanthine | xanthine dehydrogenase | General |
| | | | dehydrogenase (Xdh), mRNA. | | |
| | | | Length = 4198 | | |
| 98 | 17104 | NM_017160 | Rattus norvegicus ribosomal | ribosomal protein S6 | General |
| | | | protein S6 (Rps6), mRNA. | | |
| | | | Length = 801 | | |
| 98 | 17107 | NM_017160 | Rattus norvegicus ribosomal | ribosomal protein S6 | General |
| | | | protein S6 (Rps6), mRNA. | | |
| | | | Length = 801 | | |
| 99 | 19031 | NM_017180 | Rattus norvegicus T-cell death | T-cell death associated gene | General |
| | | | associated gene (Tdag), mRNA. | | |
| | | | Length = 1353 | | |
| 100 | 15434 | NM_017187 | Rattus norvegicus high mobility | high mobility group protein 2 | f |
| | | | group box 2 (Hmgb2), mRNA. | | |
| | | | Length = 1072 | | |
| 101 | 14696 | NM_017202 | Rattus norvegicus cytochrome c | cytochrome c oxidase, subunit IV | e |
| | | | oxidase, subunit IVa (Cox4a), | | |
| | | | mRNA. Length = 696 | | |
| 102 | 5005 | NM_017209 | Rattus norvegicus nuclear | nuclear receptor binding factor 1 | General |
| | | | receptor binding factor 1 (Nrbf1), | | |
| | | | mRNA. Length = 1302 | | |
| 103 | 20193 | NM_017232 | Rattus norvegicus prostaglandin- | prostaglandin-endoperoxide | General |
| | | | endoperoxide synthase 2 | synthase 2 | |
| | | | (Ptgs2), mRNA. Length = 1827 | | |
| 104 | 20482 | NM_017240 | Rattus norvegicus myosin heavy | myosin heavy chain, cardiac | e |
| | | | chain, cardiac muscle, fetal | muscle, fetal | |
| | | | (Myh7), mRNA. Length = 5925 | | |
| 104 | 3780 | NM_017240 | Rattus norvegicus myosin heavy | EST | e |
| | | | chain, cardiac muscle, fetal | | |
| | | | (Myh7), mRNA. Length = 5925 | | |
| 104 | 20484 | NM_017240 | Rattus norvegicus myosin heavy | myosin heavy chain, cardiac | a, c, e, f |
| | | | chain, cardiac muscle, fetal | muscle, fetal | |
| | | | (Myh7), mRNA. Length = 5925 | | |
| 105 | 17563 | NM_017245 | Rattus norvegicus eukaryotic | eukaryotic translation elongation | g, General |
| | | | translation elongation factor 2 | factor 2 | |
| | | | (Eef2), mRNA. Length = 2626 | | |
| 106 | 15301 | NM_017259 | Rattus norvegicus B-cell | B-cell translocation gene 2, anti- | General |
| | | | translocation gene 2, anti- | proliferative | |
| | | | proliferative (Btg2), mRNA. | | |
| | | | Length = 2519 | | |
| 106 | 15299 | NM_017259 | Rattus norvegicus B-cell | B-cell translocation gene 2, anti- | General |
| | | | translocation gene 2, anti- | proliferative | |
| | | | proliferative (Btg2), mRNA. | | |
| | | | Length = 2519 | | |
| 106 | 15300 | NM_017259 | Rattus norvegicus B-cell | B-cell translocation gene 2, anti- | General |
| | | | translocation gene 2, anti- | proliferative | |
| | | | proliferative (Btg2), mRNA. | | |
| | | | Length = 2519 | | |
| 107 | 3254 | NM_017282 | Rattus norvegicus proteasome | proteasome (prosome, macropain) | h |
| | | | (prosome, macropain) subunit, | subunit, alpha type 5 | |
| | | | alpha type 5 (Psma5), mRNA. | | |
| | | | Length = 970 | | |
| 108 | 82 | NM_017297 | Rattus norvegicus potassium | potassium inwardly-rectifying | i |
| | | | inwardly-rectifying channel, | channel, subfamily J, member 5 | |
| | | | subfamily J, member 5 (Kcnj5), | | |
| | | | mRNA. Length = 3156 | | |
| 109 | 1894 | NM_017320 | Rattus norvegicus cathepsin S | cathepsin S | General |
| | | | (Ctss), mRNA. Length = 1330 | | |
| 110 | 356 | NM_017334 | Rattus norvegicus transcriptional | CAMP responsive element | General |
| | | | repressor CREM (CREM), | modulator | |
| | | | mRNA. Length = 436 | | |
| 110 | 355 | NM_017334 | Rattus norvegicus transcriptional | | General |
| | | | repressor CREM (CREM), | | |
| | | | mRNA. Length = 436 | | |
| 111 | 20848 | NM_017343 | Rattus norvegicus myosin | Rat mRNA for myosin regulatory | c, g |
| | | | regulatory light chain (MRLCB), | light chain (RLC) | |
| | | | mRNA. Length = 1139 | | |
| 112 | 460 | NM_019131 | Rattus norvegicus Tropomyosin | Tropomyosin 1 (alpha) | a |
| | | | 1 (alpha) (Tpm1), mRNA. | | |
| | | | Length = 1004 | | |
| 113 | 5622 | NM_019143 | Rattus norvegicus Fibronectin 1 | Fibronectin 1 | General |
| | | | (Fn1), mRNA. Length = 8329 | | |
| 114 | 17066 | NM_019170 | Rattus norvegicus carbonyl | carbonyl reductase | e |
| | | | reductase (Cbr), mRNA. | | |
| | | | Length = 1018 | | |
| 115 | 1174 | NM_019184 | Rattus norvegicus Cytochrome | Cytochrome P450, subfamily IIC | b |
| | | | P450, subfamily IIC | (mephenytoin 4-hydroxylase) | |
| | | | (mephenytoin 4-hydroxylase) | | |
| | | | (Cyp2c), mRNA. Length = 1856 | | |
| 116 | 18569 | NM_019212 | Rattus norvegicus actin, alpha 1, | actin, alpha 1, skeletal muscle | General |
| | | | skeletal muscle (Acta1), mRNA. | | |
| | | | Length = 1134 | | |
| 117 | 20433 | NM_019232 | Rattus norvegicus | serum/glucocorticoid regulated | b, General |
| | | | serum/glucocorticoid regulated | kinase | |
| | | | kinase (Sgk), mRNA. Length = | | |
| | | | 2435 | | |
| 118 | 17908 | NM_019242 | Rattus norvegicus interferon- | interferon-related developmental | c |
| | | | related developmental regulator | regulator 1 | |
| | | | 1 (lfrd1), mRNA. Length = 1736 | | |
| 119 | 11218 | NM_019247 | Rattus norvegicus paired-like | paired-like homeodomain | General |
| | | | homeodomain transcription | transcription factor 3 | |
| | | | factor 3 (Pitx3), mRNA. Length = | | |
| | | | 1253 | | |
| 120 | 23419 | NM_019257 | Rattus norvegicus splicing factor, | splicing factor, arginine/serine-rich 5 | d |
| | | | arginine/serine-rich 5 (SRp40, | (SRp40, HRS) | |
| | | | HRS) (Sfrs5), mRNA. Length = | | |
| | | | 2781 | | |
| 121 | 21443 | NM_019262 | Rattus norvegicus complement | complement component 1, q | General |
| | | | component 1, q subcomponent, | subcomponent, beta polypeptide | |
| | | | beta polypeptide (C1qb), mRNA. | | |
| | | | Length = 1136 | | |
| 122 | 20735 | NM_019283 | Rattus norvegicus solute carrier | antigen identified by monoclonal | a, d, |
| | | | family 3 (activators of dibasic | antibodies 4F2 | General |
| | | | and neutral amino acid | | |
| | | | transport), member 2 (Slc3a2), | | |
| | | | mRNA. Length = 1940 | | |
| 122 | 20734 | NM_019283 | Rattus norvegicus solute carrier | antigen identified by monoclonal | d |
| | | | family 3 (activators of dibasic | antibodies 4F2 | |
| | | | and neutral amino acid | | |
| | | | transport), member 2 (Slc3a2), | | |
| | | | mRNA. Length = 1940 | | |
| 123 | 10016 | NM_019289 | Rattus norvegicus Actin-related | Actin-related protein complex 1b | i |
| | | | protein complex 1b (Arpc1b), | | |
| | | | mRNA. Length = 1430 | | |
| 124 | 15056 | NM_019291 | Rattus norvegicus carbonic | carbonic anhydrase 2 | General |
| | | | anhydrase 2 (Ca2), mRNA. | | |
| | | | Length = 1459 | | |
| 125 | 20750 | NM_019301 | Rattus norvegicus Complement | Complement receptor related | h |
| | | | receptor related protein (Cr1), | protein | |
| | | | mRNA. Length = 1811 | | |
| 126 | 52 | NM_019335 | Rattus norvegicus Protein | Protein kinase, interferon-inducible | e |
| | | | kinase, interferon-inducible | double stranded RNA dependent | |
| | | | double stranded RNA dependent | | |
| | | | (Prkr), mRNA. Length = 3808 | | |
| 127 | 22675 | NM_019358 | Rattus norvegicus glycoprotein | glycoprotein 38 | General |
| | | | 38 (Gp38), mRNA. Length = | | |
| | | | 1854 | | |
| 128 | 20057 | NM_019370 | Rattus norvegicus alkaline | alkaline phosphodiesterase | g |
| | | | phosphodiesterase (LOC54410), | | |
| | | | mRNA. Length = 2777 | | |
| 129 | 1324 | NM_019371 | Rattus norvegicus factor- | factor-responsive smooth muscle | General |
| | | | responsive smooth muscle | protein | |
| | | | protein (SM-20), mRNA. | | |
| | | | Length = 2825 | | |
| 130 | 22727 | NM_019383 | Rattus norvegicus ATP synthase | ATP synthase subunit d | i |
| | | | subunit d (Atp5jd), mRNA. | | |
| | | | Length = 581 | | |
| 131 | 16 | NM_019386 | Rattus norvegicus tissue-type | tissue-type transglutaminase | General |
| | | | transglutaminase (Tgm2), | | |
| | | | mRNA. Length = 3393 | | |
| 131 | 17 | NM_019386 | Rattus norvegicus tissue-type | tissue-type transglutaminase | General |
| | | | transglutaminase (Tgm2), | | |
| | | | mRNA. Length = 3393 | | |
| 132 | 574 | NM_019905 | Rattus norvegicus calpactin I | Rattus norvegicus clone BB.1.4.1 | g, i, |
| | | | heavy chain (Anxa2), mRNA. | unknown Glu-Pro dipeptide repeat | General |
| | | | Length = 1395 | protein mRNA, complete | |
| | | | | cds, calpactin I heavy | |
| | | | | chain, hydroxyacid oxidase 3 | |
| | | | | (medium-chain) | |
| 133 | 18713 | NM_020075 | Rattus norvegicus eukaryotic | eukaryotic initiation factor 5 (elF-5) | General |
| | | | initiation factor 5 (elF-5) (Eif5), | | |
| | | | mRNA. Length = 3504 | | |
| 133 | 18715 | NM_020075 | Rattus norvegicus eukaryotic | eukaryotic initiation factor 5 (elF-5) | General |
| | | | initiation factor 5 (elF-5) (Eif5), | | |
| | | | mRNA. Length = 3504 | | |
| 134 | 19696 | NM_021699 | Rattus norvegicus | serine/threonine kinase | General |
| | | | serine/threonine kinase | | |
| | | | (LOC60328), mRNA. Length = | | |
| | | | 2572 | | |
| 135 | 19710 | NM_021744 | Rattus norvegicus CD14 antigen | CD14 antigen | i, General |
| | | | (Cd14), mRNA. Length = 1591 | | |
| 136 | 20035 | NM_021754 | Rattus norvegicus Nopp140 | Nopp140 associated protein | General |
| | | | associated protein (Nap65), | | |
| | | | mRNA. Length = 1980 | | |
| 137 | 14397 | NM_021909 | Rattus norvegicus FXYD domain- | FXYD domain-containing ion | g, General |
| | | | containing ion transport regulator | transport regulator 5 | |
| | | | 5 (Fxyd5), mRNA. Length = 940 | | |
| 138 | 17100 | NM_022179 | Rattus norvegicus Hexokinase 3 | Hexokinase 3 | h, General |
| | | | (Hk3), mRNA. Length = 3692 | | |
| 138 | 17101 | NM_022179 | Rattus norvegicus Hexokinase 3 | Hexokinase 3 | General |
| | | | (Hk3), mRNA. Length = 3692 | | |
| 139 | 20204 | NM_022196 | Rattus norvegicus leukemia | leukemia inhibitory factor | i, General |
| | | | inhibitory factor (Lif), mRNA. | | |
| | | | Length = 609 | | |
| 140 | 20385 | NM_022229 | Rattus norvegicus heat shock | heat shock protein 60 (liver) | h |
| | | | protein 60 (liver) (Hsp60), | | |
| | | | mRNA. Length = 2175 | | |
| 141 | 6263 | NM_022251 | Rattus norvegicus | Rattus norvegicus aminopeptidase | b |
| | | | aminopeptidase A (Enpep), | A short variant mRNA, partial cds | |
| | | | mRNA. Length = 4075 | | |
| 142 | 6585 | NM_022266 | Rattus norvegicus connective | connective tissue growth factor | i, General |
| | | | tissue growth factor (Ctgf), | | |
| | | | mRNA. Length = 2345 | | |
| 143 | 17161 | NM_022298 | Rattus norvegicus alpha-tubulin | alpha-tubulin | g, General |
| | | | (Tuba1), mRNA. Length = 1617 | | |
| 144 | 13480 | NM_022390 | Rattus norvegicus quinoid | quinoid dihydropteridine reductase | f |
| | | | dihydropteridine reductase | | |
| | | | (Qdpr), mRNA. Length = 1307 | | |
| 145 | 22415 | NM_022392 | Rattus norvegicus growth | growth response protein (CL-6) | General |
| | | | response protein (CL-6) | | |
| | | | (LOC64194), mRNA. Length = | | |
| | | | 2410 | | |
| 146 | 23705 | NM_022396 | Rattus norvegicus guanine | guanine nucleotide binding protein | h |
| | | | nucleotide binding protein | gamma subunit 11 | |
| | | | gamma subunit 11 (Gng11), | | |
| | | | mRNA. Length = 557 | | |
| 147 | 23300 | NM_022398 | Rattus norvegicus 2-oxoglutarate | 2-oxoglutarate carrier | h |
| | | | carrier (LOC64201), mRNA. | | |
| | | | Length = 946 | | |
| 148 | 24536 | NM_022399 | Rattus norvegicus calreticulin | calreticulin | c |
| | | | (Calr), mRNA. Length = 1882 | | |
| 149 | 20915 | NM_022407 | Rattus norvegicus Aldehyde | aldehyde dehydrogenase family 1, | General |
| | | | dehydrogenase 1, subfamily A1 | subfamily A1 | |
| | | | (Aldh1a1), mRNA. Length = | | |
| | | | 2012 | | |
| 150 | 8212 | NM_022500 | Rattus norvegicus ferritin light | ferritin light chain 1 | General |
| | | | chain 1 (Ftl1), mRNA. Length = | | |
| | | | 552 | | |
| 151 | 1867 | NM_022510 | Rattus norvegicus ribosomal | ribosomal protein L4 | General |
| | | | protein L4 (Rpl4), mRNA. | | |
| | | | Length = 1387 | | |
| 152 | 4242 | NM_022521 | Rattus norvegicus ornithine | ornithine aminotransferase | f |
| | | | aminotransferase (Oat), mRNA. | | |
| | | | Length = 1938 | | |
| 153 | 6100 | NM_022531 | Rattus norvegicus desmin (Des), | desmin | f, General |
| | | | mRNA. Length = 2169 | | |
| 154 | 8598 | NM_022538 | Rattus norvegicus phosphatidate | phosphatidate phosphohydrolase | f |
| | | | phosphohydrolase type 2 | type 2 | |
| | | | (Ppap2), mRNA. Length = 871 | | |
| 154 | 8597 | NM_022538 | Rattus norvegicus phosphatidate | phosphatidate phosphohydrolase | b, General |
| | | | phosphohydrolase type 2 | type 2 | |
| | | | (Ppap2), mRNA. Length = 871 | | |
| 155 | 11039 | NM_022543 | Rattus norvegicus steroid | steroid sensitive gene-1 protein | General |
| | | | sensitive gene-1 protein (Ssg1), | | |
| | | | mRNA. Length = 3719 | | |
| 156 | 20925 | NM_022594 | Rattus norvegicus enoyl | enoyl hydratase-like protein, | e |
| | | | hydratase-like protein, | peroxisomal | |
| | | | peroxisomal (Ech1), mRNA. | | |
| | | | Length = 1097 | | |
| 157 | 20944 | NM_022597 | Rattus norvegicus cathepsin B | cathepsin B | f, General |
| | | | (Ctsb), mRNA. Length = 1904 | | |
| 158 | 21211 | NM_022607 | Rattus norvegicus MIPP65 | MIPP65 protein | b, h |
| | | | protein (Mipp65), mRNA. | | |
| | | | Length = 1468 | | |
| 159 | 2250 | NM_022643 | Rattus norvegicus Testis-specific | ESTs, Highly similar to 0506206A | h |
| | | | histone 2b (Th2b), mRNA. | histone H2B [R. norvegicus] | |
| | | | Length = 470 | | |
| 160 | 12542 | NM_022647 | Rattus norvegicus histone 2b | ESTs, Highly similar to 0506206A | f |
| | | | (H2b), mRNA. Length = 378 | histone H2B [R. norvegicus] | |
| 161 | 15727 | NM_022953 | Rattus norvegicus Slit1 (Slit1), | Slit1 | General |
| | | | mRNA. Length = 4950 | | |
| 162 | 15729 | NM_022955 | Rattus norvegicus MEGF6 | MEGF6 | General |
| | | | (Egfl3), mRNA. Length = 5523 | | |
| 163 | 21239 | NM_024125 | Rattus norvegicus Liver | Liver activating protein (LAP, also | c, General |
| | | | activating protein (LAP, also NF- | NF-IL6, nuclear factor-IL6, | |
| | | | IL6, nuclear factor-IL6, | previously designated TCF5) | |
| | | | previously designated TCF5) | | |
| | | | (Cebpb), mRNA. Length = 1408 | | |
| 164 | 353 | NM_024127 | Rattus norvegicus DNA-damage- | DNA-damage-inducible transcript 1 | i |
| | | | inducible transcript 1 (Gadd45a), | | |
| | | | mRNA. Length = 711 | | |
| 165 | 17226 | NM_024131 | Rattus norvegicus D- | D-dopachrome tautomerase | General |
| | | | dopachrome tautomerase (Ddt), | | |
| | | | mRNA. Length = 628 | | |
| 166 | 24634 | NM_024137 | Rattus norvegicus human | human immunodeficiency virus type | General |
| | | | immunodeficiency virus type I | I enhancer-binding protein 2 | |
| | | | enhancer-binding protein 2 | | |
| | | | (Hivep2), mRNA. Length = 9731 | | |
| 166 | 24632 | NM_024137 | Rattus norvegicus human | human immunodeficiency virus type | a |
| | | | immunodeficiency virus type I | I enhancer-binding protein 2 | |
| | | | enhancer-binding protein 2 | | |
| | | | (Hivep2), mRNA. Length = 9731 | | |
| 167 | 22 | NM_024388 | Rattus norvegicus immediate | immediate early gene transcription | b |
| | | | early gene transcription factor | factor NGFI-B | |
| | | | NGFI-B (Nr4a1), mRNA. | | |
| | | | Length = 2488 | | |
| 168 | 9929 | NM_024392 | Rattus norvegicus peroxisomal | peroxisomal multifunctional enzyme | g |
| | | | multifunctional enzyme type II | type II | |
| | | | (Hsd17b4), mRNA. Length = | | |
| | | | 2535 | | |
| 169 | 3582 | NM_024396 | Rattus norvegicus ATP-binding | ATP-binding cassette, sub-family A | a |
| | | | cassette, sub-family A (ABC1), | (ABC1), member 2 | |
| | | | member 2 (Abca2), mRNA. | | |
| | | | Length = 8040 | | |
| 170 | 22626 | NM_024400 | Rattus norvegicus a disintegrin | a disintegrin and metalloproteinase | General |
| | | | and metalloproteinase with | with thrombospondin motifs 1 | |
| | | | thrombospondin motifs 1 | (ADAMTS-1) | |
| | | | (ADAMTS-1) (Adamts1), mRNA. | | |
| | | | Length = 4878 | | |
| 171 | 13633 | NM_024403 | Rattus norvegicus activating | activating transcription factor ATF-4 | General |
| | | | transcription factor ATF-4 (Atf4), | | |
| | | | mRNA. Length = 1173 | | |
| 171 | 13634 | NM_024403 | Rattus norvegicus activating | activating transcription factor ATF-4 | General |
| | | | transcription factor ATF-4 (Atf4), | | |
| | | | mRNA. Length = 1173 | | |
| 172 | 17917 | NM_024488 | Rattus norvegicus CDK5 | CDK5 activator-binding protein C53 | a, b |
| | | | activator-binding proteinC53 | | |
| | | | (C53), mRNA. Length = 1865 | | |
| 173 | 26164 | NM_030836 | Rattus norvegicus leucyl-specific | leucyl-specific aminopeptidase | i |
| | | | aminopeptidase PILS (APPILS), | PILS | |
| | | | mRNA. Length = 5042 | | |
| 174 | 1221 | NM_030845 | Rattus norvegicus gro (Gro1), | gro | General |
| | | | mRNA. Length = 929 | | |
| 175 | 17342 | NM_030873 | Rattus norvegicus profilin II | profilin II | d |
| | | | (Pfn2), mRNA. Length = 1966 | | |
| 176 | 23109 | NM_031000 | Rattus norvegicus aldo-keto | aldo-keto reductase family 1, | c, f, h |
| | | | reductase family 1, member A1 | member A1 (aldehyde reductase) | |
| | | | (aldehyde reductase) (Akr1a1), | | |
| | | | mRNA. Length = 1124 | | |
| 177 | 21165 | NM_031005 | Rattus norvegicus non-muscle | non-muscle alpha-actinin 1 | General |
| | | | alpha-actinin 1 (Actn1), mRNA. | | |
| | | | Length = 2956 | | |
| 178 | 1845 | NM_031010 | Rattus norvegicus arachidonate | arachidonate 12-lipoxygenase | a, b, |
| | | | 12-lipoxygenase (Alox12), | | General |
| | | | mRNA. Length = 2048 | | |
| 178 | 25517 | NM_031010 | Rattus norvegicus arachidonate | arachidonate 12-lipoxygenase | a, b, |
| | | | 12-lipoxygenase (Alox12), | | General |
| | | | mRNA. Length = 2048 | | |
| 179 | 15886 | NM_031035 | Rattus norvegicus GTP-binding | GTP-binding protein (G-alpha-i2) | c, f |
| | | | protein (G-alpha-i2) (Gnai2), | | |
| | | | mRNA. Length = 1748 | | |
| 180 | 15957 | NM_031050 | Rattus norvegicus lumican | lumican | h |
| | | | (Lum), mRNA. Length = 1740 | | |
| 181 | 11849 | NM_031065 | Rattus norvegicus ribosomal | ribosomal protein L10a | h, General |
| | | | protein L10a (Rpl10a), mRNA. | | |
| | | | Length = 710 | | |
| 182 | 12639 | NM_031099 | Rattus norvegicus ribosomal | ribosomal protein L5 | e, General |
| | | | protein L5 (Rpl5), mRNA. | | |
| | | | Length = 1069 | | |
| 183 | 20812 | NM_031100 | Rattus norvegicus ribosomal | ribosomal protein L10 | e |
| | | | protein L10 (Rpl10), mRNA. | | |
| | | | Length = 769 | | |
| 184 | 23854 | NM_031101 | Rattus norvegicus ribosomal | ribosomal protein L13 | General |
| | | | protein L13 (Rpl13), mRNA. | | |
| | | | Length = 722 | | |
| 185 | 16938 | NM_031103 | Rattus norvegicus ribosomal | ribosomal protein L19 | e |
| | | | protein L19 (Rpl19), mRNA. | | |
| | | | Length = 703 | | |
| 186 | 22205 | NM_031105 | Rattus norvegicus large subunit | large subunit ribosomal protein | h |
| | | | ribosomal protein L36a (Rpl36a), | L36a | |
| | | | mRNA. Length = 444 | | |
| 187 | 16847 | NM_031109 | Rattus norvegicus ribosomal | ribosomal protein S10 | g, General |
| | | | protein S10 (Rps10), mRNA. | | |
| | | | Length = 610 | | |
| 188 | 10878 | NM_031110 | Rattus norvegicus ribosomal | ribosomal protein S11 | e, General |
| | | | protein S11 (Rps11), mRNA. | | |
| | | | Length = 534 | | |
| 189 | 20839 | NM_031113 | Rattus norvegicus ribosomal | ribosomal protein S27a | General |
| | | | protein S27a (Rps27a), mRNA. | | |
| | | | Length = 552 | | |
| 190 | 22749 | NM_031116 | Rattus norvegicus small | small inducible cytokine A5 | b |
| | | | inducible cytokine A5 (RANTES) | (RANTES) | |
| | | | (Scya5), mRNA. Length = 279 | | |
| 191 | 1580 | NM_031117 | Rattus norvegicus small nuclear | small nuclear ribonucleoparticle- | h |
| | | | ribonucleoparticle-associated | associated protein (snRNP) mRNA, | |
| | | | protein (snRNP) mRNA, clone | clone Sm51 | |
| | | | Sm51 (Snrpn), mRNA. Length = | | |
| | | | 1428 | | |
| 192 | 13929 | NM_031131 | Rattus norvegicus TGF beta 2 | TGF beta 2 protein | General |
| | | | protein (Tgfb2), mRNA. Length = | | |
| | | | 2880 | | |
| 193 | 15185 | NM_031140 | Rattus norvegicus vimentin | vimentin | c, General |
| | | | (Vim), mRNA. Length = 1796 | | |
| 194 | 1638 | NM_031143 | Rattus norvegicus diacylglycerol | diacylglycerol kinase | General |
| | | | kinase (Dgkz), mRNA. Length = | | |
| | | | 3560 | | |
| 195 | 21624 | NM_031144 | Rattus norvegicus cytoplasmic | cytoplasmic beta-actin | g |
| | | | beta-actin (Actx), mRNA. | | |
| | | | Length = 1128 | | |
| 196 | 15277 | NM_031237 | Rattus norvegicus ubiquitin- | ubiquitin-conjugating enzyme E2D 3 | g, i, |
| | | | conjugating enzyme E2D 3 | (homologous to yeast UBC4/5) | General |
| | | | (homologous to yeast UBC4/5) | | |
| | | | (Ube2d3), mRNA. Length = 1531 | | |
| 197 | 18597 | NM_031325 | Rattus norvegicus UDP-glucose | UDP-glucose dehydrogeanse | General |
| | | | dehydrogeanse (Ugdh), mRNA. | | |
| | | | Length = 2318 | | |
| 198 | 11258 | NM_031327 | Rattus norvegicus cysteine rich | cysteine rich protein 61 | General |
| | | | protein 61 (Cyr61), mRNA. | | |
| | | | Length = 1871 | | |
| 199 | 18654 | NM_031358 | Rattus norvegicus potassium | potassium inwardly-rectifying | i, General |
| | | | inwardly-rectifying channel, | channel, subfamily J, member 11 | |
| | | | subfamily J, member 11 | | |
| | | | (Kcnj11), mRNA. Length = 2227 | | |
| 200 | 12581 | NM_031514 | Rattus norvegicus Janus kinase | Janus kinase 2 (a protein tyrosine | i, General |
| | | | 2 (a protein tyrosine kinase) | kinase) | |
| | | | (Jak2), mRNA. Length = 3731 | | |
| 201 | 20449 | NM_031530 | Rattus norvegicus Small | Small inducible gene JE | g, General |
| | | | inducible gene JE (Scya2), | | |
| | | | mRNA. Length = 780 | | |
| 201 | 20448 | NM_031530 | Rattus norvegicus Small | Small inducible gene JE | g |
| | | | inducible gene JE (Scya2), | | |
| | | | mRNA. Length = 780 | | |
| 202 | 445 | NM_031535 | Rattus norvegicus B cell | B cell lymphoma 2 like, ESTs, | b |
| | | | lymphoma 2 like (Bcl2l), mRNA. | Moderately similar to acetolactate | |
| | | | Length = 1748 | synthase homolog [H. sapiens] | |
| 203 | 4011 | NM_031543 | Rattus norvegicus Cytochrome | Cytochrome P450, subfamily 2e1 | a |
| | | | P450, subfamily 2e1 (ethanol- | (ethanol-inducible) | |
| | | | inducible) (Cyp2e1), mRNA. | | |
| | | | Length = 1624 | | |
| 204 | 18389 | NM_031545 | Rattus norvegicus Brain | Brain natriuretic factor | i, General |
| | | | natriuretic factor (Nppb), mRNA. | | |
| | | | Length = 628 | | |
| 205 | 692 | NM_031557 | Rattus norvegicus Prostaglandin | Prostaglandin I2 (prostacyclin) | h |
| | | | I2 (prostacyclin) synthase | synthase | |
| | | | (Ptgis), mRNA. Length = 1618 | | |
| 206 | 18318 | NM_031561 | Rattus norvegicus CD36 antigen | CD36 antigen (collagen type I | h |
| | | | (collagen type I receptor, | receptor, thrombospondin receptor) | |
| | | | thrombospondin receptor) | | |
| | | | (Cd36), mRNA. Length = 2436 | | |
| 207 | 24219 | NM_031579 | Rattus norvegicus protein | protein tyrosine phosphatase 4a1 | General |
| | | | tyrosine phosphatase 4a1 | | |
| | | | (Ptp4a1), mRNA. Length = 2638 | | |
| 208 | 24235 | NM_031614 | Rattus norvegicus thioredoxin | thioredoxin reductase 1 | General |
| | | | reductase 1 (Txnrd1), mRNA. | | |
| | | | Length = 3360 | | |
| 209 | 567 | NM_031628 | Rattus norvegicus nuclear | nuclear receptor subfamily 4, group | General |
| | | | receptor subfamily 4, group A, | A, member 3 | |
| | | | member 3 (Nr4a3), mRNA. | | |
| | | | Length = 4400 | | |
| 210 | 211 | NM_031664 | Rattus norvegicus solute carrier | solute carrier family 28 (sodium- | c |
| | | | family 28 (sodium-coupled | coupled nucleoside transporter), | |
| | | | nucleoside transporter), member | member 2 | |
| | | | 2 (Slc28a2), mRNA. Length = | | |
| | | | 2881 | | |
| 211 | 18403 | NM_031677 | Rattus norvegicus four and a half | four and a half LIM domains 2 | g |
| | | | LIM domains 2 (Fhl2), mRNA. | | |
| | | | Length = 840 | | |
| 212 | 20743 | NM_031684 | Rattus norvegicus solute carrier | solute carrier family 29 (nucleoside | f, g, |
| | | | family 29 (nucleoside | transporters), member 1 | General |
| | | | transporters), member 1 | | |
| | | | (Slc29a1), mRNA. Length = 1766 | | |
| 213 | 21575 | NM_031698 | Rattus norvegicus ribophorin II | ribophorin II | b |
| | | | (Rpn2), mRNA. Length = 2234 | | |
| 214 | 16918 | NM_031709 | Rattus norvegicus ribosomal | ribosomal protein S12 | e, General |
| | | | protein S12 (Rps12), mRNA. | | |
| | | | Length = 499 | | |
| 215 | 19050 | NM_031719 | Rattus norvegicus chloride | chloride channel current inducer | General |
| | | | channel, nucleotide-sensitive, 1A | | |
| | | | (Clns1a), mRNA. Length = 1399 | | |
| 215 | 19048 | NM_031719 | Rattus norvegicus chloride | chloride channel current inducer | f |
| | | | channel, nucleotide-sensitive, 1A | | |
| | | | (Clns1a), mRNA. Length = 1399 | | |
| 216 | 3549 | NM_031723 | Rattus norvegicus signal | signal peptidase complex (18 kD) | h |
| | | | peptidase complex (18 kD) | | |
| | | | (Spc18), mRNA. Length = 643 | | |
| 217 | 14184 | NM_031776 | Rattus norvegicus guanine | guanine deaminase | General |
| | | | deaminase (Gda), mRNA. | | |
| | | | Length = 1568 | | |
| 217 | 14185 | NM_031776 | Rattus norvegicus guanine | guanine deaminase | General |
| | | | deaminase (Gda), mRNA. | | |
| | | | Length = 1568 | | |
| 218 | 1169 | NM_031789 | Rattus norvegicus NF-E2-related | NF-E2-related factor 2 | a |
| | | | factor 2 (Nfe2l2), mRNA. | | |
| | | | Length = 2307 | | |
| 219 | 17195 | NM_031814 | Rattus norvegicus G protein- | G protein-coupled receptor kinase- | g |
| | | | coupled receptor kinase- | associated ADP ribosylation factor | |
| | | | associated ADP ribosylation | GTPase-activating protein (GIT1) | |
| | | | factor GTPase-activating protein | | |
| | | | (GIT1) (Git1), mRNA. Length = | | |
| | | | 3236 | | |
| 220 | 17535 | NM_031816 | Rattus norvegicus | retinoblastoma binding protein 7 | h |
| | | | retinoblastoma binding protein 7 | | |
| | | | (Rbbp7), mRNA. Length = 1947 | | |
| 221 | 15840 | NM_031817 | Rattus norvegicus osteomodulin | osteomodulin (osteoadherin) | g, General |
| | | | (osteoadherin) (Omd), mRNA. | | |
| | | | Length = 1536 | | |
| 222 | 2655 | NM_031821 | Rattus norvegicus serum- | serum-inducible kinase | i |
| | | | inducible kinase (Snk), mRNA. | | |
| | | | Length = 2781 | | |
| 223 | 3251 | NM_031825 | Rattus norvegicus fibrillin-1 | fibrillin-1 | b |
| | | | (Fbn1), mRNA. Length = 8942 | | |
| 223 | 3252 | NM_031825 | Rattus norvegicus fibrillin-1 | fibrillin-1 | c |
| | | | (Fbn1), mRNA. Length = 8942 | | |
| 224 | 22321 | NM_031832 | Rattus norvegicus IgE binding | IgE binding protein | g, General |
| | | | protein (Lgals3), mRNA. | | |
| | | | Length = 948 | | |
| 225 | 4749 | NM_031834 | Rattus norvegicus | minoxidil sulfotransferase | c, d |
| | | | sulfotransferase family 1A, | | |
| | | | phenol-preferring, member 1 | | |
| | | | (Sult1a1), mRNA. Length = 1227 | | |
| 226 | 15603 | NM_031851 | Rattus norvegicus Prohibitin | Prohibitin | i |
| | | | (Phb), mRNA. Length = 1688 | | |
| 227 | 22300 | NM_031967 | Rattus norvegicus development- | development-related protein | c |
| | | | related protein (AF045564), | | |
| | | | mRNA. Length = 2718 | | |
| 228 | 17735 | NM_031970 | Rattus norvegicus Heat shock 27 | ESTs, Heat shock 27 kDa protein | g, i, |
| | | | kDa protein (Hsp27), | | General |
| | | | mRNA. Length = 787 | | |
| 228 | 17734 | NM_031970 | Rattus norvegicus Heat shock 27 | ESTs, Heat shock 27 kDa protein | g, i, |
| | | | kDa protein (Hsp27), | | General |
| | | | mRNA. Length = 787 | | |
| 228 | 17736 | NM_031970 | Rattus norvegicus Heat shock 27 | ESTs, Heat shock 27 kDa protein | f, g, i |
| | | | kDa protein (Hsp27), | | General |
| | | | mRNA. Length = 787 | | |
| 229 | 18500 | NM_031984 | Rattus norvegicus cerebellar Ca- | cerebellar Ca-binding protein, spot | c |
| | | | binding protein, spot 35 protein | 35 protein | |
| | | | (Calb1), mRNA. Length = 2280 | | |
| 230 | 18899 | NM_031985 | Rattus norvegicus S6 kinase | S6 kinase | a |
| | | | (Rps6kb1), mRNA. Length = | | |
| | | | 2287 | | |
| 231 | 19768 | NM_031986 | Rattus norvegicus syntenin | syntenin | i |
| | | | (Sdcbp), mRNA. Length = 2077 | | |
| 232 | 17829 | NM_033234 | Rattus norvegicus Hemoglobin, | Rat major beta-globin mRNA, | a, c |
| | | | beta (Hbb), mRNA. Length = 620 | complete cds | |
| 232 | 25468 | NM_033234 | Rattus norvegicus Hemoglobin, | | a, b |
| | | | beta (Hbb), mRNA. Length = 620 | | |
| 232 | 25469 | NM_033234 | Rattus norvegicus Hemoglobin, | | a, b, |
| | | | beta (Hbb), mRNA. Length = 620 | | General |
| 232 | 17832 | NM_033234 | Rattus norvegicus Hemoglobin, | Rat major beta-globin mRNA, | a, b, |
| | | | beta (Hbb), mRNA. Length = 620 | complete cds | General |
| 233 | 12364 | NM_033351 | Rattus norvegicus Fc receptor, | Rat MHC class I lgG Fc region | General |
| | | | lgG, alpha chain transporter | receptor large subunit p51 (FcRn) | |
| | | | (Fcgrt), mRNA. Length = 1552 | mRNA, complete cds | |
| 234 | 11714 | NM_033352 | Rattus norvegicus ATP-binding | ESTs | General |
| | | | cassette, sub-family D (ALD), | | |
| | | | member 2 (Abcd2), mRNA. | | |
| | | | Length = 5531 | | |
| 235 | 15867 | NM_053289 | Rattus norvegicus Pancreatitis- | ESTs, Weakly similar to T15062 | General |
| | | | associated protein 1 (Pap1), | hypothetical protein R02D3.2 - | |
| | | | mRNA. Length = 781 | Caenorhabditis elegans | |
| | | | | [C. elegans], Rattus norvegicus | |
| | | | | pancreatitis-associated protein | |
| | | | | precursor (pap) mRNA, complete | |
| | | | | cds | |
| 236 | 4093 | NM_053290 | Rattus norvegicus | R. norvegicus phosphoglycerate | General |
| | | | Phosphoglycerate mutase 1 | mutase B isozyme (PGAM) mRNA, | |
| | | | (Pgam1), mRNA. Length = 1754 | complete cds | |
| 237 | 16886 | NM_053291 | Rattus norvegicus | ESTs, Highly similar to PGK2 RAT | c |
| | | | phosphoglycerate kinase 1 | PHOSPHOGLYCERATE KINASE, | |
| | | | (Pgk1), mRNA. Length = 1675 | TESTIS SPECIFIC [R. norvegicus] | |
| 238 | 14928 | NM_053330 | Rattus norvegicus ribosomal | Rattus norvegicus ribosomal protein | e, h |
| | | | protein L21 (Rpl21), mRNA. | L21 mRNA, complete cds | |
| | | | Length = 554 | | |
| 239 | 1609 | NM_053338 | Rattus norvegicus Ras-related | Rattus norvegicus ras-related | c, General |
| | | | associated with diabetes (Rrad), | protein (rad) mRNA, complete cds | |
| | | | mRNA. Length = 1421 | | |
| 240 | 9353 | NM_053347 | Rattus norvegicus nuclear | Rattus norvegicus LIS1-interacting | General |
| | | | distribution gene E homolog | protein NUDE1 (Nude1) mRNA, | |
| | | | (Aspergillus) (Nude), mRNA. | complete cds | |
| | | | Length = 2153 | | |
| 241 | 6155 | NM_053356 | Rattus norvegicus procollagen, | procollagen, type I, alpha 2 | e |
| | | | type I, alpha 2 (Col1a2), mRNA. | | |
| | | | Length = 4474 | | |
| 241 | 6156 | NM_053356 | Rattus norvegicus procollagen, | procollagen, type I, alpha 2 | e |
| | | | type I, alpha 2 (Col1a2), mRNA. | | |
| | | | Length = 4474 | | |
| 242 | 16017 | NM_053401 | Rattus norvegicus brain | brain expressed X-linked 3 | i |
| | | | expressed X-linked 3 (Bex3), | | |
| | | | mRNA. Length = 519 | | |
| 243 | 2242 | NM_053433 | Rattus norvegicus flavin- | flavin-containing monooxygenase 3 | d, General |
| | | | containing monooxygenase 3 | | |
| | | | (Fmo3), mRNA. Length = 2037 | | |
| 244 | 8085 | NM_053453 | Rattus norvegicus regulator of G- | regulator of G-protein signaling | General |
| | | | protein signaling protein 2 | protein 2 | |
| | | | (Rgs2), mRNA. Length = 1629 | | |
| 245 | 11403 | NM_053464 | Rattus norvegicus spermidine | spermidine synthase | General |
| | | | synthase (Srm), mRNA. | | |
| | | | Length = 1268 | | |
| 246 | 23276 | NM_053467 | Rattus norvegicus integral | integral membrane protein Tmp21-I | General |
| | | | membrane protein Tmp21-I (p23) | (p23) | |
| | | | (Tmp21), mRNA. Length = 1314 | | |
| 247 | 15556 | NM_053483 | Rattus norvegicus karyopherin | karyopherin (importin) alpha 2 | General |
| | | | (importin) alpha 2 (Kpna2), | | |
| | | | mRNA. Length = 1886 | | |
| 248 | 16394 | NM_053485 | Rattus norvegicus calcium | calcium binding protein A6 | General |
| | | | binding protein A6 (calcyclin) | (calcyclin) | |
| | | | (S100a6), mRNA. Length = 291 | | |
| 249 | 18826 | NM_053523 | Rattus norvegicus homocysteine- | Rattus norvegicus SUP mRNA, | c |
| | | | inducible, endoplasmic reticulum | complete cds | |
| | | | stress-inducible, ubiquitin-like | | |
| | | | domain member 1 (Herpud1), | | |
| | | | mRNA. Length = 1857 | | |
| 250 | 14380 | NM_053536 | Rattus norvegicus Kruppel-like | Rattus norvegicus mRNA for | General |
| | | | factor 15 (kidney) (KIf15), mRNA. | Kruppel-like transcription factor, | |
| | | | Length = 2458 | complete cds | |
| 251 | 15708 | NM_053565 | Rattus norvegicus cytokine | Rattus norvegicus suppressor of | General |
| | | | inducible SH2-containing protein | cytokine signaling-3 (SOCS-3) | |
| | | | 3 (Cish3), mRNA. Length = 863 | mRNA, complete cds | |
| 252 | 3050 | NM_053582 | Rattus norvegicus glucocorticoid- | Rattus norvegicus gis5 mRNA for | d, General |
| | | | inducible protein (gis5), mRNA. | glucocorticoid-inducible protein, | |
| | | | Length = 1869 | complete cds | |
| 252 | 3049 | NM_053582 | Rattus norvegicus glucocorticoid- | Rattus norvegicus gis5 mRNA for | d, General |
| | | | inducible protein (gis5), mRNA. | glucocorticoid-inducible protein, | |
| | | | Length = 1869 | complete cds | |
| 253 | 21709 | NM_053596 | Rattus norvegicus endothelin- | Rat mRNA for endothelin- | General |
| | | | converting enzyme 1 (Ece1), | converting enzyme, complete cds | |
| | | | mRNA. Length = 4469 | | |
| 254 | 2103 | NM_053597 | Rattus norvegicus ribosomal | Rattus norvegicus ribosomal protein | e, h |
| | | | protein S27 (Rps27), mRNA. | S271 (S27-1) mRNA, complete cds | |
| | | | Length = 336 | | |
| 255 | 7580 | NM_053603 | Rattus norvegicus chloride | Rattus norvegicus chloride | General |
| | | | intracellular channel 5 (Clic5), | intracellular channel 5 mRNA, | |
| | | | mRNA. Length = 2140 | complete cds | |
| 256 | 20243 | NM_053615 | Rattus norvegicus casein kinase | Rattus norvegicus casein kinase 1 | h |
| | | | 1, alpha 1 (Csnk1a1), mRNA. | alpha (CKIa) mRNA, complete cds | |
| | | | Length = 978 | | |
| 257 | 13005 | NM_053623 | Rattus norvegicus fatty acid- | Rattus norvegicus mRNA for Acyl- | General |
| | | | Coenzyme A ligase, long chain 4 | CoA synthetase, complete cds | |
| | | | (Facl4), mRNA. Length = 4862 | | |
| 258 | 18644 | NM_053648 | Rattus norvegicus beta-carotene | Rattus norvegicus mRNA for beta- | c |
| | | | 15, 15′-dioxygenase (Bcdo), | carotene 15, 15′-dioxygenase, | |
| | | | mRNA. Length = 2207 | complete cds | |
| 259 | 6684 | NM_053703 | Rattus norvegicus mitogen- | Rattus norvegicus mitogen- | General |
| | | | activated protein kinase kinase 6 | activated protein kinase kinase 6 | |
| | | | (Map2k6), mRNA. Length = 1690 | (Mkk6) mRNA, complete cds | |
| 260 | 25379 | NM_053713 | Rattus norvegicus Kruppel-like | | d, General |
| | | | factor 4 (gut) (Klf4), mRNA. | | |
| | | | Length = 2393 | | |
| 260 | 22411 | NM_053713 | Rattus norvegicus Kruppel-like | ESTs | d |
| | | | factor 4 (gut) (Klf4), mRNA. | | |
| | | | Length = 2393 | | |
| 261 | 1218 | NM_053748 | Rattus norvegicus | Rattus norvegicus mRNA for | e, General |
| | | | dipeptidylpeptidase III (Dpp3), | dipeptidyl peptidase III, complete | |
| | | | mRNA. Length = 2632 | cds | |
| 262 | 9059 | NM_053783 | Rattus norvegicus interferon | Rattus norvegicus interferon- | g, General |
| | | | gamma receptor (Ifngr), mRNA. | gamma receptor mRNA, complete | |
| | | | Length = 1860 | cds | |
| 263 | 15103 | NM_053814 | Rattus norvegicus Rho | Rattus norvegicus P116RIP mRNA, | h |
| | | | interacting protein 3 (Rhoip3), | complete cds | |
| | | | mRNA. Length = 3286 | | |
| 264 | 15002 | NM_053819 | Rattus norvegicus tissue inhibitor | Rattus norvegicus tissue inhibitor of | f, g, |
| | | | of metalloproteinase 1 (Timp1), | metalloproteinase-1 (TIMP1), | General |
| | | | mRNA. Length = 740 | mRNA, complete cds | |
| 264 | 15003 | NM_053819 | Rattus norvegicus tissue inhibitor | Rattus norvegicus tissue inhibitor of | f, g, |
| | | | of metalloproteinase 1 (Timp1), | metalloproteinase-1 (TIMP1), | General |
| | | | mRNA. Length = 740 | mRNA, complete cds | |
| 265 | 17155 | NM_053835 | Rattus norvegicus clathrin, light | Rat clathrin light chain (LCB2) | e |
| | | | polypeptide (Lcb) (Cltb), mRNA. | mRNA, complete cds, Rat clathrin | |
| | | | Length = 982 | light chain (LCB3) mRNA, complete | |
| | | | | cds | |
| 266 | 20868 | NM_053843 | Rattus norvegicus Fc receptor, | Rat Fc-gamma receptor mRNA, | General |
| | | | IgG, low affinity III (Fcgr3), | complete cds | |
| | | | mRNA. Length = 1318 | | |
| 266 | 20869 | NM_053843 | Rattus norvegicus Fc receptor, | Rat Fc-gamma receptor mRNA, | General |
| | | | IgG, low affinity III (Fcgr3), | complete cds | |
| | | | mRNA. Length = 1318 | | |
| 267 | 1571 | NM_053857 | Rattus norvegicus eukaryotic | Rattus norvegicus Sprague/Dawley | d, General |
| | | | translation initiation factor 4E | PHAS-I mRNA, complete cds | |
| | | | binding protein 1 (Eif4ebp1), | | |
| | | | mRNA. Length = 843 | | |
| 268 | 18358 | NM_053864 | Rattus norvegicus valosin- | Rattus norvegicus transitional | h |
| | | | containing protein (Vcp), mRNA. | endoplasmic reticulum ATPase | |
| | | | Length = 2870 | mRNA, complete cds | |
| 269 | 17728 | NM_053867 | Rattus norvegicus tumor protein, | Rattus norvegicus lens epithelial | a, b |
| | | | translationally-controlled 1 | protein mRNA, complete cds | |
| | | | (Tpt1), mRNA. Length = 794 | | |
| 270 | 19781 | NM_053883 | Rattus norvegicus dual | Rattus norvegicus dual-specificity | General |
| | | | specificity phosphatase 6 | protein tyrosine phosphatase | |
| | | | (Dusp6), mRNA. Length = 2104 | (rVH6) mRNA, complete cds | |
| 271 | 385 | NM_053885 | Rattus norvegicus arginine- | Rattus norvegicus atrophin-1 | i, General |
| | | | glutamic acid dipeptide (RE) | related protein (rARP) mRNA, | |
| | | | repeats (Rere), mRNA. Length = | complete cds | |
| | | | 6659 | | |
| 272 | 1288 | NM_053949 | Rattus norvegicus potassium | Rattus norvegicus mRNA for | General |
| | | | voltage-gated channel, subfamily | potassium channel r-ERG | |
| | | | H (eag-related), member 2 | | |
| | | | (Kcnh2), mRNA. Length = 3889 | | |
| 273 | 19774 | NM_053961 | Rattus norvegicus endoplasmic | ESTs, Moderately similar to S12207 | a |
| | | | retuclum protein 29 (Erp29), | hypothetical protein [M. musculus] | |
| | | | mRNA. Length = 4529 | | |
| 274 | 16546 | NM_053965 | Rattus norvegicus solute carrier | R. norvegicus mRNA for | General |
| | | | family 25 (carnitine/acylcarnitine | carnitine/acylcarnitine carrier | |
| | | | translocase), member 20 | protein | |
| | | | (Slc25a20), mRNA. Length = | | |
| | | | 1231 | | |
| 275 | 19544 | NM_053982 | Rattus norvegicus ribosomal | EST, Moderately similar to | General |
| | | | protein S15a (Rps15a), mRNA. | RS1A_HUMAN 40S RIBOSOMAL | |
| | | | Length = 449 | PROTEIN S15A [R. norvegicus] | |
| 276 | 15642 | NM_053985 | Rattus norvegicus H3 histone, | R. norvegicus mRNA for histone | General |
| | | | family 3B (H3f3b), mRNA. | H3.3 | |
| | | | Length = 1107 | | |
| 277 | 17739 | NM_053995 | Rattus norvegicus 3- | Sprague-Dawley D-beta- | e |
| | | | hydroxybutyrate dehydrogenase | hydroxybutyrate dehydrogenase | |
| | | | (heart, mitochondrial) (Bdh), | mRNA, complete cds | |
| | | | mRNA. Length = 1420 | | |
| 278 | 17329 | NM_054008 | Rattus norvegicus Rgc32 protein | Rattus norvegicus RGC-32 (RGC- | General |
| | | | (Rgc32), mRNA. Length = 889 | 32) mRNA, complete cds | |
| 279 | 19658 | NM_057103 | Rattus norvegicus A kinase | Rattus norvegicus PKC binding | General |
| | | | (PRKA) anchor protein (gravin) | protein and substrate mRNA, | |
| | | | 12 (Akap12), mRNA. Length = | complete cds | |
| | | | 5236 | | |
| 280 | 968 | NM_057133 | Rattus norvegicus nuclear | Rattus norvegicus mRNA for small | h |
| | | | receptor subfamily 0, group B, | heterodimer partner homolog, | |
| | | | member 2 (Nr0b2), mRNA. | complete cds | |
| | | | Length = 1111 | | |
| 281 | 2413 | NM_057141 | Rattus norvegicus | Rattus norvegicus mRNA for dC- | e, h |
| | | | heterogeneous nuclear | stretch binding protein (CSBP), | |
| | | | ribonucleoprotein K (Hnrpk), | complete cds | |
| | | | mRNA. Length = 2563 | | |
| 281 | 2416 | NM_057141 | Rattus norvegicus | Rattus norvegicus mRNA for dC- | d |
| | | | heterogeneous nuclear | stretch binding protein (CSBP), | |
| | | | ribonucleoprotein K (Hnrpk), | complete cds | |
| | | | mRNA. Length = 2563 | | |
| 282 | 1892 | NM_057144 | Rattus norvegicus cysteine-rich | R. norvegicus mRNA for muscle LIM | g, i, |
| | | | protein 3 (Csrp3), mRNA. | protein | General |
| | | | Length = 853 | | |
| 283 | 17477 | NM_057194 | Rattus norvegicus phospholipid | Rattus norvegicus phospholipid | g, General |
| | | | scramblase 1 (Plscr1), mRNA. | scramblase PLSCR mRNA, | |
| | | | Length = 1569 | complete cds | |
| 284 | 14126 | NM_057208 | Rattus norvegicus tropomyosin | Rattus norvegicus tropomyosin non- | g |
| | | | 3, gamma (Tpm3), mRNA. | muscle isoform NM1 (TPM-gamma) | |
| | | | Length = 1101 | mRNA, complete cds, Rattus | |
| | | | | norvegicus tropomyosin non-muscle | |
| | | | | isoform NM3 (TPM-gamma) mRNA, | |
| | | | | complete cds | |
| 285 | 8641 | NM_057211 | Rattus norvegicus Kruppel-like | Rattus norvegicus mRNA for BTE | c |
| | | | factor 9 (Klf9), mRNA. Length = | binding protein, complete cds | |
| | | | 2721 | | |
| 286 | 10498 | NM_078617 | Rattus norvegicus ribosomal | R. norvegicus (Sprague-Dawley) | e, f |
| | | | protein S23 (Rps23), mRNA. | ribosomal protein S23 mRNA | |
| | | | Length = 432 | | |
| 287 | 20392 | NM_080477 | Rattus norvegicus 6- | R. norvegicus mRNA for 6- | General |
| | | | Phosphofructo-2-kinase/fructose- | phosphofructo-2-kinase/6- | |
| | | | 2,6-bisphosphatase 2 (heart) | phosphofructo-2-kinase exons | |
| | | | (Pfkfb2), mRNA. Length = 2537 | 1a′, 1b, 1c, 1d | |
| 288 | 23033 | NM_080888 | Rattus norvegicus | ESTs, Highly similar to NIPL | b, General |
| | | | BCL2/adenovirus E1B 19 kDa- | MOUSE BCL2/ADENOVIRUS E1B | |
| | | | interacting protein 3-like (Bnip3l), | 19-KDA PROTEIN-INTERACTING | |
| | | | mRNA. Length = 3219 | PROTEIN 3 LIKE [M. musculus] | |
| 289 | 15843 | NM_080910 | Rattus norvegicus | Rat AIRC mRNA for AIR | h |
| | | | phosphoribosylaminoimidazole | carboxylase-SAICAR synthetase, | |
| | | | carboxylase, | complete cds | |
| | | | phosphoribosylaminoribosylamin | | |
| | | | oimidazole succinocarboxamide | | |
| | | | synthetase (Paics), mRNA. | | |
| | | | Length = 1329 | | |
| 290 | 21391 | NM_130416 | Rattus norvegicus annexin A7 | ESTs, Weakly similar to ANX4 RAT | General |
| | | | (Anxa7), mRNA. Length = 2912 | ANNEXIN IV [R. norvegicus] | |
| 291 | 25094 | X77117 | | | c |
| 292 | 11663 | AA800803 | | ESTs, Weakly similar to | General |
| | | | | YNP5_CAEEL HYPOTHETICAL | |
| | | | | 28.3 KD PROTEIN T05G5.5 IN | |
| | | | | CHROMOSOME III [C. elegans] | |
| 293 | 25717 | X61296 | | | i |
| 294 | 5292 | AI171607 | | ESTs | d, General |
| 295 | 23998 | AI230578 | | ESTs | General |
| 296 | 5186 | AA925674 | | ESTs | d, General |
| 297 | 1754 | AA817837 | | ESTs | General |
| 298 | 6438 | AA819269 | | ESTs | General |
| 299 | 6059 | AI178245 | | ESTs, Moderately similar to | b, General |
| | | | | C17orf1 protein [H. sapiens] | |
| 300 | 18663 | U95052 | | ESTs, Weakly similar to S49172 | h |
| | | | | translation initiation factor elF-4 | |
| | | | | gamma [H. sapiens] | |
| 301 | 7414 | AI137586 | | ESTs, Highly similar to | g, General |
| | | | | IMB3_HUMAN | |
| | | | | IMPORTIN BETA-3 | |
| | | | | SUBUNIT [H. sapiens] | |
| 302 | 10019 | AI178756 | | ESTs | d, h |
| 303 | 6226 | AA818521 | | ESTs | General |
| 304 | 15571 | M27207 | | R. norvegicus mRNA for collagen | e, h |
| | | | | alpha1 type I | |
| 305 | 15089 | AI009752 | | ESTs | g |
| 306 | 17214 | AI639008 | | ESTs | a |
| 307 | 6073 | AA818818 | | EST | a |
| 308 | 17781 | AI175474 | | ESTs | a |
| 309 | 22386 | AA800844 | | ESTs, Moderately similar to LYOX | e |
| | | | | RAT PROTEIN-LYSINE 6- | |
| | | | | OXIDASE PRECURSOR | |
| | | | | [R. norvegicus] | |
| 310 | 24765 | AA900050 | | ESTs, Highly similar to | h |
| | | | | R37A_HUMAN 60S RIBOSOMAL | |
| | | | | PROTEIN L37 [R. norvegicus] | |
| 311 | 20984 | D90109 | Acyl CoA synthetase, long chain | Acyl CoA synthetase, long chain | h |
| 312 | 22873 | AA923945 | | ESTs, Weakly similar to | i, General |
| | | | | procollagen-lysine 5-dioxygenase | |
| | | | | [R. norvegicus] | |
| 313 | 2341 | AI012144 | | ESTs | a, General |
| 314 | 4861 | AA901290 | | ESTs | General |
| 315 | 22796 | AI072213 | | ESTs | General |
| 316 | 15935 | AA866345 | | ESTs | General |
| 317 | 16038 | AI177372 | | ESTs, Moderately similar to MPL3 | h |
| | | | | RAT MICROTUBULE- | |
| | | | | ASSOCIATED PROTEINS 1A/1B | |
| | | | | LIGHT CHAIN 3 [R. norvegicus] | |
| 318 | 15212 | AI229753 | ADP-ribosylation factor 2 | ADP-ribosylation factor 2 | d, General |
| 319 | 23157 | AI172489 | | ESTs, Moderately similar to STRI | a |
| | | | | RAT STRIATIN [R. norvegicus] | |
| 320 | 4710 | AI168973 | | ESTs, Weakly similar to phosducin- | General |
| | | | | like protein [R. norvegicus] | |
| 321 | 22697 | AA945996 | | ESTs | b, General |
| 322 | 15892 | AI179988 | | ESTs | a, General |
| 323 | 11404 | AI237002 | spermidine synthase | spermidine synthase | g, General |
| 324 | 23596 | AI105435 | HMm: glutaryl-Coenzyme A | ESTs, Highly similar to GCDH | c, General |
| | | | dehydrogenase | MOUSE GLUTARYL-COA | |
| | | | | DEHYDROGENASE PRECURSOR | |
| | | | | [M. musculus] | |
| 325 | 15841 | AI103465 | | ESTs, Moderately similar to | d, g, |
| | | | | ribonuclease P protein subunit p29 | General |
| | | | | [H. sapiens] | |
| 326 | 7220 | AI013098 | | ESTs | d |
| 327 | 20647 | AI639402 | | ESTs | b |
| 328 | 10829 | AI044467 | | EST | c, f |
| 329 | 22043 | AA943738 | | ESTs | f |
| 330 | 2936 | AI229843 | | ESTs, Highly similar to TNF- | General |
| | | | | induced protein GG2-1 [H. sapiens] | |
| 331 | 26133 | AI009950 | | | e |
| 332 | 5867 | AA858953 | HHs: asparaginyl-tRNA | ESTs, Highly similar to | General |
| | | | synthetase | SYN_HUMAN ASPARAGINYL- | |
| | | | | TRNA SYNTHETASE, | |
| | | | | CYTOPLASMIC [H. sapiens] | |
| 333 | 3278 | AI008988 | | ESTs, Highly similar to breakpoint | General |
| | | | | cluster region protein [H. sapiens] | |
| 334 | 21101 | AI175789 | smooth muscle alpha-actin | smooth muscle alpha-actin | g |
| 335 | 12613 | H31620 | | ESTs, Highly similar to hypothetical | General |
| | | | | protein [H. sapiens] | |
| 336 | 6927 | AI010542 | | ESTs | h |
| 337 | 8789 | AI070073 | | ESTs | h |
| 338 | 22995 | AI232047 | | ESTs | General |
| 339 | 5129 | AA925335 | | ESTs | d |
| 340 | 12462 | AI175421 | | ESTs | General |
| 341 | 1760 | U25281 | | Rattus norvegicus SH3 domain | General |
| | | | | binding protein (CR16) mRNA, | |
| | | | | complete cds | |
| 342 | 24521 | AA945636 | | ESTs, Highly similar to 60S ACIDIC | e |
| | | | | RIBOSOMAL PROTEIN P1 | |
| | | | | [R. norvegicus] | |
| 343 | 25354 | L13025 | | | e |
| 344 | 24272 | AA955948 | | Rattus norvegicus | General |
| | | | | deoxyribonuclease II mRNA, | |
| | | | | complete cds | |
| 345 | 18160 | AA799448 | | ESTs | h |
| 346 | 11738 | AI104213 | | ESTs | General |
| 347 | 15644 | AI010256 | | R. norvegicus mRNA for histone | General |
| | | | | H3.3 | |
| 348 | 10971 | AI073212 | | ESTs | e |
| 349 | 23578 | AA955042 | | ESTs, Weakly similar to folate | General |
| | | | | binding protein [R. norvegicus] | |
| 350 | 6217 | AI230381 | | ESTs | General |
| 351 | 21514 | AA851660 | | ESTs | General |
| 352 | 16909 | AA942704 | | ESTs, Moderately similar to SUR2 | e |
| | | | | MOUSE SURFEIT LOCUS | |
| | | | | PROTEIN 2 [M. musculus] | |
| 353 | 21284 | AI170842 | | ESTs, Weakly similar to CARP | General |
| | | | | [R. norvegicus] | |
| 354 | 23645 | AI144653 | | Rattus norvegicus G protein-binding | General |
| | | | | protein CRFG (Crfg) mRNA, | |
| | | | | complete cds | |
| 355 | 8230 | AI059117 | | ESTs | General |
| 356 | 9720 | AI071727 | | EST | g |
| 357 | 24012 | AA957335 | | ESTs | a, i |
| 358 | 14737 | AI008416 | | ESTs | g, General |
| 359 | 7308 | AA818407 | | ESTs | b, General |
| 360 | 6102 | AI228335 | | ESTs | General |
| 361 | 19728 | AI170394 | | ESTs | c |
| 362 | 6568 | AA817891 | | ESTs | General |
| 363 | 20891 | AI169337 | | ESTs, Highly similar to CGI-117 | General |
| | | | | protein [H. sapiens] | |
| 364 | 11375 | AI237594 | | ESTs, Weakly similar to G01614 | b, General |
| | | | | zinc finger protein 127 [H. sapiens] | |
| 365 | 1483 | U34843 | | Rattus norvegicus cell cycle | i |
| | | | | progression related D123 mRNA, | |
| | | | | complete cds | |
| 366 | 20684 | H32977 | | ESTs | General |
| 367 | 3959 | AA901338 | | ESTs, Highly similar to | g, General |
| | | | | IF2B_HUMAN EUKARYOTIC | |
| | | | | TRANSLATION INITIATION | |
| | | | | FACTOR 2 BETA SUBUNIT | |
| | | | | [H. sapiens] | |
| 368 | 19518 | AA800817 | | ESTs | a |
| 369 | 22445 | AA944530 | | ESTs, Highly similar to AF134238 1 | f |
| | | | | PL6 protein [M. musculus] | |
| 370 | 16312 | AA875032 | | ESTs | c, General |
| 371 | 19255 | M15562 | | Rat (diabetic BB) MHC class II | b, General |
| | | | | alpha chain RT1.D alpha (u) | |
| 372 | 4048 | AA851814 | | Rattus norvegicus osteoactivin | General |
| | | | | mRNA, complete cds | |
| 373 | 7471 | AI012379 | | ESTs | General |
| 374 | 21351 | AA945932 | Annexin III (Lipocortin III) | Annexin III (Lipocortin III) | d, General |
| 375 | 23963 | AA957139 | | ESTs | General |
| 376 | 9658 | AA943748 | | ESTs | d, General |
| 377 | 14384 | AI177096 | HMm: adenine phosphoribosyl | Rat adenine | c |
| | | | transferase | phosphoribosyltransferase (APRT) | |
| | | | | gene, complete cds | |
| 378 | 5531 | AI145859 | | ESTs | d |
| 379 | 3324 | AA997981 | | ESTs | e |
| 380 | 13083 | H31665 | | ESTs | d |
| 381 | 18400 | AA799991 | | ESTs | a, f, |
| | | | | | General |
| 382 | 15281 | D13623 | | ESTs | General |
| 383 | 22578 | AA924105 | | ESTs, Weakly similar to SY03 RAT | g |
| | | | | SMALL INDUCIBLE | |
| | | | | CYTOKINE A3 | |
| | | | | PRECURSOR [R. norvegicus] | |
| 384 | 12752 | AI012146 | | ESTs | General |
| 385 | 6606 | AI012308 | | ESTs | f, g, |
| | | | | | General |
| 386 | 11936 | AI228030 | | ESTs, Highly similar to SAP18 | h |
| | | | | [M. musculus] | |
| 387 | 2539 | AI111960 | | ESTs, Weakly similar to FKB5 | General |
| | | | | MOUSE 51 KDA FK506-BINDING | |
| | | | | PROTEIN [M. musculus] | |
| 388 | 6353 | AI008442 | PCTAIRE-1 protein kinase, | PCTAIRE-1 protein kinase, | f |
| | | | alternatively spliced | alternatively spliced | |
| 389 | 15786 | AI013924 | | ESTs | General |
| 390 | 16596 | AI102486 | | ESTs, Weakly similar to S37583 | General |
| | | | | RING finger protein rfp - mouse | |
| | | | | [M. musculus] | |
| 391 | 18581 | AI176160 | | ESTs | General |
| 392 | 4948 | AA924428 | | ESTs | h |
| 393 | 12098 | AI237075 | | ESTs | d |
| 394 | 20073 | AI639152 | | EST, Weakly similar to A1AT RAT | b, General |
| | | | | ALPHA-1-ANTIPROTEINASE | |
| | | | | PRECURSOR [R. norvegicus] | |
| 395 | 18456 | D00688 | | ESTs, Highly similar to 1903159A | General |
| | | | | monoamine oxidase A | |
| | | | | [R. norvegicus] | |
| 396 | 11208 | AI237586 | | ESTs, Moderately similar to INIB | General |
| | | | | RAT INTERFERON-INDUCIBLE | |
| | | | | PROTEIN [R. norvegicus] | |
| 397 | 8314 | AI059386 | | ESTs | General |
| 398 | 1893 | X51529 | phospholipase A2, group IIA | phospholipase A2, group IIA | General |
| | | | (platelets, synovial fluid) | (platelets, synovial fluid) | |
| 399 | 10821 | AI043999 | | ESTs | e |
| 400 | 20983 | AI044900 | Acyl CoA synthetase, long chain | Acyl CoA synthetase, long chain | General |
| 401 | 18472 | AI168975 | | ESTs | General |
| 402 | 21592 | AI102065 | | ESTs, Highly similar to AF117613 1 | General |
| | | | | heme-binding protein [M. musculus] | |
| 403 | 8665 | AI071965 | Heat shock protein 70-1 | ESTs, Moderately similar to T17342 | General |
| | | | | hypothetical protein | |
| | | | | DKFZp586K1924.1 | |
| | | | | [H. sapiens], Heat shock protein 70-1 | |
| 404 | 11066 | AI071602 | | ESTs | h |
| 405 | 19452 | AA964500 | | EST | e, f |
| 406 | 6509 | AI232361 | | ESTs | General |
| 407 | 22266 | AA945601 | | ESTs | a |
| 408 | 2742 | AI070173 | | ESTs | g, i, |
| | | | | | General |
| 409 | 244 | U38376 | | EST, Weakly similar to actin- | General |
| | | | | filament binding protein Frabin | |
| | | | | [R. norvegicus], Rattus norvegicus | |
| | | | | cytosolic phospholipase A2 mRNA, | |
| | | | | complete cds | |
| 410 | 4238 | AI234816 | | ESTs, Highly similar to | b |
| | | | | Y103_HUMAN HYPOTHETICAL | |
| | | | | PROTEIN KIAA0103 [H. sapiens] | |
| 411 | 9970 | AI045198 | | ESTs | General |
| 412 | 6550 | AA817947 | | ESTs | h |
| 413 | 5193 | AA925693 | | EST | General |
| 414 | 22175 | AA818999 | | ESTs, Highly similar to zinc finger | General |
| | | | | protein [H. sapiens] | |
| 415 | 16538 | AA799449 | | ESTs, Weakly similar to | h |
| | | | | nucleosome assembly protein | |
| | | | | [R. norvegicus] | |
| 416 | 1830 | AI009002 | | Rat mRNA for cdc25B, complete | a, b |
| | | | | cds | |
| 417 | 17485 | AI012352 | | ESTs | General |
| 418 | 3982 | AI175100 | | ESTs | General |
| 419 | 3965 | AI112118 | | ESTs | General |
| 420 | 5969 | AI102520 | | ESTs, Moderately similar to | c, General |
| | | | | AF161588 1 GABA-A receptor- | |
| | | | | associated protein [R. norvegicus] | |
| 421 | 15088 | AI232613 | | ESTs, Highly similar to AF151886 1 | General |
| | | | | CGI-128 protein [H. sapiens] | |
| 422 | 25691 | X53504 | | | e |
| 423 | 2984 | AA997015 | | ESTs | General |
| 424 | 11708 | AI171807 | | ESTs | General |
| 425 | 23187 | AI144591 | | ESTs | General |
| 426 | 16818 | AI072906 | | ESTs | a |
| 427 | 19298 | AI236338 | | ESTs, Weakly similar to NHPX RAT | General |
| | | | | NHP2/RS6 FAMILY PROTEIN | |
| | | | | YEL026W HOMOLOG | |
| | | | | [R. norvegicus] | |
| 428 | 14345 | AA944773 | | ESTs | e |
| 429 | 10109 | X58465 | Ribosomal protein S5 | Ribosomal protein S5 | e |
| 430 | 5596 | AI044747 | | ESTs | e, General |
| 431 | 14989 | AI177366 | Integrin, beta 1 | Integrin, beta 1 | f, i, General |
| 432 | 17320 | AI178069 | | ESTs | General |
| 433 | 13646 | X62166 | | ESTs, Highly similar to RL3 RAT | General |
| | | | | 60S RIBOSOMAL PROTEIN L3 | |
| | | | | [R. norvegicus] | |
| 434 | 18611 | X58200 | ribosomal protein L23 | ESTs, Highly similar to | e, General |
| | | | | RL23_HUMAN 60S RIBOSOMAL | |
| | | | | PROTEIN L23 [R. norvegicus] | |
| 435 | 21671 | AA900295 | | Rattus norvegicus placenta growth | General |
| | | | | factor (PIGF) mRNA, complete cds | |
| 436 | 9452 | AA955206 | | ESTs | General |
| 437 | 20901 | AA942706 | | ESTs | d |
| 438 | 21985 | AA943273 | | ESTs | a |
| 439 | 7168 | AI012739 | | ESTs | General |
| 440 | 12195 | AA899121 | | ESTs, Weakly similar to RanBPM | General |
| | | | | [H. sapiens] | |
| 441 | 15929 | AA849740 | | ESTs, Highly similar to CGI-126 | General |
| | | | | protein [H. sapiens] | |
| 442 | 16322 | AA866240 | | EST | c |
| 443 | 18047 | AI171359 | | ESTs | c |
| 444 | 15966 | AA923994 | | ESTs, Highly similar to | b |
| | | | | DRG2_HUMAN | |
| | | | | DEVELOPMENTALLY | |
| | | | | REGULATED GTP-BINDING | |
| | | | | PROTEIN 2 [H. sapiens] | |
| 445 | 13251 | AI179264 | | ESTs, Moderately similar to LZIP-1 | General |
| | | | | and LZIP-2 [M. musculus] | |
| 446 | 18095 | AI177482 | SH-PTP2 protein tyrosine | SH-PTP2 protein tyrosine | i, General |
| | | | phosphatase, non-receptor type | phosphatase, non-receptor type 11 | |
| | | | 11 | | |
| 447 | 16053 | AI228596 | | ESTs, Weakly similar to T16757 | General |
| | | | | hypothetical protein R144.3 - | |
| | | | | Caenorhabditis elegans [C. elegans] | |
| 448 | 10684 | AA849687 | | ESTs, Weakly similar to | e, f |
| | | | | ALZHEIMER'S DISEASE | |
| | | | | AMYLOID A4 PROTEIN | |
| | | | | HOMOLOG PRECURSOR | |
| | | | | [R. norvegicus] | |
| 449 | 22691 | AI177967 | | ESTs, Weakly similar to TGFB RAT | General |
| | | | | LATENT TRANSFORMING | |
| | | | | GROWTH FACTOR BETA | |
| | | | | BINDING PROTEIN 1 | |
| | | | | PRECURSOR [R. norvegicus] | |
| 450 | 15438 | AI179399 | collagen type V, alpha 2 | collagen type V, alpha 2 | e |
| 451 | 20975 | AA799599 | | ESTs | f, General |
| 452 | 15048 | M24542 | HHs: ubiquinol-cytochrome c | Rat Rieske iron-sulfur protein | c, i |
| | | | reductase, Rieske iron-sulfur | mRNA, complete cds | |
| | | | polypeptide 1 | | |
| 453 | 19379 | AI102711 | | ESTs, Moderately similar to RL3 | General |
| | | | | RAT 60S RIBOSOMAL PROTEIN | |
| | | | | L3 [R. norvegicus] | |
| 454 | 12583 | AI234251 | | ESTs | General |
| 455 | 19480 | AA944442 | | ESTs, Weakly similar to DRAL | c |
| | | | | [R. norvegicus] | |
| 456 | 7700 | AI105383 | | ESTs, Weakly similar to T19707 | General |
| | | | | hypothetical protein C34C6.5 - | |
| | | | | Caenorhabditis elegans [C. elegans] | |
| 457 | 21996 | AA946036 | | ESTs | f |
| 458 | 26148 | AI013396 | | | General |
| 459 | 9551 | AI013558 | | ESTs | d |
| 460 | 23632 | AI237273 | | ESTs | d, f, |
| | | | | | General |
| 461 | 15292 | AF012714 | multiple inositol polyphosphate | multiple inositol polyphosphate | c |
| | | | histidine phosphatase 1 | histidine phosphatase 1 | |
| 462 | 6632 | AI235277 | | ESTs | General |
| 463 | 18406 | AI236168 | | ESTs | General |
| 464 | 11865 | AA866383 | | ESTs | General |
| 465 | 20461 | AI639350 | | ESTs | I, General |
| 466 | 11728 | AI007884 | | ESTs | d |
| 467 | 6562 | AI177734 | | ESTs | d, General |
| 468 | 3046 | AA997706 | | ESTs | h |
| 469 | 25730 | X63369 | | | General |
| 470 | 23166 | AA859954 | | ESTs | General |
| 471 | 3271 | AI010303 | | ESTs | e |
| 472 | 4626 | AI104744 | | ESTs | General |
| 473 | 6430 | AI232319 | | ESTs, Highly similar to JC5221 | General |
| | | | | stomatin - mouse [M. musculus] | |
| 474 | 6262 | AI177125 | | ESTs | General |
| 475 | 8786 | AA996993 | | EST, Moderately similar to Prer | d |
| | | | | protein [H. sapiens], ESTs, Highly | |
| | | | | similar to rer [M. musculus] | |
| 476 | 12946 | AI228291 | | ESTs | General |
| 477 | 23015 | AI227724 | | ESTs | f, General |
| 478 | 15949 | AA963780 | | ESTs | i |
| 479 | 5458 | AI009756 | ALG-2 interacting protein 1 | ALG-2 interacting protein 1 | g, i, |
| | | | | | General |
| 480 | 11726 | AA849518 | | ESTs | d |
| 481 | 22197 | AI178527 | | ESTs | General |
| 482 | 21311 | AI178688 | | ESTs | General |
| 483 | 16092 | AI009012 | | ESTs, Highly similar to Wdr1 | g, i, |
| | | | | protein [M. musculus] | General |
| 484 | 15283 | AA858548 | | ESTs | i, General |
| 485 | 23601 | AA955126 | | EST, Moderately similar to MYBB | f |
| | | | | MOUSE MYB-RELATED | |
| | | | | PROTEIN | |
| | | | | B [M. musculus] | |
| 486 | 2418 | AI013790 | | ESTs, Moderately similar to T08802 | e |
| | | | | hypothetical protein | |
| | | | | DKFZp586D0623.1 [H. sapiens] | |
| 487 | 3214 | AI235483 | | ESTs | General |
| 488 | 20056 | AI639504 | | ESTs, Weakly similar to T13607 | General |
| | | | | hypothetical protein EG: 87B1.3 - | |
| | | | | fruit fly [D.melanogaster] | |
| 489 | 6431 | AA859085 | | ESTs | General |
| 490 | 17145 | M38566 | Serine protease inhibitor | Serine protease inhibitor | i |
| 491 | 15492 | AA892376 | | ESTs | h |
| 492 | 23687 | AI137958 | | ESTs | e |
| 493 | 17224 | AI179883 | | ESTs, Moderately similar to | c |
| | | | | Sid393p [M. musculus] | |
| 494 | 3682 | AA998964 | | ESTs | c |
| 495 | 21818 | AF036537 | | Rattus norvegicus homocysteine | c |
| | | | | respondent protein HCYP2 mRNA, | |
| | | | | complete cds | |
| 496 | 3434 | AI232014 | | ESTs | General |
| 497 | 15084 | AA799397 | | ESTs | a |
| 498 | 23013 | AI230743 | actin-related protein 3 | actin-related protein 3 | General |
| 429 | 25702 | X58465 | | | e |
| 499 | 3817 | AA926328 | Tyrosine 3- | Tyrosine 3- | General |
| | | | monooxygenase/tryptophan 5- | monooxygenase/tryptophan 5- | |
| | | | monooxygenase activation | monooxygenase activation protein, | |
| | | | protein, zeta polypeptide | zeta polypeptide | |
| 500 | 3669 | AA957535 | | ESTs | g, i, |
| | | | | | General |
| 501 | 11331 | AI145556 | | ESTs | d |
| 502 | 15558 | AA875537 | | ESTs, Weakly similar to A46241 | General |
| | | | | interferon response element-binding | |
| | | | | factor IREBF-2 - mouse | |
| | | | | [M. musculus] | |
| 503 | 23409 | AA956294 | | ESTs | General |
| 504 | 4652 | AA899642 | | ESTs | General |
| 505 | 14117 | AI171350 | | ESTs | General |
| 506 | 18744 | AI170407 | | ESTs | General |
| 507 | 11561 | AI233182 | | ESTs | General |
| 508 | 22711 | AA946072 | | ESTs | General |
| 509 | 3941 | AI011598 | | ESTs, Moderately similar to LMA5 | d, i, |
| | | | | MOUSE LAMININ | General |
| | | | | ALPHA-5 CHAIN | |
| | | | | [M. musculus] | |
| 510 | 23464 | AA925876 | | ESTs | General |
| 511 | 168 | AA924985 | calsequestrin 2 | calsequestrin 2 | h |
| 512 | 14944 | AA925364 | | ESTs | e |
| 513 | 24228 | AA800318 | | ESTs, Weakly similar to A1AT RAT | b |
| | | | | ALPHA-1-ANTIPROTEINASE | |
| | | | | PRECURSOR [R. norvegicus] | |
| 514 | 23822 | AA943114 | | ESTs | General |
| 515 | 12988 | AI137070 | | ESTs | b, General |
| 516 | 4499 | AA892511 | | EST, Weakly similar to CA22 RAT | e |
| | | | | CALCIUM-BINDING | |
| | | | | PROTEIN P22 | |
| | | | | [R. norvegicus] | |
| 517 | 12274 | AA964306 | | ESTs | General |
| 518 | 6094 | AA818911 | | ESTs | d |
| 519 | 19257 | AI030775 | | Rat (diabetic BB) MHC class II | b |
| | | | | alpha chain RT1.D alpha (u) | |
| 520 | 17847 | AI178214 | | ESTs | General |
| 521 | 18887 | AA892860 | | ESTs | General |
| 522 | 20740 | S69874 | | Rattus norvegicus Sprague-Dawley | c, d, |
| | | | | lipid-binding protein mRNA, | General |
| | | | | complete cds | |
| 523 | 4770 | AI235915 | | ESTs | f |
| 524 | 21705 | AA900684 | | ESTs, Moderately similar to | f |
| | | | | Y064_HUMAN HYPOTHETICAL | |
| | | | | PROTEIN KIAA0064 [H. sapiens] | |
| 525 | 18797 | AI236618 | | ESTs, Weakly similar to KIAA0597 | General |
| | | | | protein [H. sapiens] | |
| 526 | 22212 | AI236294 | | ESTs, Highly similar to translation | General |
| | | | | initiation factor eIF6 [M. musculus] | |
| 527 | 2153 | U75404 | | ESTs | General |
| 528 | 21654 | U53184 | estrogen-responsive uterine | estrogen-responsive uterine | i, General |
| | | | transcript | transcript | |
| 529 | 3191 | AI013075 | | ESTs, Weakly similar to T03454 | General |
| | | | | ALR protein [H. sapiens] | |
| 530 | 16613 | AA874784 | | ESTs | e, f, |
| | | | | | General |
| 531 | 5482 | AI180252 | | ESTs | General |
| 532 | 14337 | AI180414 | | ESTs, Moderately similar to SPA1 | General |
| | | | | MOUSE GTPASE-ACTIVATING | |
| | | | | PROTEIN SPA-1 [M. musculus] | |
| 533 | 23471 | AA955162 | | ESTs | General |
| 534 | 4098 | AI008642 | | ESTs, Weakly similar to LANP RAT | i, General |
| | | | | LEUCINE-RICH | |
| | | | | ACIDIC NUCLEAR | |
| | | | | PROTEIN [R. norvegicus] | |
| 535 | 23372 | AI031002 | | ESTs | h |
| 536 | 18217 | AA858930 | | ESTs | General |
| 537 | 3905 | AI103403 | polypyrimidine tract binding | polypyrimidine tract binding protein | General |
| | | | protein | | |
| 538 | 24375 | AI104979 | | ESTs, Moderately similar to | General |
| | | | | nucleolar protein p40 [H. sapiens] | |
| 539 | 14256 | AI176845 | | ESTs, Weakly similar to cornichon | General |
| | | | | [M. musculus] | |
| 540 | 18349 | AA799744 | | ESTs | General |
| 541 | 2310 | AI029969 | | ESTs | General |
| 542 | 3437 | AA892897 | | ESTs | General |
| 543 | 806 | AI169231 | | ESTs, Highly similar to GENE 33 | General |
| | | | | POLYPEPTIDE [R. norvegicus] | |
| 544 | 11531 | AA799773 | | ESTs, Highly similar to gamma- | g, h, i, |
| | | | | filamin [H. sapiens] | General |
| 545 | 15181 | AI235234 | | ESTs | i |
| 546 | 22689 | AA945962 | | ESTs | General |
| 547 | 9084 | AA893717 | | ESTs | f |
| 548 | 5307 | AA893529 | | ESTs, Weakly similar to | b |
| | | | | selenocysteine tRNA gene | |
| | | | | transcription activating factor | |
| | | | | [M. musculus] | |
| 549 | 25663 | X05472 | | | e |
| 550 | 18278 | AI105080 | | ESTs, Moderately similar to | General |
| | | | | SCOT_HUMAN | |
| | | | | SUCCINYL-COA: 3- | |
| | | | | KETOACID-COENZYME A | |
| | | | | TRANSFERASE PRECURSOR | |
| | | | | [H. sapiens] | |
| 551 | 6044 | AI011285 | | ESTs | a, General |
| 552 | 17361 | AA859114 | | ESTs | i, General |
| 553 | 22464 | AI009713 | | ESTs | d |
| 554 | 8137 | AA800749 | | ESTs | e |
| 555 | 16426 | X70369 | procollagen, type III, alpha 1 | procollagen, type III, alpha 1 | c, e |
| 556 | 24237 | AA817726 | | ESTs | General |
| 557 | 17950 | AI236590 | | ESTs | General |
| 558 | 18172 | AI058364 | | ESTs, Weakly similar to G02313 | d, g, |
| | | | | CDC37 homolog [H. sapiens] | General |
| 559 | 22614 | AI031004 | | ESTs, Highly similar to SX17 | d |
| | | | | MOUSE TRANSCRIPTION | |
| | | | | FACTOR SOX-17 [M. musculus] | |
| 560 | 23005 | AA942770 | | ESTs | General |
| 561 | 17285 | J02827 | Branched chain alpha-ketoacid | Branched chain alpha-ketoacid | General |
| | | | dehydrogenase subunit E1 alpha | dehydrogenase subunit E1 alpha | |
| 562 | 15885 | AA965207 | | ESTs, Highly similar to KIAA0958 | d |
| | | | | protein [H. sapiens] | |
| 563 | 5461 | AI044338 | | ESTs | General |
| 564 | 22677 | AA942718 | | ESTs | General |
| 565 | 18507 | AI175551 | | ESTs, Moderately similar to | General |
| | | | | AF145050 1 translation elongation | |
| | | | | factor 1-delta subunit | |
| | | | | [R. norvegicus] | |
| 566 | 8012 | AI058330 | decay-accelarating factor | decay-accelarating factor | General |
| 567 | 13310 | AI177119 | | ESTs, Weakly similar to C1QB RAT | b, General |
| | | | | COMPLEMENT C1Q | |
| | | | | SUBCOMPONENT, B CHAIN | |
| | | | | PRECURSOR [R. norvegicus] | |
| 568 | 6548 | AI072658 | | ESTs | d, General |
| 569 | 7941 | AI043958 | | ESTs | g, i, |
| | | | | | General |
| 570 | 13619 | AI179464 | | ESTs | General |
| 571 | 3207 | AA997466 | | ESTs | General |
| 572 | 20582 | AA859688 | | ESTs, Weakly similar to I37195 AU- | f, General |
| | | | | specific RNA-binding protein/enoyl- | |
| | | | | CoA hydratase homolog [H. sapiens] | |
| 573 | 21796 | AI012221 | | ESTs, Weakly similar to S70484 | g, General |
| | | | | RS43 protein - rat (fragment) | |
| | | | | [R. norvegicus] | |
| 574 | 19071 | AA850524 | | ESTs, Highly similar to I49257 | f |
| | | | | NF2d9 - mouse [M. musculus] | |
| 575 | 24200 | AI012356 | | ESTs | General |
| 576 | 24246 | AA963703 | | ESTs, Highly similar to cell cycle | g, General |
| | | | | protein p38-2G4 homolog | |
| | | | | [H. sapiens] | |
| 577 | 6133 | AA866234 | | ESTs, Highly similar to | c |
| | | | | EFTU_HUMAN ELONGATION | |
| | | | | FACTOR TU, MITOCHONDRIAL | |
| | | | | PRECURSOR [H. sapiens] | |
| 578 | 21632 | AI009167 | | ESTs, Highly similar to BAG-family | General |
| | | | | molecular chaperone regulator-2 | |
| | | | | [H. sapiens] | |
| 579 | 13772 | AA851926 | | ESTs, Weakly similar to A57514 | General |
| | | | | RNA helicase HEL117 - rat | |
| | | | | [R. norvegicus] | |
| 580 | 22247 | AA943537 | | Rattus norvegicus zyxin mRNA, | i, General |
| | | | | partial cds | |
| 581 | 23732 | AA957653 | | ESTs, Weakly similar to RNB6 | General |
| | | | | [R. norvegicus] | |
| 582 | 22385 | AA859805 | | ESTs, Moderately similar to LYOX | e |
| | | | | RAT PROTEIN-LYSINE 6- | |
| | | | | OXIDASE PRECURSOR | |
| | | | | [R. norvegicus] | |
| 583 | 2459 | AA964755 | | ESTs | i |
| 584 | 23964 | AI234748 | | ESTs | g, General |
| 585 | 4074 | AI175990 | | ESTs | g |
| 586 | 17204 | AI171844 | HMm: RIKEN cDNA 2410043G19 | Rattus norvegicus F1-ATPase | e, g |
| | | | gene | epsilon subunit mRNA, nuclear | |
| | | | | gene encoding mitochondrial | |
| | | | | protein, complete cds | |
| 587 | 6560 | AI230440 | | ESTs | d |
| 588 | 17883 | M11851 | | Rat heart myosin light chain 2 | a, b |
| | | | | (MLC2) mRNA, 3′ end | |
| 589 | 22937 | AA850290 | | ESTs | i |
| 590 | 4944 | AA924405 | | ESTs, Moderately similar to | g, General |
| | | | | NO56_HUMAN NUCLEOLAR | |
| | | | | PROTEIN NOP56 [H. sapiens] | |
| 591 | 3822 | AA900863 | | ESTs, Weakly similar to nuclear | g, General |
| | | | | RNA helicase [R. norvegicus] | |
| 592 | 2729 | AI170363 | | ESTs | d |
| 593 | 19282 | AA955123 | | EST | General |
| 594 | 21740 | AI176810 | | ESTs | General |
| 595 | 23773 | AA956476 | | ESTs | General |
| 596 | 6843 | AI009768 | | ESTs | f |
| 597 | 14840 | AI237698 | | ESTs | General |
| 598 | 20038 | AA899797 | | EST | c, General |
| 599 | 18541 | X14671 | | ESTs, Highly similar to RL26 RAT | e, h |
| | | | | 60S RIBOSOMAL PROTEIN L26 | |
| | | | | [R. norvegicus] | |
| 600 | 13966 | AI231421 | | ESTs | d, General |
| 601 | 17234 | AI102741 | Tissue inhibitor of | Tissue inhibitor of | h |
| | | | metalloproteinase 3 | metalloproteinase 3 | |
| 602 | 22544 | AA851245 | | ESTs, Weakly similar to KIAA0926 | General |
| | | | | protein [H. sapiens] | |
| 603 | 13023 | AI233740 | | ESTs, Weakly similar to ALDR RAT | General |
| | | | | ALDOSE REDUCTASE | |
| | | | | [R. norvegicus] | |
| 604 | 24209 | AI172423 | | ESTs | i, General |
| 605 | 23626 | AA955540 | | ESTs | General |
| 606 | 4670 | AI233714 | | ESTs | h |
| 607 | 18215 | AA943367 | | ESTs | h |
| 608 | 3014 | AI176362 | | ESTs | c, General |
| 609 | 16756 | AA818089 | | ESTs, Highly similar to glycyl-tRNA | General |
| | | | | synthetase [H. sapiens] | |
| 610 | 22605 | AA859447 | | ESTs | i |
| 611 | 5283 | AF090113 | | Rattus norvegicus glutamate | h |
| | | | | receptor interacting protein 2 | |
| | | | | (GRIP2) mRNA, complete cds | |
| 612 | 6043 | AI059588 | | ESTs, Weakly similar to 54TMp | g |
| | | | | [H. sapiens] | |
| 613 | 21474 | AA851361 | | ESTs | d |
| 614 | 23038 | AA900881 | branched chain | branched chain aminotransferase 1, | General |
| | | | aminotransferase 1, cytosolic | cytosolic | |
| 615 | 13336 | AI237070 | | ESTs, Weakly similar to | a, b, |
| | | | | dJ1042K10.4 [H. sapiens] | General |
| 616 | 3988 | AI008546 | proteasome (prosome, | proteasome (prosome, macropain) | d |
| | | | macropain) subunit, alpha type 3 | subunit, alpha type 3 | |
| 617 | 20823 | AI176302 | | ESTs, Highly similar to A47416 | d |
| | | | | ubiquitin-like protein/ribosomal | |
| | | | | protein S30 - rat [R. norvegicus] | |
| 618 | 23044 | AF034218 | hyaluronidase 2 | hyaluronidase 2 | General |
| 619 | 19013 | AI639256 | | ESTs | a |
| 620 | 16124 | AI176963 | | Rattus norvegicus transcription | c |
| | | | | factor MRG1 mRNA, complete cds | |
| 621 | 17848 | AA900460 | | ESTs, Weakly similar to TCPA RAT | General |
| | | | | T-COMPLEX PROTEIN 1, ALPHA | |
| | | | | SUBUNIT [R. norvegicus] | |
| 622 | 19074 | AI009391 | | ESTs | f |
| 623 | 3615 | AI237645 | transferrin receptor | transferrin receptor | d, General |
| 624 | 11387 | AA891677 | | ESTs | General |
| 625 | 25765 | X89706 | | | General |
| 626 | 21653 | AI237535 | estrogen-responsive uterine | estrogen-responsive uterine | General |
| | | | transcript | transcript | |
| 627 | 5228 | AI233311 | | ESTs, Highly similar to cytokine-like | General |
| | | | | factor-1 precursor [H. sapiens] | |
| 628 | 7299 | AI013911 | | ESTs, Weakly similar to CIRP | c, d, |
| | | | | [R. norvegicus] | General |
| 629 | 2093 | AI102097 | | ESTs | General |
| 630 | 9166 | AI137406 | | ESTs | i, General |
| 631 | 19723 | AA818761 | | ESTs | General |
| 632 | 2741 | AA963077 | | EST | General |
| 633 | 22667 | AA945069 | | ESTs | General |
| 634 | 17687 | AA799531 | | ESTs, Weakly similar to T23793 | a, e |
| | | | | hypothetical protein M18.3 - | |
| | | | | Caenorhabditis elegans [C. elegans] | |
| 635 | 23217 | AI235953 | | ESTs | General |
| 636 | 12135 | AI176418 | | ESTs | c |
| 637 | 19894 | AA859581 | | Rattus norvegicus late gestation | d |
| | | | | lung protein 1 (Lgl1) mRNA, | |
| | | | | complete cds | |
| 638 | 15345 | AI171587 | | ESTs | h |
| 639 | 23165 | AI231799 | | ESTs, Moderately similar to I68673 | General |
| | | | | gene X123 protein [H. sapiens] | |
| 640 | 3391 | AA998197 | | EST | General |
| 641 | 16215 | AA874999 | | ESTs, Moderately similar to | General |
| | | | | AF133910 1 ARL-6 interacting | |
| | | | | protein-3 [M. musculus] | |
| 642 | 19086 | AA892598 | | ESTs | General |
| 643 | 4725 | AA900290 | | ESTs, Highly similar to ALPHA-2- | d, General |
| | | | | MACROGLOBULIN PRECURSOR | |
| | | | | [R. norvegicus] | |
| 644 | 6727 | AI233213 | | ESTs | a, g |
| 645 | 21816 | AI231217 | | ESTs, Highly similar to S61A RAT | h, General |
| | | | | PROTEIN TRANSPORT PROTEIN | |
| | | | | SEC61 ALPHA SUBUNIT | |
| | | | | [R. norvegicus] | |
| 646 | 2069 | AI103616 | | ESTs, Weakly similar to RhoA | g, i, |
| | | | | [R. norvegicus] | General |
| 647 | 22717 | AI235948 | | ESTs, Highly similar to entactin | e, g |
| | | | | [R. norvegicus] | |
| 648 | 11205 | AI172057 | | ESTs | General |
| 649 | 22498 | AA944806 | | ESTs | c |
| 650 | 7192 | AI178530 | | ESTs | c |
| 651 | 8058 | AA818475 | | ESTs | General |
| 652 | 18800 | AI178504 | | ESTs | General |
| 653 | 12376 | AI103939 | | ESTs | i |
| 654 | 12695 | AI170948 | | ESTs, Moderately similar to S68207 | i, General |
| | | | | G protein-coupled receptor 6C.1 | |
| | | | | [H. sapiens] | |
| 655 | 5227 | AA925924 | | ESTs, Highly similar to cytokine-like | f, i, General |
| | | | | factor-1 precursor [H. sapiens] | |
| 656 | 7936 | AA875495 | | ESTs | i, General |
| 657 | 4491 | AA818798 | | Rattus norvegicus mRNA for | General |
| | | | | cathepsin Y, partial cds | |
| 658 | 13647 | AA892367 | | ESTs, Highly similar to RL3 RAT | General |
| | | | | 60S RIBOSOMAL PROTEIN L3 | |
| | | | | [R. norvegicus] | |
| 659 | 25716 | X61295 | | | e |
| 660 | 10547 | AA800186 | | EST | General |
| 661 | 7760 | AI030806 | | ESTs | b, General |
| 662 | 16687 | AI013553 | | ESTs | a |
| 663 | 24040 | AA957422 | | ESTs, Highly similar to HIGH | General |
| | | | | AFFINITY IMMUNOGLOBULIN | |
| | | | | EPSILON RECEPTOR GAMMA- | |
| | | | | SUBUNIT PRECURSOR | |
| | | | | [R. norvegicus] | |
| 664 | 16154 | AI009661 | | ESTs, Moderately similar to AF1q | i |
| | | | | [M. musculus] | |
| 665 | 14390 | AI232385 | | ESTs | g |
| 666 | 14651 | AI235919 | | ESTs | General |
| 667 | 17289 | AA891785 | HMm: isocitrate dehydrogenase 2 | ESTs, Weakly similar to IDHC RAT | b |
| | | | (NADP+), mitochondrial | ISOCITRATE DEHYDROGENASE | |
| | | | | [R. norvegicus] | |
| 668 | 12609 | AA997486 | | ESTs | d, i, |
| | | | | | General |
| 669 | 21657 | X61381 | | Rattus norvegicus interferon- | b, i, |
| | | | | inducible protein variant 10 mRNA, | General |
| | | | | complete cds | |
| 670 | 5384 | AA891041 | jun B proto-oncogene | jun B proto-oncogene | General |
| 671 | 5613 | AI044850 | | ESTs | b |
| 672 | 16136 | AI103983 | unknown Glu-Pro dipeptide | Rattus norvegicus clone BB.1.4.1 | General |
| | | | repeat protein | unknown Glu-Pro dipeptide repeat | |
| | | | | protein mRNA, complete cds | |
| 673 | 23270 | AI179601 | | ESTs | General |
| 674 | 14464 | AI104848 | | ESTs | General |
| 675 | 6053 | AA818655 | | EST | d |
| 676 | 19067 | AA859663 | | ESTs | General |
| 677 | 22877 | AI178819 | | ESTs, Moderately similar to CGI- | General |
| | | | | 137 protein [H. sapiens] | |
| 678 | 14257 | AI230460 | | ESTs | e |
| 679 | 5132 | AA925342 | | ESTs, Highly similar to SKLM | General |
| | | | | MOUSE SKELEMIN [M. musculus] | |
| 680 | 2706 | AA965083 | | ESTs | General |
| 681 | 22615 | AA945643 | | ESTs | f, General |
| 682 | 13460 | AI175375 | | ESTs | General |
| 683 | 8707 | AI059707 | | ESTs | i, General |
| 684 | 17948 | AA944581 | | ESTs, Highly similar to RAB/Rip | General |
| | | | | protein [M. musculus] | |
| 685 | 13082 | AI136848 | | ESTs, Weakly similar to T34013 | a, b |
| | | | | hypothetical protein Y4C6B.5 - | |
| | | | | Caenorhabditis elegans [C. elegans] | |
| 686 | 16074 | AA874874 | | ESTs, Highly similar to ADHX RAT | f |
| | | | | ALCOHOL DEHYDROGENASE | |
| | | | | CLASS III [R. norvegicus] | |
| 687 | 8861 | AI070306 | | ESTs | General |
| 688 | 8594 | AI012932 | | ESTs | General |
| 689 | 22311 | AI176007 | | ESTs, Highly similar to | General |
| | | | | PM5P_HUMAN PROTEIN PM5 | |
| | | | | PRECURSOR [H. sapiens] | |
| 690 | 24268 | AI172281 | | ESTs | e |
| 691 | 9073 | AA892273 | | ESTs | a, b |
| 692 | 22423 | AA893164 | | ESTs | b |
| 693 | 5550 | AI044628 | | ESTs, Weakly similar to unknown | General |
| | | | | [H. sapiens] | |
| 694 | 3895 | AA894029 | | ESTs | b |
| 695 | 19069 | AA943737 | endothelial differentiation | endothelial differentiation | General |
| | | | sphingolipid G-protein-coupled | sphingolipid G-protein-coupled | |
| | | | recep | receptor 1 | |
| 696 | 22666 | AA900974 | | ESTs | d, General |
| 697 | 2852 | AI177059 | | ESTs | e, General |
| 698 | 12108 | AA848963 | | ESTs, Moderately similar to DNA- | a |
| | | | | REPAIR PROTEIN | |
| | | | | COMPLEMENTING XP-C CELLS | |
| | | | | HOMOLOG [M. musculus] | |
| 699 | 23240 | AA893939 | | ESTs, Highly similar to Method: | i |
| | | | | conceptual translation supplied by | |
| | | | | author [M. musculus] | |
| 700 | 4161 | AI112141 | | ESTs, Weakly similar to A55169 | General |
| | | | | pre-T-cell receptor alpha chain | |
| | | | | precursor - mouse [M. musculus] | |
| 701 | 18272 | AA799294 | | ESTs, Highly similar to KIAA0740 | c, e, |
| | | | | protein [H. sapiens] | General |
| 702 | 23612 | AI170751 | | ESTs | General |
| 703 | 4790 | AA900875 | | Rattus norvegicus transcription | General |
| | | | | factor MRG2 mRNA, complete cds | |
| 704 | 16168 | AA893280 | | ESTs, Moderately similar to | General |
| | | | | adipophilin [H. sapiens] | |
| 705 | 11644 | AI235282 | | ESTs, Moderately similar to AM2 | General |
| | | | | receptor [M. musculus] | |
| 706 | 19057 | AI235094 | cortactin isoform B | cortactin isoform B | General |
| 707 | 9501 | AI638949 | | ESTs, Weakly similar to S44893 | a |
| | | | | ZK1236.7 protein - Caenorhabditis | |
| | | | | elegans [C. elegans] | |
| 708 | 24310 | AA924578 | | ESTs | e, General |
| 709 | 1283 | U61729 | | Rattus norvegicus proline rich. | General |
| | | | | protein mRNA, complete cds | |
| 710 | 7681 | AI030449 | | ESTs, Moderately similar to | General |
| | | | | methyltransferase related protein | |
| | | | | [M. musculus] | |
| 711 | 1439 | U57391 | | Rattus norvegicus FceRI gamma- | General |
| | | | | chain interacting protein SH2-B | |
| | | | | (SH2-B) mRNA, complete cds | |
| 712 | 20872 | X51707 | ribosomal protein S19 | ESTs, Highly similar to RS19 RAT | f |
| | | | | 40S RIBOSOMAL PROTEIN S19 | |
| | | | | [R. norvegicus] | |
| 713 | 21660 | AI169751 | | Rattus norvegicus interferon- | b, General |
| | | | | inducible protein variant 10 mRNA, | |
| | | | | complete cds | |
| 714 | 19454 | AA998888 | | ESTs, Weakly similar to T09013 | f |
| | | | | RING finger protein Fxy - mouse | |
| | | | | [M. musculus] | |
| 715 | 12979 | AI169177 | | ESTs, Highly similar | General |
| | | | | to RADIATION- | |
| | | | | INDUCIBLE IMMEDIATE-EARLY | |
| | | | | GENE IEX-1 [M. musculus] | |
| 716 | 5675 | AI045026 | | ESTs | General |
| 717 | 17903 | AI231083 | | ESTs, Moderately similar to | d, General |
| | | | | AF155103 1 NY-REN-25 antigen | |
| | | | | [H. sapiens] | |
| 718 | 6743 | AI231219 | | ESTs | d, General |
| 719 | 23538 | AI102727 | solute carrier family 20 | solute carrier family 20 (phosphate | General |
| | | | (phosphate transporter), member 1 | transporter), member 1 | |
| 720 | 20617 | X53581 | | | e |
| 721 | 19416 | AA850244 | | ESTs | General |
| 722 | 16191 | AA874982 | | ESTs | c |
| 723 | 8745 | AI069939 | | ESTs | d |
| 724 | 14494 | AI234222 | | ESTs | g |
| 725 | 22681 | AA944413 | | ESTs | General |
| 726 | 21579 | AI103572 | | ESTs | General |
| 727 | 23978 | AA925352 | | ESTs | h, General |
| 728 | 25742 | X80130 | | | a, b, c |
| 729 | 14458 | AI145095 | | ESTs | General |
| 730 | 6172 | AA819646 | | ESTs, Highly similar to AF055084 1 | General |
| | | | | very large G-protein coupled | |
| | | | | receptor-1 [H. sapiens] | |
| 731 | 1998 | AA817864 | | ESTs | b |
| 732 | 25545 | S77900 | | | c |
| 733 | 12999 | AI176276 | | ESTs, Highly similar to | d |
| | | | | UAP1_HUMAN UDP-N- | |
| | | | | ACETYLHEXOSAMINE | |
| | | | | PYROPHOSPHORYLASE | |
| | | | | [H. sapiens] | |
| 734 | 1159 | AA891949 | | ESTs | g, General |
| 735 | 18907 | AI178971 | | Rattus norvegicus alpha-globin | a, b, |
| | | | | (GloA) gene, complete cds | General |
| 736 | 25647 | U83119 | | | e |
| 737 | 17892 | AI228438 | | ESTs | a |
| 738 | 18447 | AA849939 | | ESTs | b, General |
| 401 | 18473 | AI168975 | | ESTs | General |
| 739 | 4222 | AA860024 | | ESTs, Highly similar to | General |
| | | | | EF1G_HUMAN ELONGATION | |
| | | | | FACTOR 1-GAMMA [H. sapiens] | |
| 740 | 15197 | AI105444 | | ESTs | General |
| 741 | 22958 | AI171374 | | ESTs, Moderately similar to | d, General |
| | | | | meningioma-expressed antigen 11 | |
| | | | | [H. sapiens] | |
| 742 | 9111 | AI073176 | | ESTs, Weakly similar to G01614 | General |
| | | | | zinc finger protein 127 [H. sapiens] | |
| 743 | 23110 | AI169640 | | ESTs, Weakly similar to | d |
| | | | | AR20_CAEEL PROBABLE ARP2/3 | |
| | | | | COMPLEX 20 KDA | |
| | | | | SUBUNIT (P20- | |
| | | | | ARC) [C. elegans] | |
| 744 | 20998 | AA799803 | | ESTs, Weakly similar to serine | General |
| | | | | protease [R. norvegicus] | |
| 745 | 23184 | AI176554 | | ESTs | d |
| 746 | 14149 | AA998172 | platelet-activating factor | platelet-activating factor | i |
| | | | acetylhydrolase alpha 2 subunit | acetylhydrolase alpha 2 subunit | |
| | | | (PAF- | (PAF-AH alpha 2) | |
| 747 | 23816 | AA956596 | | ESTs | e |
| 748 | 8494 | AI059968 | | ESTs | i, General |
| 749 | 21841 | AA996499 | | ESTs, Weakly similar to C1QB RAT | b, General |
| | | | | COMPLEMENT C1Q | |
| | | | | SUBCOMPONENT, B CHAIN | |
| | | | | PRECURSOR [R. norvegicus] | |
| 750 | 19765 | AI230945 | | ESTs, Highly similar to AF151862 1 | d |
| | | | | CGI-104 protein [H. sapiens] | |
| 751 | 6002 | AA818101 | | EST | b |
| 752 | 21945 | AI104132 | | ESTs | e |
| 753 | 4496 | AA892400 | | ESTs | i |
| 754 | 12921 | AI112636 | | ESTs, Moderately similar to | General |
| | | | | UDP_HUMAN URIDINE | |
| | | | | PHOSPHORYLASE [H. sapiens] | |
| 755 | 14516 | AI013197 | | ESTs, Weakly similar to coding | e |
| | | | | sequence of pol [R. norvegicus] | |
| 756 | 11504 | AI171652 | | ESTs | General |
| 757 | 3698 | AI172193 | | ESTs | e |
| 758 | 5886 | AI045807 | | ESTs | General |
| 759 | 4154 | AI009467 | | ESTs | General |
| 760 | 4330 | AA818747 | | ESTs | c, General |
| 390 | 16597 | AI102486 | | ESTs, Weakly similar to S37583 | General |
| | | | | RING finger protein rfp - mouse | |
| | | | | [M. musculus] | |
| 761 | 15191 | AI176456 | | Rat metallothionein-2 and | General |
| | | | | metallothionein-1 genes, complete | |
| | | | | cds | |
| 762 | 10654 | AI235991 | | Rattus norvegicus enhancer-of-split | f |
| | | | | and hairy-related protein 2 (SHARP- | |
| | | | | 2) mRNA, complete cds | |
| 763 | 14341 | AI227596 | | ESTs | General |
| 764 | 22915 | AI228299 | | ESTs, Highly similar to p97 | a, c, |
| | | | | homologous protein [H. sapiens] | General |
| 765 | 6638 | AI137579 | | ESTs | c, General |
| 766 | 25250 | D10706 | | | h |
| 767 | 24072 | AI231093 | | ESTs | e |
| 768 | 15628 | AI230338 | | ESTs, Highly similar to | General |
| | | | | RS16_HUMAN 40S RIBOSOMAL | |
| | | | | PROTEIN S1 [R. norvegicus] | |
| 769 | 18831 | AI104357 | | ESTs, Highly similar to ATRTC | c, h, |
| | | | | actin beta - rat [R. norvegicus] | General |
| 770 | 8343 | AI059502 | | ESTs | i |
| 771 | 7749 | AA848804 | | ESTs, Highly similar to BTF3 | General |
| | | | | MOUSE TRANSCRIPTION | |
| | | | | FACTOR BTF3 [M. musculus] | |
| 772 | 9984 | AA955536 | | ESTs, Weakly similar to ELL | General |
| | | | | MOUSE RNA POLYMERASE II | |
| | | | | ELONGATION FACTOR ELL | |
| | | | | [M. musculus] | |
| 773 | 17264 | D25233 | Retinoblastoma 1 (including | Retinoblastoma 1 (including | General |
| | | | osteosarcoma) | osteosarcoma) | |
| 774 | 5356 | AI177813 | | ESTs, Moderately similar to S27962 | General |
| | | | | modulator recognition factor 1 | |
| | | | | [H. sapiens] | |
| 775 | 8626 | AI059449 | | ESTs | General |
| 776 | 2522 | AA964957 | | ESTs | a |
| 777 | 6291 | AI144797 | | ESTs | h |
| 642 | 19085 | AA892598 | | ESTs | General |
| 778 | 18366 | AA799442 | | ESTs | General |
| 779 | 1686 | AI179971 | Hemoglobin, alpha 1 | Hemoglobin, alpha 1 | b, General |
| 780 | 20004 | AI031036 | | ESTs | c |
| 781 | 4969 | AI113008 | | ESTs, Moderately similar to | f, g, |
| | | | | megakaryocyte stimulating factor | General |
| | | | | [H. sapiens] | |
| 782 | 6697 | AI045340 | | ESTs | General |
| 783 | 13420 | AA892042 | | ESTs | d, h, |
| | | | | | General |
| 784 | 21527 | AA851733 | | ESTs | f |
| 785 | 22017 | AA944209 | | ESTs, Moderately similar to PIM1 | General |
| | | | | RAT PROTO-ONCOGENE | |
| | | | | SERINE/THREONINE-PROTEIN | |
| | | | | KINASE PIM-1 [R. norvegicus] | |
| 786 | 9388 | AA894173 | | ESTs | b |
| 787 | 23197 | AI172159 | | ESTs | General |
| 788 | 3518 | AA944584 | | ESTs | b, f |
| 422 | 18606 | X53504 | | ESTs, Highly similar to 60S | e, General |
| | | | | RIBOSOMAL PROTEIN L12 | |
| | | | | [R. norvegicus] | |
| 789 | 23123 | AA924794 | | ESTs | g, General |
| 790 | 3081 | AA999171 | Signal transducer and activator | Signal transducer and activator of | h |
| | | | of transcription 1 | transcription 1 | |
| 791 | 17358 | AI179147 | | ESTs, Highly similar to | e, h |
| | | | | ETFB_HUMAN ELECTRON | |
| | | | | TRANSFER FLAVOPROTEIN | |
| | | | | BETA-SUBUNIT [H. sapiens] | |
| 792 | 2435 | AI639411 | | ESTs, Highly similar to dJ149A16.6 | h |
| | | | | [H. sapiens] | |
| 793 | 24651 | M83678 | RAB13 | RAB13 | b |
| 794 | 17675 | AI137597 | | ESTs | f |
| 795 | 4407 | H33528 | | ESTs | General |
| 796 | 4874 | AA923850 | | ESTs | General |
| 797 | 16001 | AA866452 | actin, alpha, cardiac | actin, alpha, cardiac | a, b, c, g, |
| | | | | | General |
| 798 | 18883 | AA799992 | | ESTs | General |
| 799 | 10972 | AI233956 | | ESTs | General |
| 800 | 1114 | AI029917 | | Rat brain neuron-specific enolase | f |
| | | | | mRNA, complete cds | |
| 801 | 15382 | AI172302 | | ESTs, Weakly similar to S43056 | General |
| | | | | hypothetical protein - mouse | |
| | | | | [M. musculus] | |
| 802 | 15568 | AI010257 | | ESTs | General |
| 803 | 10532 | AI009602 | | ESTs | g, General |
| 804 | 11873 | AI232326 | | ESTs | General |
| 805 | 13161 | AI138093 | | ESTs | e |
| 806 | 11485 | AI235348 | | ESTs, Weakly similar to polycomb- | f |
| | | | | group protein [R. norvegicus] | |
| 807 | 25689 | X52815 | | | g, General |
| 808 | 1831 | AI071137 | | Rat mRNA for cdc25B, complete | a, b, |
| | | | | cds | General |
| 809 | 25846 | AI638988 | | | General |
| 810 | 20086 | AI013260 | lamin | lamin | g |
| 811 | 20041 | AI112161 | | ESTs | i, General |
| 812 | 18867 | D88250 | | Rattus norvegicus mRNA for serine | a, b, |
| | | | | protease, complete cds | General |
| 813 | 7615 | AI030163 | | ESTs | d |
| 814 | 23833 | AI113026 | | ESTs, Highly similar to AGP2_RAT | General |
| | | | | ANGIOPOIETIN-2 (ANG-2) | |
| | | | | [R. norvegicus] | |
| 815 | 23731 | AA894004 | | ESTs, Weakly similar to AF099929 | f, g, |
| | | | | 1 pervin [R. norvegicus] | General |
| 816 | 21400 | M36410 | sepiapterin reductase | sepiapterin reductase | General |
| 817 | 20763 | AA925516 | | ESTs | b |
| 818 | 15218 | AI102495 | | ESTs, Moderately similar to purine | General |
| | | | | nucleoside phosphorylase | |
| | | | | [M. musculus] | |
| 819 | 3088 | AI013369 | | ESTs | c |
| 820 | 10317 | AI172339 | | ESTs, Weakly similar to T21860 | General |
| | | | | hypothetical protein F36F2.1 - | |
| | | | | Caenorhabditis elegans [C. elegans] | |
| 821 | 17468 | AA892545 | | ESTs, Highly similar to multi- | d |
| | | | | membrane spanning polyspecific | |
| | | | | transporter [M. musculus] | |
| 822 | 23566 | AA955482 | | ESTs, Moderately similar to | General |
| | | | | AF132950 1 CGI-16 protein | |
| | | | | [H. sapiens] | |
| 823 | 15510 | AA875428 | | ESTs | g, General |
| 824 | 17408 | AA818971 | | ESTs | h |
| 825 | 16608 | AA996840 | | ESTs | h |
| 826 | 15308 | AI072896 | | ESTs, Weakly similar to neural | h |
| | | | | plakophilin related arm-repeat | |
| | | | | protein [M. musculus] | |
| 827 | 7397 | AI104244 | | Rattus norvegicus mRNA for | h |
| | | | | oligomycin sensitivity conferring | |
| | | | | protein, complete cds | |
| 828 | 18542 | AA893493 | | ESTs, Highly similar to RL26 RAT | e |
| | | | | 60S RIBOSOMAL PROTEIN L26 | |
| | | | | [R. norvegicus] | |
| 829 | 19094 | AI232021 | | ESTs, Highly similar to Human | e |
| | | | | Translation Initiation Factor Eif1, | |
| | | | | Nmr, 29 Structures [H. sapiens] | |
| 830 | 14449 | AI234152 | | ESTs | General |
| 831 | 12096 | AA819415 | | ESTs | i |
| 832 | 6135 | AA819065 | | ESTs | d |
| 833 | 21785 | AI177312 | | ESTs | g, General |
| 834 | 18454 | AI231776 | | ESTs, Highly similar to mRNA | General |
| | | | | cleavage factor I 25 kDa subunit | |
| | | | | [H. sapiens] | |
| 835 | 21581 | AA944828 | | ESTs | c, General |
| 836 | 15383 | AA955358 | | ESTs | General |
| 837 | 16071 | AA997821 | | ESTs | General |
| 838 | 16533 | AA998174 | | ESTs | g, General |
| 839 | 5950 | AI169266 | | ESTs, Moderately similar to | General |
| | | | | AR16_HUMAN | |
| | | | | ARP2/3 COMPLEX | |
| | | | | 16 KDA SUBUNIT [H. sapiens] | |
| 840 | 15004 | AI235224 | | Rattus norvegicus tissue inhibitor of | f, g, |
| | | | | metalloproteinase-1 (TIMP1), | General |
| | | | | mRNA, complete cds | |
| 798 | 18881 | AA799992 | | ESTs | a, General |
| 841 | 21185 | AI170056 | | ESTs | General |
| 842 | 5049 | D10655 | dihydrolipoamide | dihydrolipoamide acetyltransferase | e |
| | | | acetyltransferase | | |
| 843 | 17810 | AA799778 | | Rat F-0-ATPase subunit b mRNA, | i |
| | | | | complete cds | |
| 844 | 10071 | AI639058 | | ESTs, Moderately similar to | i, General |
| | | | | dJ718J7.1 [H. sapiens] | |
| 845 | 2913 | AI232272 | | ESTs, Weakly similar to T25417 | General |
| | | | | hypothetical protein T28D6.9 - | |
| | | | | Caenorhabditis elegans [C. elegans] | |
| 846 | 5703 | AI045136 | | ESTs | i, General |
| 847 | 3143 | AI232408 | | ESTs | General |
| 848 | 10780 | AI136555 | | Rattus norvegicus mRNA for | g, General |
| | | | | Castration Induced Prostatic | |
| | | | | Apoptosis Related protein-1 (CIPAR- | |
| | | | | 1) | |
| 849 | 5357 | AI179507 | | ESTs | b, General |
| 850 | 18909 | AA849426 | | ESTs, Weakly similar to | General |
| | | | | YLC4_CAEEL HYPOTHETICAL | |
| | | | | 81.0 KD PROTEIN C35D10.4 IN | |
| | | | | CHROMOSOME III [C. elegans] | |
| 321 | 22696 | AA945996 | | ESTs | General |
| 851 | 9815 | AA848218 | | ESTs | General |
| 852 | 20761 | AA900331 | | ESTs | General |
| 853 | 18686 | D00729 | dodecenoyl-Coenzyme A delta | Rat mRNA for delta3, delta2-enoyl- | e, h |
| | | | isomerase (3, 2 trans-enoyl- | CoA isomerase, dodecenoyl- | |
| | | | Coenyme A | Coenzyme A delta isomerase (3, 2 | |
| | | | | trans-enoyl-Coenyme A isomerase) | |
| 854 | 2378 | AA964501 | | ESTs | d |
| 855 | 17307 | H32747 | | ESTs | h |
| 856 | 10837 | AI072144 | | ESTs | General |
| 857 | 4130 | AA800298 | | ESTs | e, f, |
| | | | | | General |
| 858 | 18529 | AI230716 | | ESTs | g, General |
| 859 | 7362 | AI029026 | | ESTs | General |
| 860 | 2730 | AI012962 | | ESTs, Weakly similar to putative | General |
| | | | | prenylated protein [H. sapiens] | |
| 861 | 21766 | AA850916 | | ESTs | General |
| 862 | 13240 | AI169311 | | ESTs | d, General |
| 863 | 12664 | AA999110 | | ESTs, Weakly similar to | General |
| | | | | MAPE_HUMAN MELANOMA | |
| | | | | ANTIGEN PREFERENTIALLY | |
| | | | | EXPRESSED IN TUMORS | |
| | | | | [H. sapiens] | |
| 864 | 24520 | L20869 | | Rat pancreatitis associated protein | General |
| | | | | III (PAPIII0) mRNA, complete cds | |
| 865 | 10549 | AA801255 | | ESTs | General |
| 866 | 8737 | AI071926 | | ESTs | h |
| 867 | 12674 | AI235358 | | ESTs, Highly similar to UCR2 RAT | a, c |
| | | | | UBIQUINOL-CYTOCHROME C | |
| | | | | REDUCTASE COMPLEX CORE | |
| | | | | PROTEIN 2 PRECURSOR | |
| | | | | [R. norvegicus] | |
| 868 | 8032 | AI058398 | | ESTs | f |
| 869 | 2587 | AI232103 | | ESTs | General |
| 870 | 13547 | M63983 | Hypoxanthine phosphoribosyl | ESTs, Moderately similar to ICA2 | i |
| | | | transferase | MOUSE INTERCELLULAR | |
| | | | | ADHESION MOLECULE-2 | |
| | | | | PRECURSOR | |
| | | | | [M. musculus], Hypoxanthine | |
| | | | | phosphoribosyl transferase | |
| 871 | 2135 | AI010195 | | ESTs | General |
| 872 | 6881 | AI010169 | | ESTs | General |
| 873 | 5494 | AI044425 | | ESTs | General |
| 874 | 16834 | AA963324 | | ESTs | d |
| 875 | 5699 | AI045128 | | ESTs, Moderately similar to T50615 | General |
| | | | | hypothetical protein | |
| | | | | DKFZp761G1515.1 [H. sapiens] | |
| 876 | 6282 | AA819523 | | ESTs | c |
| 877 | 18598 | AI070775 | | ESTs | General |
| 878 | 5297 | AI170379 | | ESTs | General |
| 879 | 7867 | AI043695 | HHs: phosphoribosyl | Rattus norvegicus mRNA for | d |
| | | | pyrophosphate amidotransferase | amidophosphoribosyltransferase | |
| 880 | 6097 | AA818929 | | ESTs | General |
| 881 | 10115 | AI058890 | | ESTs | d |
| 882 | 3738 | AA998256 | | ESTs | General |
| 883 | 9770 | AI060016 | | ESTs | General |
| 884 | 18830 | AA964496 | | ESTs, Highly similar to ATRTC | g, h, |
| | | | | actin beta - rat [R. norvegicus] | General |
| 885 | 7161 | AI233407 | | ESTs, Weakly similar to S44853 | General |
| | | | | K12H4.3 protein - Caenorhabditis | |
| | | | | elegans [C. elegans] | |
| 886 | 19729 | AA818910 | | ESTs | General |
| 887 | 15524 | D13124 | HMm: RIKEN cDNA | Rattus norvegicus P2 mRNA for | b |
| | | | 1810041M08 gene | ATP synthase subunit c, complete | |
| | | | | cds | |
| 888 | 16679 | AI105268 | | ESTs | d |
| 889 | 19184 | AI178025 | | ESTs, Highly similar to TGIF | General |
| | | | | MOUSE 5′-TG-3′ INTERACTING | |
| | | | | FACTOR [M. musculus] | |
| 890 | 6015 | AA818158 | | ESTs | f |
| 891 | 21659 | AI010584 | | Rattus norvegicus interferon- | General |
| | | | | inducible protein variant 10 mRNA, | |
| | | | | complete cds | |
| 892 | 18612 | AI228624 | | ESTs, Highly similar to | General |
| | | | | RL23_HUMAN 60S RIBOSOMAL | |
| | | | | PROTEIN L23 [R. norvegicus] | |
| 893 | 19291 | AA963933 | | EST | General |
| 732 | 21583 | S77900 | | ESTs | c, General |
| 894 | 7888 | AI233583 | HHs: arginyl-tRNA synthetase | ESTs, Moderately similar to | h |
| | | | | SYR_HUMAN ARGINYL-TRNA | |
| | | | | SYNTHETASE [H. sapiens] | |
| 895 | 21079 | AA800245 | | ESTs, Weakly similar to T09013 | f |
| | | | | RING finger protein Fxy - mouse | |
| | | | | [M. musculus] | |
| 896 | 17949 | AI011480 | | ESTs, Highly similar to RAB/Rip | g, i |
| | | | | protein [M. musculus] | |
| 897 | 4089 | AI180251 | | ESTs | d |
| 898 | 3823 | AI233147 | | ESTs, Weakly similar to nuclear | General |
| | | | | RNA helicase [R. norvegicus] | |
| 899 | 17506 | AI070068 | | ESTs, Weakly similar to 2104282A | General |
| | | | | Gadd45 gene [R. norvegicus] | |
| 900 | 21667 | AI013773 | | ESTs | General |
| 901 | 8919 | AI103388 | | ESTs, Weakly similar to | g, i, |
| | | | | ARF6_HUMAN ADP- | General |
| | | | | RIBOSYLATION FACTOR 6 | |
| | | | | [R. norvegicus] | |
| 902 | 24064 | AA957480 | | ESTs | h |
| 903 | 5902 | AI045871 | | ESTs, Moderately similar to | i |
| | | | | inhibitor of MyoD family-a | |
| | | | | [M. musculus] | |
| 904 | 7913 | AI043849 | | ESTs, Weakly similar to ELL | General |
| | | | | MOUSE RNA POLYMERASE II | |
| | | | | ELONGATION FACTOR ELL | |
| | | | | [M. musculus] | |
| 905 | 2980 | AI177761 | | ESTs, Weakly similar to LMP1 RAT | b |
| | | | | LYSOSOME-ASSOCIATED | |
| | | | | MEMBRANE GLYCOPROTEIN 1 | |
| | | | | PRECURSOR [R. norvegicus] | |
| 906 | 8477 | AI179167 | | ESTs | General |
| 907 | 11203 | AA892554 | | ESTs, Highly similar to ras-GTPase- | General |
| | | | | activating protein SH3-domain | |
| | | | | binding protein [M. musculus] | |
| 908 | 22150 | AA893607 | | ESTs | General |
| 909 | 10185 | H33426 | | ESTs | g |
| 910 | 15626 | X17665 | | ESTs, Highly similar to | f, h, |
| | | | | RS16_HUMAN 40S RIBOSOMAL | General |
| | | | | PROTEIN S1 [R. norvegicus] | |
| 911 | 2096 | AI233801 | | ESTs | h |
| 912 | 1809 | AA946503 | | Rat mRNA for alpha-2u globulin- | f |
| | | | | related protein | |
| 913 | 23501 | AA955251 | | ESTs | General |
| 914 | 23927 | AA957007 | glutathione S-transferase, mu 5 | glutathione S-transferase, mu 5 | e |
| 915 | 6205 | AI104907 | TEMO | TEMO | e |
| 916 | 8822 | AI070158 | | ESTs, Moderately similar to AD08 | i |
| | | | | MOUSE ADAM 8 PRECURSOR | |
| | | | | [M. musculus] | |
| 917 | 5754 | AA850738 | | ESTs | General |
| 918 | 21822 | AI228642 | | ESTs, Highly similar to seven | h |
| | | | | transmembrane domain protein | |
| | | | | [H. sapiens] | |
| 919 | 19075 | AI236473 | | ESTs, Moderately similar to | General |
| | | | | cysteine-rich hydrophobic 1 | |
| | | | | [M. musculus] | |
| 920 | 2361 | AA875512 | | R. norvegicus epsilon 3 globin gene | a |
| 921 | 4420 | AI171916 | | ESTs | g, General |
| 922 | 3819 | AI176209 | Tyrosine 3- | Tyrosine 3- | General |
| | | | monooxygenase/tryptophan 5- | monooxygenase/tryptophan 5- | |
| | | | monooxygenase activation | monooxygenase activation protein, | |
| | | | protein, zeta polypeptide | zeta polypeptide | |
| 923 | 22042 | AA946476 | | ESTs | i, General |
| 924 | 3016 | AA818069 | | Rat mRNA for polyubiquitin (four | a, f |
| | | | | repetitive ubiquitins in tandem), | |
| | | | | complete cds | |
| 925 | 22487 | AI102578 | | ESTs, Highly similar to I49523 | General |
| | | | | Mouse primary response gene B94 | |
| | | | | mRNA, 3′end - mouse [M. musculus] | |
| 926 | 5507 | AI176584 | | Rattus norvegicus insulin-like | General |
| | | | | growth factor binding protein 5 | |
| | | | | mRNA, 3′ UTR | |
| 927 | 22077 | AI177099 | | ESTs, Highly similar to serine | General |
| | | | | protease [H. sapiens] | |
| 928 | 25386 | L37970 | | | b |
| 929 | 13930 | AA800613 | | Rattus norvegicus gene for TIS11, | General |
| | | | | complete cds | |
| 930 | 11484 | AI179612 | | ESTs, Moderately similar to MY16 | General |
| | | | | MOUSE MYELOID | |
| | | | | DIFFERENTIATION PRIMARY | |
| | | | | RESPONSE PROTEIN MYD116 | |
| | | | | [M. musculus], Rattus norvegicus | |
| | | | | progression elevated gene 3 protein | |
| | | | | mRNA, complete cds | |
| 931 | 3417 | AI012337 | | ESTs, Highly similar to NHPX RAT | g, General |
| | | | | NHP2/RS6 FAMILY PROTEIN | |
| | | | | YEL026W HOMOLOG | |
| | | | | [R. norvegicus] | |
| 932 | 14677 | AI234620 | | EST | c |
| 933 | 2134 | AA894212 | | ESTs, Weakly similar to T20899 | a |
| | | | | hypothetical protein F14F3.3 - | |
| | | | | Caenorhabditis elegans [C. elegans] | |
| 586 | 17203 | AI171844 | HMm: RIKEN cDNA 2410043G19 | Rattus norvegicus F1-ATPase | b, e |
| | | | gene | epsilon subunit mRNA, nuclear | |
| | | | | gene encoding mitochondrial | |
| | | | | protein, complete cds | |
| 934 | 13291 | AI137286 | | ESTs | General |
| 935 | 22899 | AA956555 | | ESTs | General |
| 936 | 19913 | AI639155 | | ESTs | General |
| 937 | 1097 | AF016296 | | Rattus norvegicus neuropilin | General |
| | | | | mRNA, complete cds | |
| 938 | 4594 | Y07704 | | Rattus norvegicus mRNA Best5 | e, General |
| | | | | protein | |
| 939 | 22146 | AA893980 | | ESTs | General |
| 940 | 13382 | AI101527 | | ESTs, Highly similar to | a, g |
| | | | | SR19_HUMAN SIGNAL | |
| | | | | RECOGNITION PARTICLE 19 KD | |
| | | | | PROTEIN [H. sapiens] | |
| 941 | 2399 | AI030247 | | ESTs | General |
| 942 | 17644 | AA924036 | | ESTs | General |
| 943 | 895 | AI009614 | | ESTs | General |
| 944 | 1802 | AA817841 | | ESTs | c |
| 945 | 3003 | AA997330 | | ESTs | d, General |
| 946 | 9757 | AI170693 | | ESTs | c |
| 947 | 23583 | AI234819 | | ESTs | General |
| 948 | 8886 | AI233766 | | ESTs, Weakly similar to protease | f, General |
| | | | | [R. norvegicus] | |
| 949 | 24185 | AA893708 | | ESTs, Highly similar to T00333 | h |
| | | | | hypothetical protein KIAA0560 | |
| | | | | [H. sapiens] | |
| 950 | 22929 | AI071578 | | ESTs, Moderately similar to | General |
| | | | | NEURONAL PROTEIN 3.1 | |
| | | | | [M. musculus] | |
| 951 | 18271 | AA925267 | | ESTs, Highly similar to KIAA0740 | e |
| | | | | protein [H. sapiens] | |
| 952 | 3773 | AA998356 | | ESTs, Weakly similar to | General |
| | | | | BCL3_HUMAN B-CELL | |
| | | | | LYMPHOMA 3-ENCODED | |
| | | | | PROTEIN [H. sapiens] | |
| 953 | 18108 | X62528 | | R.rattus mRNA for ribonuclease | f |
| | | | | inhibitor | |
| 954 | 6176 | AA819657 | | EST | a |
| 955 | 14250 | AA799729 | Phosphodiesterase 4B, cAMP- | ESTs, Phosphodiesterase 4B, | General |
| | | | specific (dunce (Drosophila)- | cAMP-specific (dunce (Drosophila)- | |
| | | | homolog phosphodiesterase E4) | homolog phosphodiesterase E4) | |
| 956 | 22948 | AI105421 | | ESTs | d |
| 957 | 10991 | AI104342 | | ESTs | b |
| 958 | 11563 | AI102560 | | ESTs | a |
| 959 | 25669 | X07314 | | | a, b |
| 960 | 9277 | AI072424 | | ESTs, Highly similar to AF155568 1 | i |
| | | | | NSAP1 protein [H. sapiens] | |
| 961 | 13467 | AI138034 | UDP-glucose: ceramide | UDP-glucose: ceramide | General |
| | | | glycosyltransferase | glycosyltransferase | |
| 962 | 11516 | AI103962 | | ESTs | h |
| 963 | 20110 | AF071204 | stem cell factor KL-1 precursor | stem cell factor KL-1 precursor | b |
| 964 | 19320 | AA891666 | melanoma antigen, family D, 1 | melanoma antigen, family D, 1 | i |
| 965 | 13029 | AI179391 | | ESTs | d, General |
| 966 | 10348 | AI069934 | | EST | e |
| 967 | 8495 | AI059971 | | ESTs, Weakly similar to TNRC | d, g, i, |
| | | | | MOUSE LYMPHOTOXIN-BETA | General |
| | | | | RECEPTOR PRECURSOR | |
| | | | | [M. musculus] | |
| 968 | 14968 | K02815 | | Rat mRNA for RT1.B-1(alpha) chain | General |
| | | | | of integral membrane protein | |
| 969 | 19358 | AI009675 | | EST | a, b, |
| | | | | General | |
| 970 | 4031 | AI176989 | | ESTs | a |
| 971 | 19005 | AI175875 | | ESTs | d |
| 972 | 21661 | AI176479 | | Rattus norvegicus interferon- | b, f, |
| | | | | inducible protein variant 10 mRNA, | General |
| | | | | complete cds | |
| 973 | 13596 | U90261 | SH3 domain-containing adapter | SH3 domain-containing adapter | c, h |
| | | | protein | protein | |
| 974 | 11724 | AI102812 | | ESTs | a, b, |
| | | | | | General |
| 975 | 2042 | AI101921 | | ESTs | General |
| 976 | 25632 | U75405 | | | e |
| 977 | 10227 | AI102248 | | ESTs | h, General |
| 978 | 7224 | AI009820 | | ESTs | h |
| 979 | 2131 | AI171091 | | ESTs, Weakly similar to T19999 | h |
| | | | | hypothetical protein C47D12.2 - | |
| | | | | Caenorhabditis elegans [C. elegans] | |
| 980 | 20055 | AI230762 | | ESTs, Weakly similar to CLP3 RAT | General |
| | | | | CALPONIN, ACIDIC ISOFORM | |
| | | | | [R. norvegicus] | |
| 981 | 18897 | AA875207 | | Rat major beta-globin mRNA, | d |
| | | | | complete cds | |
| 982 | 18468 | AA859966 | | ESTs, Weakly similar to Edp1 | General |
| | | | | protein [M. musculus] | |
| 983 | 22820 | AA848315 | HMm: inosine 5′-phosphate | ESTs, Weakly similar to guanosine | General |
| | | | dehydrogenase 2 | monophosphate reductase | |
| | | | | [R. norvegicus] | |
| 984 | 17901 | AA944355 | | ESTs, Moderately similar to | General |
| | | | | AF155103 1 NY-REN-25 antigen | |
| | | | | [H. sapiens] | |
| 985 | 8305 | AI111695 | | ESTs | a |
| 986 | 14842 | AI237724 | | ESTs | General |
| | 26167 | AI044866 | | | General |
| 987 | 10097 | AI639425 | | ESTs | f, General |
| 988 | 6861 | AI176970 | | ESTs | h |
| 989 | 18859 | AA799467 | | ESTs | g |
| 990 | 21510 | AA851620 | | ESTs | General |
| 991 | 3561 | AB020504 | | Rattus norvegicus mRNA for | General |
| | | | | PMF31, complete cds | |
| 992 | 22898 | AA901107 | | ESTs | General |
| 993 | 14668 | AI012185 | | ESTs | c |
| 994 | 23035 | AA945712 | | ESTs | d |
| 995 | 6330 | AI170426 | | ESTs | b |
| 996 | 3808 | AI008643 | | ESTs, Weakly similar to heat shock | General |
| | | | | protein hsp40-3 [M. musculus] | |
| 997 | 10334 | AI060295 | | ESTs, Moderately similar to small | a |
| | | | | GTP-binding protein rab5 | |
| | | | | [R. norvegicus] | |
| 998 | 22762 | D89730 | | ESTs, Weakly similar to AF137350 | c, General |
| | | | | 1 embryonic vascular EGF repeat- | |
| | | | | containing protein EVEC | |
| | | | | [R. norvegicus] | |
| 999 | 15837 | AI010411 | | ESTs, ESTs, Moderately similar to | g |
| | | | | NADH-ubiquinone oxidoreductase | |
| | | | | B14.5B subunit [H. sapiens] | |
| 1000 | 4169 | AI009669 | | ESTs, Weakly similar to HIGH | General |
| | | | | AFFINITY IMMUNOGLOBULIN | |
| | | | | EPSILON RECEPTOR BETA- | |
| | | | | SUBUNIT [R. norvegicus] | |
| 1001 | 22596 | AA955298 | | ESTs | General |
| 1002 | 11483 | AF020618 | | ESTs, Moderately similar to MY16 | General |
| | | | | MOUSE MYELOID | |
| | | | | DIFFERENTIATION PRIMARY | |
| | | | | RESPONSE PROTEIN MYD116 | |
| | | | | [M. musculus], Rattus norvegicus | |
| | | | | progression elevated gene 3 protein | |
| | | | | mRNA, complete cds | |
| 1003 | 17540 | AA955914 | | EST, EST, Moderately similar to | General |
| | | | | FBRL MOUSE FIBRILLARIN | |
| | | | | [M. musculus], ESTs, Highly similar | |
| | | | | to FBRL MOUSE FIBRILLARIN | |
| | | | | [M. musculus] | |
| 1004 | 26030 | M34331 | | | e |
| 1005 | 15569 | Z78279 | | R. norvegicus mRNA for collagen | c, e |
| | | | | alpha 1 type I | |
| 1006 | 22378 | AA944212 | | ESTs | General |
| 1007 | 23155 | AA926016 | | ESTs, Weakly similar to RING | i |
| | | | | finger protein terf [R. norvegicus] | |
| 1008 | 4832 | AA800190 | | ESTs, Highly similar to glycogen | General |
| | | | | phosphorylase [R. norvegicus] | |
| 1009 | 18774 | AI010279 | | ESTs | a, i |
| 1010 | 21798 | AA926365 | | ESTs, Moderately similar to | b |
| | | | | AF151827 1 CGI-69 protein | |
| | | | | [H. sapiens] | |
| 1011 | 3392 | AA998205 | | ESTs | d |
| 1012 | 9796 | AI071990 | | Rattus norvegicus pEachy mRNA, | General |
| | | | | complete cds | |
| 1013 | 16314 | AA859348 | | ESTs | General |
| 1014 | 2757 | AA997851 | | ESTs | c, General |
| 1015 | 4636 | AA899491 | HMm: tryptophanyl-tRNA | ESTs, Highly similar to SYW | i |
| | | | synthetase | MOUSE TRYPTOPHANYL-TRNA | |
| | | | | SYNTHETASE [M. musculus] | |
| 1016 | 21644 | AI179609 | | ESTs | General |
| 480 | 11727 | AA849518 | | ESTs | d |
| 1017 | 345 | AI059916 | Signal transducer and activator | Signal transducer and activator of | f |
| | | | of transcription 3 | transcription 3 | |
| 1018 | 22773 | AA859885 | | ESTs | c |
| 1019 | 2767 | AA963201 | | ESTs | General |
| 1020 | 22036 | AI070141 | | ESTs, Highly similar to CD97 | General |
| | | | | protein [M. musculus] | |
| 1021 | 8580 | AI060314 | | EST | c |
| 1022 | 9362 | AI102706 | | ESTs | General |
| 1023 | 10659 | AI103059 | | ESTs | General |
| 1024 | 16549 | AA892270 | | ESTs | a |
| 1025 | 2254 | AI235325 | | ESTs | h, General |
| 1026 | 12366 | AI232088 | | ESTs | General |
| 1027 | 20897 | AI175812 | | ESTs, Highly similar to Copa | General |
| | | | | protein [M. musculus] | |
| 1028 | 13270 | AI228760 | | ESTs | d |
| 1029 | 12031 | AA893860 | HHs: threonyl-tRNA synthetase | ESTs, Moderately similar to | General |
| | | | | SYTC_HUMAN | |
| | | | | THREONYL-TRNA | |
| | | | | SYNTHETASE, CYTOPLASMIC | |
| | | | | [H. sapiens] | |
| 1030 | 16859 | AI236753 | | ESTs | General |
| 1031 | 6982 | AI170793 | | ESTs, Highly similar to titin-like | General |
| | | | | protein [H. sapiens] | |
| 1032 | 15518 | AI072945 | | ESTs | General |
| 1033 | 8612 | AI058527 | | ESTs | a, b, |
| | | | | | General |
| 1034 | 14289 | AA891838 | | ESTs, Highly similar to muscle | b |
| | | | | protein 684 [M. musculus] | |
| 580 | 22248 | AA943537 | | Rattus norvegicus zyxin mRNA, | General |
| | | | | partial cds | |
| 1035 | 12303 | AA944786 | | ESTs | d |
| 1036 | 17632 | AI102472 | | ESTs | c |
| 1037 | 19722 | AI013508 | | Rattus norvegicus Hsp70 binding | General |
| | | | | protein HspBP mRNA, complete | |
| | | | | cds | |
| 1038 | 15051 | AI236332 | | ESTs, Highly similar to ATDA | General |
| | | | | MOUSE DIAMINE | |
| | | | | ACETYLTRANSFERASE | |
| | | | | [M. musculus] | |
| 1039 | 7273 | AI234712 | | ESTs | f |
| 1040 | 15879 | AI228313 | | ESTs | General |
| 1041 | 10020 | AI045632 | | ESTs | f, General |
| 1042 | 22432 | AI171263 | | ESTs, Highly similar to FBRL | g, General |
| | | | | MOUSE FIBRILLARIN | |
| | | | | [M. musculus] | |
| 1043 | 25705 | X59375 | | | General |
| 1044 | 4696 | AI237009 | | ESTs | c |
| 1045 | 18673 | AA849028 | | ESTs, Highly similar to PRC8 RAT | d |
| | | | | PROTEASOME COMPONENT C8 | |
| | | | | [R. norvegicus] | |
| 1046 | 13166 | AI178736 | | ESTs | b |
| 1047 | 24128 | AA849766 | | ESTs, Highly similar to hypothetical | c |
| | | | | protein [H. sapiens] | |
| 1048 | 24042 | AI230002 | | ESTs | General |
| 1049 | 9339 | AI101160 | | ESTs, Weakly similar to S46930 | General |
| | | | | teg292 protein - mouse | |
| | | | | [M. musculus] | |
| 1050 | 15190 | AI102562 | | Rat metallothionein-i (mt-1) mrna | General |
| 1051 | 3433 | AI030339 | | ESTs | General |
| 1052 | 4459 | AA891721 | | ESTs | General |
| 1053 | 19012 | AI172056 | | ESTs | General |
| 1054 | 2802 | D85435 | | Rattus norvegicus mRNA for protein | c |
| | | | | kinase C delta-bindig protein, | |
| | | | | complete cds | |
| 1055 | 14763 | AA944481 | | ESTs, Weakly similar to FIBA RAT | General |
| | | | | FIBRINOGEN ALPHA/ALPHA-E | |
| | | | | CHAIN PRECURSOR | |
| | | | | [R. norvegicus] | |
| 1056 | 9583 | AI071185 | | ESTs | d, General |
| 1057 | 10394 | AA943564 | | ESTs, Weakly similar to T27118 | General |
| | | | | hypothetical protein Y53C10A.5 - | |
| | | | | Caenorhabditis elegans [C. elegans] | |
| 1058 | 16821 | AA999042 | | ESTs | General |
| 1059 | 3718 | AI229643 | | ESTs | General |
| 1060 | 8022 | AI058357 | | ESTs | General |
| 1061 | 19840 | AA899197 | | Rattus norvegicus mRNA for | g, General |
| | | | | poly(A) binding protein (pabp1 | |
| | | | | gene) | |
| 1062 | 15215 | AI060260 | | ESTs | d |
| 1063 | 8053 | AI175033 | | ESTs | General |
| 1064 | 12086 | AA801116 | | ESTs | c |
| 1065 | 21447 | AI009608 | | ESTs, Highly similar to orf | d |
| | | | | [M. musculus] | |
| 1066 | 4232 | AI012958 | | ESTs | General |
| 1067 | 12978 | X96437 | | ESTs, Highly similar | General |
| | | | | to RADIATION- | |
| | | | | INDUCIBLE IMMEDIATE-EARLY | |
| | | | | GENE IEX-1 [M. musculus] | |
| 1068 | 1031 | AA892863 | | ESTs | d |
| 1069 | 18682 | AI059499 | | ESTs, Highly similar to chaperonin | h |
| | | | | containing TCP-1 theta subunit | |
| | | | | [M. musculus] | |
| 1070 | 25806 | E00898 | | | g |
| 1071 | 4432 | AI101851 | | ESTs | d |
| 1072 | 2997 | AI030545 | | ESTs | i, General |
| 1073 | 6936 | AI010593 | | ESTs | d |
| 1074 | 11684 | AI010917 | | ESTs | g, General |
| 1075 | 804 | AI169756 | | ESTs, Highly similar to GENE 33 | General |
| | | | | POLYPEPTIDE [R. norvegicus] | |
| 1076 | 15192 | AI101099 | | Rat metallothionein-2 and | General |
| | | | | metallothionein-1 genes, complete | |
| | | | | cds | |
| 1077 | 19145 | AA964613 | | ESTs | f |
| 1078 | 3887 | AA893307 | | EST | a |
| 1079 | 13539 | AI177280 | | ESTs, Moderately similar to A45304 | General |
| | | | | granulocyte/macrophage colony- | |
| | | | | stimulating factor receptor, low | |
| | | | | affinity, precursor - mouse | |
| | | | | [M. musculus] | |
| 1080 | 5258 | AA926089 | | ESTs, Highly similar to similar to | d |
| | | | | human DNA-binding protein 5 | |
| | | | | [H. sapiens] | |
| 1081 | 17380 | AA799612 | ubiquitin conjugating enzyme | ubiquitin conjugating enzyme | h |
| 544 | 11530 | AA799773 | | ESTs, Highly similar to gamma- | i |
| | | | | filamin [H. sapiens] | |
| 1082 | 8541 | AI112310 | | ESTs, Highly similar to hypothetical | d |
| | | | | protein [H. sapiens] | |
| 1083 | 15876 | AA892582 | | ESTs, Highly similar to | e, h |
| | | | | RL8_HUMAN 60S RIBOSOMAL | |
| | | | | PROTEIN L [R. norvegicus] | |
| 1084 | 7806 | AA818421 | | ESTs | General |
| 1085 | 19011 | AI102618 | | ESTs | General |
| 1086 | 8938 | AI070590 | | ESTs | General |
| 1087 | 13266 | AI172326 | | ESTs | General |
| 1088 | 22765 | AI176265 | | ESTs | General |
| 1089 | 22196 | U21719 | | ESTs | General |
| 1090 | 6789 | AA998207 | | ESTs | i, General |
| 1091 | 1287 | AI172299 | | ESTs | f, General |
| 1092 | 13985 | AA819429 | | EST | e |
| 1093 | 5887 | AI179099 | | ESTs, Moderately similar to Vanin-1 | b |
| | | | | [M. musculus] | |
| 1094 | 15189 | M11794 | | | General |
| 1095 | 657 | AI010262 | | Rattus norvegicus mRNA for | General |
| | | | | inetrleukin-4 receptor (membrane- | |
| | | | | bound form), complete cds | |
| 1096 | 14554 | U48828 | | R. norvegicus H1SHR mRNA, Rattus | f, i, General |
| | | | | norvegicus retroviral-like ovarian | |
| | | | | specific transcript 30-1 mRNA | |
| 1097 | 23096 | AA924352 | | ESTs, Weakly similar to Prostatic | a, b |
| | | | | Acid Phosphatase [R. norvegicus] | |
| 1098 | 7120 | AI012393 | | ESTs, Weakly similar to RING | a, b |
| | | | | finger protein terf [R. norvegicus] | |
| 1099 | 13033 | AI113076 | | ESTs | c |
| 1100 | 12745 | AI011799 | | ESTs | a, General |
| 1101 | 8759 | AI237646 | | ESTs | General |
| 950 | 22930 | AI071578 | | ESTs, Moderately similar to | e |
| | | | | NEURONAL PROTEIN 3.1 | |
| | | | | [M. musculus] | |
| 1102 | 4490 | AA851184 | | Rattus norvegicus mRNA for | General |
| | | | | cathepsin Y, partial cds | |
| 1103 | 600 | AA800222 | | ESTs | c, e |
| 779 | 1687 | AI179971 | Hemoglobin, alpha 1 | Hemoglobin, alpha 1 | a, b, |
| | | | | | General |
| 1104 | 23139 | AI180040 | | ESTs | d, f |
| 1105 | 11876 | AI009321 | | ESTs, Highly similar to similar to | General |
| | | | | human DNA-binding protein 5 | |
| | | | | [H. sapiens] | |
| 1106 | 13568 | AA800169 | | ESTs | General |
| 1107 | 25671 | X07686 | | | e |
| 1108 | 2410 | AA964589 | | EST | General |
| 1109 | 23230 | AI236146 | | ESTs | d |
| 766 | 15134 | D10706 | | ESTs, Highly similar to OAZ RAT | c |
| | | | | ORNITHINE DECARBOXYLASE | |
| | | | | ANTIZYME [R. norvegicus] | |
| 1110 | 6166 | AI136516 | | ESTs | General |
| 1111 | 12066 | AA800566 | | ESTs | c |
| 684 | 17947 | AA944581 | | ESTs, Highly similar to RAB/Rip | i |
| | | | | protein [M. musculus] | |
| 371 | 19256 | M15562 | | Rat (diabetic BB) MHC class II | f, General |
| | | | | alpha chain RT1.D alpha (u) | |
| 1112 | 23210 | H31420 | | ESTs | h |
| 1113 | 5844 | AA944367 | | ESTs, Highly similar to N- | General |
| | | | | acetylglucosamine-phosphate | |
| | | | | mutase [H. sapiens] | |
| 1114 | 2992 | AI171776 | | ESTs | General |
| 1115 | 8515 | AA849917 | | ESTs | a, b |
| 1116 | 21974 | AA945769 | | ESTs, Highly similar to T43483 | a |
| | | | | translation initiation factor IF-2 | |
| | | | | homolog [H. sapiens] | |
| 1117 | 11896 | AA997845 | | ESTs, Highly similar to protein | a |
| | | | | kinase Dyrk2 [H. sapiens] | |
| 1118 | 10820 | AI009411 | | ESTs, Highly similar to RS3 | e, General |
| | | | | MOUSE 40S RIBOSOMAL | |
| | | | | PROTEIN S3 [R. norvegicus] | |
| 1119 | 6532 | AI234105 | | ESTs | General |
| 1120 | 6274 | AA819206 | | ESTs | General |
| 1121 | 23099 | AI112365 | | ESTs, Highly similar to mm-Mago | General |
| | | | | [M. musculus] | |
| 1122 | 12898 | AI105049 | | ESTs | General |
| 1123 | 22011 | AI176212 | | ESTs, Weakly similar to T25165 | General |
| | | | | hypothetical protein T23D8.3 - | |
| | | | | Caenorhabditis elegans [C. elegans] | |
| 1124 | 14693 | AI234830 | | ESTs, Weakly similar to | General |
| | | | | YK61_YEAST HYPOTHETICAL | |
| | | | | 39.6 KDA PROTEIN IN MTD1- | |
| | | | | NUP133 INTERGENIC REGION | |
| | | | | [S. cerevisiae] | |
| 1125 | 12107 | AI172460 | | ESTs | d |
| 1126 | 3799 | AF002281 | | Rattus norvegicus alpha-actinin-2 | f, General |
| | | | | associated LIM protein mRNA, | |
| | | | | complete cds | |
| 1127 | 14955 | AA945750 | | ESTs | d |
| 1128 | 14862 | AI236048 | | EST | f |
| 1129 | 9317 | AI029174 | | ESTs | h |
| 1130 | 24028 | AI180239 | | ESTs | General |
| 1131 | 16081 | AI179610 | Heme oxygenase | Heme oxygenase | General |
| 1132 | 25918 | AI639204 | | | e |
| 1133 | 15042 | AI179422 | | ESTs | General |
| 1134 | 11242 | AI179260 | | ESTs | General |
| 1135 | 18018 | D12771 | | Rattus norvegicus mRNA for | i |
| | | | | mitochondrial adenine nuleotide | |
| | | | | translocator | |
| 1136 | 6655 | AA850688 | | ESTs | f |
| 1137 | 10182 | AI176185 | | ESTs, Highly similar to P55-C-FOS | General |
| | | | | PROTO-ONCOGENE PROTEIN | |
| | | | | [R. norvegicus] | |
| 1138 | 20990 | AI176509 | | ESTs, Weakly similar to | i |
| | | | | KPCN_HUMAN | |
| | | | | PROTEIN KINASE | |
| | | | | C, NU TYPE (NPKC-NU) | |
| | | | | (PROTEIN KINASE EPK2) | |
| | | | | [H. sapiens] | |
| 1139 | 12118 | AA892775 | Lysozyme | Lysozyme | g |
| 1140 | 4355 | AI103410 | | ESTs | General |

TABLE 2
Attorney Docket No. 44921-5090US Document No. 1828250.1

| Seq. ID | | GenBank | | Model |
|---|---|---|---|---|
| No. | Identifier | Acc. No. | Pathways | Code |
| 894 | 7888 | AI233583 | Aminoacyl-tRNA biosynthesis, Arginine and | h |
| | | | proline metabolism | |
| 1029 | 12031 | AA893860 | Aminoacyl-tRNA biosynthesis, Glycine, serine | General |
| | | | and threonine metabolism | |
| 1015 | 4636 | AA899491 | Aminoacyl-tRNA biosynthesis, Tryptophan | i |
| | | | metabolism | |
| 138 | 17100 | NM_022179 | Aminosugars metabolism, Fructose and | h, General |
| | | | mannose metabolism, Galactose | |
| | | | metabolism, Glycolysis/ | |
| | | | Gluconeogenesis, Starch and sucrose | |
| | | | metabolism | |
| 138 | 17101 | NM_022179 | Aminosugars metabolism, Fructose and | General |
| | | | mannose metabolism, Galactose | |
| | | | metabolism, Glycolysis/ | |
| | | | Gluconeogenesis, Starch and sucrose | |
| | | | metabolism | |
| 168 | 9929 | NM_024392 | Androgen and estrogen metabolism | g |
| 56 | 19392 | NM_012998 | Arginine and proline metabolism | i |
| 149 | 20915 | NM_022407 | Arginine and proline metabolism, Ascorbate and | General |
| | | | aldarate metabolism, Bile acid | |
| | | | biosynthesis, Butanoate metabolism, Fatty acid | |
| | | | metabolism, Glycerolipid metabolism, Histidine | |
| | | | metabolism, Lysine degradation, Propanoate | |
| | | | metabolism, Pyruvate metabolism, Tryptophan | |
| | | | metabolism, Valine, leucine and isoleucine | |
| | | | degradation, beta-Alanine metabolism | |
| 16 | 4468 | NM_012529 | Arginine and proline metabolism, Urea cycle and | e |
| | | | metabolism of amino groups | |
| 25 | 23523 | NM_012615 | Arginine and proline metabolism, Urea cycle and | General |
| | | | metabolism of amino groups | |
| 152 | 4242 | NM_022521 | Arginine and proline metabolism, Urea cycle and | f |
| | | | metabolism of amino groups | |
| 13 | 7063 | NM_012495 | Carbon fixation, Fructose and mannose | c |
| | | | metabolism, Glycolysis/ | |
| | | | Gluconeogenesis, Inositol metabolism, Pentose | |
| | | | phosphate cycle | |
| 667 | 17289 | AA891785 | Citrate cycle (TCA cycle), Glutathione | b |
| | | | metabolism, Reductive carboxylate cycle (CO2 | |
| | | | fixation) | |
| 92 | 24106 | NM_017141 | DNA polymerase, Purine metabolism, Pyrimidine | c |
| | | | metabolism | |
| 92 | 24107 | NM_017141 | DNA polymerase, Purine metabolism, Pyrimidine | General |
| | | | metabolism | |
| 200 | 12581 | NM_031514 | epo, ifn_gamma, il3, il6, interact6-1, pdgf, tpo | i, General |
| 257 | 13005 | NM_053623 | Fatty acid metabolism | General |
| 311 | 20984 | D90109 | Fatty acid metabolism | h |
| 400 | 20983 | AI044900 | Fatty acid metabolism | General |
| 853 | 18686 | D00729 | Fatty acid metabolism | e, h |
| 324 | 23596 | AI105435 | Fatty acid metabolism, Lysine | c, General |
| | | | degradation, Tryptophan metabolism | |
| 115 | 1174 | NM_019184 | Fatty acid metabolism, Tryptophan metabolism | b |
| 203 | 4011 | NM_031543 | Fatty acid metabolism, Tryptophan metabolism | a |
| 144 | 13480 | NM_022390 | Folate biosynthesis | f |
| 816 | 21400 | M36410 | Folate biosynthesis | General |
| 12 | 15511 | NM_012498 | Fructose and mannose metabolism, Galactose | h |
| | | | metabolism, Glycerolipid metabolism, Pentose | |
| | | | and glucuronate interconversions, Pyruvate | |
| | | | metabolism | |
| 68 | 1321 | NM_013098 | Galactose metabolism, Glycolysis/ | a |
| | | | Gluconeogenesis, Starch and sucrose | |
| | | | metabolism | |
| 86 | 11152 | NM_017073 | Glutamate metabolism, Nitrogen metabolism | General |
| 86 | 11153 | NM_017073 | Glutamate metabolism, Nitrogen metabolism | General |
| 879 | 7867 | AI043695 | Glutamate metabolism, Purine metabolism | d |
| 40 | 961 | NM_012796 | Glutathione metabolism | e |
| 914 | 23927 | AA957007 | Glutathione metabolism | e |
| 194 | 1638 | NM_031143 | Glycerolipid metabolism | General |
| 746 | 14149 | AA998172 | Glycerolipid metabolism | i |
| 176 | 23109 | NM_031000 | Glycerolipid metabolism, Glycolysis/ | c, f, h |
| | | | Gluconeogenesis, Pentose and glucuronate | |
| | | | interconversions | |
| 398 | 1893 | X51529 | Glycerolipid metabolism, Phospholipid | General |
| | | | degradation, Prostaglandin and leukotriene | |
| | | | metabolism | |
| 78 | 16448 | NM_013197 | Glycine, serine and threonine metabolism | a, b, |
| | | | | General |
| 236 | 4093 | NM_053290 | Glycolysis/Gluconeogenesis | General |
| 18 | 19408 | NM_012554 | Glycolysis/Gluconeogenesis, Phenylalanine, | h |
| | | | tyrosine and tryptophan biosynthesis | |
| 213 | 21575 | NM_031698 | Glycoprotein biosynthesis | b |
| 132 | 574 | NM_019905 | Glyoxylate and dicarboxylate metabolism | g, i |
| | | | | General |
| 23 | 24716 | NM_012589 | il6, interact6-1 | General |
| 163 | 21239 | NM_024125 | il6, interact6-1 | c, General |
| 77 | 1714 | NM_013187 | Inositol phosphate metabolism | g, i, |
| | | | | General |
| 15 | 15741 | NM_012520 | Methane metabolism, Tryptophan metabolism | c, g |
| 71 | 2005 | NM_013127 | Nicotinate and nicotinamide metabolism | c |
| 124 | 15056 | NM_019291 | Nitrogen metabolism | General |
| 197 | 18597 | NM_031325 | Nucleotide sugars metabolism, Pentose and | General |
| | | | glucuronate interconversions, Starch and | |
| | | | sucrose metabolism | |
| 39 | 449 | NM_012786 | Oxidative phosphorylation | a, b |
| 101 | 14696 | NM_017202 | Oxidative phosphorylation | e |
| 452 | 15048 | M24542 | Oxidative phosphorylation | c, i |
| 130 | 22727 | NM_019383 | Oxidative phosphorylation, Type III protein | i |
| | | | secretion system | |
| 586 | 17204 | AI171844 | Oxidative phosphorylation, Type III protein | e, g |
| | | | secretion system | |
| 887 | 15524 | D13124 | Oxidative phosphorylation, Type III protein | b |
| | | | secretion system | |
| 586 | 17203 | AI171844 | Oxidative phosphorylation, Type III protein | b, e |
| | | | secretion system | |
| 614 | 23038 | AA900881 | Pantothenate and CoA biosynthesis, Valine, | General |
| | | | leucine and isoleucine biosynthesis, Valine, | |
| | | | leucine and isoleucine degradation | |
| 20 | 16080 | NM_012580 | Porphyrin and chlorophyll metabolism | General |
| 1131 | 16081 | AI179610 | Porphyrin and chlorophyll metabolism | General |
| 103 | 20193 | NM_017232 | Prostaglandin and leukotriene metabolism | General |
| 114 | 17066 | NM_019170 | Prostaglandin and leukotriene metabolism | e |
| 178 | 1845 | NM_031010 | Prostaglandin and leukotriene metabolism | a, b, |
| | | | | General |
| 178 | 25517 | NM_031010 | Prostaglandin and leukotriene metabolism | a, b, |
| | | | | General |
| 205 | 692 | NM_031557 | Prostaglandin and leukotriene metabolism | h |
| 107 | 3254 | NM_017282 | Proteasome | h |
| 616 | 3988 | AI008546 | Proteasome | d |
| 88 | 8888 | NM_017090 | Purine metabolism | General |
| 97 | 21975 | NM_017154 | Purine metabolism | General |
| 217 | 14184 | NM_031776 | Purine metabolism | General |
| 217 | 14185 | NM_031776 | Purine metabolism | General |
| 289 | 15843 | NM_080910 | Purine metabolism | h |
| 377 | 14384 | AI177096 | Purine metabolism | c |
| 870 | 13547 | M63983 | Purine metabolism | i |
| 955 | 14250 | AA799729 | Purine metabolism | General |
| 983 | 22820 | AA848315 | Purine metabolism | General |
| 79 | 20826 | NM_013218 | Pyrimidine metabolism | h |
| 208 | 24235 | NM_031614 | Pyrimidine metabolism | General |
| 561 | 17285 | J02827 | Valine, leucine and isoleucine degradation | General |

TABLE 3
Attorney Docket No. 44921-5090US Document No. 1828250.1

| Seq. ID | | GenBank | Homologous Human | | Model |
|---|---|---|---|---|---|
| No. | Identifier | Acc. No. | Genes | Homologous Human Cluster Title | Code |
| 1 | 25319 | J01435 | | EST, Moderately similar to 0806162C protein | a, c, f |
| | | | | COI [M. musculus], EST, Moderately similar to | |
| | | | | 810024C cytochrome oxidase I [H. sapiens], | |
| | | | | ESTs, Moderately similar to 0806162C protein | |
| | | | | COI [M. musculus], ESTs, Moderately similar to | |
| | | | | 810024C cytochrome oxidase I [H. sapiens] | |
| 1 | 25050 | J01435 | | EST, Moderately similar to 0806162C protein | c, f, g |
| | | | | COI [M. musculus], EST, Moderately similar to | |
| | | | | 810024C cytochrome oxidase I [H. sapiens], | |
| | | | | ESTs, Moderately similar to 0806162C protein | |
| | | | | COI [M. musculus], ESTs, Moderately similar to | |
| | | | | 810024C cytochrome oxidase I [H. sapiens] | |
| 2 | 25051 | J01436 | | | a, c |
| 3 | 25439 | M35826 | | | a, c, f, g |
| 4 | 16132 | AA850885 | | EST, Moderately similar to 0806162C protein | a, c |
| | | | | COI [M. musculus], EST, Moderately similar to | |
| | | | | 810024C cytochrome oxidase I [H. sapiens], | |
| | | | | ESTs, Moderately similar to 0806162C protein | |
| | | | | COI [M. musculus], ESTs, Moderately similar to | |
| | | | | 810024C cytochrome oxidase I [H. sapiens] | |
| 5 | 6153 | AA875531 | collagen, type I, alpha 2, | EST, Moderately similar to 0806162B | e |
| | | | procollagen, type I, alpha 2 | cytochrome b [M. musculus], EST, Moderately | |
| | | | | similar to 810024B cytochrome b [H. sapiens], | |
| | | | | EST, Weakly similar to 0806162B cytochrome | |
| | | | | b [M. musculus], EST, Weakly similar to | |
| | | | | 0812187A cytochrome b [R. norvegicus], EST, | |
| | | | | Weakly similar to 810024M URF 6 [H. sapiens], | |
| | | | | EST, Weakly similar to [Segment 1 of 2] | |
| | | | | COLLAGEN ALPHA 1(I) CHAIN [R. norvegicus], | |
| | | | | ESTs, Weakly similar to CA1F_HUMAN | |
| | | | | COLLAGEN ALPHA 1(XVI) CHAIN | |
| | | | | PRECURSO [H. sapiens], Mus musculus, | |
| | | | | Similar to RIKEN cDNA 1700051I12 gene, | |
| | | | | clone MGC: 28741 IMAGE: 4481590, mRNA, | |
| | | | | complete cds, RIKEN cDNA 1700051I12 gene, | |
| | | | | chordin, collagen, type XVI, alpha 1, expressed | |
| | | | | sequence AI835009, hypothetical protein | |
| | | | | FLJ14547, procollagen, type II, alpha 1 | |
| 6 | 15666 | AA892248 | | | a, f |
| 7 | 19421 | AA945152 | dimethylarginine | EST, Moderately similar to 0806162A ATPase | a, c |
| | | | dimethylaminohydrolase 1 | 6 [M. musculus], EST, Weakly similar to | |
| | | | | 0806162A ATPase 6 [M. musculus], ESTs, | |
| | | | | Moderately similar to DDH1 RAT NG, NG- | |
| | | | | DIMETHYLARGININE | |
| | | | | DIMETHYLAMINOHYDROLASE 1 | |
| | | | | [R. norvegicus], ESTs, Weakly similar to DDH1 | |
| | | | | RAT NG, NG-DIMETHYLARGININE | |
| | | | | DIMETHYLAMINOHYDROLASE 1 | |
| | | | | [R. norvegicus], dimethylarginine | |
| | | | | dimethylaminohydrolase 1 | |
| 8 | 14981 | AI103396 | | EST, Moderately similar to 0806162B | a, c, f |
| | | | | cytochrome b [M. musculus], EST, Moderately | |
| | | | | similar to 810024B cytochrome b [H. sapiens], | |
| | | | | EST, Weakly similar to 0806162B cytochrome | |
| | | | | b [M. musculus], EST, Weakly similar to | |
| | | | | 0812187A cytochrome b [R. norvegicus], EST, | |
| | | | | Weakly similar to 810024M URF 6 [H. sapiens] | |
| 9 | 14982 | AI171355 | | EST, Moderately similar to 0806162B | a, f |
| | | | | cytochrome b [M. musculus], EST, Moderately | |
| | | | | similar to 810024B cytochrome b [H sapiens], | |
| | | | | EST, Weakly similar to 0806162B cytochrome | |
| | | | | b [M. musculus], EST, Weakly similar to | |
| | | | | 0812187A cytochrome b [R. norvegicus], EST, | |
| | | | | Weakly similar to 810024M URF 6 [H. sapiens] | |
| 10 | 14983 | AI179150 | | EST, Moderately similar to 0806162B | a, c, f |
| | | | | cytochrome b [M. musculus], EST, Moderately | |
| | | | | similar to 810024B cytochrome b [H. sapiens], | |
| | | | | EST, Weakly similar to 0806162B cytochrome | |
| | | | | b [M. musculus], EST, Weakly similar to | |
| | | | | 0812187A cytochrome b [R. norvegicus], EST, | |
| | | | | Weakly similar to 810024M URF 6 [H. sapiens] | |
| 11 | 21152 | X14848 | golgi SNAP receptor | Homo sapiens, Similar to golgi SNAP receptor | a, b, c, |
| | | | complex member 1 | complex member 1, clone MGC: 13657 | f, g |
| | | | | IMAGE: 4250494, mRNA, complete cds, golgi | |
| | | | | SNAP receptor complex member 1 | |
| 12 | 15511 | NM_012498 | aldo-keto reductase | | h |
| | | | family 1, member B1 | | |
| | | | (aldose reductase), aldo- | | |
| | | | keto reductase family 1, | | |
| | | | member B3 (aldose | | |
| | | | reductase) | | |
| 13 | 7063 | NM_012495 | aldolase 1, A isoform, | EST, Moderately similar to ADHUA fructose- | c |
| | | | aldolase A, fructose- | bisphosphate aldolase [H. sapiens], EST, | |
| | | | bisphosphate | Weakly similar to ADHUA fructose- | |
| | | | | bisphosphate aldolase [H. sapiens], EST, | |
| | | | | Weakly similar to I39435 fructose-bisphosphate | |
| | | | | aldolase [H. sapiens], ESTs, Moderately similar | |
| | | | | to aldolase A [M. musculus], Homo sapiens, | |
| | | | | aldolase 1, A isoform, clone MGC: 18171 | |
| | | | | IMAGE: 4155253, mRNA, complete cds, RIKEN | |
| | | | | cDNA 4933425L11 gene, aldolase 1, A isoform, | |
| | | | | aldolase 3, C isoform, aldolase A, fructose- | |
| | | | | bisphosphate | |
| 14 | 7427 | NM_012515 | benzodiazapine receptor | Human DNA sequence from clone 34B21 on | h |
| | | | (peripheral), | chromosome 6p12.1-21.1. Contains part of a | |
| | | | benzodiazepine receptor, | gene for a novel protein with ZU5 domain | |
| | | | peripheral | similar to part of Tight Junction Protein ZO1 | |
| | | | | (TJP1) and UNC5 Homologs, the gene for a | |
| | | | | novel BZRP (peripheral benzodiazapine | |
| | | | | receptor (MBR, PBR, PBKS, IBP, Isoquinoline- | |
| | | | | binding protein)) LIKE protein, the gene for a | |
| | | | | novel protein similar to part of APOBEC1 | |
| | | | | (Phorbolin 1, Apolipoprotein B mRNA editing | |
| | | | | protein), and the NFYA gene for nuclear | |
| | | | | transcription factor Y, alpha (CCAAT-Binding | |
| | | | | transcription factor subunit B, CBF-B, CAAT- | |
| | | | | Box DNA binding protein subunit A). Contains | |
| | | | | ESTs, STSs, GSSs, two putative CpG islands | |
| | | | | and a ca repeat polymorphism, RIKEN cDNA | |
| | | | | 25127D2 gene, benzodiazapine receptor | |
| | | | | (peripheral), benzodiazepine receptor, | |
| | | | | peripheral | |
| 15 | 15741 | NM_012520 | catalase, catalase 1 | Homo sapiens cDNA FLJ1272 fis, clone | c, g |
| | | | | HEMBB12371, catalase, catalase 1 | |
| 16 | 4468 | NM_012529 | creatine kinase, brain | EST, Moderately similar to CREATINE | e |
| | | | | KINASE, B CHAIN [R. norvegicus], EST, | |
| | | | | Weakly similar to KIHUCB creatine kinase | |
| | | | | [H. sapiens], creatine kinase, brain | |
| 17 | 23869 | NM_012551 | early growth response 1 | ESTs, Weakly similar to 153869 zinc finger | General |
| | | | | protein - mouse [M. musculus], early growth | |
| | | | | response 1, repressor of GATA, testis zinc | |
| | | | | finger protein | |
| 18 | 19408 | NM_012554 | enolase 1, (alpha), | EST, Moderately similar to ALPHA ENOLASE | h |
| | | | enolase 1, alpha non- | [R. norvegicus], EST, Weakly similar to A2917 | |
| | | | neuron | phosphopyruvate hydratase [H. sapiens], Homo | |
| | | | | sapiens cDNA FLJ12774 fis, clone | |
| | | | | NT2RP21663, moderately similar to ENOLASE | |
| | | | | (EC 4.2.1.11), enolase 1, (alpha), enolase 1, | |
| | | | | alpha non-neuron, enolase alpha, lung-specific, | |
| | | | | expressed sequence AI42712 | |
| 19 | 6477 | NM_012559 | fibrinogen, gamma | EST, Moderately similar to FGHUGB fibrinogen | g |
| | | | polypeptide | gamma-B chain precursor [H. sapiens], EST, | |
| | | | | Moderately similar to Recombinant Human | |
| | | | | Gamma-Fibrinogen Carboxyl Terminal | |
| | | | | Fragment [H. sapiens], ESTs, Weakly similar to | |
| | | | | FIBG RAT FIBRINOGEN GAMMA-A/-B CHAIN | |
| | | | | PRECURSOR [R. norvegicus], RIKEN cDNA | |
| | | | | 1117F23 gene, expressed sequence AI33526, | |
| | | | | fibrinogen, gamma polypeptide, fibrinogen-like | |
| | | | | 1, ficolin A, ficolin B | |
| 20 | 16080 | NM_012580 | heme oxygenase | heme oxygenase (decycling) 1 | General |
| | | | (decycling) 1 | | |
| 21 | 20313 | NM_012585 | 5-hydroxytryptamine | 5-hydroxytryptamine (serotonin) receptor 1A, | General |
| | | | (serotonin) receptor 1A | protein “A” | |
| 22 | 15098 | NM_012588 | insulin-like growth factor | insulin-like growth factor binding protein 3, | c |
| | | | binding protein 3 | protease, serine, 11 (lgf binding) | |
| 23 | 24716 | NM_012589 | interleukin 6, interleukin 6 | interleukin 6, interleukin 6 (interferon, beta 2) | General |
| | | | (interferon, beta 2) | | |
| 24 | 2629 | NM_012603 | myelocytomatosis | RIKEN cDNA 292K7 gene, myelocytomatosis | General |
| | | | oncogene, v-myc avian | oncogene, v-myc avian myelocytomatosis viral | |
| | | | myelocytomatosis viral | oncogene homolog | |
| | | | oncogene homolog | | |
| 25 | 23523 | NM_012615 | ornithine decarboxylase | ESTs, Weakly similar to A Chain A, Crystal | General |
| | | | 1, ornithine | Structure Ornithine Decarboxylase From | |
| | | | decarboxylase, structural | Mouse, Truncated 37 Residues From The C- | |
| | | | | Terminus, To 1.6 Angstrom Resolution | |
| | | | | [M. musculus], ESTs, Weakly similar to | |
| | | | | ORNITHINE DECARBOXYLASE | |
| | | | | [R. norvegicus], ornithine decarboxylase 1, | |
| | | | | ornithine decarboxylase antizyme inhibitor, | |
| | | | | ornithine decarboxylase, structural, ornithine | |
| | | | | decarboxylase-like protein | |
| 26 | 20589 | NM_012618 | S1 calcium binding | S1 calcium binding protein A3, S1 calcium- | General |
| | | | protein A4, S1 calcium- | binding protein A2, S1 calcium-binding protein | |
| | | | binding protein A4 | A3, S1 calcium-binding protein A4, S1 calcium- | |
| | | | (calcium protein, | binding protein A4 (calcium protein, calvasculin, | |
| | | | calvasculin, metastasin, | metastasin, murine placental homolog) | |
| | | | murine placental | | |
| | | | homolog) | | |
| 27 | 15540 | NM_012620 | serine (or cysteine) | serine (or cysteine) proteinase inhibitor, clade E | General |
| | | | proteinase inhibitor, clade | (nexin, plasminogen activator inhibitor type 1), | |
| | | | E (nexin, plasminogen | member 1 | |
| | | | activator inhibitor type 1), | | |
| | | | member 1 | | |
| 28 | 18725 | NM_012645 | | | c |
| 28 | 18726 | NM_012645 | | | General |
| 29 | 9423 | NM_012649 | syndecan 4, syndecan 4 | syndecan 4, syndecan 4 (amphiglycan, | General |
| | | | (amphiglycan, ryudocan) | ryudocan) | |
| 29 | 9424 | NM_012649 | syndecan 4, syndecan 4 | syndecan 4, syndecan 4 (amphiglycan, | General |
| | | | (amphiglycan, ryudocan) | ryudocan) | |
| 30 | 16224 | NM_012656 | secreted acidic cysteine | EST, Moderately similar to SPARC | c |
| | | | rich glycoprotein, | PRECURSOR [R. norvegicus], SPARC-like 1 | |
| | | | secreted protein, acidic, | (mast9, hevin), secreted acidic cysteine rich | |
| | | | cysteine-rich | glycoprotein, secreted protein, acidic, cysteine- | |
| | | | (osteonectin) | rich (osteonectin) | |
| 31 | 1514 | NM_012678 | | ESTs, Highly similar to S7282 tropomyosin | c, h, |
| | | | | TM3-pl, fibroblast [H. sapiens], ESTs, | General |
| | | | | Moderately similar to alpha-tropomyosin slow | |
| | | | | [M. musculus], RIKEN cDNA 18159A23 gene, | |
| | | | | tropomyosin 4 | |
| 32 | 20776 | NM_012680 | tuberous sclerosis 2 | DKFZp566D133 protein, EST, Moderately | General |
| | | | | similar to TSC2_HUMAN TUBERIN | |
| | | | | [H. sapiens], Homo sapiens cDNA FLJ12339 fis, | |
| | | | | clone MAMMA1225, KIAA1272 protein, tuberin- | |
| | | | | like protein 1, tuberous sclerosis 2 | |
| 33 | 25650 | NM_012736 | glycerol phosphate | expressed sequence AA48484, expressed | f |
| | | | dehydrogenase 1, | sequence AI448216, glycerol phosphate | |
| | | | mitochondrial, glycerol-3- | dehydrogenase 1, mitochondrial, glycerol-3- | |
| | | | phosphate | phosphate dehydrogenase 2 (mitochondrial) | |
| | | | dehydrogenase 2 | | |
| | | | (mitochondrial) | | |
| 34 | 344 | NM_012747 | | Signal transducer and activator of transcription | b |
| | | | | 1, signal transducer and activator of | |
| | | | | transcription 1, signal transducer and activator | |
| | | | | of transcription 1, 91 kD, signal transducer and | |
| | | | | activator of transcription 3, signal transducer | |
| | | | | and activator of transcription 3 (acute-phase | |
| | | | | response factor) | |
| 35 | 8829 | NM_012749 | | | h, General |
| 36 | 3601 | NM_012751 | | ESTs, Highly similar to GTR4_HUMAN | d |
| | | | | GLUCOSE TRANSPORTER TYPE 4, INSULIN- | |
| | | | | RESPONSIV [H. sapiens], solute carrier family | |
| | | | | 2 (facilitated glucose transporter), member 1, | |
| | | | | solute carrier family 2 (facilitated glucose | |
| | | | | transporter), member 4, solute carrier family 2 | |
| | | | | (facilitated glucose transporter), member 6 | |
| 37 | 15174 | NM_012756 | | ESTs, Highly similar to 1312358A IGF II | b |
| | | | | receptor [H. sapiens], expressed sequence | |
| | | | | AI661837, insulin-like growth factor 2 receptor | |
| 38 | 1349 | NM_012776 | adrenergic receptor | G protein-coupled receptor kinase 2, groucho | General |
| | | | kinase, beta 1, | gene related (Drosophila), G protein-coupled | |
| | | | adrenergic, beta, receptor | receptor kinase 5, G protein-coupled receptor | |
| | | | kinase 1 | kinase 6, adrenergic, beta, receptor kinase 1, | |
| | | | | rhodopsin kinase | |
| 39 | 449 | NM_012786 | | EST, Moderately similar to CYTOCHROME C | a, b |
| | | | | OXIDASE POLYPEPTIDE VIII-HEART | |
| | | | | PRECURSOR [R. norvegicus], EST, Weakly | |
| | | | | similar to COXQ MOUSE CYTOCHROME C | |
| | | | | OXIDASE POLYPEPTIDE VIII-HEART | |
| | | | | PRECURSOR [M. musculus], cytochrome c | |
| | | | | oxidase subunit VIII, cytochrome c oxidase, | |
| | | | | subunit VIIIb | |
| 40 | 961 | NM_012796 | glutathione S-transferase | ESTs, Highly similar to GTT2 RAT | e |
| | | | theta 2, glutathione S- | GLUTATHIONE S-TRANSFERASE YRS-YRS | |
| | | | transferase, theta 2 | [R. norvegicus], Homo sapiens mRNA; cDNA | |
| | | | | DKFZp762N226 (from clone DKFZp762N226), | |
| | | | | expressed sequence AI266894, glutathione S- | |
| | | | | transferase theta 2, glutathione S-transferase, | |
| | | | | theta 2, hypothetical protein | |
| 41 | 15032 | NM_012816 | | alpha-methylacyl-CoA racemase, hypothetical | g, i |
| | | | | protein FLJ1188 | |
| 42 | 18109 | NM_012823 | | EST, Weakly similar to ANXA_HUMAN | General |
| | | | | ANNEXIN XI [H. sapiens], annexin A1, annexin | |
| | | | | A3 | |
| 43 | 11137 | NM_012839 | cytochrome c, | ESTs, Highly similar to 63485A cytochrome c | g |
| | | | cytochrome c, somatic | [H. sapiens], ESTs, Weakly similar to | |
| | | | | CYTOCHROME C, SOMATIC [M. musculus], | |
| | | | | Homo sapiens pseudogene for cytochrome c- | |
| | | | | like protein, clone pHGC4E1, Human DNA | |
| | | | | sequence from clone RP11-169O17 on | |
| | | | | chromosome 13 Contains ESTs, GSSs, STSs | |
| | | | | and four CpG islands. Contains a novel protein | |
| | | | | similar to cytochrome c, part of a novel gene | |
| | | | | similar to TPTE encoding a transmembrane | |
| | | | | phosphatase with tensin homology and the | |
| | | | | ADPRTL1 gene encoding ADP- | |
| | | | | ribosyltransferase (NAD+; poly (ADP-ribose) | |
| | | | | polymerase)-like protein 1 (vault protein, | |
| | | | | KIAA177), cytochrome c, cytochrome c, | |
| | | | | somatic | |
| 44 | 17541 | NM_012844 | epoxide hydrolase 1, | EST, Moderately similar to HYEP_HUMAN | a, c, |
| | | | microsomal, epoxide | EPOXIDE HYDROLASE 1 [H. sapiens], ESTs, | General |
| | | | hydrolase 1, microsomal | Highly similar to HYEP_HUMAN EPOXIDE | |
| | | | (xenobiotic) | HYDROLASE 1 [H. sapiens], epoxide hydrolase | |
| | | | | 1, microsomal, epoxide hydrolase 1, | |
| | | | | microsomal (xenobiotic) | |
| 45 | 17305 | NM_012876 | ribosomal protein S29 | EST, Highly similar to 4S RIBOSOMAL | b, e, g |
| | | | | PROTEIN S29 [R. norvegicus], EST, | |
| | | | | Moderately similar to RS29_HUMAN 4S | |
| | | | | RIBOSOMAL PROTEIN S29 [M. musculus], | |
| | | | | EST, Weakly similar to 4S RIBOSOMAL | |
| | | | | PROTEIN S29 [R. norvegicus], ESTs, Highly | |
| | | | | similar to RS29_HUMAN 4S RIBOSOMAL | |
| | | | | PROTEIN S29 [H. sapiens], ESTs, Weakly | |
| | | | | similar to RS29_HUMAN 4S RIBOSOMAL | |
| | | | | PROTEIN S29 [H. sapiens], ribosomal protein | |
| | | | | S29 | |
| 46 | 1748 | NM_012889 | vascular cell adhesion | immunoglobulin superfamily, member 1, mRNA | a, General |
| | | | molecule 1 | for 1-gicerin/MUC18, mucosal vascular | |
| | | | | addressin cell adhesion molecule 1, vascular | |
| | | | | cell adhesion molecule 1 | |
| 47 | 16721 | NM_012891 | acyl-Coenzyme A | EST, Weakly similar to ACYL-COA | General |
| | | | dehydrogenase, very long | DEHYDROGENASE, VERY-LONG-CHAIN | |
| | | | chain | SPECIFIC PRECURSOR [R. norvegicus], | |
| | | | | ESTs, Weakly similar to ACYL-COA | |
| | | | | DEHYDROGENASE, VERY-LONG-CHAIN | |
| | | | | SPECIFIC PRECURSOR [R. norvegicus], NPD2 | |
| | | | | protein, RIKEN cDNA 2617P15 gene, acyl | |
| | | | | Coenzyme A dehydrogenase, very long chain | |
| 48 | 16274 | NM_012898 | alpha-2-HS-glycoprotein | alpha-2-HS-glycoprotein | a |
| 49 | 24783 | NM_012914 | ATPase, Ca++ | ATPase, Ca++ transporting, cardiac muscle, | h, General |
| | | | transporting, ubiquitous | fast twitch 1, ATPase, Ca++ transporting, | |
| | | | | cardiac muscle, slow twitch 2, ATFase, Ca++ | |
| | | | | transporting, ubiquitous, expressed sequence | |
| | | | | AW61228 | |
| 50 | 18695 | NM_012931 | | CD2-associated protein, HEF like Protein, | b, General |
| | | | | Homo sapiens cDNA FLJ14854 fis, clone | |
| | | | | PLACE1972, breast cancer anti-estrogen | |
| | | | | resistance 1, enhancer of filamentation 1 (cas- | |
| | | | | like docking; Crk-associated substrate related), | |
| | | | | neural precursor cell expressed, | |
| | | | | developmentally down-regulated gene 9, v-crk- | |
| | | | | associated tyrosine kinase substrate | |
| 51 | 2555 | NM_012967 | intercellular adhesion | Homo sapiens mRNA; cDNA DKFZp434E516 | General |
| | | | molecule, intercellular | (from clone DKFZp434E516), intercellular | |
| | | | adhesion molecule 1 | adhesion molecule, intercellular adhesion | |
| | | | (CD54), human rhinovirus | molecule 1 (CD54), human rhinovirus receptor, | |
| | | | receptor | intercellular adhesion molecule 3, intercellular | |
| | | | | adhesion molecule 5, telencephalin | |
| 52 | 24528 | NM_012973 | potassium voltage-gated | potassium voltage-gated channel, lsk-related | h |
| | | | channel, lsk-related | family, member 1, potassium voltage-gated | |
| | | | family, member 1, | channel, lsk-related subfamily, member 1 | |
| | | | potassium voltage-gated | | |
| | | | channel, lsk-related | | |
| | | | subfamily, member 1 | | |
| 53 | 956 | NM_012976 | | ESTs, Highly similar to LEG9 RAT GALECTIN- | a, General |
| | | | | 9 [R. norvegicus], ESTs, Highly similar to | |
| | | | | LEG9_HUMAN GALECTIN-9 [H. sapiens], | |
| | | | | ESTs, Weakly similar to LEG9 RAT GALECTIN- | |
| | | | | 9 [R. norvegicus], expressed sequence | |
| | | | | AI265545, lectin, galactose binding, soluble 12, | |
| | | | | lectin, galactose binding, soluble 9, lectin, | |
| | | | | galactoside-binding, soluble, 9 (galectin 9) | |
| 54 | 958 | NM_012977 | | ESTs, Highly similar to LEG9 RAT GALECTIN- | General |
| | | | | 9 [R. norvegicus], ESTs, Highly similar to | |
| | | | | LEG9_HUMAN GALECTIN-9 [H. sapiens], | |
| | | | | ESTs, Weakly similar to LEG9 RAT GALECTIN- | |
| | | | | 9 [R. norvegicus], expressed sequence | |
| | | | | AI265545, lectin, galactose binding, soluble 12, | |
| | | | | lectin, galactose binding, soluble 9, lectin, | |
| | | | | galactoside-binding, soluble, 9 (galectin 9) | |
| 55 | 17394 | NM_012992 | nucleophosmin (nucleolar | ESTs, Moderately similar to NPM_HUMAN | General |
| | | | phosphoprotein B23, | NUCLEOPHOSMIN [H. sapiens], ESTs, Weakly | |
| | | | numatrin), nucleophosmin 1 | similar to NPM_HUMAN NUCLEOPHOSMIN | |
| | | | | [H. sapiens], nucleophosmin (nucleolar | |
| | | | | phosphoprotein B23, numatrin), nucleophosmin | |
| | | | | 1, nucleophosmin/nucleoplasmin 3, | |
| | | | | nucleoplasmin 3 | |
| 56 | 19392 | NM_012998 | procollagen-proline, 2- | RIKEN cDNA 177B13 gene, RIKEN cDNA | i |
| | | | oxoglutarate 4- | 18141F13 gene, RIKEN cDNA 18147B9 gene, | |
| | | | dioxygenase (proline 4- | expressed sequence AI661267, procollagen- | |
| | | | hydroxylase), beta | proline, 2-oxoglutarate 4-dioxygenase (proline 4 | |
| | | | polypeptide (protein | hydroxylase), beta polypeptide (protein disulfide | |
| | | | disulfide isomerase; | isomerase; thyroid hormone binding protein | |
| | | | thyroid hormone binding | p55) | |
| | | | protein p55), prolyl 4- | | |
| | | | hydroxylase, beta | | |
| | | | polypeptide | | |
| 57 | 24264 | NM_012999 | paired basic amino acid | EST, Highly similar to PAC4 RAT SUBTILISIN- | e |
| | | | cleaving system 4, | LIKE PROTEASE PACE4 PRECURSOR | |
| | | | proprotein convertase | [R. norvegicus], Homo sapiens clone 1 | |
| | | | subtilisin/kexin type 6 | thrombospondin mRNA, complete cds, RIKEN | |
| | | | | cDNA A9329K19 gene, paired basic amino acid | |
| | | | | cleaving system 4, proprotein convertase | |
| | | | | subtilisin/kexin type 6 | |
| 58 | 3405 | NM_013011 | | EST, Weakly similar to 143B_HUMAN 14-3-3 | i, General |
| | | | | PROTEIN BETA/ALPHA [H. sapiens], ESTs, | |
| | | | | Highly similar to A Chain A, 14-3-3 | |
| | | | | ZetaPHOSPHOPEPTIDE COMPLEX | |
| | | | | [H. sapiens], makorin, ring finger protein, 3, | |
| | | | | stratifin, tyrosine 3-monooxgenase/tryptophan 5 | |
| | | | | monooxgenase activation protein, beta | |
| | | | | polypeptide, tyrosine 3- | |
| | | | | monooxygenase/tryptophan 5-monooxygenase | |
| | | | | activation protein, zeta polypeptide | |
| 59 | 20229 | NM_013018 | RAB3A, member RAS | Homo sapiens, clone MGC: 4711 | General |
| | | | oncogene family | IMAGE: 3534915, mRNA, complete cds, | |
| | | | | RAB3A, member RAS oncogene family, | |
| | | | | RAB3C, member RAS oncogene family, RIKEN | |
| | | | | cDNA 261528C18 gene | |
| 60 | 1606 | NM_013039 | ATP-binding cassette, | ATP-binding cassette, sub-family C | g |
| | | | sub-family C | (CFTR/MRP), member 8, ATP-binding | |
| | | | (CFTR/MRP), member 8 | cassette, sub-family C (CFTR/MRP), member | |
| | | | | 9, EST, Moderately similar to ACC8 RAT | |
| | | | | SULFONYLUREA RECEPTOR 1 | |
| | | | | [R. norvegicus], EST, Weakly similar to MRP5 | |
| | | | | MOUSE MULTIDRUG RESISTANCE- | |
| | | | | ASSOCIATED PROTEIN 5 [M. musculus], | |
| | | | | ESTs, Highly similar to ACC8_HUMAN | |
| | | | | SULFONYLUREA RECEPTOR 1 [H. sapiens], | |
| | | | | ESTs, Weakly similar to T42728 sulfonylurea | |
| | | | | receptor 2, isoform B - mouse [M. musculus], | |
| | | | | Mus musculus adult male pituitary gland cDNA, | |
| | | | | RIKEN full-length enriched library, | |
| | | | | clone: 533439B14, full insert sequence | |
| 61 | 730 | NM_013040 | ATP-binding cassette, | ATP-binding cassette, sub-family C | h |
| | | | sub-family C | (CFTR/MRP), member 8, ATP-binding | |
| | | | (CFTR/MRP), member 9 | cassette, sub-family C (CFTR/MRP), member | |
| | | | | 9, ESTs, Highly similar to ACC8_HUMAN | |
| | | | | SULFONYLUREA RECEPTOR 1 [H. sapiens], | |
| | | | | ESTs, Weakly similar to T42728 sulfonylurea | |
| | | | | receptor 2, isoform B - mouse [M. musculus], | |
| | | | | Mus musculus adult male pituitary gland cDNA, | |
| | | | | RIKEN full-length enriched library, | |
| | | | | clone: 533439B14, full insert sequence | |
| 62 | 17401 | NM_013043 | transforming growth factor | ESTs, Moderately similar to DIP_HUMAN DIP | g, General |
| | | | beta 1 induced transcript | PROTEIN [H. sapiens], delta sleep inducing | |
| | | | 4, transforming growth | peptide, immunoreactor, glucocorticoid-induced | |
| | | | factor beta-stimulated | leucine zipper, transforming growth factor beta | |
| | | | protein TSC-22 | 1 induced transcript 4, transforming growth | |
| | | | | factor beta-stimulated protein TSC-22 | |
| 63 | 11114 | NM_013046 | thyrotropin releasing | thyrotropin releasing hormone, thyrotropin- | f, General |
| | | | hormone, thyrotropin- | releasing hormone | |
| | | | releasing hormone | | |
| 63 | 11113 | NM_013046 | thyrotropin releasing | | f |
| | | | hormone, thyrotropin- | | |
| | | | releasing hormone | | |
| 64 | 16924 | NM_013069 | | CD74 antigen (invariant polypeptide of major | b |
| | | | | histocompatibility complex, class II antigen- | |
| | | | | associated), la-associated invariant chain, | |
| | | | | KIAA275 gene product, sparc/osteonectin, | |
| | | | | cwcv and kazal-like domains proteoglycan | |
| | | | | (testican), sparc/osteonectin, cwcv and kazal- | |
| | | | | like domains proteoglycan 1, sparc/osteonectin, | |
| | | | | cwcv and kazal-like domains proteoglycan 2 | |
| 64 | 25676 | NM_013069 | | | b |
| 64 | 16925 | NM_013069 | | CD74 antigen (invariant polypeptide of major | b |
| | | | | histocompatibility complex, class II antigen- | |
| | | | | associated), la-associated invariant chain, | |
| | | | | KIAA275 gene product, sparc/osteonectin, | |
| | | | | cwcv and kazal-like domains proteoglycan | |
| | | | | (testican), sparc/osteonectin, cwcv and kazal- | |
| | | | | like domains proteoglycan 1, sparc/osteonectin, | |
| | | | | cwcv and kazal-like domains proteoglycan 2 | |
| 65 | 357 | NM_013086 | | | General |
| 66 | 1521 | NM_013091 | | tumor necrosis factor receptor superfamily, | i |
| | | | | member 12, tumor necrosis factor receptor | |
| | | | | superfamily, member 12 (translocating chain- | |
| | | | | association membrane protein), tumor necrosis | |
| | | | | factor receptor superfamily, member 1A, tumor | |
| | | | | necrosis factor receptor superfamily, member | |
| | | | | 1a, tumor necrosis factor receptor superfamily, | |
| | | | | member 1a-like 1, tumor necrosis factor | |
| | | | | receptor superfamily, member 1a-like 2 | |
| 67 | 1684 | NM_013096 | hemoglobin alpha, adult | EST, Moderately similar to HART1 hemoglobin | a, b |
| | | | chain 2, hemoglobin, | alpha-1 chain - rat [R. norvegicus], ESTs, | |
| | | | alpha 1 | Moderately similar to HART1 hemoglobin alpha- | |
| | | | | 1 chain - rat [R. norvegicus], RIKEN cDNA | |
| | | | | 25142H12 gene, hemoglobin alpha, adult chain | |
| | | | | 1, hemoglobin, alpha 1, hemoglobin, alpha 2 | |
| 67 | 26150 | NM_013096 | | | a, b |
| 67 | 1689 | NM_013096 | hemoglobin alpha, adult | EST, Moderately similar to HART1 hemoglobin | a, b, |
| | | | chain 2, hemoglobin, | alpha-1 chain - rat [R. norvegicus], ESTs, | General |
| | | | alpha 1 | Moderately similar to HART1 hemoglobin alpha- | |
| | | | | 1 chain - rat [R. norvegicus], RIKEN cDNA | |
| | | | | 25142H12 gene, hemoglobin alpha, adult chain | |
| | | | | 1, hemoglobin, alpha 1, hemoglobin, alpha 2 | |
| 67 | 1685 | NM_013096 | hemoglobin alpha, adult | | a |
| | | | chain 2, hemoglobin, | | |
| | | | alpha 1 | | |
| 67 | 1688 | NM_013096 | hemoglobin alpha, adult | | a, b |
| | | | chain 2, hemoglobin, | | |
| | | | alpha 1 | | |
| 68 | 1321 | NM_013098 | | ESTs, Weakly similar to G6PT RAT GLUCOSE- | a |
| | | | | 6-PHOSPHATASE [R. norvegicus], Homo | |
| | | | | sapiens, clone IMAGE: 35476, mRNA, partial | |
| | | | | cds, RIKEN cDNA 711K1 gene, expressed | |
| | | | | sequence AW545836, glucose-6-phosphatase, | |
| | | | | catalytic, glucose-6-phosphatase, catalytic | |
| | | | | (glycogen storage disease type I, von Gierke | |
| | | | | disease), glucose-6-phosphatase, catalytic, | |
| | | | | related sequence, islet-specific glucose-6- | |
| | | | | phosphatase catalytic subunit-related protein | |
| 69 | 19949 | NM_013106 | guanine nucleotide | guanine nucleotide binding protein (G protein), | General |
| | | | binding protein (G | alpha inhibiting activity polypeptide 3, guanine | |
| | | | protein), alpha inhibiting | nucleotide binding protein, alpha inhibiting 3 | |
| | | | activity polypeptide 3, | | |
| | | | guanine nucleotide | | |
| | | | binding protein, alpha | | |
| | | | inhibiting 3 | | |
| 70 | 23709 | NM_013113 | | ATPase, Na+/K+ transporting, beta 1 | g |
| | | | | polypeptide | |
| 71 | 2005 | NM_013127 | | CD38 antigen, CD38 antigen (p45), Homo | c |
| | | | | sapiens cDNA: FLJ22754 fis, clone KAIA615 | |
| 72 | 14299 | NM_013129 | interleukin 15 | interleukin 15 | General |
| 73 | 16982 | NM_013144 | | insulin-like growth factor binding protein 1, | General |
| | | | | protease, serine, 11 (IGF binding) | |
| 74 | 21683 | NM_013154 | CCAAT/enhancer binding | CCAAT/enhancer binding protein (C/EBP), | General |
| | | | protein (C/EBP), delta | delta | |
| 74 | 21682 | NM_013154 | CCAAT/enhancer binding | CCAAT/enhancer binding protein (C/EBP), | General |
| | | | protein (C/EBP), delta | delta | |
| 75 | 24867 | NM_013155 | | EST, Weakly similar to LDVR RAT VERY LOW- | General |
| | | | | DENSITY LIPOPROTEIN RECEPTOR | |
| | | | | PRECURSOR [R. norvegicus], ESTs, Weakly | |
| | | | | similar to LDVR RAT VERY LOW-DENSITY | |
| | | | | LIPOPROTEIN RECEPTOR PRECURSOR | |
| | | | | [R. norvegicus], low density lipoprotein receptor- | |
| | | | | related protein 8, apolipoprotein e receptor, | |
| | | | | very low density lipoprotein receptor | |
| 76 | 25567 | NM_013156 | | | d, General |
| 76 | 3431 | NM_013156 | cathepsin L | ESTs, Weakly similar to CATHEPSIN L | General |
| | | | | PRECURSOR [M. musculus], RIKEN cDNA | |
| | | | | 23151M13 gene, RIKEN cDNA 493486L24 | |
| | | | | gene, cathepsin 7, cathepsin F, cathepsin L, | |
| | | | | cathepsin L2, cathepsin O | |
| 76 | 3430 | NM_013156 | cathepsin L | ESTs, Weakly similar to CATHEPSIN L | d, General |
| | | | | PRECURSOR [M. musculus], RIKEN cDNA | |
| | | | | 23151M13 gene, RIKEN cDNA 493486L24 | |
| | | | | gene, cathepsin 7, cathepsin F, cathepsin L, | |
| | | | | cathepsin L2, cathepsin O | |
| 77 | 1714 | NM_013187 | | Homo sapiens, Similar to cortactin isoform B, | g, i, |
| | | | | clone MGC: 3591 IMAGE: 3637586, mRNA, | General |
| | | | | complete cds, cell differentiation and embryonic | |
| | | | | development, ems1 sequence (mammary | |
| | | | | tumor and squamous cell carcinoma-associated | |
| | | | | (p8/85 src substrate), expressed sequence | |
| | | | | AI89414, phospholipase C, gamma 1 (formerly | |
| | | | | subtype 148) | |
| 78 | 16448 | NM_013197 | | EST, Highly similar to HEM RAT 5- | a, b, |
| | | | | AMINOLEVULINIC ACID SYNTHASE, | General |
| | | | | ERYTHROID-SPECIFIC, MITOCHONDRIAL | |
| | | | | PRECURSOR [R. norvegicus], ESTs, Highly | |
| | | | | similar to SYHUAE 5-aminolevulinate synthase | |
| | | | | [H. sapiens], aminolevulinate, delta-, synthase 2 | |
| | | | | (sideroblastic/hypochromic anemia), | |
| | | | | aminolevulinic acid synthase 1, aminolevulinic | |
| | | | | acid synthase 2, erythroid, glycine C- | |
| | | | | acetyltransferase (2-amino-3-ketobutyrate | |
| | | | | coenzyme A ligase), glycine C- | |
| | | | | acetyltransferase (2-amino-3-ketobutyrate- | |
| | | | | coenzyme A ligase) | |
| 79 | 20826 | NM_013218 | | ESTs, Weakly similar to KAD3_HUMAN | h |
| | | | | GTP: AMP PHOSPHOTRANSFERASE | |
| | | | | MITOCHONDRIAL [H. sapiens], RIKEN cDNA | |
| | | | | 1718L2 gene, adenylate kinase 3 alpha like | |
| 80 | 18313 | NM_013220 | ankyrin-like repeat | ESTs, Highly similar to A44437 regenerating | f, g, i, |
| | | | protein, cardiac ankyrin | liver inhibitory factor RL/IF-1 - rat | General |
| | | | repeat protein | [R. norvegicus], ESTs, Highly similar to A57291 | |
| | | | | cytokine inducible nuclear protein C193 | |
| | | | | [H. sapiens], ESTs, Weakly similar to A57291 | |
| | | | | cytokine inducible nuclear protein C193 | |
| | | | | [H. sapiens], ESTs, Weakly similar to I-kappa B | |
| | | | | alpha chain [M. musculus], RIKEN cDNA | |
| | | | | 11158D9 gene, RIKEN cDNA 169D24 gene, | |
| | | | | RIKEN cDNA 493539L19 gene, ankyrin repeat | |
| | | | | domain 2 (stretch responsive muscle), cardiac | |
| | | | | ankyrin repeat protein, expressed sequence | |
| | | | | AA489, nuclear factor of kappa light chain gene | |
| | | | | enhancer in B-cells inhibitor, alpha, nuclear | |
| | | | | factor of kappa light polypeptide gene enhancer | |
| | | | | in B-cells inhibitor, alpha | |
| 81 | 815 | NM_013224 | | ESTs, Highly similar to RS26_HUMAN 4S | e, General |
| | | | | RIBOSOMAL PROTEIN S26 [H. sapiens], | |
| | | | | Homo sapiens, clone IMAGE: 41953, mRNA, | |
| | | | | Human DNA sequence from PAC 384D21 on | |
| | | | | chromosome X contains ribosomal protein S26 | |
| | | | | pseudogene, STS, polymerase (RNA) II (DNA | |
| | | | | directed) polypeptide D, ribosomal protein S26 | |
| 82 | 15239 | NM_016989 | | EST, Weakly similar to RL15 RAT 6S | h |
| | | | | RIBOSOMAL PROTEIN L15 [R. norvegicus], | |
| | | | | ESTs, Highly similar to RL15_HUMAN 6S | |
| | | | | RIBOSOMAL PROTEIN L15 [H. sapiens], | |
| | | | | ESTs, Moderately similar to RL15 RAT 6S | |
| | | | | RIBOSOMAL PROTEIN L15 [R. norvegicus], | |
| | | | | RIKEN cDNA 2518H7 gene, ribosomal protein | |
| | | | | L15 | |
| 83 | 3202 | NM_017039 | protein phosphatase 2 | | h |
| | | | (formerly 2A), catalytic | | |
| | | | subunit, alpha isoform, | | |
| | | | protein phosphatase 2a, | | |
| | | | catalytic subunit, alpha | | |
| | | | isoform | | |
| 83 | 3203 | NM_017039 | protein phosphatase 2 | protein phosphatase 2 (formerly 2A), catalytic | h |
| | | | (formerly 2A), catalytic | subunit, alpha isoform, protein phosphatase 2a, | |
| | | | subunit, alpha isoform, | catalytic subunit, alpha isoform | |
| | | | protein phosphatase 2a, | | |
| | | | catalytic subunit, alpha | | |
| | | | isoform | | |
| 84 | 24596 | NM_017040 | protein phosphatase 2 | Mus musculus adult female placenta cDNA, | h |
| | | | (formerly 2A), catalytic | RIKEN full-length enriched library, | |
| | | | subunit, beta isoform, | clone: 1617J22, full insert sequence, RIKEN | |
| | | | protein phosphatase 2a, | cDNA 2313C1 gene, expressed sequence | |
| | | | catalytic subunit, beta | AI115466, protein phosphatase 2 (formerly 2A), | |
| | | | isoform | catalytic subunit, beta isoform, protein | |
| | | | | phosphatase 2a, catalytic subunit, beta isoform, | |
| | | | | protein phosphatase 4, catalytic subunit | |
| 85 | 6654 | NM_017068 | lysosomal membrane | CD68 antigen, ESTs, Highly similar to LMP2 | a |
| | | | glycoprotein 2, lysosomal- | RAT LYSOSOME-ASSOCIATED MEMBRANE | |
| | | | associated membrane | GLYCOPROTEIN 2 PRECURSOR | |
| | | | protein 2 | [R. norvegicus], ESTs, Weakly similar to A4842 | |
| | | | | lysosomal membrane glycoprotein lamp-2 | |
| | | | | homolog [H. sapiens], lysosomal membrane | |
| | | | | glycoprotein 2, lysosomal-associated | |
| | | | | membrane protein 2 | |
| 86 | 11152 | NM_017073 | glutamate-ammonia | glutamate-ammonia ligase (glutamine | General |
| | | | ligase (glutamine | synthase), glutamine synthetase | |
| | | | synthase), glutamine | | |
| | | | synthetase | | |
| 86 | 11153 | NM_017073 | glutamate-ammonia | glutamate-ammonia ligase (glutamine | General |
| | | | ligase (glutamine | synthase), glutamine synthetase | |
| | | | synthase), glutamine | | |
| | | | synthetase | | |
| 87 | 22552 | NM_017087 | biglycan | ESTs, Weakly similar to BONE/CARTILAGE | General |
| | | | | PROTEOGLYCAN I PRECURSOR | |
| | | | | [R. norvegicus], ESTs, Weakly similar to | |
| | | | | PGS1_HUMAN BONE/CARTILAGE | |
| | | | | PROTEOGLYCAN I PRECURSOR [H. sapiens], | |
| | | | | asporin, asporin (LRR class 1), biglycan, opticin | |
| 88 | 8888 | NM_017090 | guanylate cyclase 1, | ESTs, Weakly similar to ANPA MOUSE | General |
| | | | soluble, alpha 3 | ATRIAL NATRIURETIC PEPTIDE RECEPTOR | |
| | | | | A PRECURSOR [M. musculus], ESTs, Weakly | |
| | | | | similar to GUANYLATE CYCLASE SOLUBLE, | |
| | | | | ALPHA-1 CHAIN [R. norvegicus], guanylate | |
| | | | | cyclase 1, soluble, alpha 2, guanylate cyclase | |
| | | | | 1, soluble, alpha 3, guanylate cyclase 1, | |
| | | | | soluble, beta 3, natriuretic peptide receptor 1, | |
| | | | | soluble guanylyl cyclase alpha2 subunit | |
| 89 | 1435 | NM_017125 | CD63 antigen (melanoma | CD63 antigen (melanoma 1 antigen), Cd63 | g, General |
| | | | 1 antigen), Cd63 antigen | antigen, DNA segment, Chr 14, ERATO Doi | |
| | | | | 226, expressed, EST, Weakly similar to CD63 | |
| | | | | ANTIGEN [R. norvegicus], EST, Weakly similar | |
| | | | | to I3816 melanoma-associated antigen CD63 | |
| | | | | [H. sapiens], ESTs, Weakly similar to A46493 | |
| | | | | metastasis suppressor KAI1 [H. sapiens], Homo | |
| | | | | sapiens cDNA FLJ221 fis, clone COLF121, | |
| | | | | RIKEN cDNA 131A2 gene, RIKEN cDNA | |
| | | | | 22121G21 gene, transmembrane 4 superfamily | |
| | | | | member 2 | |
| 90 | 21663 | NM_017126 | ferredoxin 1 | ESTs, Moderately similar to AXHU adrenodoxin | General |
| | | | | precursor [H. sapiens], Homo sapiens, Similar | |
| | | | | to RIKEN cDNA B23118G17 gene, clone | |
| | | | | MGC: 1964 IMAGE: 3622817, mRNA, complete | |
| | | | | cds, RIKEN cDNA 136N24 gene, RIKEN cDNA | |
| | | | | B23118G17 gene, ferredoxin 1 | |
| 91 | 24886 | NM_017138 | laminin receptor 1 (67 kD, | EST, Moderately similar to 4S RIBOSOMAL | General |
| | | | ribosomal protein SA) | PROTEIN SA [R. norvegicus], ESTs, Highly | |
| | | | | similar to A31233 ribosomal protein RS.4K, | |
| | | | | cytosolic [H. sapiens], ESTs, Highly similar to | |
| | | | | A5688 laminin receptor, 67 K [H. sapiens], | |
| | | | | ESTs, Moderately similar to A29395 ribosomal | |
| | | | | protein RS.4 K - mouse [M. musculus], ESTs, | |
| | | | | Weakly similar to 14534A protein 4 kD | |
| | | | | [M. musculus], Homo sapiens laminin receptor- | |
| | | | | like protein LAMRL5 mRNA, complete cds, | |
| | | | | laminin receptor 1 (67 kD, ribosomal protein SA) | |
| 92 | 24106 | NM_017141 | DNA polymerase beta, | EST, Weakly similar to A27112 DNA-directed | c |
| | | | polymerase (DNA | DNA polymerase [R. norvegicus], Mus | |
| | | | directed), beta | musculus, Similar to DNA polymerase beta, | |
| | | | | clone MGC: 6386 IMAGE: 3581916, mRNA, | |
| | | | | complete cds, polymerase (DNA directed), | |
| | | | | beta, polymerase (DNA directed), lambda | |
| 92 | 24107 | NM_017141 | DNA polymerase beta, | EST, Weakly similar to A27112 DNA-directed | General |
| | | | polymerase (DNA | DNA polymerase [R. norvegicus], Mus | |
| | | | directed), beta | musculus, Similar to DNA polymerase beta, | |
| | | | | clone MGC: 6386 IMAGE: 3581916, mRNA, | |
| | | | | complete cds, polymerase (DNA directed), | |
| | | | | beta, polymerase (DNA directed), lambda | |
| 93 | 20859 | NM_017144 | troponin I, cardiac | EST, Moderately similar to TROPONIN I, | a, b, f |
| | | | | CARDIAC MUSCLE [R. norvegicus], troponin I, | |
| | | | | cardiac, troponin I, skeletal, slow 1 | |
| 94 | 15365 | NM_017147 | cofilin 1 (non-muscle), | EST, Moderately similar to COF1_HUMAN | g, h, |
| | | | cofilin 1, non-muscle | COFILIN, NON-MUSCLE ISOFOR [H. sapiens], | General |
| | | | | ESTs, Highly similar to DEST_HUMAN | |
| | | | | DESTRIN [H. sapiens], ESTs, Moderately | |
| | | | | similar to COF1 RAT COFILIN, NON-MUSCLE | |
| | | | | ISOFORM [R. norvegicus], ESTs, Moderately | |
| | | | | similar to COF1_HUMAN COFILIN, NON- | |
| | | | | MUSCLE ISOFOR [H. sapiens], cofilin 1 (non- | |
| | | | | muscle), cofilin 1, non-muscle, cofilin 2 | |
| | | | | (muscle), cofilin 2, muscle, destrin | |
| 95 | 5351 | NM_017150 | ribosomal protein L29 | EST, Moderately similar to 6S RIBOSOMAL | General |
| | | | | PROTEIN L29 [R. norvegicus], EST, Weakly | |
| | | | | similar to S65784 ribosomal protein L29, | |
| | | | | cytosolic [H. sapiens], ESTs, Highly similar to | |
| | | | | S65784 ribosomal protein L29, cytosolic | |
| | | | | [H. sapiens], ESTs, Weakly similar to 6S | |
| | | | | RIBOSOMAL PROTEIN L29 [M. musculus], | |
| | | | | Human DNA sequence from clone RP4-595K12 | |
| | | | | on chromosome 1p31.2-31.3 Contains a | |
| | | | | pseudogene similar to 6S RPL29 (ribosomal | |
| | | | | protein L29 (cell surface heparin binding protein | |
| | | | | HIP)), a chromosome 1 specific mRNA | |
| | | | | (KIAA499), a novel mRNA (KIAA433), ESTs, | |
| | | | | STSs, GSSs and a CpG Island, ribosomal | |
| | | | | protein L29 | |
| 96 | 16953 | NM_017151 | ribosomal protein S15 | EST, Moderately similar to R3HU15 ribosomal | e, h |
| | | | | protein S15, cytosolic [H. sapiens], EST, | |
| | | | | Weakly similar to 4S RIBOSOMAL PROTEIN | |
| | | | | S15 [R. norvegicus], ESTs, Highly similar to | |
| | | | | R3HU15 ribosomal protein S15, cytosolic | |
| | | | | [H. sapiens], Homo sapiens, clone | |
| | | | | IMAGE: 44798, mRNA, partial cds, ribosomal | |
| | | | | protein S15 | |
| 97 | 21975 | NM_017154 | xanthene dehydrogenase, | EST, Weakly similar to XDH RAT XANTHINE | General |
| | | | xanthine dehydrogenase | DEHYDROGENASE/OXIDASE [R. norvegicus], | |
| | | | | RIKEN cDNA 1211D3 gene, RIKEN cDNA | |
| | | | | 2313G12 gene, aldehyde oxidase (female | |
| | | | | form), aldehyde oxidase 1, xanthene | |
| | | | | dehydrogenase, xanthine dehydrogenase | |
| 98 | 17104 | NM_017160 | ribosomal protein S6 | EST, Moderately similar to R3HU6 ribosomal | General |
| | | | | protein S6, cytosolic [H. sapiens], EST, Weakly | |
| | | | | similar to 4S RIBOSOMAL PROTEIN S6 | |
| | | | | [R. norvegicus], ESTs, Moderately similar to 4S | |
| | | | | RIBOSOMAL PROTEIN S6 [M. musculus], | |
| | | | | Homo sapiens cDNA: FLJ23534 fis, clone | |
| | | | | LNG6974, highly similar to HUMRPS6A Human | |
| | | | | ribosomal protein S6 mRNA, RIKEN cDNA | |
| | | | | 58345M2 gene, ribosomal protein S6 | |
| 98 | 17107 | NM_017160 | ribosomal protein S6 | EST, Moderately similar to R3HU6 ribosomal | General |
| | | | | protein S6, cytosolic [H. sapiens], EST, Weakly | |
| | | | | similar to 4S RIBOSOMAL PROTEIN S6 | |
| | | | | [R. norvegicus], ESTs, Moderately similar to 4S | |
| | | | | RIBOSOMAL PROTEIN S6 [M. musculus], | |
| | | | | Homo sapiens cDNA: FLJ23534 fis, clone | |
| | | | | LNG6974, highly similar to HUMRPS6A Human | |
| | | | | ribosomal protein S6 mRNA, RIKEN cDNA | |
| | | | | 58345M2 gene, ribosomal protein S6 | |
| 99 | 19031 | NM_017180 | T-cell death associated | ESTs, Weakly similar to S58222 PQ-rich | General |
| | | | gene, pleckstrin | protein [H. sapiens], MARCKS-like protein, Mus | |
| | | | homology-like domain, | musculus 8 days embryo cDNA, RIKEN full- | |
| | | | family A, member 1 | length enriched library, clone: 573519L1, full | |
| | | | | insert sequence, Myristoylated alanine-rich | |
| | | | | protein kinase C substrate, myristoylated | |
| | | | | alanine rich protein kinase C substrate, | |
| | | | | myristoylated alanine-rich protein kinase C | |
| | | | | substrate (MARCKS, 8K-L), pleckstrin | |
| | | | | homology-like domain, family A, member 1, | |
| | | | | pleckstrin homology-like domain, family A, | |
| | | | | member 3, tumor suppressing subtransferable | |
| | | | | candidate 3 | |
| 100 | 15434 | NM_017187 | | EST, Moderately similar to HMG2 RAT HIGH | f |
| | | | | MOBILITY GROUP PROTEIN HMG2 | |
| | | | | [R. norvegicus], EST, Weakly similar to HMG2 | |
| | | | | RAT HIGH MOBILITY GROUP PROTEIN | |
| | | | | HMG2 [R. norvegicus], ESTs, Weakly similar to | |
| | | | | 21363A high mobility group protein 2 | |
| | | | | [H. sapiens], ESTs, Weakly similar to HMG2 | |
| | | | | RAT HIGH MOBILITY GROUP PROTEIN | |
| | | | | HMG2 [R. norvegicus], Human DNA sequence | |
| | | | | from clone RP3-527B1 on | |
| | | | | chromosome 6q25.1-25.3 | |
| | | | | Contains a pseudogene similar to HMG | |
| | | | | (high mobility group) protein, STSs and GSSs, | |
| | | | | Human DNA sequence from clone RP5-17G16 | |
| | | | | on chromosome 1p34.2-35.3. Contains part of | |
| | | | | the gene for a novel CUB and Sushi (SCR | |
| | | | | repeat) domain protein, a novel high-mobility | |
| | | | | group (nonhistone chromosomal) protein 2 | |
| | | | | (HMG2) like protein (pseudo) gene, a heat | |
| | | | | shock 6 kD protein 1 (chaperonin) (HSPD1) | |
| | | | | pseudogene, ESTs, STSs and GSSs, RIKEN | |
| | | | | cDNA 26121J1 gene, expressed sequence | |
| | | | | AI326135, expressed sequence C8539, high | |
| | | | | mobility group box 2, high-mobility group | |
| | | | | (nonhistone chromosomal) protein 2 | |
| 101 | 14696 | NM_017202 | | | e |
| 102 | 5005 | NM_017209 | | Alcohol dehydrogenase 3, CGI-63 protein, | General |
| | | | | ESTs, Weakly similar to QOR MOUSE | |
| | | | | QUINONE OXIDOREDUCTASE [M. musculus], | |
| | | | | Homo sapiens, clone MGC: 183 | |
| | | | | IMAGE: 3897118, mRNA, complete cds, | |
| | | | | KIAA1576 protein, RIKEN cDNA 22147J23 | |
| | | | | gene, alcohol dehydrogenase 1, complex, | |
| | | | | alcohol dehydrogenase 1C (class I), gamma | |
| | | | | polypeptide, crystallin, zeta, expressed | |
| | | | | sequence AI194826, membrane protein of | |
| | | | | cholinergic synaptic vesicles | |
| 103 | 20193 | NM_017232 | prostaglandin- | prostaglandin-endoperoxide synthase 2, | General |
| | | | endoperoxide synthase 2, | prostaglandin-endoperoxide synthase 2 | |
| | | | prostaglandin- | (prostaglandin G/H synthase and | |
| | | | endoperoxide synthase 2 | cyclooxygenase) | |
| | | | (prostaglandin G/H | | |
| | | | synthase and | | |
| | | | cyclooxygenase) | | |
| 104 | 20482 | NM_017240 | myosin heavy chain, | EST, Highly similar to MYOSIN HEAVY CHAIN, | e |
| | | | cardiac muscle, adult, | CARDIAC MUSCLE BETA ISOFORM | |
| | | | myosin heavy chain, | [R. norvegicus], EST, Moderately similar to | |
| | | | cardiac muscle, fetal, | MYOSIN HEAVY CHAIN, CARDIAC MUSCLE | |
| | | | myosin, heavy | BETA ISOFORM [R. norvegicus], ESTs, Highly | |
| | | | polypeptide 6, cardiac | similar to MYOSIN HEAVY CHAIN, CARDIAC | |
| | | | muscle, alpha | MUSCLE BETA ISOFORM [R. norvegicus], | |
| | | | (cardiomyopathy, | ESTs, Highly similar to MYSA MOUSE | |
| | | | hypertrophic 1), myosin, | MYOSIN HEAVY CHAIN, CARDIAC MUSCLE | |
| | | | heavy polypeptide 7, | ALPHA ISOFORM [M. musculus], KIAA1512 | |
| | | | cardiac muscle, beta | protein, myosin heavy chain, cardiac muscle, | |
| | | | | adult, myosin heavy chain, cardiac muscle, | |
| | | | | fetal, myosin, heavy polypeptide 2, skeletal | |
| | | | | muscle, adult, myosin, heavy polypeptide 7, | |
| | | | | cardiac muscle, beta, tuftelin 1 | |
| 104 | 3780 | NM_017240 | | | e |
| 104 | 20484 | NM_017240 | myosin heavy chain, | EST, Highly similar to MYOSIN HEAVY CHAIN, | a, c, e, f |
| | | | cardiac muscle, adult, | CARDIAC MUSCLE BETA ISOFORM | |
| | | | myosin heavy chain, | [R. norvegicus], EST, Moderately similar to | |
| | | | cardiac muscle, fetal, | MYOSIN HEAVY CHAIN, CARDIAC MUSCLE | |
| | | | myosin, heavy | BETA ISOFORM [R. norvegicus], ESTs, Highly | |
| | | | polypeptide 6, cardiac | similar to MYOSIN HEAVY CHAIN, CARDIAC | |
| | | | muscle, alpha | MUSCLE BETA ISOFORM [R. norvegicus], | |
| | | | (cardiomyopathy, | ESTs, Highly similar to MYSA MOUSE | |
| | | | hypertrophic 1), myosin, | MYOSIN HEAVY CHAIN, CARDIAC MUSCLE | |
| | | | heavy polypeptide 7, | ALPHA ISOFORM [M. musculus], KIAA1512 | |
| | | | cardiac muscle, beta | protein, myosin heavy chain, cardiac muscle, | |
| | | | | adult, myosin heavy chain, cardiac muscle, | |
| | | | | fetal, myosin, heavy polypeptide 2, skeletal | |
| | | | | muscle, adult, myosin, heavy polypeptide 7, | |
| | | | | cardiac muscle, beta, tuftelin 1 | |
| 105 | 17563 | NM_017245 | eukaryotic translation | EST, Weakly similar to EFHU2 translation | g, General |
| | | | elongation factor 2 | elongation factor eEF-2 [H. sapiens], ESTs, | |
| | | | | Highly similar to ELONGATION FACTOR 2 | |
| | | | | [R. norvegicus], ESTs, Weakly similar to | |
| | | | | ELONGATION FACTOR 2 [R. norvegicus], G1 | |
| | | | | to phase transition 1, G1 to phase transition 2, | |
| | | | | RIKEN cDNA 493594C11 gene, U5 snRNP- | |
| | | | | specific protein, 116 kD, eukaryotic translation | |
| | | | | elongation factor 1 alpha 1, eukaryotic | |
| | | | | translation elongation factor 2, expressed | |
| | | | | sequence AI45134, hypothetical protein | |
| | | | | FLJ21661 | |
| 106 | 15301 | NM_017259 | B-cell translocation gene | B-cell translocation gene 2, anti-proliferative, B- | General |
| | | | 2, anti-proliferative, BTG | cell translocation gene 3, B-cell translocation | |
| | | | family, member 2 | gene 4, BTG family, member 2, BTG family, | |
| | | | | member 3, BTG family, member 4, ESTs, | |
| | | | | Highly similar to BTG2_HUMAN BTG2 | |
| | | | | PROTEIN PRECURSOR [H. sapiens] | |
| 106 | 15299 | NM_017259 | B-cell translocation gene | B-cell translocation gene 2, anti-proliferative, B- | General |
| | | | 2, anti-proliferative, BTG | cell translocation gene 3, B-cell translocation | |
| | | | family, member 2 | gene 4, BTG family, member 2, BTG family, | |
| | | | | member 3, BTG family, member 4, ESTs, | |
| | | | | Highly similar to BTG2_HUMAN BTG2 | |
| | | | | PROTEIN PRECURSOR [H. sapiens] | |
| 106 | 15300 | NM_017259 | B-cell translocation gene | B-cell translocation gene 2, anti-proliferative, B- | General |
| | | | 2, anti-proliferative, BTG | cell translocation gene 3, B-cell translocation | |
| | | | family, member 2 | gene 4, BTG family, member 2, BTG family, | |
| | | | | member 3, BTG family, member 4, ESTs, | |
| | | | | Highly similar to BTG2_HUMAN BTG2 | |
| | | | | PROTEIN PRECURSOR [H. sapiens] | |
| 107 | 3254 | NM_017282 | proteasome (prosome, | expressed sequence AA4947, proteasome | h |
| | | | macropain) subunit, alpha | (prosome, macropain) subunit, alpha type 5, | |
| | | | type 5, proteasome | proteasome (prosome, macropain) subunit, | |
| | | | (prosome, macropain) | alpha type, 5 | |
| | | | subunit, alpha type, 5 | | |
| 108 | 82 | NM_017297 | potassium inwardly- | potassium inwardly-rectifying channel, | i |
| | | | rectifying channel, | subfamily J, member 5 | |
| | | | subfamily J, member 5 | | |
| 109 | 1894 | NM_017320 | | cathepsin S | General |
| 110 | 356 | NM_017334 | | | General |
| 110 | 355 | NM_017334 | | | General |
| 111 | 20848 | NM_017343 | | ESTs, Weakly similar to MOHULP myosin | c, g |
| | | | | regulatory light chain, placental [H. sapiens], | |
| | | | | RIKEN cDNA 2973G15 gene, expressed | |
| | | | | sequence C77744, myosin, light polypeptide, | |
| | | | | regulatory, non-sarcomeric (2 kD) | |
| 112 | 460 | NM_019131 | tropomyosin 1 (alpha), | ESTs, Moderately similar to alpha-tropomyosin | a |
| | | | tropomyosin 1, alpha | slow [M. musculus], tropomyosin 4 | |
| 113 | 5622 | NM_019143 | fibronectin 1 | | General |
| 114 | 17066 | NM_019170 | | ESTs, Weakly similar to JC5284 carbonyl | e |
| | | | | reductase (NADPH) (EC 1.1.1.184), inducible - | |
| | | | | rat [R. norvegicus], Homo sapiens, clone | |
| | | | | MGC: 2328 IMAGE: 463754, mRNA, complete | |
| | | | | cds, RIKEN cDNA 1111J5 gene, RIKEN cDNA | |
| | | | | 94359D4 gene, carbonyl reductase 1, carbonyl | |
| | | | | reductase 3, expressed sequence C81353 | |
| 115 | 1174 | NM_019184 | | RIKEN cDNA 2131M18 gene, RIKEN cDNA | b |
| | | | | 21318C6 gene, RIKEN cDNA 2219K14 gene, | |
| | | | | cytochrome P45, 2c29, cytochrome P45, | |
| | | | | subfamily IIC (mephenytoin 4-hydroxylase), | |
| | | | | polypeptide 18 | |
| 116 | 18569 | NM_019212 | actin, alpha 1, skeletal | BAF53, BRG1/brm-associated factor 53A, EST, | General |
| | | | muscle | Weakly similar to ACTIN, ALPHA SKELETAL | |
| | | | | MUSCLE [R. norvegicus], ESTs, Weakly similar | |
| | | | | to ATHU actin alpha 1, skeletal muscle | |
| | | | | [H. sapiens], Homo sapiens FKSG73 (FKSG73) | |
| | | | | mRNA, complete cds, RIKEN cDNA 6141G9 | |
| | | | | gene, actin, alpha 1, skeletal muscle, actin-like | |
| | | | | 6, actin-related protein 3 | |
| 117 | 20433 | NM_019232 | serum/glucocorticoid | 3-phosphoinositide dependent protein kinase-1, | b, General |
| | | | regulated kinase | EST, Weakly similar to SGK RAT | |
| | | | | SERINE/THREONINE-PROTEIN KINASE SGK | |
| | | | | [R. norvegicus], Mus musculus serine/threonine | |
| | | | | protein kinase CISK (Cisk) mRNA, complete | |
| | | | | cds, RIKEN cDNA 1196F7 gene, | |
| | | | | serum/glucocorticoid regulated kinase, | |
| | | | | serum/glucocorticoid regulated kinase 2, | |
| | | | | serum/glucocorticoid regulated kinase-like, | |
| | | | | thymoma viral proto-oncogene 3 | |
| 118 | 17908 | NM_019242 | interferon-related | ESTs, Weakly similar to INTERFERON- | c |
| | | | developmental regulator 1 | RELATED PROTEIN PC4 [R. norvegicus], | |
| | | | | interferon-related developmental regulator 1, | |
| | | | | interferon-related developmental regulator 2 | |
| 119 | 11218 | NM_019247 | paired-like homeodomain | paired-like homeodomain transcription factor 1, | General |
| | | | transcription factor 3 | paired-like homeodomain transcription factor 2, | |
| | | | | paired-like homeodomain transcription factor 3 | |
| 120 | 23419 | NM_019257 | splicing factor, | EST, Weakly similar to SPLICING FACTOR, | d |
| | | | arginine/serine-rich 5, | ARGININE/SERINE-RICH 5 [R. norvegicus], | |
| | | | splicing factor, | RIKEN cDNA 1211E11 gene, splicing factor, | |
| | | | arginine/serine-rich 5 | arginine/serine-rich 4, splicing factor, | |
| | | | (SRp4, HRS) | arginine/serine-rich 4 (SRp75), splicing factor, | |
| | | | | arginine/serine-rich 5, splicing factor, | |
| | | | | arginine/serine-rich 5 (SRp4, HRS), splicing | |
| | | | | factor, arginine/serine-rich 6 | |
| 121 | 21443 | NM_019262 | complement component | C1q-related factor, Homo sapiens, Similar to | General |
| | | | 1, q subcomponent, beta | complement component 1, q subcomponent, c | |
| | | | polypeptide | polypeptide, clone MGC: 17279 | |
| | | | | IMAGE: 4212772, mRNA, complete cds, | |
| | | | | complement component 1, q subcomponent, | |
| | | | | beta polypeptide, complement component 1, q | |
| | | | | subcomponent, c polypeptide, expressed | |
| | | | | sequence AI385742 | |
| 122 | 20735 | NM_019283 | solute carrier family 3 | EST, Moderately similar to SAHU4F cell | a, d, |
| | | | (activators of dibasic and | surface antigen 4F2 heavy chain [H. sapiens], | General |
| | | | neutral amino acid | ESTs, Moderately similar to 191425A AA | |
| | | | transport), member 2 | transporter [H. sapiens], ESTs, Moderately | |
| | | | | similar to SAHU4F cell surface antigen 4F2 | |
| | | | | heavy chain [H. sapiens], putative L-type neutral | |
| | | | | amino acid transporter, solute carrier family 3 | |
| | | | | (activators of dibasic and neutral amino acid | |
| | | | | transport), member 2, solute carrier family 3 | |
| | | | | (cystine, dibasic and neutral amino acid | |
| | | | | transporters, activator of cystine, dibasic and | |
| | | | | neutral amino acid transport), member 1, solute | |
| | | | | carrier family 3, member 1 | |
| 122 | 20734 | NM_019283 | solute carrier family 3 | EST, Moderately similar to SAHU4F cell | d |
| | | | (activators of dibasic and | surface antigen 4F2 heavy chain [H. sapiens], | |
| | | | neutral amino acid | ESTs, Moderately similar to 191425A AA | |
| | | | transport), member 2 | transporter [H. sapiens], ESTs, Moderately | |
| | | | | similar to SAHU4F cell surface antigen 4F2 | |
| | | | | heavy chain [H. sapiens], putative L-type neutral | |
| | | | | amino acid transporter, solute carrier family 3 | |
| | | | | (activators of dibasic and neutral amino acid | |
| | | | | transport), member 2, solute carrier family 3 | |
| | | | | (cystine, dibasic and neutral amino acid | |
| | | | | transporters, activator of cystine, dibasic and | |
| | | | | neutral amino acid transport), member 1, solute | |
| | | | | carrier family 3, member 1 | |
| 123 | 10016 | NM_019289 | | EST, Highly similar to AR41_HUMAN ARP2/3 | i |
| | | | | COMPLEX 41 KDA SUBUNIT [H. sapiens], | |
| | | | | ESTs, Moderately similar to AR41_HUMAN | |
| | | | | ARP2/3 COMPLEX 41 KDA SUBUNIT | |
| | | | | [H. sapiens], actin related protein 2/3 complex, | |
| | | | | subunit 1A (41 kD), actin related protein 2/3 | |
| | | | | complex, subunit 1A (41 kDa), actin related | |
| | | | | protein 2/3 complex, subunit 1B (41 kDa), | |
| | | | | expressed sequence AA4864, suppressor of | |
| | | | | profilin/p41 of actin-related complex 2/3 | |
| 124 | 15056 | NM_019291 | | EST, Moderately similar to CARBONIC | General |
| | | | | ANHYDRASE II [R. norvegicus], EST, Weakly | |
| | | | | similar to CARBONIC ANHYDRASE II | |
| | | | | [M. musculus], carbonic anhydrase 2, carbonic | |
| | | | | anhydrase 7, carbonic anhydrase I, carbonic | |
| | | | | anhydrase II | |
| 125 | 20750 | NM_019301 | | CUB and Sushi multiple domains 1, ESTs, | h |
| | | | | Highly similar to I7312 complement C3b/C4b | |
| | | | | receptor, membrane-bound form precursor | |
| | | | | [H. sapiens], ESTs, Weakly similar to JC254 | |
| | | | | complement regulatory protein, 5I2 antigen | |
| | | | | precursor - rat [R. norvegicus], Mus musculus 8 | |
| | | | | days embryo cDNA, RIKEN full-length enriched | |
| | | | | library, clone: 573478H2, full insert sequence, | |
| | | | | complement component (3b/4b) receptor 1, | |
| | | | | including Knops blood group system, | |
| | | | | complement receptor related protein, decay | |
| | | | | accelerating factor 1, decay accelerating factor | |
| | | | | 2, membrane cofactor protein | |
| 126 | 52 | NM_019335 | | NIMA (never in mitosis gene a)-related | e |
| | | | | expressed kinase 4, RIKEN cDNA 261528K9 | |
| | | | | gene, eukaryotic translation initiation factor 2 | |
| | | | | alpha kinase 2, eukaryotic translation initiation | |
| | | | | factor 2-alpha kinase 3, protein kinase, | |
| | | | | interferon-inducible double stranded RNA | |
| | | | | dependent, serine/threonine kinase 31 | |
| 127 | 22675 | NM_019358 | | glycoprotein 38, lung type-I cell membrane- | General |
| | | | | associated glycoprotein | |
| 128 | 20057 | NM_019370 | | EST, Weakly similar to A578 cell surface | g |
| | | | | antigen RB13-6 - rat [R. norvegicus], ESTs, | |
| | | | | Weakly similar to A55144 autotaxin precursor | |
| | | | | [H. sapiens], ESTs, Weakly similar to A578 cell | |
| | | | | surface antigen RB13-6 - rat [R. norvegicus], | |
| | | | | Mus musculus, Similar to alkaline | |
| | | | | phosphodiesterase, clone MGC: 6548 | |
| | | | | IMAGE: 2655671, mRNA, complete cds, | |
| | | | | ectonucleotide | |
| | | | | pyrophosphatase/phosphodiesterase 1, | |
| | | | | ectonucleotide | |
| | | | | pyrophosphatase/phosphodiesterase 2, | |
| | | | | ectonucleotide | |
| | | | | pyrophosphatase/phosphodiesterase 3, | |
| | | | | ectonucleotide | |
| | | | | pyrophosphatase/phosphodiesterase 4 | |
| | | | | (putative function), ectonucleotide | |
| | | | | pyrophosphatase/phosphodiesterase 5 | |
| | | | | (putative function), expressed sequence | |
| | | | | AI876438 | |
| 129 | 1324 | NM_019371 | | EGL nine (C. elegans) homolog 1, EGL nine | General |
| | | | | (C. elegans) homolog 2, EGL nine (C. elegans) | |
| | | | | homolog 3, EGL nine homolog 3 (C. elegans), | |
| | | | | ESTs, Moderately similar to A5377 growth | |
| | | | | factor-responsive protein, vascular smooth | |
| | | | | muscle - rat [R. norvegicus], ESTs, Weakly | |
| | | | | similar to A5377 growth factor-responsive | |
| | | | | protein, vascular smooth muscle - rat | |
| | | | | [R. norvegicus], SCAN domain-containing 2 | |
| 130 | 22727 | NM_019383 | | ATP synthase, H+ transporting, mitochondrial F | i |
| | | | | complex, subunit d, EST, Weakly similar to | |
| | | | | ATP SYNTHASE D CHAIN, MITOCHONDRIAL | |
| | | | | [R. norvegicus], EST, Weakly similar to | |
| | | | | ATPQ_HUMAN ATP SYNTHASE D CHAIN, | |
| | | | | MITOCHONDRIAL [H. sapiens], Homo sapiens | |
| | | | | clone 23e8 My32 protein mRNA, complete cds, | |
| | | | | RIKEN cDNA 619D1 gene | |
| 131 | 16 | NM_019386 | | EST, Weakly similar to TGLC MOUSE | General |
| | | | | PROTEIN-GLUTAMINE GAMMA- | |
| | | | | GLUTAMYLTRANSFERASE [M. musculus], | |
| | | | | Homo sapiens mRNA; cDNA DKFZp667H46 | |
| | | | | (from clone DKFZp667H46), Tissue | |
| | | | | transglutaminase homologue {alternatively | |
| | | | | spliced} [human, erythroleukemia cell line HEL | |
| | | | | GM6141A, mRNA, 2362 nt], transglutaminase 2 | |
| | | | | (C polypeptide, protein-glutamine-gamma- | |
| | | | | glutamyltransferase), transglutaminase 2, C | |
| | | | | polypeptide, transglutaminase 5 | |
| 131 | 17 | NM_019386 | | EST, Weakly similar to TGLC MOUSE | General |
| | | | | PROTEIN-GLUTAMINE GAMMA- | |
| | | | | GLUTAMYLTRANSFERASE [M. musculus], | |
| | | | | Homo sapiens mRNA; cDNA DKFZp667H46 | |
| | | | | (from clone DKFZp667H46), Tissue | |
| | | | | transglutaminase homologue {alternatively | |
| | | | | spliced} [human, erythroleukemia cell line HEL | |
| | | | | GM6141A, mRNA, 2362 nt], transglutaminase 2 | |
| | | | | (C polypeptide, protein-glutamine-gamma- | |
| | | | | glutamyltransferase), transglutaminase 2, C | |
| | | | | polypeptide, transglutaminase 5 | |
| 132 | 574 | NM_019905 | hydroxyacid oxidase | ESTs, Highly similar to (S)-2-HYDROXY-ACID | g, i, |
| | | | (glycolate oxidase) 3, | OXIDASE, PEROXISOMAL [R. norvegicus], | General |
| | | | hydroxyacid oxidase 3 | ESTs, Highly similar to LUHU36 annexin II | |
| | | | (medium-chain) | [H. sapiens], RIKEN cDNA 1113P15 gene, | |
| | | | | RIKEN cDNA B43311C9 gene, annexin A2, | |
| | | | | annexin A2 pseudogene 2, caspase recruitment | |
| | | | | domain family, member 6, expressed sequence | |
| | | | | AW215814, hydroxyacid oxidase (glycolate | |
| | | | | oxidase) 3, hydroxyacid oxidase 1, liver, | |
| | | | | hydroxyacid oxidase 2 (long chain), nucleolar | |
| | | | | protein 3 (apoptosis repressor with CARD | |
| | | | | domain) | |
| 133 | 18713 | NM_020075 | eukaryotic translation | DNA segment, Chr 12, ERATO Doi 549, | General |
| | | | initiation factor 5 | expressed, KIAA1856 protein, eukaryotic | |
| | | | | translation initiation factor 5 | |
| 133 | 18715 | NM_020075 | eukaryotic translation | DNA segment, Chr 12, ERATO Doi 549, | General |
| | | | initiation factor 5 | expressed, KIAA1856 protein, eukaryotic | |
| | | | | translation initiation factor 5 | |
| 134 | 19696 | NM_021699 | | ELKL motif kinase, ESTs, Weakly similar to | General |
| | | | | G125 serine/threonine protein kinase | |
| | | | | [H. sapiens], ESTs, Weakly similar to | |
| | | | | SN1L_RAT PROBABLE SERINE/THREONINE | |
| | | | | PROTEIN KINASE SNF1LK (SALT- | |
| | | | | INDUCIBLE PROTEIN KINASE) (PROTEIN | |
| | | | | KINASE KID2) [R. norvegicus], Homo sapiens | |
| | | | | mRNA; cDNA DKFZp434P81 (from clone | |
| | | | | DKFZp434P81), MAP/microtubule affinity- | |
| | | | | regulating kinase 1, MAP/microtubule affinity- | |
| | | | | regulating kinase 3, RIKEN cDNA 2419P21 | |
| | | | | gene, RIKEN cDNA 573525O22 gene, SNF1- | |
| | | | | like kinase, expressed sequence AW49115, | |
| | | | | salt-inducible protein kinase | |
| 135 | 19710 | NM_021744 | | CD14 antigen, ESTs, Weakly similar to CD14 | i, General |
| | | | | RAT MONOCYTE DIFFERENTIATION | |
| | | | | ANTIGEN CD14 PRECURSOR [R. norvegicus] | |
| 136 | 20035 | NM_021754 | | DKFZP566J153 protein, ESTs, Moderately | General |
| | | | | similar to T17299 hypothetical protein | |
| | | | | DKFZp564H2171.1 [H. sapiens], nucleolar | |
| | | | | protein NOP5/NOP58 | |
| 137 | 14397 | NM_021909 | FXYD domain-containing | EST, Weakly similar to A43932 mucin 2 | g, General |
| | | | ion transport regulator 5 | precursor, intestinal [H. sapiens], ESTs, Weakly | |
| | | | | similar to A43932 mucin 2 precursor, intestinal | |
| | | | | [H. sapiens], FXYD domain-containing ion | |
| | | | | transport regulator 5, Homo sapiens cDNA | |
| | | | | FLJ12566 fis, clone NT2RM4852 | |
| 138 | 17100 | NM_022179 | | EST, Moderately similar to HXK3_HUMAN | h, General |
| | | | | HEXOKINASE TYPE III [H. sapiens], EST, | |
| | | | | Weakly similar to HXK2 MOUSE | |
| | | | | HEXOKINASE TYPE II [M. musculus], ESTs, | |
| | | | | Moderately similar to HXK2 MOUSE | |
| | | | | HEXOKINASE TYPE II [M. musculus], ESTs, | |
| | | | | Weakly similar to HXK2 MOUSE | |
| | | | | HEXOKINASE TYPE II [M. musculus], | |
| | | | | hexokinase 2, hexokinase 3 (white cell) | |
| 138 | 17101 | NM_022179 | | EST, Moderately similar to HXK3_HUMAN | General |
| | | | | HEXOKINASE TYPE III [H. sapiens], EST, | |
| | | | | Weakly similar to HXK2 MOUSE | |
| | | | | HEXOKINASE TYPE II [M. musculus], ESTs, | |
| | | | | Moderately similar to HXK2 MOUSE | |
| | | | | HEXOKINASE TYPE II [M. musculus], ESTs, | |
| | | | | Weakly similar to HXK2 MOUSE | |
| | | | | HEXOKINASE TYPE II [M. musculus], | |
| | | | | hexokinase 2, hexokinase 3 (white cell) | |
| 139 | 20204 | NM_022196 | | leukemia inhibitory factor, leukemia inhibitory | i, General |
| | | | | factor (cholinergic differentiation factor) | |
| 140 | 20385 | NM_022229 | | EST, Moderately similar to CH6 MOUSE 6 KDA | h |
| | | | | HEAT SHOCK PROTEIN, MITOCHONDRIAL | |
| | | | | PRECURSOR [M. musculus], EST, Moderately | |
| | | | | similar to CH6_HUMAN 6 KDA HEAT SHOCK | |
| | | | | PROTEIN, MITOCHONDRIAL PRECURSOR | |
| | | | | [H. sapiens], ESTs, Highly similar to I5342 | |
| | | | | chaperonin [H. sapiens], ESTs, Weakly similar | |
| | | | | to I5342 chaperonin [H. sapiens], Homo | |
| | | | | sapiens, clone IMAGE: 4155316, mRNA, partial | |
| | | | | cds, heat shock 6 kD protein 1 (chaperonin), | |
| | | | | heat shock protein, 6 kDa | |
| 141 | 6263 | NM_022251 | | EST, Weakly similar to AMPN RAT | b |
| | | | | AMINOPEPTIDASE N [R. norvegicus], Leucine | |
| | | | | arylaminopeptidase 1, alanyl (membrane) | |
| | | | | aminopeptidase, alanyl (membrane) | |
| | | | | aminopeptidase (aminopeptidase N, | |
| | | | | aminopeptidase M, microsomal | |
| | | | | aminopeptidase, CD13, p15), aminopeptidase, | |
| | | | | glutamyl aminopeptidase, glutamyl | |
| | | | | aminopeptidase (aminopeptidase A), type 1 | |
| | | | | tumor necrosis factor receptor shedding | |
| | | | | aminopeptidase regulator | |
| 142 | 6585 | NM_022266 | | WNT1 inducible signaling pathway protein 1, | i, General |
| | | | | WNT1 inducible signaling pathway protein 2, | |
| | | | | WNT1 inducible signaling pathway protein 3, | |
| | | | | connective tissue growth factor | |
| 143 | 17161 | NM_022298 | | ESTs, Highly similar to A2335 tubulin alpha | g, General |
| | | | | chain [H. sapiens], tubulin alpha 1, tubulin alpha | |
| | | | | 2, tubulin alpha 3, tubulin alpha 6, tubulin, alpha | |
| | | | | 2, tubulin, alpha 3, tubulin, alpha, ubiquitous | |
| 144 | 13480 | NM_022390 | | RIKEN cDNA 2618L4 gene, quinoid | f |
| | | | | dihydropteridine reductase | |
| 145 | 22415 | NM_022392 | | EST, Highly similar to ISI1_HUMAN INSULIN- | General |
| | | | | INDUCED PROTEIN 1 [H. sapiens], ESTs, | |
| | | | | Highly similar to INSULIN-INDUCED GROWTH | |
| | | | | RESPONSE PROTEIN CL-6 [R. norvegicus], | |
| | | | | Mus musculus, clone MGC: 1894 | |
| | | | | IMAGE: 424711, mRNA, complete cds, insulin | |
| | | | | induced gene 1, insulin induced protein 2 | |
| 146 | 23705 | NM_022396 | | EST, Weakly similar to I39159 GTP-binding | h |
| | | | | regulatory protein gamma-11 chain [H. sapiens], | |
| | | | | ESTs, Moderately similar to GBGB_HUMAN | |
| | | | | GUANINE NUCLEOTIDE-BINDING PROTEIN | |
| | | | | G(l)/G(S)/G(O) GAMMA-11 SUBUNIT | |
| | | | | [R. norvegicus], ESTs, Weakly similar to | |
| | | | | GBGB_HUMAN GUANINE NUCLEOTIDE- | |
| | | | | BINDING PROTEIN G(l)/G(S)/G(O) GAMMA- | |
| | | | | 11 SUBUNIT [R. norvegicus], RIKEN cDNA | |
| | | | | 6137B21 gene, expressed sequence AV96488, | |
| | | | | guanine nucleotide binding protein (G protein), | |
| | | | | gamma 1 subunit, guanine nucleotide binding | |
| | | | | protein (G protein), gamma transducing activity | |
| | | | | polypeptide 1, guanine nucleotide binding | |
| | | | | protein (G protein), gamma transducing activity | |
| | | | | polypeptide 2, guanine nucleotide binding | |
| | | | | protein 11 | |
| 147 | 23300 | NM_022398 | | ESTs, Weakly similar to M2OM RAT | h |
| | | | | MITOCHONDRIAL 2- | |
| | | | | OXOGLUTARATE/MALATE CARRIER | |
| | | | | PROTEIN [R. norvegicus], ESTs, Weakly similar | |
| | | | | to M2OM_HUMAN MITOCHONDRIAL 2- | |
| | | | | OXOGLUTARATE/MALATE CARRIER | |
| | | | | PROTEIN [H. sapiens], RIKEN cDNA | |
| | | | | 363241G24 gene, RIKEN cDNA 4933433D23 | |
| | | | | gene, solute carrier family 25 (mitochondrial | |
| | | | | carrier, brain), member 14, solute carrier family | |
| | | | | 25 (mitochondrial carrier; dicarboxylate | |
| | | | | transporter), member 1, solute carrier family 25 | |
| | | | | (mitochondrial carrier; oxoglutarate carrier), | |
| | | | | member 11 | |
| 148 | 24536 | NM_022399 | | ESTs, Weakly similar to CALRETICULIN | c |
| | | | | PRECURSOR [R. norvegicus], Homo sapiens, | |
| | | | | Similar to RIKEN cDNA 1731L1 gene, clone | |
| | | | | MGC: 26577 IMAGE: 48221, mRNA, complete | |
| | | | | cds, RIKEN cDNA 1731L1 gene, RIKEN cDNA | |
| | | | | 633586I2 gene, calreticulin | |
| 149 | 20915 | NM_022407 | aldehyde dehydrogenase | EST, Moderately similar to A Chain A, Human | General |
| | | | 1 family, member A1, | Mitochondrial Aldehyde Dehydrogenase | |
| | | | aldehyde dehydrogenase | Complexed With Nad+ And Mn2+ {SUB 24-517 | |
| | | | family 1, subfamily A1 | [H. sapiens], ESTs, Moderately similar to | |
| | | | | ALDEHYDE DEHYDROGENASE, | |
| | | | | MITOCHONDRIAL PRECURSOR | |
| | | | | [R. norvegicus], RIKEN cDNA 277F14 gene, | |
| | | | | aldehyde dehydrogenase 1 family, member B1, | |
| | | | | aldehyde dehydrogenase 2 family | |
| | | | | (mitochondrial), aldehyde dehydrogenase 2, | |
| | | | | mitochondrial, aldehyde dehydrogenase 9 | |
| | | | | family, member A1, aldehyde dehydrogenase | |
| | | | | 9, subfamily A1 | |
| 150 | 8212 | NM_022500 | ferritin light chain 1, | ESTs, Highly similar to FRHUL ferritin light | General |
| | | | ferritin, light polypeptide | chain [H. sapiens], ESTs, Moderately similar to | |
| | | | | FRHUL ferritin light chain [H. sapiens], PRO47 | |
| | | | | protein, RIKEN cDNA 4933416E14 gene, | |
| | | | | ferritin light chain 2, ferritin, light polypeptide | |
| 151 | 1867 | NM_022510 | | ESTs, Moderately similar to RL4_HUMAN 6S | General |
| | | | | RIBOSOMAL PROTEIN L4 [H. sapiens], H19, | |
| | | | | imprinted maternally expressed untranslated | |
| | | | | mRNA, Homo sapiens cDNA: FLJ23366 fis, | |
| | | | | clone HEP15665 | |
| 152 | 4242 | NM_022521 | | ESTs, Highly similar to ORNITHINE | f |
| | | | | AMINOTRANSFERASE PRECURSOR | |
| | | | | [R. norvegicus], RIKEN cDNA 1319H2 gene, | |
| | | | | RIKEN cDNA 296B13 gene, hypothetical | |
| | | | | protein MGC15875, ornithine aminotransferase, | |
| | | | | ornithine aminotransferase (gyrate atrophy) | |
| 153 | 6100 | NM_022531 | | | f, General |
| 154 | 8598 | NM_022538 | | | f |
| 154 | 8597 | NM_022538 | | | b, General |
| 155 | 11039 | NM_022543 | | | General |
| 156 | 20925 | NM_022594 | enoyl Coenzyme A | AU RNA-binding protein/enoyl-coenzyme A | e |
| | | | hydratase 1, peroxisomal, | hydratase, EST, Weakly similar to | |
| | | | enoyl coenzyme A | ECH1_HUMAN DELTA3, 5-DELTA2, 4- | |
| | | | hydratase 1, peroxisomal | DIENOYL-COA ISOMERASE PRECURSO | |
| | | | | [H. sapiens], RIKEN cDNA 2619M2 gene, enoyl | |
| | | | | Coenzyme A hydratase 1, peroxisomal, enoyl | |
| | | | | coenzyme A hydratase 1, peroxisomal, | |
| | | | | expressed sequence AA617331 | |
| 157 | 20944 | NM_022597 | | cathepsin B, lipocalin 7, tubulointerstitial | f, General |
| | | | | nephritis antigen | |
| 158 | 21211 | NM_022607 | | EST, Weakly similar to S23 neurofilament | b, h |
| | | | | triplet H protein - rat [R. norvegicus], ESTs, | |
| | | | | Weakly similar to S23 neurofilament triplet H | |
| | | | | protein - rat [R. norvegicus], KIAA182 protein, | |
| | | | | NADH dehydrogenase (ubiquinone) | |
| | | | | flavoprotein 3 (1 kD) | |
| 159 | 2250 | NM_022643 | | H2B histone family, member D, H2B histone | h |
| | | | | family, member G, H2B histone family, member | |
| | | | | K, RIKEN cDNA 26122J1 gene, expressed | |
| | | | | sequence AI413321, expressed sequence | |
| | | | | R74621 | |
| 160 | 12542 | NM_022647 | | ESTs, Weakly similar to I4841 histone H2b - | f |
| | | | | mouse [M. musculus], H2B histone family, | |
| | | | | member A, H2B histone family, member G, | |
| | | | | H2B histone family, member K, expressed | |
| | | | | sequence AI413321, expressed sequence | |
| | | | | R74621 | |
| 161 | 15727 | NM_022953 | slit (Drosophila) homolog | ESTs, Highly similar to T42626 secreted | General |
| | | | 1, slit homolog 1 | leucine-rich repeat-containing protein SLIT2 - | |
| | | | (Drosophila) | mouse (fragment) [M. musculus], ESTs, Weakly | |
| | | | | similar to T42626 secreted leucine-rich repeat- | |
| | | | | containing protein SLIT2 - mouse (fragment) | |
| | | | | [M. musculus], Homo sapiens mRNA; cDNA | |
| | | | | DKFZp434N435 (from clone DKFZp434N435); | |
| | | | | partial cds, glycoprotein IX (platelet), | |
| | | | | glycoprotein lb (platelet), beta polypeptide, | |
| | | | | glycoprotein lb, beta polypeptide, slit | |
| | | | | (Drosophila) homolog 1, slit (Drosophila) | |
| | | | | homolog 2, slit (Drosophila) homolog 3, slit | |
| | | | | homolog 1 (Drosophila), slit homolog 2 | |
| | | | | (Drosophila), slit homolog 3 (Drosophila) | |
| 162 | 15729 | NM_022955 | | EGF-like-domain, multiple 3, EST, Highly | General |
| | | | | similar to A443 notch protein homolog TAN-1 | |
| | | | | precursor [H. sapiens], EST, Weakly similar to | |
| | | | | A443 notch protein homolog TAN-1 precursor | |
| | | | | [H. sapiens], ESTs, Weakly similar to JH771 | |
| | | | | protein-tyrosine kinase [M. musculus], ESTs, | |
| | | | | Weakly similar to T13954 MEGF6 protein - rat | |
| | | | | [R. norvegicus], Notch (Drosophila) homolog 1 | |
| | | | | (translocation-associated), Notch (Drosophila) | |
| | | | | homolog 2, Notch gene homolog 3, | |
| | | | | (Drosophila), Rattus norvegicus CBL2 (CBL2) | |
| | | | | mRNA, complete cds | |
| 163 | 21239 | NM_024125 | CCAAT/enhancer binding | CCAAT/enhancer binding protein (C/EBP), beta | c, General |
| | | | protein (C/EBP), beta | | |
| 164 | 353 | NM_024127 | growth arrest and DNA- | growth arrest and DNA-damage-inducible 45 | i |
| | | | damage-inducible 45 | alpha, growth arrest and DNA-damage- | |
| | | | alpha, growth arrest and | inducible 45 beta, growth arrest and DNA- | |
| | | | DNA-damage-inducible, | damage-inducible, alpha | |
| | | | alpha | | |
| 165 | 17226 | NM_024131 | D-dopachrome | D-dopachrome tautomerase, expressed | General |
| | | | tautomerase | sequence C78655 | |
| 166 | 24634 | NM_024137 | human immunodeficiency | ESTs, Weakly similar to Z177_HUMAN ZINC | General |
| | | | virus type I enhancer | FINGER PROTEIN 177 [H. sapiens], KIAA1555 | |
| | | | binding protein 2, human | protein, human immunodeficiency virus type I | |
| | | | immunodeficiency virus | enhancer-binding protein 2, human | |
| | | | type I enhancer-binding | immunodeficiency virus type I enhancer-binding | |
| | | | protein 2 | protein 3, kappa B and Rss recognition | |
| | | | | component | |
| 166 | 24632 | NM_024137 | human immunodeficiency | ESTs, Weakly similar to Z177_HUMAN ZINC | a |
| | | | virus type I enhancer | FINGER PROTEIN 177 [H. sapiens], KIAA1555 | |
| | | | binding protein 2, human | protein, human immunodeficiency virus type I | |
| | | | immunodeficiency virus | enhancer-binding protein 2, human | |
| | | | type I enhancer-binding | immunodeficiency virus type I enhancer-binding | |
| | | | protein 2 | protein 3, kappa B and Rss recognition | |
| | | | | component | |
| 167 | 22 | NM_024388 | | nuclear receptor subfamily 4, group A, member 1 | b |
| 168 | 9929 | NM_024392 | | Homo sapiens cDNA FLJ13261 fis, clone | g |
| | | | | OVARC1885, weakly similar to | |
| | | | | OXIDOREDUCTASE UCPA (EC 1.—.—.—), | |
| | | | | Human DNA sequence from clone 168E13 on | |
| | | | | chromosome 2p11.21-12.3. Contains two | |
| | | | | putative novel genes, the gene for a novel | |
| | | | | protein similar to bovine SCP2 (Sterol Carrier | |
| | | | | Protein 2) and part of HSD17B4 | |
| | | | | (hydroxysteroid (17-beta) dehydrogenase 4), an | |
| | | | | EEF1A1 (eukaryotic translation elongation | |
| | | | | factor 1 alpha 1) pseudogene, ESTs, STSs and | |
| | | | | GSSs, RIKEN cDNA 11129G7 gene, RIKEN | |
| | | | | cDNA 171M22 gene, RIKEN cDNA 18126B4 | |
| | | | | gene, RIKEN cDNA 26127I16 gene, expressed | |
| | | | | sequence AW2883, hydroxysteroid (17-beta) | |
| | | | | dehydrogenase 4, hydroxysteroid 17-beta | |
| | | | | dehydrogenase 4, hypothetical protein | |
| | | | | MGC194, oxidoreductase UCPA, retinal short- | |
| | | | | chain dehydrogenase/reductase 1 | |
| 169 | 3582 | NM_024396 | | ATP-binding cassette, sub-family A (ABC1), | a |
| | | | | member 2, ATP-binding cassette, sub-family A | |
| | | | | (ABC1), member 3, ATP-binding cassette, sub- | |
| | | | | family A (ABC1), member 4, ATP-binding | |
| | | | | cassette, sub-family A (ABC1), member 7, ATP- | |
| | | | | binding cassette, sub-family A (ABC1), member | |
| | | | | 8, ESTs, Weakly similar to ABC2 MOUSE ATP- | |
| | | | | BINDING CASSETTE TRANSPORTER 2 | |
| | | | | [M. musculus], Homo sapiens mRNA for | |
| | | | | KIAA1888 protein, partial cds, hypothetical | |
| | | | | protein FLJ14297, hypothetical protein | |
| | | | | PRO2543 | |
| 170 | 22626 | NM_024400 | | ESTs, Weakly similar to T47158 hypothetical | General |
| | | | | protein DKFZp762C111.1 [H. sapiens], Mus | |
| | | | | musculus papilin mRNA, complete cds, Mus | |
| | | | | musculus, Similar to a disintegrin and | |
| | | | | metalloproteinase with thrombospondin motifs 1 | |
| | | | | (ADAMTS-1), clone IMAGE: 3491991, mRNA, | |
| | | | | partial cds, RIKEN cDNA 672426B9 gene, | |
| | | | | RIKEN cDNA A938K15 gene, a disintegrin-like | |
| | | | | and metalloprotease (reprolysin type) with | |
| | | | | thrombospondin type 1 motif, 1, a disintegrin- | |
| | | | | like and metalloprotease (reprolysin type) with | |
| | | | | thrombospondin type 1 motif, 4, a disintegrin- | |
| | | | | like and metalloprotease (reprolysin type) with | |
| | | | | thrombospondin type 1 motif, 8, a disintegrin- | |
| | | | | like and metalloprotease (reprolysin type) with | |
| | | | | thrombospondin type 1 motif, 9 | |
| 171 | 13633 | NM_024403 | | EST, Weakly similar to ATF4_HUMAN CYCLIC- | General |
| | | | | AMP-DEPENDENT TRANSCRIPTION | |
| | | | | FACTOR ATF-4 [H. sapiens], activating | |
| | | | | transcription factor 4 (tax-responsive enhancer | |
| | | | | element B67), activating transcription factor 5 | |
| 171 | 13634 | NM_024403 | | EST, Weakly similar to ATF4_HUMAN CYCLIC- | General |
| | | | | AMP-DEPENDENT TRANSCRIPTION | |
| | | | | FACTOR ATF-4 [H. sapiens], activating | |
| | | | | transcription factor 4 (tax-responsive enhancer | |
| | | | | element B67), activating transcription factor 5 | |
| 172 | 17917 | NM_024488 | | | a, b |
| 173 | 26164 | NM_030836 | | ESTs, Weakly similar to PSA MOUSE | i |
| | | | | PUROMYCIN-SENSITIVE AMINOPEPTIDASE | |
| | | | | [M. musculus], aminopeptidase, glutamyl | |
| | | | | aminopeptidase, leucyl/cystinyl | |
| | | | | aminopeptidase, thyrotropin-releasing hormone | |
| | | | | degrading ectoenzyme, type 1 tumor necrosis | |
| | | | | factor receptor shedding aminopeptidase | |
| | | | | regulator | |
| 174 | 1221 | NM_030845 | | GRO1 oncogene, GRO2 oncogene, Rattus | General |
| | | | | norvegicus CXC chemokine RTCK1 (Rtck1) | |
| | | | | mRNA, complete cds, interleukin 8, platelet | |
| | | | | factor 4, pro-platelet basic protein, pro-platelet | |
| | | | | basic protein (includes platelet basic protein, | |
| | | | | beta-thromboglobulin, connective tissue- | |
| | | | | activating peptide III, neutrophil-activating | |
| | | | | peptide-2) | |
| 175 | 17342 | NM_030873 | | EST, Weakly similar to A Chain A, Human | d |
| | | | | Platelet Profilin Complexed With The L-Pro1 | |
| | | | | Peptide {SUB 3-14 [H. sapiens], ESTs, Weakly | |
| | | | | similar to profilin [R. norvegicus], RIKEN cDNA | |
| | | | | 1712P12 gene, profilin, profilin 1, profilin 2 | |
| 176 | 23109 | NM_031000 | | EST, Weakly similar to ALCOHOL | c, f, h |
| | | | | DEHYDROGENASE [R. norvegicus], RIKEN | |
| | | | | cDNA 2315E1 gene, aldo-keto reductase family | |
| | | | | 1, member A1 (aldehyde reductase), aldo-keto | |
| | | | | reductase family 1, member A4 (aldehyde | |
| | | | | reductase), aldo-keto reductase family 1, | |
| | | | | member E1 | |
| 177 | 21165 | NM_031005 | actinin, alpha 1 | ESTs, Weakly similar to FAHUAA alpha-actinin | General |
| | | | | 1 [H. sapiens], Homo sapiens, Similar to | |
| | | | | cysteine and histidine-rich protein, clone | |
| | | | | IMAGE: 3945559, mRNA, partial cds, RIKEN | |
| | | | | cDNA 31123F1 gene, actinin alpha 2, actinin | |
| | | | | alpha 3, actinin, alpha 1, actinin, alpha 2, | |
| | | | | actinin, alpha 3, actinin, alpha 4, alpha actinin | |
| | | | | 4, beta-spectrin 2, non-erythrocytic, beta- | |
| | | | | spectrin 3 | |
| 178 | 1845 | NM_031010 | | ESTs, Moderately similar to ARACHIDONATE | a, b, |
| | | | | 12-LIPOXYGENASE [R. norvegicus], | General |
| | | | | arachidonate 12-lipoxygenase, arachidonate 12- | |
| | | | | lipoxygenase pseudogene 2, arachidonate 15- | |
| | | | | lipoxygenase, expressed sequence AW259591 | |
| 178 | 25517 | NM_031010 | | ESTs, Moderately similar to ARACHIDONATE | a, b, |
| | | | | 12-LIPOXYGENASE [R. norvegicus], | General |
| | | | | arachidonate 12-lipoxygenase, arachidonate 12- | |
| | | | | lipoxygenase pseudogene 2, arachidonate 15- | |
| | | | | lipoxygenase, expressed sequence AW259591 | |
| 179 | 15886 | NM_031035 | | guanine nucleotide binding protein (G protein), | c, f |
| | | | | alpha inhibiting activity polypeptide 2, guanine | |
| | | | | nucleotide binding protein, alpha inhibiting 2, | |
| | | | | guanine nucleotide binding protein, alpha | |
| | | | | inhibiting 3 | |
| 180 | 15957 | NM_031050 | | ESTs, Weakly similar to LUM RAT LUMICAN | h |
| | | | | PRECURSOR [R. norvegicus], RIKEN cDNA | |
| | | | | 73349J17 gene, dermatan sulphate | |
| | | | | proteoglycan 3, lumican, osteoglycin | |
| | | | | (osteoinductive factor, mimecan), | |
| | | | | osteomodulin, protein phosphatase 1, | |
| | | | | regulatory (inhibitor) subunit 7 | |
| 181 | 11849 | NM_031065 | | EST, Moderately similar to R1A MOUSE 6S | h, General |
| | | | | RIBOSOMAL PROTEIN L1A [M. musculus], | |
| | | | | EST, Weakly similar to 6S RIBOSOMAL | |
| | | | | PROTEIN L1A [R. norvegicus], ESTs, Highly | |
| | | | | similar to R1A_HUMAN 6S RIBOSOMAL | |
| | | | | PROTEIN L1A [H. sapiens], ribosomal protein | |
| | | | | L1A, ribosomal protein L1a | |
| 182 | 12639 | NM_031099 | | ESTs, Weakly similar to S55912 ribosomal | e, General |
| | | | | protein L5, cytosolic [H. sapiens], ribosomal | |
| | | | | protein L5 | |
| 183 | 20812 | NM_031100 | | EST, Moderately similar to 6S RIBOSOMAL | e |
| | | | | PROTEIN L1 [M. musculus], EST, Moderately | |
| | | | | similar to 6S RIBOSOMAL PROTEIN L1 | |
| | | | | [R. norvegicus], ESTs, Highly similar to A42735 | |
| | | | | ribosomal protein L1, cytosolic [H. sapiens], | |
| | | | | Homo sapiens, Similar to ribosomal protein L1, | |
| | | | | clone MGC: 22634 IMAGE: 3935452, mRNA, | |
| | | | | complete cds, Human DNA sequence from | |
| | | | | clone RP3-334F4 on chromosome 6 Contains | |
| | | | | ESTs, STSs and GSSs. Contains a LAMR1 | |
| | | | | (laminin receptor 1, ribosomal protein SA) | |
| | | | | pseudogene and an RPL1 (ribosomal protein | |
| | | | | L1) pseudogene, Mouse 24.6 kda protein | |
| | | | | mRNA, complete cds, ribosomal protein L1 | |
| 184 | 23854 | NM_031101 | | EST, Moderately similar to JC2368 ribosomal | General |
| | | | | protein L13 - rat [R. norvegicus], EST, | |
| | | | | Moderately similar to S23753 ribosomal protein | |
| | | | | L13, cytosolic [H. sapiens], EST, Weakly similar | |
| | | | | to JC2368 ribosomal protein L13 - rat | |
| | | | | [R. norvegicus], ESTs, Highly similar to S23753 | |
| | | | | ribosomal protein L13, cytosolic [H. sapiens], | |
| | | | | ESTs, Moderately similar to RL13 MOUSE 6S | |
| | | | | RIBOSOMAL PROTEIN L13 [M. musculus], | |
| | | | | ribosomal protein L13 | |
| 185 | 16938 | NM_031103 | | ESTs, Weakly similar to 6S RIBOSOMAL | e |
| | | | | PROTEIN L19 [R. norvegicus], Homo sapiens | |
| | | | | mRNA; cDNA DKFZp434D115 (from clone | |
| | | | | DKFZp434D115), ribosomal protein L19 | |
| 186 | 22205 | NM_031105 | | ESTs, Highly similar to RL44_HUMAN 6S | h |
| | | | | RIBOSOMAL PROTEIN L44 [H. sapiens], | |
| | | | | RIKEN cDNA 24138A3 gene, ribosomal protein | |
| | | | | L36a, ribosomal protein L44 | |
| 187 | 16847 | NM_031109 | | DNA segment, Chr 4, ERATO Doi 429, | g, General |
| | | | | expressed, ESTs, Highly similar to S55918 | |
| | | | | ribosomal protein S1, cytosolic [H. sapiens], | |
| | | | | Homo sapiens cDNA: FLJ217 fis, clone | |
| | | | | COL9849, highly similar to HSU14972 Human | |
| | | | | ribosomal protein S1 mRNA, RIKEN cDNA | |
| | | | | 22142A9 gene, ribosomal protein S1 | |
| 188 | 10878 | NM_031110 | | EST, Weakly similar to 4S RIBOSOMAL | e, General |
| | | | | PROTEIN S11 [R. norvegicus], Homo sapiens | |
| | | | | mRNA; cDNA DKFZp434A326 (from clone | |
| | | | | DKFZp434A326), Human DNA sequence from | |
| | | | | clone RP5-16K6 on chromosome 2p12.1-13. | |
| | | | | Contains an RPS11 (4S ribosomal protein S11) | |
| | | | | pseudogene, ESTs, STSs and GSSs, RAD21 | |
| | | | | homolog (S. pombe), ribosomal protein S11 | |
| 189 | 20839 | NM_031113 | | EST, Moderately similar to S12583 | General |
| | | | | polyubiquitin 4 - mouse [M. musculus], EST, | |
| | | | | Moderately similar to ubiquitin/ribosomal | |
| | | | | protein S27a [R. norvegicus], EST, Weakly | |
| | | | | similar to R27A_HUMAN 4S RIBOSOMAL | |
| | | | | PROTEIN S27A {SUB 77-156 [H. sapiens], | |
| | | | | Homo sapiens cDNA FLJ1163 fis, clone | |
| | | | | HEMBA13926, RIKEN cDNA 616J14 gene, | |
| | | | | expressed sequence AI132487, ribosomal | |
| | | | | protein S27a, ubiquitin C | |
| 190 | 22749 | NM_031116 | | small inducible cytokine A5, small inducible | b |
| | | | | cytokine A5 (RANTES), small inducible | |
| | | | | cytokine subfamily A (Cys-Cys), member 17, | |
| | | | | small inducible cytokine subfamily A (Cys-Cys), | |
| | | | | member 26 | |
| 191 | 1580 | NM_031117 | | EST, Weakly similar to A3327 snRNP protein N | h |
| | | | | [H. sapiens], Homo sapiens RT-LI mRNA, | |
| | | | | complete sequence, RIKEN cDNA 24145I1 | |
| | | | | gene, SNRPN upstream reading frame, WW | |
| | | | | domain binding protein 11, ribosomal protein | |
| | | | | S1, small nuclear ribonucleoprotein B, small | |
| | | | | nuclear ribonucleoprotein N, small nuclear | |
| | | | | ribonucleoprotein polypeptide N, small nuclear | |
| | | | | ribonucleoprotein polypeptides B and B1 | |
| 192 | 13929 | NM_031131 | | Transforming growth factor, beta 3, | General |
| | | | | transforming growth factor, beta 2, transforming | |
| | | | | growth factor, beta 3 | |
| 193 | 15185 | NM_031140 | | EST, Moderately similar to A2574 vimentin | c, General |
| | | | | [H. sapiens], EST, Weakly similar to A2574 | |
| | | | | vimentin [H. sapiens], ESTs, Moderately similar | |
| | | | | to VIME RAT VIMENTIN [R. norvegicus], ESTs, | |
| | | | | Weakly similar to A2574 vimentin [H. sapiens], | |
| | | | | ESTs, Weakly similar to VIME RAT VIMENTIN | |
| | | | | [R. norvegicus], vimentin | |
| 194 | 1638 | NM_031143 | | EST, Weakly similar to JC6124 diacylglycerol | General |
| | | | | kinase (EC 2.7.1.17) IV - rat [R. norvegicus], | |
| | | | | ESTs, Highly similar to KDGZ_HUMAN | |
| | | | | DIACYLGLYCEROL KINASE, ZETA | |
| | | | | (DIGLYCERIDE KINASE) (DGK-ZETA) (DAG | |
| | | | | KINASE ZETA) [H. sapiens], ESTs, Weakly | |
| | | | | similar to JC6124 diacylglycerol kinase (EC | |
| | | | | 2.7.1.17) IV - rat [R. norvegicus], ESTs, Weakly | |
| | | | | similar to KDGA MOUSE DIACYLGLYCEROL | |
| | | | | KINASE, ALPHA [M. musculus], diacylglycerol | |
| | | | | kinase, alpha (8 kDa), diacylglycerol kinase, | |
| | | | | iota, diacylglycerol kinase, zeta (14 kD) | |
| 195 | 21624 | NM_031144 | | EST, Weakly similar to ACTB_HUMAN ACTIN, | g |
| | | | | CYTOPLASMIC 1 [R. norvegicus], ESTs, Highly | |
| | | | | similar to ATHUB actin beta [H. sapiens], ESTs, | |
| | | | | Weakly similar to ACTB_HUMAN ACTIN, | |
| | | | | CYTOPLASMIC 1 [R. norvegicus], Homo | |
| | | | | sapiens FKSG3 (FKSG3) mRNA, complete | |
| | | | | cds, RIKEN cDNA 1752K15 gene, RIKEN | |
| | | | | cDNA 1761J2 gene, actin, beta, actin-like 7a, | |
| | | | | actin-related protein 3-beta, melanoma X-actin | |
| 196 | 15277 | NM_031237 | | EST, Moderately similar to UB5B_HUMAN | g, i, |
| | | | | UBIQUITIN-CONJUGATING ENZYME E2-17 | General |
| | | | | KD 2 [R.norvegicus], ESTs, Moderately similar | |
| | | | | to I59365 ubiquitin conjugating enzyme | |
| | | | | [H. sapiens], ESTs, Moderately similar to | |
| | | | | UB5B_HUMAN UBIQUITIN-CONJUGATING | |
| | | | | ENZYME E2-17 KD 2 [M. musculus], RIKEN | |
| | | | | cDNA 111F19 gene, RIKEN cDNA 1628I17 | |
| | | | | gene, RIKEN cDNA 2784L22 gene, Rattus | |
| | | | | norvegicus clone ubc2e ubiquitin conjugating | |
| | | | | enzyme (E217 kB) mRNA, complete cds, | |
| | | | | expressed sequence AL22654, ubiquitin- | |
| | | | | conjugating enzyme E2D 1 (homologous to | |
| | | | | yeast UBC4/5), ubiquitin-conjugating enzyme | |
| | | | | E2D 2, ubiquitin-conjugating enzyme E2D 2 | |
| | | | | (homologous to yeast UBC4/5), ubiquitin- | |
| | | | | conjugating enzyme E2D 3 (homologous to | |
| | | | | yeast UBC4/5) | |
| 197 | 18597 | NM_031325 | | UDP-glucose dehydrogenase | General |
| 198 | 11258 | NM_031327 | | ESTs, Moderately similar to CYR6 MOUSE | General |
| | | | | CYR61 PROTEIN PRECURSOR [M. musculus], | |
| | | | | cysteine rich protein 61, cysteine-rich, | |
| | | | | angiogenic inducer, 61 | |
| 199 | 18654 | NM_031358 | | expressed sequence AI842722, potassium | i, General |
| | | | | inwardly rectifying channel, subfamily J, | |
| | | | | member 11, potassium inwardly-rectifying | |
| | | | | channel, subfamily J, member 11 | |
| 200 | 12581 | NM_031514 | Janus kinase 2, Janus | ESTs, Weakly similar to JAK2 RAT TYROSINE- | i, General |
| | | | kinase 2 (a protein | PROTEIN KINASE JAK2 [R. norvegicus], Janus | |
| | | | tyrosine kinase) | kinase 2, Janus kinase 2 (a protein tyrosine | |
| | | | | kinase), expressed sequence C81284 | |
| 201 | 20449 | NM_031530 | small inducible cytokine | EST, Weakly similar to SY2 RAT SMALL | g, General |
| | | | A2, small inducible | INDUCIBLE CYTOKINE A2 PRECURSOR | |
| | | | cytokine A2 (monocyte | [R. norvegicus], expressed sequence AI323594, | |
| | | | chemotactic protein 1, | expressed sequence AW987545, small | |
| | | | homologous to mouse Sig | inducible cytokine A2, small inducible cytokine | |
| | | | je) | A24, small inducible cytokine A7 (monocyte | |
| | | | | chemotactic protein 3), small inducible cytokine | |
| | | | | subfamily A (Cys-Cys), member 17 | |
| 201 | 20448 | NM_031530 | small inducible cytokine | EST, Weakly similar to SY2 RAT SMALL | g |
| | | | A2, small inducible | INDUCIBLE CYTOKINE A2 PRECURSOR | |
| | | | cytokine A2 (monocyte | [R. norvegicus], expressed sequence AI323594, | |
| | | | chemotactic protein 1, | expressed sequence AW987545, small | |
| | | | homologous to mouse Sig | inducible cytokine A2, small inducible cytokine | |
| | | | je) | A24, small inducible cytokine A7 (monocyte | |
| | | | | chemotactic protein 3), small inducible cytokine | |
| | | | | subfamily A (Cys-Cys), member 17 | |
| 202 | 445 | NM_031535 | BCL2-like 1, Bcl2-like | BCL2-like 1, BCL2-like 2, Bcl-2-related ovarian | b |
| | | | | killer protein, Bcl-2-related ovarian killer protein- | |
| | | | | like, Bcl-w protein, Bcl2-like, Bcl2-like 2 | |
| 203 | 4011 | NM_031543 | cytochrome P45, 2e1, | cytochrome P45, 2e1, ethanol inducible, | a |
| | | | ethanol inducible, | cytochrome P45, subfamily IIE (ethanol- | |
| | | | cytochrome P45, | inducible) | |
| | | | subfamily IIE (ethanol- | | |
| | | | inducible) | | |
| 204 | 18389 | NM_031545 | natriuretic peptide | expressed sequence AA48272, natriuretic | i, General |
| | | | precursor B, natriuretic | peptide precursor B, natriuretic peptide | |
| | | | peptide precursor type B | precursor type B | |
| 205 | 692 | NM_031557 | prostaglandin I2 | EST, Highly similar to PTGI_RAT | h |
| | | | (prostacyclin) synthase | PROSTACYCLIN SYNTHASE | |
| | | | | (PROSTAGLANDIN 12 SYNTHASE) | |
| | | | | [R. norvegicus], cytochrome P45, 8b1, sterol 12 | |
| | | | | alpha-hydrolase, cytochrome P45, subfamily | |
| | | | | VIIIB (sterol 12-alpha-hydroxylase), polypeptide | |
| | | | | 1, prostaglandin I2 (prostacyclin) synthase | |
| 206 | 18318 | NM_031561 | CD36 antigen, CD36 | CD36 antigen, CD36 antigen (collagen type I | h |
| | | | antigen (collagen type I | receptor, thrombospondin receptor) | |
| | | | receptor, thrombospondin | | |
| | | | receptor) | | |
| 207 | 24219 | NM_031579 | protein tyrosine | protein tyrosine phosphatase 4a1, protein | General |
| | | | phosphatase 4a1, protein | tyrosine phosphatase 4a2, protein tyrosine | |
| | | | tyrosine phosphatase | phosphatase type IVA, member 1, protein | |
| | | | type IVA, member 1 | tyrosine phosphatase type IVA, member 2, | |
| | | | | protein tyrosine phosphatase type IVA, member 3 | |
| 208 | 24235 | NM_031614 | thioredoxin reductase 1 | Mus musculus adult male small intestine cDNA, | General |
| | | | | RIKEN full-length enriched library, clone: 211F3, | |
| | | | | full insert sequence, glutathione reductase 1, | |
| | | | | thioredoxin reductase 1, thioredoxin reductase | |
| | | | | 2, thioredoxin reductase beta | |
| 209 | 567 | NM_031628 | nuclear receptor | ESTs, Moderately similar to NR42 MOUSE | General |
| | | | subfamily 4, group A, | ORPHAN NUCLEAR RECEPTOR NURR1 | |
| | | | member 3 | [M. musculus], nuclear receptor subfamily 4, | |
| | | | | group A, member 2, nuclear receptor subfamily | |
| | | | | 4, group A, member 3 | |
| 210 | 211 | NM_031664 | solute carrier family 28 | solute carrier family 28 (sodium-coupled | c |
| | | | (sodium-coupled | nucleoside transporter), member 2, solute | |
| | | | nucleoside transporter), | carrier family 28 (sodium-coupled nucleoside | |
| | | | member 2 | transporter), member 3 | |
| 211 | 18403 | NM_031677 | four and a half LIM | EST, Weakly similar to SLI3 RAT SKELETAL | g |
| | | | domains 2 | MUSCLE LIM-PROTEIN 3 [R. norvegicus], | |
| | | | | RIKEN cDNA 23126N9 gene, activator of | |
| | | | | CREM in testis, four and a half LIM domains 2, | |
| | | | | four and a half LIM domains 3, vascular Rab- | |
| | | | | GAP/TBC-containing | |
| 212 | 20743 | NM_031684 | solute carrier family 29 | ESTs, Weakly similar to ENT1_RAT | f, g, |
| | | | (nucleoside transporters), | EQUILIBRATIVE NUCLEOSIDE | General |
| | | | member 1 | TRANSPORTER 1 (EQUILIBRATIVE | |
| | | | | NITROBENZYLMERCAPTOPURINE | |
| | | | | RIBOSIDE-SENSITIVE NUCLEOSIDE | |
| | | | | TRANSPORTER) (EQUILIBRATIVE NBMPR- | |
| | | | | SENSITIVE NUCLEOSIDE TRANSPORTER) | |
| | | | | (NUCLEOSIDE TRANSPORTER, ES-TYPE) | |
| | | | | [R. norvegicus], RIKEN cDNA 4933435C21 | |
| | | | | gene, hypothetical protein FLJ1116, solute | |
| | | | | carrier family 29 (nucleoside transporters), | |
| | | | | member 1, solute carrier family 29 (nucleoside | |
| | | | | transporters), member 2 | |
| 213 | 21575 | NM_031698 | ribophorin II | EST, Moderately similar to RIB2_HUMAN | b |
| | | | | DOLICHYL-DIPHOSPHOOLIGOSACCHARIDE - | |
| | | | | PROTEIN GLYCOSYLTRANSFERASE 63 | |
| | | | | KDA SUBUNIT PRECURSOR [H. sapiens], | |
| | | | | ESTs, Moderately similar to DOLICHYL- | |
| | | | | DIPHOSPHOOLIGOSACCHARIDE - PROTEIN | |
| | | | | GLYCOSYLTRANSFERASE 63 KD SUBUNIT | |
| | | | | PRECURSOR [R. norvegicus], ESTs, | |
| | | | | Moderately similar to RIB2_HUMAN | |
| | | | | DOLICHYL-DIPHOSPHOOLIGOSACCHARIDE - | |
| | | | | PROTEIN GLYCOSYLTRANSFERASE 63 | |
| | | | | KDA SUBUNIT PRECURSOR [H. sapiens], | |
| | | | | ribophorin 2, related sequence 1, ribophorin II | |
| 214 | 16918 | NM_031709 | | ESTs, Highly similar to R3HU12 ribosomal | e, General |
| | | | | protein S12, cytosolic [H. sapiens], | |
| | | | | mitochondrial ribosomal protein L5, ribosomal | |
| | | | | protein S12 | |
| 215 | 19050 | NM_031719 | | ESTs, Highly similar to ICLN_HUMAN | General |
| | | | | CHLORIDE CONDUCTANCE REGULATORY | |
| | | | | PROTEIN ICLN [H. sapiens], RIKEN cDNA | |
| | | | | 26136D6 gene, RIKEN cDNA 2611O4 gene, | |
| | | | | chloride channel, nucleotide-sensitive, 1A | |
| 215 | 19048 | NM_031719 | | ESTs, Highly similar to ICLN_HUMAN | f |
| | | | | CHLORIDE CONDUCTANCE REGULATORY | |
| | | | | PROTEIN ICLN [H. sapiens], RIKEN cDNA | |
| | | | | 26136D6 gene, RIKEN cDNA 2611O4 gene, | |
| | | | | chloride channel, nucleotide-sensitive, 1A | |
| 216 | 3549 | NM_031723 | | RIKEN cDNA 18129G24 gene, Rattus | h |
| | | | | norvegicus mRNA for signal peptidase 21 kDa | |
| | | | | subunit, complete cds, signal peptidase | |
| | | | | complex (18 kD), similar to signal peptidase | |
| | | | | complex (18 kD) | |
| 217 | 14184 | NM_031776 | | guanine deaminase | General |
| 217 | 14185 | NM_031776 | | guanine deaminase | General |
| 218 | 1169 | NM_031789 | | ESTs, Highly similar to NFL2 RAT NUCLEAR | a |
| | | | | FACTOR ERYTHROID 2 RELATED FACTOR | |
| | | | | 2 [R. norvegicus], ESTs, Weakly similar to NFL2 | |
| | | | | RAT NUCLEAR FACTOR ERYTHROID 2 | |
| | | | | RELATED FACTOR 2 [R. norvegicus], nuclear | |
| | | | | factor (erythroid-derived 2)-like 2, nuclear, | |
| | | | | factor, erythroid derived 2, like 2 | |
| 219 | 17195 | NM_031814 | G protein-coupled | EST, Weakly similar to T42627 ADP- | g |
| | | | receptor kinase-interactor 1 | ribosylation factor-directed GTPase activating | |
| | | | | protein, isoform a - mouse [M. musculus], ESTs, | |
| | | | | Highly similar to T42627 ADP-ribosylation | |
| | | | | factor-directed GTPase activating protein, | |
| | | | | isoform a - mouse [M. musculus], G protein- | |
| | | | | coupled receptor kinase-interactor 1, G protein- | |
| | | | | coupled receptor kinase-interactor 2, Homo | |
| | | | | sapiens p95 paxillin-kinase linker mRNA, | |
| | | | | complete cds, RIKEN cDNA 173C1 gene, | |
| | | | | development and differentiation enhancing | |
| 220 | 17535 | NM_031816 | | ESTs, Weakly similar to GBLP_HUMAN | h |
| | | | | GUANINE NUCLEOTIDE-BINDING PROTEIN | |
| | | | | BETA SUBUNIT-LIKE PROTEIN 12.3 | |
| | | | | [R. norvegicus], Homo sapiens cDNA: | |
| | | | | FLJ21913 fis, clone HEP3888, Homo sapiens, | |
| | | | | clone IMAGE: 35217, mRNA, partial cds, RAE1 | |
| | | | | (RNA export 1, S.pombe) homolog, WD repeat | |
| | | | | domain 1, expressed sequence AI173248, | |
| | | | | expressed sequence AI54353, guanine | |
| | | | | nucleotide binding protein (G protein), beta | |
| | | | | polypeptide 2-like 1, guanine nucleotide binding | |
| | | | | protein, beta 2, related sequence 1, | |
| | | | | retinoblastoma binding protein 7, | |
| | | | | retinoblastoma-binding protein 7, transducin | |
| | | | | (beta)-like 2 | |
| 221 | 15840 | NM_031817 | osteomodulin | fibromodulin, fibronectin leucine rich | g, General |
| | | | | transmembrane protein 3, keratocan, | |
| | | | | osteomodulin, proline arginine-rich end leucine- | |
| | | | | rich repeat protein | |
| 222 | 2655 | NM_031821 | serum-inducible kinase | ESTs, Highly similar to A57286 probable | i |
| | | | | serine/threonine protein kinase [M. musculus], | |
| | | | | ESTs, Highly similar to SNK_RAT | |
| | | | | SERINE/THREONINE-PROTEIN KINASE SNK | |
| | | | | (SERUM INDUCIBLE KINASE) [R. norvegicus], | |
| | | | | ESTs, Weakly similar to SNK_RAT | |
| | | | | SERINE/THREONINE-PROTEIN KINASE SNK | |
| | | | | (SERUM INDUCIBLE KINASE) [R. norvegicus], | |
| | | | | cytokine-inducible kinase, serine/threonine | |
| | | | | kinase 18, serum-inducible kinase, tousled-like | |
| | | | | kinase 2 (Arabidopsis) | |
| 223 | 3251 | NM_031825 | | ESTs, Highly similar to 171348A fibrillin | b |
| | | | | [H. sapiens], ESTs, Weakly similar to TGFB | |
| | | | | RAT LATENT TRANSFORMING GROWTH | |
| | | | | FACTOR BETA BINDING PROTEIN 1 | |
| | | | | PRECURSOR [R. norvegicus], RIKEN cDNA | |
| | | | | 1315B4 gene, RIKEN cDNA 23146A13 gene, | |
| | | | | fibrillin 1, fibrillin 1 (Marfan syndrome), latent | |
| | | | | transforming growth factor beta binding protein | |
| | | | | 1, transforming growth factor-beta (TGF-beta) | |
| | | | | masking protein large subunit | |
| 223 | 3252 | NM_031825 | | ESTs, Highly similar to 171348A fibrillin | c |
| | | | | [H. sapiens], ESTs, Weakly similar to TGFB | |
| | | | | RAT LATENT TRANSFORMING GROWTH | |
| | | | | FACTOR BETA BINDING PROTEIN 1 | |
| | | | | PRECURSOR [R. norvegicus], RIKEN cDNA | |
| | | | | 1315B4 gene, RIKEN cDNA 23146A13 gene, | |
| | | | | fibrillin 1, fibrillin 1 (Marfan syndrome), latent | |
| | | | | transforming growth factor beta binding protein | |
| | | | | 1, transforming growth factor-beta (TGF-beta) | |
| | | | | masking protein large subunit | |
| 224 | 22321 | NM_031832 | lectin, galactose binding, | EST, Weakly similar to X-Ray Crystal Structure | g, General |
| | | | soluble 3, lectin, | Of The Human Galectin-3 Carbohydrate | |
| | | | galactoside-binding, | Recognition Domain [H. sapiens], galectin- | |
| | | | soluble, 3 (galectin 3) | related inter-fiber protein | |
| 225 | 4749 | NM_031834 | | expressed sequence AI26689, expressed | c, d |
| | | | | sequence AI853643, sulfotransferase family | |
| | | | | 1A, phenol-preferring, member 1, | |
| | | | | sulfotransferase family 4A, member 1, | |
| | | | | sulfotransferase family, cytosolic, 1A, phenol- | |
| | | | | preferring, member 1, sulfotransferase family, | |
| | | | | cytosolic, 1A, phenol-preferring, member 2, | |
| | | | | sulfotransferase family, cytosolic, 1A, phenol- | |
| | | | | preferring, member 3 | |
| 226 | 15603 | NM_031851 | prohibitin | B-cell receptor associated protein 37 (BAP-37) | i |
| | | | | mRNA, partial cds and 3′ untranslated | |
| | | | | sequence, B-cell receptor-associated protein | |
| | | | | 37, EST, Moderately similar to PROHIBITIN | |
| | | | | [R. norvegicus], EST, Weakly similar to PHB | |
| | | | | MOUSE PROHIBITIN [M. musculus], ESTs, | |
| | | | | Moderately similar to PHB MOUSE | |
| | | | | PROHIBITIN [M. musculus], ESTs, Weakly | |
| | | | | similar to PHB_HUMAN PROHIBITI | |
| | | | | [H. sapiens], Homo sapiens, clone MGC: 2874 | |
| | | | | IMAGE: 4547239, mRNA, complete cds, | |
| | | | | hypothetical protein MGC1371, prohibitin | |
| 227 | 22300 | NM_031967 | | N-myc downstream regulated, N-myc | c |
| | | | | downstream regulated 1, N-myc downstream | |
| | | | | regulated 2, N-myc downstream regulated-like, | |
| | | | | N-myc downstream-regulated gene 2, NDRG | |
| | | | | family, member 4, expressed sequence | |
| | | | | R74996 | |
| 228 | 17735 | NM_031970 | | EST, Weakly similar to HHHU27 heat shock | g, i, |
| | | | | protein 27 [H. sapiens], ESTs, Moderately | General |
| | | | | similar to HHHU27 heat shock protein 27 | |
| | | | | [H. sapiens], crystallin, alpha C, heat shock | |
| | | | | 27 kD protein 1, heat shock 27 kD protein 3 | |
| 228 | 17734 | NM_031970 | | EST, Weakly similar to HHHU27 heat shock | g, i, |
| | | | | protein 27 [H. sapiens], ESTs, Moderately | General |
| | | | | similar to HHHU27 heat shock protein 27 | |
| | | | | [H. sapiens], crystallin, alpha C, heat shock | |
| | | | | 27 kD protein 1, heat shock 27 kD protein 3 | |
| 228 | 17736 | NM_031970 | | EST, Weakly similar to HHHU27 heat shock | f, g, i, |
| | | | | protein 27 [H. sapiens], ESTs, Moderately | General |
| | | | | similar to HHHU27 heat shock protein 27 | |
| | | | | [H. sapiens], crystallin, alpha C, heat shock | |
| | | | | 27 kD protein 1, heat shock 27 kD protein 3 | |
| 229 | 18500 | NM_031984 | calbindin 1, (28 kD), | ESTs, Moderately similar to CALBINDIN | c |
| | | | calbindin-28 K | [M. musculus], calbindin 1, (28 kD), calbindin- | |
| | | | | 28 K | |
| 230 | 18899 | NM_031985 | | EST, Highly similar to TVRTK6 ribosomal | a |
| | | | | protein S6 kinase [R. norvegicus], ESTs, Highly | |
| | | | | similar to K6B1_HUMAN RIBOSOMAL | |
| | | | | PROTEIN S6 KINASE [H. sapiens], ESTs, | |
| | | | | Moderately similar to K6B2_MOUSE | |
| | | | | RIBOSOMAL PROTEIN S6 KINASE BETA 2 | |
| | | | | (S6K-BETA 2) (7 KDA RIBOSOMAL PROTEIN | |
| | | | | S6 KINASE 2) (P7-S6KB) (P7 RIBOSOMAL S6 | |
| | | | | KINASE BETA) (P7 S6KBETA) (S6K2) | |
| | | | | [M. musculus], RIKEN cDNA 261318I15 gene, | |
| | | | | expressed sequence AW319595, hypothetical | |
| | | | | protein FLJ14813, ribosomal protein S6 kinase, | |
| | | | | 7 kD, polypeptide 1, ribosomal protein S6 | |
| | | | | kinase, 7 kD, polypeptide 2 | |
| 231 | 19768 | NM_031986 | | ESTs, Highly similar to APB1 RAT AMYLOID | i |
| | | | | BETA A4 PRECURSOR PROTEIN-BINDING | |
| | | | | FAMILY A MEMBER 1 [R. norvegicus], Mus | |
| | | | | musculus, Similar to hypothetical protein, clone | |
| | | | | MGC: 1174 IMAGE: 3964815, mRNA, complete | |
| | | | | cds, RIKEN cDNA 2318D1 gene, amyloid beta | |
| | | | | (A4) precursor protein-binding, family A, | |
| | | | | APBA1: amyloid beta (A4) precursor protein- | |
| | | | | binding, family A, member 1 (X11), amyloid | |
| | | | | beta (A4) precursor protein-binding, family A, | |
| | | | | member 1 (X11), amyloid beta (A4) precursor | |
| | | | | protein-binding, family A, member 3, syndecan | |
| | | | | binding protein, syndecan binding protein | |
| | | | | (syntenin), syntenin-2 protein | |
| 232 | 17829 | NM_033234 | hemoglobin beta chain | | a, c |
| | | | complex, hemoglobin, | | |
| | | | beta | | |
| 232 | 25468 | NM_033234 | | | a, b |
| 232 | 25469 | NM_033234 | | | a, b |
| | | | | | General |
| 232 | 17832 | NM_033234 | hemoglobin beta chain | | a, b |
| | | | complex, hemoglobin, | | General |
| | | | beta | | |
| 233 | 12364 | NM_033351 | Fc fragment of IgG, | EST, Weakly similar to A Chain A, Crystal | General |
| | | | receptor, transporter, | Structure Of The Human Mhc-Related Fc | |
| | | | alpha, Fc receptor, IgG, | Receptor {SUB 24-29 [H. sapiens] , Fc fragment | |
| | | | alpha chain transporter | of IgG, receptor, transporter, alpha, Fc | |
| | | | | receptor, IgG, alpha chain transporter | |
| 234 | 11714 | NM_033352 | | | General |
| 235 | 15867 | NM_053289 | pancreatitis-associated | ESTs, Highly similar to PAP1_HUMAN | General |
| | | | protein, poly(A) | PANCREATITIS-ASSOCIATED PROTEIN 1 | |
| | | | polymerase alpha | PRECURSOR [H. sapiens], islet neogenesis | |
| | | | | associated protein-related protein, pancreatitis- | |
| | | | | associated protein | |
| 236 | 4093 | NM_053290 | phosphoglycerate mutase | | General |
| | | | 1, phosphoglycerate | | |
| | | | mutase 1 (brain) | | |
| 237 | 16886 | NM_053291 | | EST, Highly similar to PGK2 RAT | c |
| | | | | PHOSPHOGLYCERATE KINASE, TESTIS | |
| | | | | SPECIFIC [R. norvegicus], Phosphoglycerate | |
| | | | | kinase {alternatively spliced} [human, | |
| | | | | phosphoglycerate kinase deficient patient with | |
| | | | | episodes of muscl, mRNA Partial Mutant, 37 | |
| | | | | nt], phosphoglycerate kinase 1, | |
| | | | | phosphoglycerate kinase 2 | |
| 238 | 14928 | NM_053330 | ribosomal protein L21 | EST, Moderately similar to 21132B ribosomal | e, h |
| | | | | protein L21 [H. sapiens], EST, Moderately | |
| | | | | similar to RL21 MOUSE 6S RIBOSOMAL | |
| | | | | PROTEIN L21 [M. musculus], EST, Weakly | |
| | | | | similar to 21132B ribosomal protein L21 | |
| | | | | [H. sapiens], EST, Weakly similar to RL21 | |
| | | | | MOUSE 6S RIBOSOMAL PROTEIN L21 | |
| | | | | [M. musculus], EST, Weakly similar to | |
| | | | | RL21_HUMAN 6S RIBOSOMAL PROTEIN L21 | |
| | | | | [H. sapiens], ESTs, Moderately similar to RL21 | |
| | | | | RAT 6S RIBOSOMAL PROTEIN L21 | |
| | | | | [R. norvegicus], RIKEN cDNA 2785M18 gene, | |
| | | | | ribosomal protein L21 | |
| 239 | 1609 | NM_053338 | Ras-related associated | ESTs, Weakly similar to RAD RAT GTP- | c, General |
| | | | | BINDING PROTEIN RAD [R. norvegicus], GTP | |
| | | | | binding protein (gene overexpressed in skeletal | |
| | | | | muscle), Ras-related associated with diabetes | |
| 240 | 9353 | NM_053347 | LIS1-interacting protein | Homo sapiens cDNA FLJ1349 fis, clone | General |
| | | | NUDE1, rat homolog, | NT2RP31428, highly similar to | |
| | | | nuclear distribution gene | NUCLEOPROTEIN TPR, Leman coiled-coil | |
| | | | E homolog (Aspergillus) | protein, RIKEN cDNA 4932416D9 gene, Rho- | |
| | | | | associated coiled-coil forming kinase 2, nuclear | |
| | | | | distribution gene E homolog (Aspergillus), | |
| | | | | translocated promoter region (to activated MET | |
| | | | | oncogene) | |
| 241 | 6155 | NM_053356 | collagen, type I, alpha 2, | ESTs, Weakly similar to CGHU2S collagen | e |
| | | | procollagen, type I, alpha 2 | alpha 2(l) chain precursor [H. sapiens], KIAA126 | |
| | | | | protein, RIKEN cDNA 1113G5 gene, RIKEN | |
| | | | | cDNA 9349G11 gene, collagen, type I, alpha 1, | |
| | | | | collagen, type I, alpha 2, collagen, type II, alpha | |
| | | | | 1 (primary osteoarthritis, spondyloepiphyseal | |
| | | | | dysplasia, congenital), hypothetical protein | |
| | | | | FLJ2654, nischarin, procollagen, type I, alpha | |
| | | | | 1, procollagen, type I, alpha 2, procollagen, | |
| | | | | type II, alpha 1 | |
| 241 | 6156 | NM_053356 | collagen, type I, alpha 2, | ESTs, Weakly similar to CGHU2S collagen | e |
| | | | procollagen, type I, alpha 2 | alpha 2(I) chain precursor [H. sapiens], KIAA126 | |
| | | | | protein, RIKEN cDNA 1113G5 gene, RIKEN | |
| | | | | cDNA 9349G11 gene, collagen, type I, alpha 1, | |
| | | | | collagen, type I, alpha 2, collagen, type II, alpha | |
| | | | | 1 (primary osteoarthritis, spondyloepiphyseal | |
| | | | | dysplasia, congenital), hypothetical protein | |
| | | | | FLJ2654, nischarin, procollagen, type I, alpha | |
| | | | | 1, procollagen, type I, alpha 2, procollagen, | |
| | | | | type II, alpha 1 | |
| 242 | 16017 | NM_053401 | | X-linked protein, brain expressed, X-linked 1, | i |
| | | | | hypothetical protein FLJ197, nerve growth | |
| | | | | factor receptor (TNFRSF16) associated protein 1 | |
| 243 | 2242 | NM_053433 | | ESTs, Highly similar to FMO3_HUMAN | d, General |
| | | | | DIMETHYLANILINE MONOOXYGENASE | |
| | | | | [H. sapiens], Flavin-containing monooxygenase | |
| | | | | 1, Homo sapiens DNA sequence from PAC | |
| | | | | 127D3 on chromosome 1q23-25. Contains | |
| | | | | FMO2 and FMO3 genes for Flavin-containing | |
| | | | | Monooxygenase 2 and Flavin-containing | |
| | | | | Monooxygenase 3 (Dimethylaniline | |
| | | | | Monooxygenase (N-Oxide 3, EC1.14.13.8, | |
| | | | | Dimethylaniline Oxidase 3, FMO II, FMO 3), | |
| | | | | and a gene for another, unknown, Flavin- | |
| | | | | containing Monooxygenase family protein. | |
| | | | | Contains ESTs and GSSs, flavin containing | |
| | | | | monooxygenase 1, flavin containing | |
| | | | | monooxygenase 2, flavin containing | |
| | | | | monooxygenase 3, hypothetical protein | |
| | | | | PRO1257 | |
| 244 | 8085 | NM_053453 | | ESTs, Moderately similar to RGS8 RAT | General |
| | | | | REGULATOR OF G-PROTEIN SIGNALING 8 | |
| | | | | [R. norvegicus], regulator of G-protein signaling | |
| | | | | 18, regulator of G-protein signaling 2, regulator | |
| | | | | of G-protein signaling 8, regulator of G-protein | |
| | | | | signalling 13, regulator of G-protein signalling 2, | |
| | | | | 24 kD, regulator of G-protein signalling 8 | |
| 245 | 11403 | NM_053464 | | Human DNA sequence from clone RP5-157D4 | General |
| | | | | on chromosome 2 Contains a spermidine | |
| | | | | synthase (SPDSY) pseudogene, a CpG island, | |
| | | | | ESTs, STSs and GSSs, spermidine synthase | |
| 246 | 23276 | NM_053467 | | ESTs, Weakly similar to G1159 protein | General |
| | | | | trafficking protein tmp21-l [H. sapiens], ESTs, | |
| | | | | Weakly similar to P24_MOUSE COP-COATED | |
| | | | | VESICLE MEMBRANE PROTEIN P24 | |
| | | | | PRECURSOR (P24A) [M. musculus], RIKEN | |
| | | | | cDNA 1812N21 gene, RIKEN cDNA 243B6 | |
| | | | | gene, RIKEN cDNA 39341E15 gene, coated | |
| | | | | vesicle membrane protein, gp25L2 protein, | |
| | | | | transmembrane trafficking protein | |
| 247 | 15556 | NM_053483 | | ESTs, Weakly similar to A Chain A, Importin | General |
| | | | | Alpha, Mouse [M. musculus], expressed | |
| | | | | sequence AW146299, karyopherin (importin) | |
| | | | | alpha 2, karyopherin alpha 2 (RAG cohort 1, | |
| | | | | importin alpha 1) | |
| 248 | 16394 | NM_053485 | | EST, Moderately similar to CALCYCLIN | General |
| | | | | [R. norvegicus], S1 calcium-binding protein A6 | |
| | | | | (calcyclin), calcium binding protein A6 | |
| | | | | (calcyclin) | |
| 249 | 1886 | NM_053523 | | EST, Moderately similar to Y25_HUMAN | c |
| | | | | HYPOTHETICAL PROTEIN KIAA25 | |
| | | | | [H. sapiens], RIKEN cDNA 5314M7 gene, | |
| | | | | homocysteine-inducible, endoplasmic reticulum | |
| | | | | stress-inducible, ubiquitin-like domain member | |
| | | | | 1, hypothetical protein FLJ22313 | |
| 250 | 14380 | NM_053536 | Kruppel-like factor 15 | Kruppel-like factor 15, Kruppel-like factor 5, | General |
| | | | | Kruppel-like factor 9, TGFB inducible early | |
| | | | | growth response, basic transcription element | |
| | | | | binding protein 1 | |
| 251 | 15708 | NM_053565 | | | General |
| 252 | 3050 | NM_053582 | | ESTs, Highly similar to JC7189 tubulointerstitial | d, General |
| | | | | nephritis antigen [H. sapiens], P3ECSL, | |
| | | | | cathepsin B, lipocalin 7, tubulointerstitial | |
| | | | | nephritis antigen | |
| 252 | 3049 | NM_053582 | | ESTs, Highly similar to JC7189 tubulointerstitial | d, General |
| | | | | nephritis antigen [H. sapiens], P3ECSL, | |
| | | | | cathepsin B, lipocalin 7, tubulointerstitial | |
| | | | | nephritis antigen | |
| 253 | 21709 | NM_053596 | | KIAA64 gene product, endothelin converting | General |
| | | | | enzyme 1, endothelin converting enzyme-like 1, | |
| | | | | expressed sequence AW3225, expressed | |
| | | | | sequence BB127715, mel transforming | |
| | | | | oncogene-like 1, membrane metallo | |
| | | | | endopeptidase | |
| 254 | 2103 | NM_053597 | | 4S ribosomal protein S27 isoform, EST, Weakly | e, h |
| | | | | similar to RS27_HUMAN 4S RIBOSOMAL | |
| | | | | PROTEIN S27 [H. sapiens], ESTs, Moderately | |
| | | | | similar to RS27_HUMAN 4S RIBOSOMAL | |
| | | | | PROTEIN S27 [H. sapiens], RIKEN cDNA | |
| | | | | 18134D23 gene, RIKEN cDNA 321M24 gene, | |
| | | | | ribosomal protein S27 (metallopanstimulin 1) | |
| 255 | 7580 | NM_053603 | | EST X83352, ESTs, Highly similar to T17226 | General |
| | | | | hypothetical protein DKFZp566G223.1 | |
| | | | | [H. sapiens], ESTs, Weakly similar to S7484 | |
| | | | | RS43 protein - rat (fragment) [R. norvegicus], | |
| | | | | Homo sapiens, Similar to chloride intracellular | |
| | | | | channel 4, clone MGC: 8812 IMAGE: 3861372, | |
| | | | | mRNA, complete cds, RIKEN cDNA | |
| | | | | 573531E12 gene, chloride intracellular channel | |
| | | | | 1, chloride intracellular channel 2, chloride | |
| | | | | intracellular channel 4, chloride intracellular | |
| | | | | channel 4 (mitochondrial), intracellular chloride | |
| | | | | ion channel protein p64H1 | |
| 256 | 20243 | NM_053615 | casein kinase 1, alpha 1 | | h |
| 257 | 13005 | NM_053623 | fatty acid-Coenzyme A | | General |
| | | | ligase, long chain 4, fatty- | | |
| | | | acid-Coenzyme A ligase, | | |
| | | | long-chain 4 | | |
| 258 | 18644 | NM_053648 | beta-carotene 15, 15′- | EST, Moderately similar to 86162D protein COII | c |
| | | | dioxygenase, beta- | [M. musculus], EST, Weakly similar to 8124D | |
| | | | carotene 15, 15′- | cytochrome oxidase II [H. sapiens] | |
| | | | dioxygenase | | |
| 259 | 6684 | NM_053703 | | Mus musculus 12 days embryo head cDNA, | General |
| | | | | RIKEN full-length enriched library, clone: 32B1, | |
| | | | | full insert sequence, mitogen activated protein | |
| | | | | kinase kinase 5, mitogen activated protein | |
| | | | | kinase kinase 6, mitogen-activated protein | |
| | | | | kinase kinase 3, mitogen-activated protein | |
| | | | | kinase kinase 4, mitogen-activated protein | |
| | | | | kinase kinase 6 | |
| 260 | 25379 | NM_053713 | | | d, General |
| 260 | 22411 | NM_053713 | | ESTs, Moderately similar to CPBP RAT CORE | d |
| | | | | PROMOTER ELEMENT-BINDING PROTEIN | |
| | | | | [R. norvegicus], Kruppel-like factor 4 (gut), | |
| | | | | RIKEN cDNA 7427M5 gene, core promoter | |
| | | | | element binding protein | |
| 261 | 1218 | NM_053748 | | expressed sequence C86324, hypothetical | e, General |
| | | | | protein FLJ2359 | |
| 262 | 9059 | NM_053783 | | interferon gamma receptor, interferon gamma | g, General |
| | | | | receptor 1 | |
| 263 | 15103 | NM_053814 | | EST, Moderately similar to MYSO_HUMAN | h |
| | | | | MYOSIN HEAVY CHAIN, NONMUSCLE TYPE | |
| | | | | B [H. sapiens], EST, Weakly similar to T3868 | |
| | | | | RhoA-binding protein p116Rip - mouse | |
| | | | | [M. musculus], ESTs, Weakly similar to JC5421 | |
| | | | | smooth muscle myosin heavy chain 2 - mouse | |
| | | | | [M. musculus], Homo sapiens cDNA: FLJ23324 | |
| | | | | fis, clone HEP12482, highly similar to | |
| | | | | HUMMYOHCB Human nonmuscle myosin | |
| | | | | heavy chain-B (MYH1) mRNA, LIS1-interacting | |
| | | | | protein NUDE1, rat homolog, PiUS protein, | |
| | | | | RIKEN cDNA 177J1 gene, Rho interacting | |
| | | | | protein 3, cingulin, expressed sequence | |
| | | | | AI428493, expressed sequence AI647528, | |
| | | | | hypothetical protein FLJ13881 | |
| 264 | 15002 | NM_053819 | | EST, Moderately similar to TIM1 RAT | f, g, |
| | | | | METALLOPROTEINASE INHIBITOR 1 | General |
| | | | | PRECURSOR [R. norvegicus], EST, Weakly | |
| | | | | similar to TIM1 RAT METALLOPROTEINASE | |
| | | | | INHIBITOR 1 PRECURSOR [R. norvegicus], | |
| | | | | tissue inhibitor of metalloproteinase, tissue | |
| | | | | inhibitor of metalloproteinase 1 (erythroid | |
| | | | | potentiating activity, collagenase inhibitor) | |
| 264 | 15003 | NM_053819 | | EST, Moderately similar to TIM1 RAT | f, g, |
| | | | | METALLOPROTEINASE INHIBITOR 1 | General |
| | | | | PRECURSOR [R. norvegicus], EST, Weakly | |
| | | | | similar to TIM1 RAT METALLOPROTEINASE | |
| | | | | INHIBITOR 1 PRECURSOR [R. norvegicus], | |
| | | | | tissue inhibitor of metalloproteinase, tissue | |
| | | | | inhibitor of metalloproteinase 1 (erythroid | |
| | | | | potentiating activity, collagenase inhibitor) | |
| 265 | 17155 | NM_053835 | | | e |
| 266 | 20868 | NM_053843 | | | General |
| 266 | 20869 | NM_053843 | | | General |
| 267 | 1571 | NM_053857 | | EST, Moderately similar to 221415A initiation | d, General |
| | | | | factor 4E-binding protein: ISOTYPE, ESTs, | |
| | | | | Weakly similar to A55258 insulin-stimulated | |
| | | | | phosphoprotein PHAS-l - rat [R. norvegicus], | |
| | | | | RIKEN cDNA 1114O12 gene, eukaryotic | |
| | | | | translation initiation factor 4E binding protein 1, | |
| | | | | eukaryotic translation initiation factor 4E | |
| | | | | binding protein 2, eukaryotic translation | |
| | | | | initiation factor 4E binding protein 3 | |
| 268 | 18358 | NM_053864 | | EST, Highly similar to TERA HUMAN | h |
| | | | | [H. sapiens], EST, Weakly similar to T46437 | |
| | | | | hypothetical protein DKFZp434K126.1 | |
| | | | | [H. sapiens], ESTs, Weakly similar to T46437 | |
| | | | | hypothetical protein DKFZp434K126.1 | |
| | | | | [H. sapiens], ESTs, Weakly similar to TERA | |
| | | | | HUMAN [H. sapiens], ESTs, Weakly similar to | |
| | | | | TERA RAT TRANSITIONAL ENDOPLASMIC | |
| | | | | RETICULUM ATPASE [R. norvegicus], RIKEN | |
| | | | | cDNA 4833413G1 gene, RIKEN cDNA | |
| | | | | 543414H2 gene, spermatogenesis associated | |
| | | | | factor, valosin containing protein, valosin- | |
| | | | | containing protein | |
| 269 | 17728 | NM_053867 | | EST, Moderately similar to S659 IgE-dependent | a, b |
| | | | | histamine-releasing factor [H. sapiens], Homo | |
| | | | | sapiens mRNA full length insert cDNA clone | |
| | | | | EUROIMAGE 926491, hypothetical protein | |
| | | | | FLJ23, translationally regulated transcript (21 kDa), | |
| | | | | tumor protein, translationally-controlled 1 | |
| 270 | 19781 | NM_053883 | | ESTs, Moderately similar to DUS6 RAT DUAL | General |
| | | | | SPECIFICITY PROTEIN PHOSPHATASE 6 | |
| | | | | [R. norvegicus], ESTs, Weakly similar to DUS6 | |
| | | | | RAT DUAL SPECIFICITY PROTEIN | |
| | | | | PHOSPHATASE 6 [R. norvegicus], dual | |
| | | | | specificity phosphatase 1, dual specificity | |
| | | | | phosphatase 13, dual specificity phosphatase | |
| | | | | 14, dual specificity phosphatase 6, dual | |
| | | | | specificity phosphatase 9, expressed sequence | |
| | | | | BB14621, mitogen-activated protein kinase | |
| | | | | phosphatase x, protein tyrosine phosphatase, | |
| | | | | non-receptor type 16 | |
| 271 | 385 | NM_053885 | | | i, General |
| 272 | 1288 | NM_053949 | | EST, Weakly similar to I38465 probable | General |
| | | | | potassium channel subunit [H. sapiens], | |
| | | | | potassium voltage-gated channel, subfamily H | |
| | | | | (eag-related), member 5 | |
| 273 | 19774 | NM_053961 | | | a |
| 274 | 16546 | NM_053965 | | ESTs, Weakly similar to MCAT RAT | General |
| | | | | MITOCHONDRIAL | |
| | | | | CARNITINE/ACYLCARNITINE CARRIER | |
| | | | | PROTEIN [R. norvegicus], ESTs, Weakly similar | |
| | | | | to MCAT_HUMAN MITOCHONDRIAL | |
| | | | | CARNITINE/ACYLCARNITINE CARRIER | |
| | | | | PROTEIN [H. sapiens], expressed sequence | |
| | | | | W51672, solute carrier family 25 | |
| | | | | (carnitine/acylcarnitine translocase), member 2, | |
| | | | | solute carrier family 25 (mitochondrial | |
| | | | | carnitine/acylcarnitine translocase), member 2 | |
| 275 | 19544 | NM_053982 | | EST, Highly similar to 4S RIBOSOMAL | General |
| | | | | PROTEIN S15A [R. norvegicus], ESTs, | |
| | | | | Moderately similar to S2251 ribosomal protein | |
| | | | | S15a.e [H. sapiens], ribosomal protein S15a | |
| 276 | 15642 | NM_053985 | | ESTs, Highly similar to HISTONE H3.3 | General |
| | | | | [R. norvegicus], H3 histone, family 3A, H3 | |
| | | | | histone, family 3B, H3 histone, family 3B | |
| | | | | (H3.3B) | |
| 277 | 17739 | NM_053995 | | | e |
| 278 | 17329 | NM_054008 | | RGC32 protein, RIKEN cDNA 1192H23 gene | General |
| 279 | 19658 | NM_057103 | A kinase (PRKA) anchor | | General |
| | | | protein (gravin) 12 | | |
| 280 | 968 | NM_057133 | | | h |
| 281 | 2413 | NM_057141 | | ESTs, Weakly similar to ROK_HUMAN | e, h |
| | | | | HETEROGENEOUS NUCLEAR | |
| | | | | RIBONUCLEOPROTEIN K [R. norvegicus], | |
| | | | | heterogeneous nuclear ribonucleoprotein K, | |
| | | | | poly(rC)-binding protein 4 | |
| 281 | 2416 | NM_057141 | | ESTs, Weakly similar to ROK_HUMAN | d |
| | | | | HETEROGENEOUS NUCLEAR | |
| | | | | RIBONUCLEOPROTEIN K [R. norvegicus], | |
| | | | | heterogeneous nuclear ribonucleoprotein K, | |
| | | | | poly(rC)-binding protein 4 | |
| 282 | 1892 | NM_057144 | | cysteine and glycine-rich protein 3 (cardiac LIM | g, i, |
| | | | | protein), cysteine rich protein, cysteine-rich | General |
| | | | | protein 2, cysteine-rich protein 3, thymus LIM | |
| | | | | protein | |
| 283 | 17477 | NM_057194 | | EST, Weakly similar to B36298 proline-rich | g, General |
| | | | | protein PRB3S [H. sapiens], EST, Weakly | |
| | | | | similar to CGHU3B collagen alpha 3(IV) chain | |
| | | | | precursor, long splice form [H. sapiens], EST, | |
| | | | | Weakly similar to D475 proline-rich protein | |
| | | | | PRB1/2S [H. sapiens], EST, Weakly similar to | |
| | | | | JE284 Mm-1 cell derived transplantability- | |
| | | | | associated protein 1b [H. sapiens], galectin- | |
| | | | | related inter-fiber protein, murine leukemia viral | |
| | | | | (bmi-1) oncogene homolog, phospholipid | |
| | | | | scramblase 1, phospholipid scramblase 2, | |
| | | | | phospholipid scramblase 3 | |
| 284 | 14126 | NM_057208 | | | g |
| 285 | 8641 | NM_057211 | | BTE-binding protein 4, ESTs, Weakly similar to | c |
| | | | | ZN42_HUMAN ZINC FINGER PROTEIN 42 | |
| | | | | (MYELOID ZINC FINGER 1) (MZF-1) | |
| | | | | [H. sapiens], Kruppel-like factor 13, Kruppel-like | |
| | | | | factor 15 (kidney), Kruppel-like factor 9, TGFB | |
| | | | | inducible early growth response, basic | |
| | | | | transcription element binding protein 1, | |
| | | | | expressed sequence AI843742 | |
| 286 | 10498 | NM_078617 | | EST, Moderately similar to RS23_HUMAN 4S | e, f |
| | | | | RIBOSOMAL PROTEIN S2 [R. norvegicus], | |
| | | | | EST, Weakly similar to RS23_HUMAN 4S | |
| | | | | RIBOSOMAL PROTEIN S2 [R. norvegicus], | |
| | | | | ESTs, Weakly similar to RS23_HUMAN 4S | |
| | | | | RIBOSOMAL PROTEIN S2 [R. norvegicus], | |
| | | | | Mus musculus, Similar to mitochondrial | |
| | | | | ribosomal protein S12, clone MGC: 13892 | |
| | | | | IMAGE: 429358, mRNA, complete cds, RIKEN | |
| | | | | cDNA 24144J15 gene, expressed sequence | |
| | | | | AI327385, mitochondrial ribosomal protein S12, | |
| | | | | ribosomal protein S23 | |
| 287 | 20392 | NM_080477 | | | General |
| 288 | 23033 | NM_080888 | | BCL2/adenovirus E1B 19 kDa-interacting | b, General |
| | | | | protein 3-like, BCL2/adenovirus E1B 19 kD- | |
| | | | | interacting protein 3-like | |
| 289 | 15843 | NM_080910 | | ESTs, Moderately similar to PUR6_HUMAN | h |
| | | | | MULTIFUNCTIONAL PROTEIN ADE2 | |
| | | | | [H. sapiens], RIKEN cDNA 261511I9 gene, | |
| | | | | multifunctional polypeptide similar to SAICAR | |
| | | | | synthetase and AIR carboxylase | |
| 290 | 21391 | NM_130416 | | ESTs, Highly similar to LUHU7 annexin VII, | General |
| | | | | long form [H. sapiens], ESTs, Moderately similar | |
| | | | | to ANX4 MOUSE ANNEXIN IV [M. musculus], | |
| | | | | ZAP 36/annexin IV, annexin A4, annexin A7 | |
| 291 | 25094 | X77117 | | | c |
| 292 | 11663 | AA800803 | | | General |
| 293 | 25717 | X61296 | | | i |
| 294 | 5292 | AI171607 | | | d, General |
| 295 | 23998 | AI230578 | | | General |
| 296 | 5186 | AA925674 | | | d, General |
| 297 | 1754 | AA817837 | | potassium channel, subfamily K, member 1 | General |
| | | | | (TWIK-1), potassium channel, subfamily K, | |
| | | | | member 3 (TASK-1), potassium channel, | |
| | | | | subfamily K, member 6 (TWIK-2), potassium | |
| | | | | channel, subfamily K, member 7 | |
| 298 | 6438 | AA819269 | | | General |
| 299 | 6059 | AI178245 | | ESTs, Highly similar to T13963 formin related | b, General |
| | | | | protein, lymphocyte specific - mouse | |
| | | | | [M. musculus], ESTs, Moderately similar to | |
| | | | | T13963 formin related protein, lymphocyte | |
| | | | | specific - mouse [M. musculus], Homo sapiens | |
| | | | | mRNA; cDNA DKFZp762B245 (from clone | |
| | | | | DKFZp762B245); partial cds, KIAA192 protein, | |
| | | | | formin-like | |
| 300 | 18663 | U95052 | | eukaryotic translation initiation factor 4 gamma, 3 | h |
| 301 | 7414 | AI137586 | | EST, Weakly similar to IMB3_HUMAN | g, General |
| | | | | IMPORTIN BETA-3 SUBUNIT [H. sapiens], | |
| | | | | Homo sapiens cDNA FLJ12978 fis, clone | |
| | | | | NT2RP26321, RAN binding protein 6, | |
| | | | | karyopherin (importin) beta 3 | |
| 302 | 10019 | AI178756 | | | d, h |
| 303 | 6226 | AA818521 | | | General |
| 304 | 15571 | M27207 | collagen, type I, alpha 1, | EST, Highly similar to CA1B_HUMAN | e, h |
| | | | procollagen, type I alpha 1 | COLLAGEN ALPHA 1(XI) CHAIN | |
| | | | | PRECURSOR [H. sapiens], EST, Weakly | |
| | | | | similar to CA11_HUMAN COLLAGEN ALPHA | |
| | | | | 1(I) CHAIN PRECURSOR [H. sapiens], EST, | |
| | | | | Weakly similar to CA25_HUMAN COLLAGEN | |
| | | | | ALPHA 2(V) CHAIN PRECURSO [H. sapiens], | |
| | | | | EST, Weakly similar to CGHU1S collagen | |
| | | | | alpha 1(I) chain precursor [H. sapiens], ESTs, | |
| | | | | Weakly similar to CA11_HUMAN COLLAGEN | |
| | | | | ALPHA 1(I) CHAIN PRECURSOR [H. sapiens], | |
| | | | | Homo sapiens cDNA FLJ2845 fis, clone | |
| | | | | ADKA191, Homo sapiens mRNA for putative | |
| | | | | emu2 protein, collagen, type I, alpha 1 | |
| 305 | 15089 | AI009752 | | | g |
| 306 | 17214 | AI639008 | | | a |
| 307 | 6073 | AA818818 | | | a |
| 308 | 17781 | AI175474 | | | a |
| 309 | 22386 | AA800844 | | ESTs, Weakly similar to PROTEIN-LYSINE 6- | e |
| | | | | OXIDASE PRECURSOR [R. norvegicus], Lysyl | |
| | | | | oxidase, lysyl oxidase, lysyl oxidase-like, lysyl | |
| | | | | oxidase-like 1, lysyl oxidase-like 2 | |
| 310 | 24765 | AA900050 | | EST, Moderately similar to 6S RIBOSOMAL | h |
| | | | | PROTEIN L37A [R. norvegicus], EST, | |
| | | | | Moderately similar to JN875 ribosomal protein | |
| | | | | L37a [H. sapiens], EST, Weakly similar to 6S | |
| | | | | RIBOSOMAL PROTEIN L37A [R. norvegicus], | |
| | | | | EST, Weakly similar to JN875 ribosomal | |
| | | | | protein L37a [H. sapiens], ESTs, Weakly similar | |
| | | | | to 6S RIBOSOMAL PROTEIN L37A | |
| | | | | [R. norvegicus], ESTs, Weakly similar to JN875 | |
| | | | | ribosomal protein L37a [H. sapiens], RIKEN | |
| | | | | cDNA 28125M15 gene, ribosomal protein L37a | |
| 311 | 20984 | D90109 | fatty acid Coenzyme A | ESTs, Weakly similar to LCFB MOUSE LONG- | h |
| | | | ligase, long chain 2, fatty- | CHAIN-FATTY-ACID-COA LIGASE 2 | |
| | | | acid-Coenzyme A ligase, | [M. musculus], Mus musculus, Similar to fatty- | |
| | | | long-chain 2 | acid-Coenzyme A ligase, long-chain 6, clone | |
| | | | | MGC: 28744 IMAGE: 4481949, mRNA, complete | |
| | | | | cds, Rattus norvegicus gonadotropin-regulated | |
| | | | | long chain acyl-CoA synthetase (GR-LACS) | |
| | | | | mRNA, complete cds, fatty acid Coenzyme A | |
| | | | | ligase, long chain 2, fatty acid Coenzyme A | |
| | | | | ligase, long chain 5, fatty-acid-Coenzyme A | |
| | | | | ligase, long-chain 1, fatty-acid-Coenzyme A | |
| | | | | ligase, long-chain 2, lipidosin | |
| 312 | 22873 | AA923945 | | RIKEN cDNA 24142F5 gene, procollagen- | i, General |
| | | | | lysine, 2-oxoglutarate 5-dioxygenase (lysine | |
| | | | | hydroxylase) 2, procollagen-lysine, 2- | |
| | | | | oxoglutarate 5-dioxygenase (lysine | |
| | | | | hydroxylase, Ehlers-Danlos syndrome type VI), | |
| | | | | procollagen-lysine, 2-oxoglutarate 5- | |
| | | | | dioxygenase 1 | |
| 313 | 2341 | AI012144 | | | a, General |
| 314 | 4861 | AA901290 | | | General |
| 315 | 22796 | AI072213 | | | General |
| 316 | 15935 | AA866345 | | | General |
| 317 | 16038 | AI177372 | | GABA(A) receptor-associated protein, GABA(A) | h |
| | | | | receptor-associated protein like 1, GABA(A) | |
| | | | | receptor-associated protein-like 2, GABA(A) | |
| | | | | receptors associated protein like 3, RIKEN | |
| | | | | cDNA 111H21 gene, RIKEN cDNA 492251H4 | |
| | | | | gene, microtubule-associated protein 1 light | |
| | | | | chain 3, microtubule-associated proteins 1A/1B | |
| | | | | light chain 3 | |
| 318 | 15212 | AI229753 | | ADP-ribosylation factor 1, ADP-ribosylation | d, General |
| | | | | factor 2, ARF protein, Homo sapiens PRO2667 | |
| | | | | mRNA, complete cds, RIKEN cDNA 2318D22 | |
| | | | | gene, expressed sequence T25534 | |
| 319 | 23157 | AI172489 | | ESTs, Highly similar to STRI RAT STRIATIN | a |
| | | | | [R. norvegicus], ESTs, Weakly similar to STRI | |
| | | | | RAT STRIATIN [R. norvegicus], Mus musculus, | |
| | | | | Similar to RIKEN cDNA 1541N16 gene, clone | |
| | | | | MGC: 1266 IMAGE: 378188, mRNA, complete | |
| | | | | cds, RIKEN cDNA 2514D7 gene, expressed | |
| | | | | sequence AA48785, nuclear autoantigen, | |
| | | | | platelet-activating factor acetylhydrolase, | |
| | | | | isoform 1b, beta1 subunit, striatin, striatin, | |
| | | | | calmodulin-binding protein, zinedin | |
| 320 | 4710 | AI168973 | | EST, Moderately similar to PHLP_HUMAN | General |
| | | | | PHOSDUCIN-LIKE PROTEIN [H. sapiens], | |
| | | | | RIKEN cDNA 11161A19 gene, RIKEN cDNA | |
| | | | | 1211E13 gene, hypothetical protein MGC362, | |
| | | | | phosducin-like, phosducin-like 2, phosducin-like | |
| | | | | protein, rod photoreceptor 1 | |
| 321 | 22697 | AA945996 | | | b, General |
| 322 | 15892 | AI179988 | | | a, General |
| 323 | 11404 | AI237002 | | Human DNA sequence from clone RP5-157D4 | g, General |
| | | | | on chromosome 2 Contains a spermidine | |
| | | | | synthase (SPDSY) pseudogene, a CpG island, | |
| | | | | ESTs, STSs and GSSs, spermidine synthase | |
| 324 | 23596 | AI105435 | | expressed sequence D17825, glutaryl- | c, General |
| | | | | Coenzyme A dehydrogenase | |
| 325 | 15841 | AI103465 | | POP4 (processing of precursor, S. cerevisiae) | d, g, |
| | | | | homolog | General |
| 326 | 7220 | AI013098 | | | d |
| 327 | 20647 | AI639402 | | | b |
| 328 | 10829 | AI044467 | | | c, f |
| 329 | 22043 | AA943738 | | | f |
| 330 | 2936 | AI229843 | | | General |
| 331 | 26133 | AI009950 | | | e |
| 332 | 5867 | AA858953 | | asparaginyl-tRNA synthetase, hypothetical | Geneal |
| | | | | protein FLJ23441 | |
| 333 | 3278 | AI008988 | | ESTs, Highly similar to 117276A gene bcr | General |
| | | | | [H. sapiens], ESTs, Highly similar to N- | |
| | | | | CHIMAERIN [R. norvegicus], RIKEN cDNA | |
| | | | | 17112L9 gene, RIKEN cDNA 51334C9 gene, | |
| | | | | RIKEN cDNA 57343H17 gene, SH3-domain | |
| | | | | binding protein 1, breakpoint cluster region, | |
| | | | | chimerin (chimaerin) 1, expressed sequence | |
| | | | | AI84366, expressed sequence AU42359, | |
| | | | | hypothetical protein DKFZp434K191 | |
| 334 | 21101 | AI175789 | | Actin, alpha, cardiac, ESTs, Highly similar to | g |
| | | | | ACTC_HUMAN ACTIN, ALPHA CARDIA | |
| | | | | [R. norvegicus], ESTs, Weakly similar to | |
| | | | | ACTA_HUMAN ACTIN, AORTIC SMOOTH | |
| | | | | MUSCLE [R. norvegicus], Homo sapiens cDNA: | |
| | | | | FLJ21441 fis, clone COL4422, actin, alpha 2, | |
| | | | | smooth muscle, aorta, actin, alpha, cardiac, | |
| | | | | actin, alpha, cardiac muscle | |
| 335 | 12613 | H31620 | | Homo sapiens cDNA FLJ13436 fis, clone | General |
| | | | | PLACE12598, weakly similar to | |
| | | | | OLIGORIBONUCLEASE (EC 3.1.—.—), small | |
| | | | | fragment nuclease | |
| 336 | 6927 | AI010542 | | | h |
| 337 | 8789 | AI070073 | | | h |
| 338 | 22995 | AI232047 | | | General |
| 339 | 5129 | AA925335 | | | d |
| 340 | 12462 | AI175421 | | | General |
| 341 | 1760 | U25281 | | EST, Weakly similar to G3PT MOUSE | General |
| | | | | GLYCERALDEHYDE 3-PHOSPHATE | |
| | | | | DEHYDROGENASE, TESTIS-SPECIFIC | |
| | | | | [M. musculus], ESTs, Moderately similar to | |
| | | | | WASP-family protein [H. sapiens], ESTs, | |
| | | | | Weakly similar to Y269_HUMAN | |
| | | | | HYPOTHETICAL PROLINE-RICH PROTEIN | |
| | | | | KIAA269 [H. sapiens], ESTs, Weakly similar to | |
| | | | | p14mDia [M. musculus], ESTs, Weakly similar | |
| | | | | to similar to CR16, SH3 domain binding protein | |
| | | | | [H. sapiens], expressed sequence AI842788 | |
| 342 | 24521 | AA945636 | | EST, Weakly similar to R6HUP1 acidic | e |
| | | | | ribosomal protein P1, cytosolic [H. sapiens], | |
| | | | | ESTs, Highly similar to R6HUP1 acidic | |
| | | | | ribosomal protein P1, cytosolic [H. sapiens], | |
| | | | | RIKEN cDNA 2749I22 gene, ribosomal protein, | |
| | | | | large, P1 | |
| 343 | 25354 | L13025 | | | e |
| 344 | 24272 | AA955948 | | Homo sapiens DNA from chromosome 19p13.2 | General |
| | | | | cosmids R3124, R3272 and R28549 containing | |
| | | | | the EKLF, GCDH, CRTC, and RAD23A genes, | |
| | | | | genomic sequence, deoxyribonuclease DLAD, | |
| | | | | deoxyribonuclease II alpha, deoxyribonuclease | |
| | | | | II beta, deoxyribonuclease II, lysosomal, | |
| | | | | expressed sequence AI526873 | |
| 345 | 18160 | AA799448 | | | h |
| 346 | 11738 | AI104213 | | | General |
| 347 | 15644 | AI010256 | | ESTs, Highly similar to HISTONE H3.3 | General |
| | | | | [R. norvegicus], H3 histone, family 3A, H3 | |
| | | | | histone, family 3B, H3 histone, family 3B | |
| | | | | (H3.3B) | |
| 348 | 10971 | AI073212 | | | e |
| 349 | 23578 | AA955042 | | folate receptor 2 (fetal) | General |
| 350 | 6217 | AI230381 | | | General |
| 351 | 21514 | AA851660 | | | General |
| 352 | 16909 | AA942704 | | EST, Weakly similar to SUR2_HUMAN | e |
| | | | | SURFEIT LOCUS PROTEIN 2 [H. sapiens], | |
| | | | | surfeit 2, surfeit gene 2 | |
| 353 | 21284 | AI170842 | | ESTs, Highly similar to A57291 cytokine | General |
| | | | | inducible nuclear protein C193 [H. sapiens], | |
| | | | | ESTs, Weakly similar to A57291 cytokine | |
| | | | | inducible nuclear protein C193 [H. sapiens], | |
| | | | | ankyrin repeat domain 2 (stretch responsive | |
| | | | | muscle), cardiac ankyrin repeat protein | |
| 354 | 23645 | AI144653 | | | General |
| 355 | 8230 | AI059117 | | | General |
| 356 | 9720 | AI071727 | | | g |
| 357 | 24012 | AA957335 | | | a, i |
| 358 | 14737 | AI008416 | | | g, General |
| 359 | 7308 | AA818407 | | | b, General |
| 360 | 6102 | AI228335 | | | General |
| 361 | 19728 | AI170394 | | | c |
| 362 | 6568 | AA817891 | | | General |
| 363 | 20891 | AI169337 | | hypothetical protein | General |
| 364 | 11375 | AI237594 | | makorin, ring finger protein, 1, makorin, ring | b, General |
| | | | | finger protein, 2, makorin, ring finger protein, 3, | |
| | | | | makorin, ring finger protein, 4 | |
| 365 | 1483 | U34843 | | | i |
| 366 | 20684 | H32977 | | | General |
| 367 | 3959 | AA901338 | | | g, General |
| 368 | 19518 | AA800817 | | | a |
| 369 | 22445 | AA944530 | | PL6 protein | f |
| 370 | 16312 | AA875032 | | | c, General |
| 371 | 19255 | M15562 | | expressed sequence AI323765, | b, General |
| | | | | histocompatibility 2, class II antigen E alpha, | |
| | | | | major histocompatibility complex, class II, DP | |
| | | | | alpha 2 (pseudogene), major histocompatibility | |
| | | | | complex, class II, DR alpha | |
| 372 | 4048 | AA851814 | | EST, Moderately similar to PM17 MOUSE | General |
| | | | | MELANOCYTE PROTEIN PMEL 17 | |
| | | | | PRECURSOR [M. musculus], Homo sapiens, | |
| | | | | Similar to glycoprotein (transmembrane) nmb, | |
| | | | | clone MGC: 1696 IMAGE: 3345861, mRNA, | |
| | | | | complete cds, glycoprotein (transmembrane) | |
| | | | | nmb, silver | |
| 373 | 7471 | AI012379 | | | General |
| 374 | 21351 | AA945932 | | annexin A1, annexin A3 | d, General |
| 375 | 23963 | AA957139 | | | General |
| 376 | 9658 | AA943748 | | | d, General |
| 377 | 14384 | AI177096 | | EST, Moderately similar to APT RAT ADENINE | c |
| | | | | PHOSPHORIBOSYLTRANSFERASE | |
| | | | | [R. norvegicus], Mouse adenine | |
| | | | | phosphoribosyltransferase (APRT), complete | |
| | | | | cds, adenine phosphoribosyl transferase, | |
| | | | | adenine phosphoribosyltransferase, expressed | |
| | | | | sequence C85684 | |
| 378 | 5531 | AI145859 | | | d |
| 379 | 3324 | AA997981 | | | e |
| 380 | 13083 | H31665 | | | d |
| 381 | 18400 | AA799991 | | | a, f, |
| | | | | | General |
| 382 | 15281 | D13623 | | | General |
| 383 | 22578 | AA924105 | | Macrophage inflammatory protein 1 alpha | g |
| | | | | (Small inducible cytokine A3), small inducible | |
| | | | | cytokine A3, small inducible cytokine A3 | |
| | | | | (homologous to mouse Mip-1a), small inducible | |
| | | | | cytokine A3-like 1, small inducible cytokine A6, | |
| | | | | small inducible cytokine subfamily A (Cys-Cys), | |
| | | | | member 23 | |
| 384 | 12752 | AI012146 | | | General |
| 385 | 6606 | AI012308 | | | f, g, |
| | | | | | General |
| 386 | 11936 | AI228030 | | EST, Weakly similar to SP18_HUMAN SIN3 | h |
| | | | | ASSOCIATED POLYPEPTIDE P18 | |
| | | | | [H. sapiens], Sin3-associated polypeptide 18, | |
| | | | | sin3-associated polypeptide, l8 kD | |
| 387 | 2539 | AI111960 | | | General |
| 388 | 6353 | AI008442 | | EST, Weakly similar to JC5111 cyclin- | f |
| | | | | dependent kinase-related protein 1b - rat | |
| | | | | [R. norvegicus], EST, Weakly similar to S1889 | |
| | | | | proline-rich protein [H. sapiens], ESTs, Highly | |
| | | | | similar to JC5111 cyclin-dependent kinase- | |
| | | | | related protein 1b - rat [R. norvegicus], Homo | |
| | | | | sapiens ALS2CR7 mRNA, complete cds, | |
| | | | | PCTAIRE protein kinase 1, PCTAIRE-motif | |
| | | | | protein kinase 1, PFTAIRE protein kinase 1 | |
| 389 | 15786 | AI013924 | | | General |
| 390 | 16596 | AI102486 | | DNA segment, Chr 7, ERATO Doi 486, | General |
| | | | | expressed, EST, Weakly similar to S37583 | |
| | | | | RING finger protein rfp - mouse [M. musculus], | |
| | | | | expressed sequence AI894158, expressed | |
| | | | | sequence AW46487, hypothetical gene | |
| | | | | MGC1127 | |
| 391 | 18581 | AI176160 | | | General |
| 392 | 4948 | AA924428 | | | h |
| 393 | 12098 | AI237075 | | | d |
| 394 | 20073 | AI639152 | | | b, General |
| 395 | 18456 | D00688 | monoamine oxidase A | EST, Highly similar to AOFA_HUMAN AMINE | General |
| | | | | OXIDASE [H. sapiens], ESTs, Weakly similar to | |
| | | | | JH817 amine oxidase [H. sapiens], Homo | |
| | | | | sapiens interleukin-4 induced gene-1 protein | |
| | | | | (FIG. 1) mRNA, complete cds, KIAA61 protein, | |
| | | | | monoamine oxidase A | |
| 396 | 11208 | AI237586 | | EST, Weakly similar to JC1241 beta-interferon- | General |
| | | | | induced protein - rat [R. norvegicus], ESTs, | |
| | | | | Moderately similar to JC1241 beta-interferon- | |
| | | | | induced protein - rat [R. norvegicus], RIKEN | |
| | | | | cDNA 11136C17 gene, RIKEN cDNA | |
| | | | | 4933438K12 gene, interferon induced | |
| | | | | transmembrane protein 2 (1-8D) | |
| 397 | 8314 | AI059386 | | | General |
| 398 | 1893 | X51529 | phospholipase A2, group | | General |
| | | | IIA (platelets, synovial | | |
| | | | fluid) | | |
| 399 | 10821 | AI043999 | | | e |
| 400 | 20983 | AI044900 | fatty acid Coenzyme A | ESTs, Weakly similar to LCFB MOUSE LONG- | General |
| | | | ligase, long chain 2, fatty- | CHAIN-FATTY-ACID--COA LIGASE 2 | |
| | | | acid-Coenzyme A ligase, | [M. musculus], Mus musculus, Similar to fatty- | |
| | | | long-chain 2 | acid-Coenzyme A ligase, long-chain 6, clone | |
| | | | | MGC: 28744 IMAGE: 4481949, mRNA, complete | |
| | | | | cds, Rattus norvegicus gonadotropin-regulated | |
| | | | | long chain acyl-CoA synthetase (GR-LACS) | |
| | | | | mRNA, complete cds, fatty acid Coenzyme A | |
| | | | | ligase, long chain 2, fatty acid Coenzyme A | |
| | | | | ligase, long chain 5, fatty-acid-Coenzyme A | |
| | | | | ligase, long-chain 1, fatty-acid-Coenzyme A | |
| | | | | ligase, long-chain 2, lipidosin | |
| 401 | 18472 | AI168975 | | | General |
| 402 | 21592 | AI102065 | | heme-binding protein | General |
| 403 | 8665 | AI071965 | | ESTs, Highly similar to T17342 hypothetical | General |
| | | | | protein DKFZp586K1924.1 [H. sapiens], ESTs, | |
| | | | | Moderately similar to T17342 hypothetical | |
| | | | | protein DKFZp586K1924.1 [H. sapiens] | |
| 404 | 11066 | AI071602 | | | h |
| 405 | 19452 | AA964500 | | | e, f |
| 406 | 6509 | AI232361 | | | General |
| 407 | 22266 | AA945601 | | | a |
| 408 | 2742 | AI070173 | | | g, i, |
| | | | | | General |
| 409 | 244 | U38376 | | ESTs, Highly similar to FGD1_HUMAN | General |
| | | | | PUTATIVE RHO/RAC GUANINE | |
| | | | | NUCLEOTIDE EXCHANGE FACTOR | |
| | | | | [H. sapiens], ESTs, Weakly similar to B39898 | |
| | | | | phospholipase A2 [M. musculus], ESTs, Weakly | |
| | | | | similar to FGD1 MOUSE PUTATIVE RHO/RAC | |
| | | | | GUANINE NUCLEOTIDE EXCHANGE | |
| | | | | FACTOR [M. musculus], ESTs, Weakly similar | |
| | | | | to FGD1_HUMAN PUTATIVE RHO/RAC | |
| | | | | GUANINE NUCLEOTIDE EXCHANGE | |
| | | | | FACTOR [H. sapiens], FGD1 family, member 3, | |
| | | | | RIKEN cDNA 583461L1 gene, faciogenital | |
| | | | | dysplasia (Aarskog-Scott syndrome), | |
| | | | | faciogenital dysplasia homolog, faciogenital | |
| | | | | dysplasia homolog 2 (human) | |
| 410 | 4238 | AI234816 | | KIAA13 gene product | b |
| 411 | 9970 | AI045198 | | | General |
| 412 | 6550 | AA817947 | | | h |
| 413 | 5193 | AA925693 | | | General |
| 414 | 22175 | AA818999 | | | General |
| 415 | 16538 | AA799449 | | ESTs, Moderately similar to NPL4_HUMAN | h |
| | | | | NUCLEOSOME ASSEMBLY PROTEIN 1-LIKE | |
| | | | | 4 [H. sapiens], ESTs, Weakly similar to | |
| | | | | NPL4_HUMAN NUCLEOSOME ASSEMBLY | |
| | | | | PROTEIN 1-LIKE 4 [H. sapiens], SET | |
| | | | | translocation, nucleosome assembly protein 1- | |
| | | | | like 1, nucleosome assembly protein 1-like 4 | |
| 416 | 1830 | AI009002 | | cell division cycle 25 homolog B (S. cerevisiae), | a, b |
| | | | | cell division cycle 25 homolog C (S. cerevisiae), | |
| | | | | cell division cycle 25B, cell division cycle 25C, | |
| | | | | expressed sequence AI64853 | |
| 417 | 17485 | AI012352 | | | General |
| 418 | 3982 | AI175100 | | | General |
| 419 | 3965 | AI112118 | | | General |
| 420 | 5969 | AI102520 | | ESTs, Weakly similar to GEF2_HUMAN | c, General |
| | | | | GANGLIOSIDE EXPRESSION FACTOR 2 | |
| | | | | [R. norvegicus], GABA(A) receptor-associated | |
| | | | | protein-like 2, RIKEN cDNA 6112F2 gene, | |
| | | | | gamma-aminobutyric acid (GABA(A)) receptor- | |
| | | | | associated protein-like 1, ganglioside | |
| | | | | expression factor 2 | |
| 421 | 15088 | AI232613 | | | General |
| 422 | 25691 | X53504 | | | e |
| 423 | 2984 | AA997015 | | | General |
| 424 | 11708 | AI171807 | | | General |
| 425 | 23187 | AI144591 | | | General |
| 426 | 16818 | AI072906 | | | a |
| 427 | 19298 | AI236338 | | ESTs, Highly similar to NHPX RAT NHP2/RS6 | General |
| | | | | FAMILY PROTEIN YEL26W HOMOLOG | |
| | | | | [R. norvegicus], RIKEN cDNA 24113M7 gene, | |
| | | | | non-histone chromosome protein 2 (S. | |
| | | | | cerevisiae)-like 1, nucleolar protein family A, | |
| | | | | member 2 (H/ACA small nucleolar RNPs), | |
| | | | | sperm specific antigen 1 | |
| 428 | 14345 | AA944773 | | DKFZP586L724 protein, Homo sapiens cDNA: | e |
| | | | | FLJ21737 fis, clone COLF3396 | |
| 429 | 10109 | X58465 | ribosomal protein S5 | EST, Moderately similar to 4S RIBOSOMAL | e |
| | | | | PROTEIN S5 [R. norvegicus], ESTs, Weakly | |
| | | | | similar to S55916 ribosomal protein S5, | |
| | | | | cytosolic [H. sapiens], ribosomal protein S5 | |
| 430 | 5596 | AI044747 | | | e, General |
| 431 | 14989 | AI177366 | | ESTs, Highly similar to B2779 fibronectin | f, i, |
| | | | | receptor beta chain precursor [H. sapiens], | General |
| | | | | integrin beta 1 (fibronectin receptor beta), | |
| | | | | integrin beta 2, integrin, beta 1 (fibronectin | |
| | | | | receptor, beta polypeptide, antigen CD29 | |
| | | | | includes MDF2, MSK12), integrin, beta 2 | |
| | | | | (antigen CD18 (p95), lymphocyte function- | |
| | | | | associated antigen 1; macrophage antigen 1 | |
| | | | | (mac-1) beta subunit) | |
| 432 | 17320 | AI178069 | | | General |
| 433 | 13646 | X62166 | | EST, Weakly similar to 6S RIBOSOMAL | General |
| | | | | PROTEIN L3 [R. norvegicus], EST, Weakly | |
| | | | | similar to I8451 ribosomal protein L3 | |
| | | | | [H. sapiens], ESTs, Moderately similar to 6S | |
| | | | | RIBOSOMAL PROTEIN L3 [R. norvegicus], | |
| | | | | ESTs, Moderately similar to I8451 ribosomal | |
| | | | | protein L3 [H. sapiens], ESTs, Weakly similar to | |
| | | | | RL3 MOUSE 6S RIBOSOMAL PROTEIN L3 | |
| | | | | [M. musculus], RIKEN cDNA 11157H16 gene, | |
| | | | | ribosomal protein L3, ribosomal protein L3-like | |
| 434 | 18611 | X58200 | ribosomal protein L29 | EST, Moderately similar to 6S RIBOSOMAL | e, General |
| | | | | PROTEIN L29 [R. norvegicus], EST, Weakly | |
| | | | | similar to S65784 ribosomal protein L29, | |
| | | | | cytosolic [H. sapiens], ESTs, Highly similar to | |
| | | | | S65784 ribosomal protein L29, cytosolic | |
| | | | | [H. sapiens], ESTs, Weakly similar to 6S | |
| | | | | RIBOSOMAL PROTEIN L29 [M. musculus], | |
| | | | | Human DNA sequence from clone RP4-595K12 | |
| | | | | on chromosome 1p31.2-31.3 Contains a | |
| | | | | pseudogene similar to 6S RPL29 (ribosomal | |
| | | | | protein L29 (cell surface heparin binding protein | |
| | | | | HIP)), a chromosome 1 specific mRNA | |
| | | | | (KIAA499), a novel mRNA (KIAA433), ESTs, | |
| | | | | STSs, GSSs and a CpG Island, ribosomal | |
| | | | | protein L29 | |
| 435 | 21671 | AA900295 | | placental growth factor, placental growth factor, | General |
| | | | | vascular endothelial growth factor-related | |
| | | | | protein | |
| 436 | 9452 | AA955206 | | | General |
| 437 | 20901 | AA942706 | | | d |
| 438 | 21985 | AA943273 | | | a |
| 439 | 7168 | AI012739 | | | General |
| 440 | 12195 | AA899121 | | | General |
| 441 | 15929 | AA849740 | | hypothetical protein | General |
| 442 | 16322 | AA866240 | | | c |
| 443 | 18047 | AI171359 | | | c |
| 444 | 15966 | AA923994 | | developmentally regulated GTP-binding protein 2 | b |
| 445 | 13251 | AI179264 | | | General |
| 446 | 18095 | AI177482 | | ESTs, Weakly similar to PTNB MOUSE | i, General |
| | | | | PROTEIN-TYROSINE PHOSPHATASE SYP | |
| | | | | [M. musculus], Homo sapiens clone L48 | |
| | | | | polyadenylated HERV LTR sequence and | |
| | | | | flanking cellular sequence, mRNA, expressed | |
| | | | | sequence AA589446, hemopoietic cell | |
| | | | | phosphatase, protein tyrosine phosphatase, | |
| | | | | non-receptor type 11, protein tyrosine | |
| | | | | phosphatase, non-receptor type 13, protein | |
| | | | | tyrosine phosphatase, non-receptor type 6 | |
| 447 | 16053 | AI228596 | | | General |
| 448 | 10684 | AA849687 | | ESTs, Highly similar to I39451 amyloid-beta | e, f |
| | | | | protein [H. sapiens], collagen, type VI, alpha 3 | |
| 449 | 22691 | AI177967 | | EST, Weakly similar to TGFB_HUMAN | General |
| | | | | LATENT TRANSFORMING GROWTH | |
| | | | | FACTOR BETA BINDING PROTEIN 1 | |
| | | | | PRECURSOR [H. sapiens], RIKEN cDNA | |
| | | | | 23146A13 gene, hypothetical protein MGC131, | |
| | | | | latent transforming growth factor beta binding | |
| | | | | protein 1, latent transforming growth factor beta | |
| | | | | binding protein 2, latent transforming growth | |
| | | | | factor beta binding protein 3, transforming | |
| | | | | growth factor-beta (TGF-beta) masking protein | |
| | | | | large subunit | |
| 450 | 15438 | AI179399 | collagen, type V, alpha 2, | EST, Highly similar to CA13_HUMAN | e |
| | | | procollagen, type V, | COLLAGEN ALPHA 1(III) CHAIN | |
| | | | alpha 2 | PRECURSOR [H. sapiens], EST, Weakly | |
| | | | | similar to CA25_HUMAN COLLAGEN ALPHA | |
| | | | | 2(V) CHAIN PRECURSO [H. sapiens], EST, | |
| | | | | Weakly similar to I4967 procollagen type V | |
| | | | | alpha 2 - mouse [M. musculus], RIKEN cDNA | |
| | | | | 2812D19 gene, collagen, type III, alpha 1 | |
| | | | | (Ehlers-Danlos syndrome type IV, autosomal | |
| | | | | dominant), collagen, type V, alpha 2, | |
| | | | | hypothetical protein DKFZp434F318, | |
| | | | | hypothetical protein MGC12921, procollagen, | |
| | | | | type III, alpha 1, procollagen, type V, alpha 2, | |
| | | | | procollagen, type XIII, alpha 1 | |
| 451 | 20975 | AA799599 | | | f, General |
| 452 | 15048 | M24542 | | EST, Moderately similar to UCRI RAT | c, i |
| | | | | UBIQUINOL-CYTOCHROME C REDUCTASE | |
| | | | | IRON-SULFUR SUBUNIT, MITOCHONDRIAL | |
| | | | | PRECURSOR [R. norvegicus], EST, Weakly | |
| | | | | similar to UCRI_HUMAN UBIQUINOL- | |
| | | | | CYTOCHROME C REDUCTASE IRON- | |
| | | | | SULFUR SUBUNIT, MITOCHONDRIAL | |
| | | | | PRECURSOR [H. sapiens], ESTs, Moderately | |
| | | | | similar to UCRI_HUMAN UBIQUINOL- | |
| | | | | CYTOCHROME C REDUCTASE IRON- | |
| | | | | SULFUR SUBUNIT, MITOCHONDRIAL | |
| | | | | PRECURSOR [H. sapiens], Human DNA | |
| | | | | sequence from clone RP1-228J4 on | |
| | | | | chromosome 6 Contains a pseudogene similar | |
| | | | | to UQCRFS1 (ubiquinol-cytochrome c | |
| | | | | reductase, Rieske iron-sulfur polypeptide 1), | |
| | | | | ESTs, an STS and GSSs, RIKEN cDNA | |
| | | | | 44342G14 gene, expressed sequence | |
| | | | | AI87555, ubiquinol-cytochrome c reductase, | |
| | | | | Rieske iron-sulfur polypeptide 1 | |
| 453 | 19379 | AI102711 | | EST, Moderately similar to S34195 ribosomal | General |
| | | | | protein L3, cytosolic [H. sapiens], ESTs, Highly | |
| | | | | similar to S34195 ribosomal protein L3, | |
| | | | | cytosolic [H. sapiens], ESTs, Weakly similar to | |
| | | | | RL3 MOUSE 6S RIBOSOMAL PROTEIN L3 | |
| | | | | [M. musculus] | |
| 454 | 12583 | AI234251 | | | General |
| 455 | 19480 | AA944442 | | ESTs, Moderately similar to JC2324 LIM | c |
| | | | | protein [H. sapiens], Homo sapiens cDNA | |
| | | | | FLJ13238 fis, clone OVARC144, LIM and | |
| | | | | senescent cell antigen-like domains 1, | |
| | | | | expressed sequence AV278559, expressed | |
| | | | | sequence AW18311, four and a half LIM | |
| | | | | domains 2, paxillin, pinch-2, transforming | |
| | | | | growth factor beta 1 induced transcript 1, | |
| | | | | vascular Rab-GAP/TBC-containing | |
| 456 | 7700 | AI105383 | | | General |
| 457 | 21996 | AA946036 | | | f |
| 458 | 26148 | AI013396 | | | General |
| 459 | 9551 | AI013558 | | | d |
| 460 | 23632 | AI237273 | | | d, f, |
| | | | | | General |
| 461 | 15292 | AF012714 | multiple inositol | | c |
| | | | polyphosphate histidine | | |
| | | | phosphatase 1, multiple | | |
| | | | inositol polyphosphate | | |
| | | | histidine phosphatase, 1 | | |
| 462 | 6632 | AI235277 | | | General |
| 463 | 18406 | AI236168 | | | General |
| 464 | 11865 | AA866383 | | | General |
| 465 | 20461 | AI639350 | | | i, General |
| 466 | 11728 | AI007884 | | | d |
| 467 | 6562 | AI177734 | | | d, General |
| 468 | 3046 | AA997706 | | | h |
| 469 | 25730 | X63369 | | | General |
| 470 | 23166 | AA859954 | | | General |
| 471 | 3271 | AI010303 | | | e |
| 472 | 4626 | AI104744 | | | General |
| 473 | 6430 | AI232319 | | RIKEN cDNA 18115E19 gene, erythrocyte | General |
| | | | | membrane protein band 7.2 (stomatin), | |
| | | | | expressed sequence AI79225 | |
| 474 | 6262 | AI177125 | | | General |
| 475 | 8786 | AA996993 | | IK cytokine, down-regulator of HLA II, Mus | d |
| | | | | musculus, Similar to IK cytokine, down- | |
| | | | | regulator of HLA II, clone MGC: 2558 | |
| | | | | IMAGE: 492184, mRNA, complete cds | |
| 476 | 12946 | AI228291 | | | General |
| 477 | 23015 | AI227724 | | | f, General |
| 478 | 15949 | AA963780 | | | i |
| 479 | 5458 | AI009756 | programmed cell death 6 | EST, Moderately similar to T14756 hypothetical | g, i, |
| | | | interacting protein, | protein DKFZp564F923.1 [H. sapiens], EST, | General |
| | | | programmed cell death 6- | Weakly similar to A28996 proline-rich protein | |
| | | | interacting protein | M14 precursor - mouse [M. musculus], EST, | |
| | | | | Weakly similar to PRP4_HUMAN SALIVARY | |
| | | | | PROLINE-RICH PROTEIN PO PRECURSOR | |
| | | | | [H. sapiens], expressed sequence AI462446, | |
| | | | | poly(A)-binding protein, nuclear 1, proline rich | |
| | | | | protein, proline rich protein 2, proline-rich | |
| | | | | protein BstNI subfamily 4, protein tyrosine | |
| | | | | phosphatase, non-receptor type 23 | |
| 480 | 11726 | AA849518 | | | d |
| 481 | 22197 | AI178527 | | | General |
| 482 | 21311 | AI178688 | | | General |
| 483 | 16092 | AI009012 | | ESTs, Moderately similar to T13152 WDR1 | g, i, |
| | | | | protein [H. sapiens], WD repeat domain 1 | General |
| 484 | 15283 | AA858548 | | | i, General |
| 485 | 23601 | AA955126 | | ESTs, Moderately similar to MYB-RELATED | f |
| | | | | PROTEIN B [M. musculus], myeloblastosis | |
| | | | | oncogene-like 2, v-myb avian myeloblastosis | |
| | | | | viral oncogene homolog-like 2 | |
| 486 | 2418 | AI013790 | | DKFZP586D623 protein, hypothetical protein | e |
| | | | | MGC123 | |
| 487 | 3214 | AI235483 | | | General |
| 488 | 20056 | AI639504 | | | General |
| 489 | 6431 | AA859085 | | | General |
| 490 | 17145 | M38566 | | serine (or cysteine) proteinase inhibitor, clade F | i |
| | | | | (alpha-2 antiplasmin, pigment epithelium | |
| | | | | derived factor), member 2, serine protease | |
| | | | | inhibitor 2-2 | |
| 491 | 15492 | AA892376 | | | h |
| 492 | 23687 | AI137958 | | | e |
| 493 | 17224 | AI179883 | | ESTs, Highly similar to MG15_HUMAN | c |
| | | | | TRANSCRIPTION FACTOR-LIKE PROTEIN | |
| | | | | MRG15 (MORF-RELATED GENE 15 | |
| | | | | PROTEIN) (MSL3-1 PROTEIN) (PROTEIN | |
| | | | | HSPC8/HSPC61) [H. sapiens], MORF-related | |
| | | | | gene 15 | c |
| 494 | 3682 | AA998964 | | | c |
| 495 | 21818 | AF036537 | | ESTs, Highly similar to A55318 | c |
| | | | | serine/threonine protein kinase [M. musculus], | |
| | | | | ESTs, Weakly similar to RIP MOUSE | |
| | | | | SERINE/THREONINE PROTEIN KINASE RIP | |
| | | | | [M. musculus], Human DNA sequence from | |
| | | | | clone RP5-1182A14 on chromosome 1 | |
| | | | | Contains part of a gene similar to rat Espin, a | |
| | | | | pseudogene similar to KIAA454, a gene similar | |
| | | | | to MST1 (macrophage stimulating 1 | |
| | | | | (hepatocyte growth factor-like)), a pseudogene | |
| | | | | similar to KIAA445, two isoforms of a novel | |
| | | | | gene (isoform 2 is the gene for KIAA1245 | |
| | | | | protein), ESTs, STSs, GSSs and CpG islands, | |
| | | | | ankyrin repeat domain 3, cerebral cavernous | |
| | | | | malformations 1, mitogen activated protein | |
| | | | | kinase kinase kinase 12, mitogen-activated | |
| | | | | protein kinase kinase kinase 12, receptor | |
| | | | | (TNFRSF)-interacting serine-threonine kinase | |
| | | | | 1, receptor interacting protein 3, receptor- | |
| | | | | interacting serine-threonine kinase 2 | |
| 496 | 3434 | AI232014 | | | General |
| 497 | 15084 | AA799397 | | | a |
| 498 | 23013 | AI230743 | ARP3 (actin-related | ARP3 (actin-related protein 3, yeast) homolog, | General |
| | | | protein 3, yeast) homolog, | EST, Weakly similar to ARP3_HUMAN ACTIN- | |
| | | | actin-related protein 3 | LIKE PROTEIN 3 [H. sapiens], Homo sapiens | |
| | | | homolog (yeast) | FKSG73 (FKSG73) mRNA, complete cds, | |
| | | | | RIKEN cDNA 6141G9 gene, actin, alpha 1, | |
| | | | | skeletal muscle, actin-related protein 3-beta | |
| 429 | 25702 | X58465 | ribosomal protein S5 | EST, Moderately similar to 4S RIBOSOMAL | e |
| | | | | PROTEIN S5 [R. norvegicus], ESTs, Weakly | |
| | | | | similar to S55916 ribosomal protein S5, | |
| | | | | cytosolic [H. sapiens], ribosomal protein S5 | |
| 499 | 3817 | AA926328 | | EST, Weakly similar to 143B_HUMAN 14-3-3 | General |
| | | | | PROTEIN BETA/ALPHA [H. sapiens], ESTs, | |
| | | | | Highly similar to A Chain A, 14-3-3 | |
| | | | | ZetaPHOSPHOPEPTIDE COMPLEX | |
| | | | | [H. sapiens], makorin, ring finger protein, 3, | |
| | | | | stratifin, tyrosine 3-monooxgenase/tryptophan 5 | |
| | | | | monooxgenase activation protein, beta | |
| | | | | polypeptide, tyrosine 3- | |
| | | | | monooxygenase/tryptophan 5-monooxygenase | |
| | | | | activation protein, zeta polypeptide | |
| 500 | 3669 | AA957535 | | | g, i, |
| | | | | | General |
| 501 | 11331 | AI145556 | | | d |
| 502 | 15558 | AA875537 | | FUS-interacting protein (serine-arginine rich) 1, | General |
| | | | | FUS-interacting protein (serine-arginine rich) 2, | |
| | | | | Homo sapiens SRp46 splicing factor | |
| | | | | transcribed retropseudogene, Mus musculus | |
| | | | | splicing factor Sc35 (Pr264) mRNA, partial cds, | |
| | | | | alternatively spliced, Splicing factor, | |
| | | | | arginine/serine-rich, 46 kD, splicing factor, | |
| | | | | arginine/serine-rich 2 (SC-35) | |
| 503 | 23409 | AA956294 | | | General |
| 504 | 4652 | AA899642 | | | General |
| 505 | 14117 | AI171350 | | | General |
| 506 | 18744 | AI170407 | | | General |
| 507 | 11561 | AI233182 | | | General |
| 508 | 22711 | AA946072 | | | General |
| 509 | 3941 | AI011598 | | ESTs, Moderately similar to 2113291A | d, i, |
| | | | | laminin: SUBUNIT, Usher syndrome 2A | General |
| | | | | (autosomal recessive, mild) homolog (human), | |
| | | | | hypothetical protein, MGC: 8159, laminin, alpha 5 | |
| 510 | 23464 | AA925876 | | | General |
| 511 | 168 | AA924985 | calsequestrin 2, | EST, Weakly similar to CAQC RAT | h |
| | | | calsequestrin 2 (cardiac | CALSEQUESTRIN, CARDIAC MUSCLE | |
| | | | muscle) | ISOFORM PRECURSOR [R. norvegicus], | |
| | | | | ESTs, Moderately similar to CAQC RAT | |
| | | | | CALSEQUESTRIN, CARDIAC MUSCLE | |
| | | | | ISOFORM PRECURSOR [R. norvegicus], | |
| | | | | calsequestrin 1, calsequestrin 1 (fast-twitch, | |
| | | | | skeletal muscle), calsequestrin 2, calsequestrin | |
| | | | | 2 (cardiac muscle) | |
| 512 | 14944 | AA925364 | | | e |
| 513 | 24228 | AA800318 | | serine (or cysteine) proteinase inhibitor, clade F | b |
| | | | | (alpha-2 antiplasmin, pigment epithelium | |
| | | | | derived factor), member 2, serine (or cysteine) | |
| | | | | proteinase inhibitor, clade G (C1 inhibitor), | |
| | | | | member 1 | |
| 514 | 23822 | AA943114 | | | General |
| 515 | 12988 | AI37070 | | | b, General |
| 516 | 4499 | AA892511 | | RIKEN cDNA2111P9 gene, calcium binding | e |
| | | | | protein P22, hepatocellular carcinoma antigen | |
| | | | | gene 52, hypothetical protein FLJ267, tescalcin | |
| 517 | 12274 | AA964306 | | | General |
| 518 | 6094 | AA818911 | | | d |
| 519 | 19257 | AI030775 | | expressed sequence AI323765, | b |
| | | | | histocompatibility 2, class II antigen E alpha, | |
| | | | | major histocompatibility complex, class II, DP | |
| | | | | alpha 2 (pseudogene), major histocompatibility | |
| | | | | complex, class II, DR alpha | |
| 520 | 17847 | AI178214 | | | General |
| 521 | 18887 | AA892860 | | | General |
| 522 | 20740 | S69874 | | EST, Weakly similar to FABE HUMAN FATTY | c, d, |
| | | | | ACID-BINDING PROTEIN, EPIDERMAL | General |
| | | | | [H. sapiens], fatty acid binding protein 5, | |
| | | | | epidermal | |
| 523 | 4770 | AI235915 | | | f |
| 524 | 21705 | AA900684 | | EST, Weakly similar to Y64_HUMAN | f |
| | | | | HYPOTHETICAL PROTEIN KIAA64 | |
| | | | | [H. sapiens], ESTs, Moderately similar to | |
| | | | | Y64_HUMAN HYPOTHETICAL PROTEIN | |
| | | | | KIAA64 [H. sapiens], ESTs, Weakly similar to | |
| | | | | Y64_HUMAN HYPOTHETICAL PROTEIN | |
| | | | | KIAA64 [H. sapiens], sorting nexin 17 | |
| 525 | 18797 | AI236618 | | Homo sapiens cDNA FLJ13574 fis, clone | General |
| | | | | PLACE18625, hypothetical protein, hypothetical | |
| | | | | protein FLJ2668, similar to S. cerevisiaeSSM4 | |
| 526 | 22212 | AI236294 | | EST, Weakly similar to IF6_HUMAN | General |
| | | | | EUKARYOTIC TRANSLATION INITIATION | |
| | | | | FACTOR 6 [H. sapiens], Mus musculus 1 days | |
| | | | | neonate cerebellum cDNA, RIKEN full-length | |
| | | | | enriched library, clone: 65342L5, full insert | |
| | | | | sequence, integrin beta 4 binding protein | |
| 527 | 2153 | U75404 | A kinase (PRKA) anchor | | General |
| | | | protein (gravin) 12 | | |
| 528 | 21654 | U53184 | | | i, General |
| 529 | 3191 | AI013075 | | ESTs, Weakly similar to T3454 ALR protein | General |
| | | | | [H. sapiens], chromatin-specific transcription | |
| | | | | elongation factor, 14 kDa subunit, hypothetical | |
| | | | | protein FLJ2144 similar to Rbig1, | |
| | | | | myeloid/lymphoid or mixed-lineage leukemia 2 | |
| 530 | 16613 | AA874784 | | | e, f, |
| | | | | | General |
| 531 | 5482 | AI180252 | | | General |
| 532 | 14337 | AI180414 | | EST, Highly similar to T1416 probable GTPase- | General |
| | | | | activating protein SPA-1 - rat [R. norvegicus], | |
| | | | | ESTs, Highly similar to T1416 probable | |
| | | | | GTPase-activating protein SPA-1 - rat | |
| | | | | [R. norvegicus], KIAA44 protein, RIKEN cDNA | |
| | | | | 261511M17 gene, expressed sequence | |
| | | | | AW213287, signal-induced proliferation | |
| | | | | associated gene 1 | |
| 533 | 23471 | AA955162 | | | General |
| 534 | 4098 | AI008642 | | Acid nuclear phosphoprotein 32 (leucine rich), | i, General |
| | | | | ESTs, Weakly similar to LEUCINE-RICH | |
| | | | | ACIDIC NUCLEAR PROTEIN [R. norvegicus], | |
| | | | | acidic nuclear phosphoprotein 32, acidic protein | |
| | | | | rich in leucines, cerebellar ataxia 3, pp32 | |
| | | | | related 1, pp32 related 2, putative human HLA | |
| | | | | class II associated protein I | |
| 535 | 23372 | AI031002 | | | h |
| 536 | 18217 | AA858930 | | | General |
| 537 | 3905 | AI103403 | neural polypyrimidine | ESTs, Moderately similar to S15552 | General |
| | | | tract binding protein, | polypyrimidine tract-binding protein 1 - rat | |
| | | | polypyrimidine tract | [R. norvegicus], ESTs, Weakly similar to | |
| | | | binding protein | S15552 polypyrimidine tract-binding protein 1 - | |
| | | | | rat [R. norvegicus], RIKEN cDNA 28136L13 | |
| | | | | gene, expressed sequence AW17884, | |
| | | | | heterogeneous nuclear ribonucleoprotein L, | |
| | | | | neural polypyrimidine tract binding protein, | |
| | | | | polypyrimidine tract binding protein, | |
| | | | | polypyrimidine tract binding protein 2 | |
| 538 | 24375 | AI104979 | | | General |
| 539 | 14256 | AI176845 | | HSPC163 protein, RIKEN cDNA D533D3 gene, | General |
| | | | | cornichon homolog (Drosophila) | |
| 540 | 18349 | AA799744 | | | General |
| 541 | 2310 | AI029969 | | | General |
| 542 | 3437 | AA892897 | | | General |
| 543 | 806 | AI169231 | | Gene 33/Mig-6, RIKEN cDNA 132F13 gene | General |
| 544 | 11531 | AA799773 | | EST, Moderately similar to ABP2_HUMAN | g, h, i, |
| | | | | ENDOTHELIAL ACTIN-BINDING PROTEIN | General |
| | | | | [H. sapiens], ESTs, Moderately similar to | |
| | | | | ABP2_HUMAN ENDOTHELIAL ACTIN- | |
| | | | | BINDING PROTEIN [H. sapiens], filamin A, | |
| | | | | alpha (actin-binding protein-28), filamin B, beta | |
| | | | | (actin-binding protein-278) | |
| 545 | 15181 | AI235234 | | | i |
| 546 | 22689 | AA945962 | | | General |
| 547 | 9084 | AA893717 | | | f |
| 548 | 5307 | AA893529 | | DNA segment, Chr 12, ERATO Doi 748, | b |
| | | | | expressed, General transcription factor IIIA | |
| 549 | 25663 | X05472 | | | e |
| 550 | 18278 | AI105080 | | 3-oxoacid CoA transferase, hypothetical protein | General |
| | | | | FKSG25 | |
| 551 | 6044 | AI011285 | | | a, General |
| 552 | 17361 | AA859114 | | | i, General |
| 553 | 22464 | AI009713 | | | d |
| 554 | 8137 | AA800749 | | | e |
| 555 | 16426 | X70369 | collagen, type III, alpha 1 | EST, Highly similar to CA13_HUMAN | c, e |
| | | | (Ehlers-Danlos syndrome | COLLAGEN ALPHA 1(III) CHAIN | |
| | | | type IV, autosomal | PRECURSOR [H. sapiens], EST, Moderately | |
| | | | dominant), procollagen, | similar to CA13_HUMAN COLLAGEN ALPHA | |
| | | | type III, alpha 1 | 1(III) CHAIN PRECURSOR [H. sapiens], EST, | |
| | | | | Weakly similar to CGHU7L collagen alpha 1(III) | |
| | | | | chain precursor [H. sapiens], ESTs, Highly | |
| | | | | similar to CA21_HUMAN COLLAGEN ALPHA | |
| | | | | 2(l) CHAIN PRECURSOR [H. sapiens], collagen | |
| | | | | type V, alpha 2, collagen, type III, alpha 1 | |
| | | | | (Ehlers-Danlos syndrome type IV, autosomal | |
| | | | | dominant), macrophage receptor with | |
| | | | | collagenous structure, procollagen, type III, | |
| | | | | alpha 1 | |
| 556 | 24237 | AA817726 | | | General |
| 557 | 17950 | AI236590 | | | General |
| 558 | 18172 | AI058364 | | CDC37 (cell division cycle 37, S. cerevisiae, | d, g, |
| | | | | homolog), ESTs, Moderately similar to G2313 | General |
| | | | | CDC37 homolog [H. sapiens], Hsp9-associating | |
| | | | | relative of Cdc37 | |
| 559 | 22614 | AI031004 | | | d |
| 560 | 23005 | AA942770 | | | General |
| 561 | 17285 | J02827 | branched chain keto acid | | General |
| | | | dehydrogenase E1, alpha | | |
| | | | polypeptide (maple syrup | | |
| | | | urine disease), branched | | |
| | | | chain ketoacid | | |
| | | | dehydrogenase E1, alpha | | |
| | | | polypeptide | | |
| 562 | 15885 | AA965207 | | EST, Highly similar to T14795 hypothetical | d |
| | | | | protein DKFZp434E171.1 [H. sapiens] | |
| 563 | 5461 | AI044338 | | | General |
| 564 | 22677 | AA942718 | BCL2-like 1, Bcl2-like | BCL2-like 1, BCL2-like 2, Bcl-2-related ovarian | General |
| | | | | killer protein, Bcl-2-related ovarian killer protein- | |
| | | | | like, Bcl-w protein, Bcl2-like, Bcl2-like 2 | |
| 565 | 18507 | AI175551 | | ESTs, Highly similar to S25432 translation | General |
| | | | | elongation factor eEF-1 beta chain [H. sapiens], | |
| | | | | eukaryotic translation elongation factor 1 beta 2 | |
| 566 | 8012 | AI058330 | | Complement component 4 binding protein, | General |
| | | | | alpha, ESTs, Moderately similar to NBHUC4 | |
| | | | | C4b-binding protein alpha chain precursor | |
| | | | | [H. sapiens], ESTs, Weakly similar to | |
| | | | | C4BP_HUMAN C4B-BINDING PROTEIN | |
| | | | | ALPHA CHAIN PRECURSOR [H. sapiens], Mus | |
| | | | | musculus decay accelerating factor | |
| | | | | glycosylphoshatidylinositol-anchored form | |
| | | | | (DAF) mRNA, partial cds, complement | |
| | | | | component 4-binding protein, alpha, decay | |
| | | | | accelerating factor 1, decay accelerating factor | |
| | | | | for complement (CD55, Cromer blood group | |
| | | | | system), expressed sequence AI323748 | |
| 567 | 13310 | AI177119 | | ESTs, Weakly similar to COMPLEMENT C1Q | b, General |
| | | | | SUBCOMPONENT, C CHAIN PRECURSOR | |
| | | | | [M. musculus], Homo sapiens, Similar to | |
| | | | | complement component 1, q subcomponent, c | |
| | | | | polypeptide, clone MGC: 17279 | |
| | | | | IMAGE: 4212772, mRNA, complete cds, | |
| | | | | complement component 1, q subcomponent, | |
| | | | | beta polypeptide, complement component 1, q | |
| | | | | subcomponent, c polypeptide, expressed | |
| | | | | sequence AI385742 | |
| 568 | 6548 | AI072658 | | | d, General |
| 569 | 7941 | AI043958 | | | g, i, |
| | | | | | General |
| 570 | 13619 | AI179464 | | | General |
| 571 | 3207 | AA997466 | | | General |
| 572 | 20582 | AA859688 | | AU RNA-binding protein/enoyl-Coenzyme A | f, General |
| | | | | hydratase, EST, Weakly similar to I37195 AU- | |
| | | | | specific RNA-binding protein/enoyl-CoA | |
| | | | | hydratase homolog [H. sapiens], hypothetical | |
| | | | | protein FLJ1948, uncharacterized | |
| | | | | hypothalamus protein HCDASE | |
| 573 | 21796 | AI012221 | | EST X83352, ESTs, Highly similar to T17226 | g, General |
| | | | | hypothetical protein DKFZp566G223.1 | |
| | | | | [H. sapiens], Homo sapiens, Similar to chloride | |
| | | | | intracellular channel 4, clone MGC: 8812 | |
| | | | | IMAGE: 3861372, mRNA, complete cds, RIKEN | |
| | | | | cDNA 573531E12 gene, chloride intracellular | |
| | | | | channel 1, chloride intracellular channel 4, | |
| | | | | chloride intracellular channel 4 (mitochondrial), | |
| | | | | hypothetical protein DKFZp434N127, | |
| | | | | intracellular chloride ion channel protein p64H1 | |
| 574 | 19071 | AA850524 | | ESTs, Weakly similar to B5625 transcription | f |
| | | | | factor LBP1b [H. sapiens], Mus musculus, clone | |
| | | | | MGC: 785 IMAGE: 349986, mRNA, complete | |
| | | | | cds, upstream binding protein 1, upstream | |
| | | | | binding protein 1 (LBP-1a) | |
| 575 | 24200 | AI012356 | | | General |
| 576 | 24246 | AA963703 | | | g, General |
| 577 | 6133 | AA866234 | | elongation factor for selenoprotein translation | c |
| 578 | 21632 | AI009167 | | BCL2-associated athanogene 2, ESTs, Highly | General |
| | | | | similar to T8764 hypothetical protein | |
| | | | | DKFZp586C21.1 [H. sapiens] | |
| 579 | 13772 | AA851926 | | DEAD (aspartate-glutamate-alanine-aspartate) | General |
| | | | | box polypeptide 5, DEAD-box protein, DEAD/H | |
| | | | | (Asp-Glu-Ala-Asp/His) box polypeptide 5 (RNA | |
| | | | | helicase, 68 kD), ESTs, Highly similar to | |
| | | | | 146327A growth regulated nuclear 68 protein | |
| | | | | [H. sapiens], Homo sapiens, Similar to RIKEN | |
| | | | | cDNA 23161O4 gene, clone MGC: 21583 | |
| | | | | IMAGE: 4479998, mRNA, complete cds, | |
| | | | | KIAA81 gene product, RIKEN cDNA 2617K22 | |
| | | | | gene, RIKEN cDNA 492156D17 gene, RIKEN | |
| | | | | cDNA 91343L19 gene, RNA helicase, Rattus | |
| | | | | norvegicus RNA helicase with arginine-serine- | |
| | | | | rich domain mRNA, complete cds, expressed | |
| | | | | sequence AI32543 | |
| 580 | 22247 | AA943537 | | ajuba, expressed sequence AI48116, | i, General |
| | | | | expressed sequence R75157, zyxin | |
| 581 | 23732 | AA957653 | | ESTs, Moderately similar to JC5614 RNB6 | General |
| | | | | protein - rat [R. norvegicus], ESTs, Weakly | |
| | | | | similar to NPC DERIVED PROLINE RICH | |
| | | | | PROTEIN 1 [M. musculus], Ena-vasodilator | |
| | | | | stimulated phosphoprotein, NPC derived | |
| | | | | proline rich protein 1, RNB6, enabled homolog | |
| | | | | (Drosophila), likely ortholog of mouse NPC | |
| | | | | derived proline rich protein 1 | |
| 582 | 22385 | AA859805 | | ESTs, Weakly similar to PROTEIN-LYSINE 6- | e |
| | | | | OXIDASE PRECURSOR [R. norvegicus], Lysyl | |
| | | | | oxidase, lysyl oxidase, lysyl oxidase-like, lysyl | |
| | | | | oxidase-like 1, lysyl oxidase-like 2 | |
| 583 | 2459 | AA964755 | | | i |
| 584 | 23964 | AI234748 | | | g, General |
| 585 | 4074 | AI175990 | | | g |
| 586 | 17204 | AI171844 | | ATP synthase, H+ transporting, mitochondrial | e, g |
| | | | | F1 complex, epsilon subunit, RIKEN cDNA | |
| | | | | 24143G19 gene, expressed sequence AV645 | |
| 587 | 6560 | AI230440 | | | d |
| 588 | 17883 | M11851 | | | a, b |
| 589 | 22937 | AA850290 | | ATPase, Class V, type 1B, ATPase, Class V, | i |
| | | | | type 1D, ATPase, class V, type 1A, EST, | |
| | | | | Highly similar to AT5B_HUMAN POTENTIAL | |
| | | | | PHOSPHOLIPID-TRANSPORTING ATPASE | |
| | | | | VB [H. sapiens], ESTs, Weakly similar to AT5A | |
| | | | | MOUSE POTENTIAL PHOSPHOLIPID- | |
| | | | | TRANSPORTING ATPASE VA [M. musculus] | |
| 590 | 4944 | AA924405 | | ESTs, Weakly similar to NEUROFILAMENT | g, General |
| | | | | TRIPLET H PROTEIN [M. musculus], RIKEN | |
| | | | | cDNA 619L18 gene, RIKEN cDNA 133A17 | |
| | | | | gene, RIKEN cDNA 241142G14 gene, | |
| | | | | nucleolar protein (KKE/D repeat) | |
| 591 | 3822 | AA900863 | | DNA segment, Chr 17, human D6S81E 1, | g, General |
| | | | | ESTs, Highly similar to S33681 translation | |
| | | | | initiation factor eIF-4A.I [H. sapiens], HLA-B | |
| | | | | associated transcript 1, Human clone 23933 | |
| | | | | mRNA sequence, eukaryotic translation | |
| | | | | initiation factor 4A, isoform 1, mitochondrial | |
| | | | | DEAD-box polypeptide 28, nuclear RNA | |
| | | | | helicase, DECD variant of DEAD box family | |
| 592 | 2729 | AI170363 | | | d |
| 593 | 19282 | AA955123 | | | General |
| 594 | 21740 | AI176810 | | | General |
| 595 | 23773 | AA956476 | | | General |
| 596 | 6843 | AI009768 | | | f |
| 597 | 14840 | AI237698 | | | General |
| 598 | 20038 | AA899797 | | | c, General |
| 599 | 18541 | X14671 | | ESTs, Highly similar to RL26_HUMAN 6S | e, h |
| | | | | RIBOSOMAL PROTEIN L26 [H. sapiens], | |
| | | | | ESTs, Highly similar to S33713 ribosomal | |
| | | | | protein L26, cytosolic [H. sapiens], ESTs, | |
| | | | | Weakly similar to 6S RIBOSOMAL PROTEIN | |
| | | | | L26 [R. norvegicus], ribosomal protein L26, | |
| | | | | ribosomal protein L26 pseudogene 1 | |
| 600 | 13966 | AI231421 | | | d, General |
| 601 | 17234 | AI102741 | tissue inhibitor of | tissue inhibitor of metalloproteinase 3, tissue | h |
| | | | metalloproteinase 3, | inhibitor of metalloproteinase 3 (Sorsby fundus | |
| | | | tissue inhibitor of | dystrophy, pseudoinflammatory) | |
| | | | metalloproteinase 3 | | |
| | | | (Sorsby fundus | | |
| | | | dystrophy, | | |
| | | | pseudoinflammatory) | | |
| 602 | 22544 | AA851245 | | apoptosis-associated speck-like protein | General |
| | | | | containing a CARD, death effector filament- | |
| | | | | forming Ced-4-like apoptosis protein, | |
| | | | | hypothetical protein PP144, tumor up-regulated | |
| | | | | CARD-containing antagonist of caspase nine | |
| 603 | 13023 | AI233740 | | Aldehyde reductase 1 (low Km aldose | General |
| | | | | reductase) (5.8 kb Pstl fragment, probably the | |
| | | | | functional gene), EST, Highly similar to Aldose | |
| | | | | Reductase (E.C.1.1.1.21) [H. sapiens], ESTs, | |
| | | | | Moderately similar to ALDOSE REDUCTASE | |
| | | | | [R. norvegicus], ESTs, Moderately similar to | |
| | | | | ALDOSE REDUCTASE-RELATED PROTEIN 2 | |
| | | | | [M. musculus], Homo sapiens, Similar to RIKEN | |
| | | | | cDNA 11118J12 gene, clone IMAGE: 3865164, | |
| | | | | mRNA, partial cds, RIKEN cDNA 2315E1 gene, | |
| | | | | aldo-keto reductase family 1, member A4 | |
| | | | | (aldehyde reductase), aldo-keto reductase | |
| | | | | family 1, member B1 (aldose reductase), aldo- | |
| | | | | keto reductase family 1, member B1 (aldose | |
| | | | | reductase), aldo-keto reductase family 1, | |
| | | | | member B3 (aldose reductase), fibroblast | |
| | | | | growth factor regulated protein | |
| 604 | 24209 | AI172423 | | | i, General |
| 605 | 23626 | AA955540 | | | General |
| 606 | 4670 | AI233714 | | | h |
| 607 | 18215 | AA943367 | | | h |
| 608 | 3014 | AI176362 | | | c, General |
| 609 | 16756 | AA818089 | | glycyl-tRNA synthetase | General |
| 610 | 22605 | AA859447 | | | i |
| 611 | 5283 | AF090113 | | ESTs, Highly similar to T32733 AMPA | h |
| | | | | glutamate receptor interacting protein GRIP - | |
| | | | | rat [R. norvegicus], Glutamate receptor | |
| | | | | interacting protein, KIAA1365 protein, Lin-7b | |
| | | | | protein; likely ortholog of mouse LIN-7B; | |
| | | | | mammalian LIN-7 protein 2, Mus musculus | |
| | | | | grip1 mRNA for glutamate receptor interacting | |
| | | | | protein 1a-L, complete cds, Vertebrate LIN7 | |
| | | | | homolog 1, Tax interaction protein 33, channel- | |
| | | | | interacting PDZ domain protein, discs, large | |
| | | | | (Drosophila) homolog 2 (chapsyn-11), multiple | |
| | | | | PDZ domain protein, semaF cytoplasmic | |
| | | | | domain associated protein 3, syntaxin binding | |
| | | | | protein 4 | |
| 612 | 6043 | AI059588 | | | g |
| 613 | 21474 | AA851361 | | | d |
| 614 | 23038 | AA900881 | branched chain | Homo sapiens cDNA: FLJ2127 fis, clone | General |
| | | | aminotransferase 1, | COL1749, branched chain aminotransferase 1, | |
| | | | cytosolic | cytosolic | |
| 615 | 13336 | AI237070 | | | a, b, |
| | | | | | General |
| 616 | 3988 | AI008546 | proteasome (prosome, | EST, Weakly similar to SNHUC8 multicatalytic | d |
| | | | macropain) subunit, alpha | endopeptidase complex [H. sapiens], ESTs, | |
| | | | type 3, proteasome | Highly similar to PRC8 MOUSE | |
| | | | (prosome, macropain) | PROTEASOME COMPONENT C8 | |
| | | | subunit, alpha type, 3 | [M. musculus], ESTs, Highly similar to | |
| | | | | PROTEASOME COMPONENT C8 | |
| | | | | [R. norvegicus], ESTs, Weakly similar to | |
| | | | | SNHUC8 multicatalytic endopeptidase complex | |
| | | | | [H sapiens], proteasome (prosome, macropain) | |
| | | | | subunit, alpha type 3, proteasome (prosome, | |
| | | | | macropain) subunit, alpha type, 3 | |
| 617 | 20823 | AI176302 | | EST, Weakly similar to UBIM_HUMAN | d |
| | | | | UBIQUITIN-LIKE PROTEIN FUBI {SUB 1-74 | |
| | | | | [H. sapiens], EST, Weakly similar to ubiquitin- | |
| | | | | like protein/ribosomal protein S3 | |
| | | | | [R. norvegicus], Finkel-Biskis-Reilly murine | |
| | | | | sarcoma virus (FBR-MuSV) ubiquitously | |
| | | | | expressed (fox derived), Finkel-Biskis-Reilly | |
| | | | | murine sarcoma virus (FBR-MuSV) ubiquitously | |
| | | | | expressed (fox derived); ribosomal protein S3 | |
| 618 | 23044 | AF034218 | | RIKEN cDNA 4632428M18 gene, | General |
| | | | | hyaluronidase 1, hyaluronidase 2, | |
| | | | | hyaluronoglucosaminidase 1, | |
| | | | | hyaluronoglucosaminidase 2, | |
| | | | | hyaluronoglucosaminidase 3, sperm adhesion | |
| | | | | molecule | |
| 619 | 19013 | AI639256 | | | a |
| 620 | 16124 | AI176963 | | Cbp/p3-interacting transactivator, with Glu/Asp- | c |
| | | | | rich carboxy-terminal domain, 2, Cbp/p3- | |
| | | | | interacting transactivator, with Glu/Asp-rich | |
| | | | | carboxy-terminal domain, 4, ESTs, Weakly | |
| | | | | similar to MRG1_HUMAN MSG-RELATED | |
| | | | | PROTEIN 1 [H. sapiens], expressed sequence | |
| | | | | AW742964 | |
| 621 | 17848 | AA900460 | | T-complex 1, chaperonin containing TCP1, | General |
| | | | | subunit 7 (eta), chaperonin subunit 7 (eta), | |
| | | | | expressed sequence AI528772, expressed | |
| | | | | sequence AL22769, t-complex 1, t-complex | |
| | | | | protein 1 | |
| 622 | 19074 | AI009391 | | | f |
| 623 | 3615 | AI237645 | | Homo sapiens mRNA; cDNA DKFZp434M2227 | d, General |
| | | | | (from clone DKFZp434M2227), Homo sapiens | |
| | | | | prostate-specific membrane antigen PSM | |
| | | | | mRNA, exon 6 alternative splice variant, partial | |
| | | | | cds, RIKEN cDNA 26128K12 gene, folate | |
| | | | | hydrolase, transferrin receptor, transferrin | |
| | | | | receptor (p9, CD71), transferrin receptor 2 | |
| 624 | 11387 | AA891677 | | | General |
| 625 | 25765 | X89706 | | | General |
| 626 | 21653 | AI237535 | | | General |
| 627 | 5228 | AI233311 | | | General |
| 628 | 7299 | AI013911 | | H.sapiensPABII pseudogene, Human DNA | c, d, |
| | | | | sequence from PAC 56B9 on chromosome | General |
| | | | | 1q24-1q25. Contains profilin-like pseudogene, | |
| | | | | 6S ribosomal protein L4 pseudogene RNA | |
| | | | | binding protein, ESTs, GSS, Mus musculus | |
| | | | | adult male tongue cDNA, RIKEN full-length | |
| | | | | enriched library, clone: 23174E15, full insert | |
| | | | | sequence, RNA binding motif protein 3, RNA | |
| | | | | binding motif protein, X chromosome, RNA | |
| | | | | binding motif protein, X chromosome retrogene, | |
| | | | | cold inducible RNA-binding protein, testes- | |
| | | | | specific heterogenous nuclear | |
| | | | | ribonucleoprotein G-T | |
| 629 | 2093 | AI102097 | | | General |
| 630 | 9166 | AI137406 | | protein C receptor, endothelial, protein C | i, General |
| | | | | receptor, endothelial (EPCR) | |
| 631 | 19723 | AA818761 | | | General |
| 632 | 2741 | AA963077 | | | General |
| 633 | 22667 | AA945069 | | | General |
| 634 | 17687 | AA799531 | | | a, e |
| 635 | 23217 | AI235953 | | | General |
| 636 | 12135 | AI176418 | | | c |
| 637 | 19894 | AA859581 | | EST, Highly similar to SPERM-COATING | d |
| | | | | GLYCOPROTEIN PRECURSOR | |
| | | | | [R. norvegicus], ESTs, Weakly similar to | |
| | | | | JC4131 glioma pathogenesis-related protein | |
| | | | | [H. sapiens], Human DNA sequence from clone | |
| | | | | RP5-881L22 on chromosome 2 Contains ESTs, | |
| | | | | GSSs, STSs and CpG islands. Contains a gene | |
| | | | | for a novel protein similar to a trypsin inhibitor | |
| | | | | and four other genes for novel proteins, RIKEN | |
| | | | | cDNA 18149K24 gene, RIKEN cDNA | |
| | | | | 241114O14 gene, RIKEN cDNA 923112K8 | |
| | | | | gene, acidic epididymal glycoprotein 1, acidic | |
| | | | | epididymal glycoprotein 2, epididymal | |
| | | | | glycoprotein, glioma pathogenesis-related | |
| | | | | protein | |
| 638 | 15345 | AI171587 | | | h |
| 639 | 23165 | AI231799 | | Friedreich ataxia region gene X123 | General |
| 640 | 3391 | AA998197 | | | General |
| 641 | 16215 | AA874999 | | protein translocation complex beta | General |
| 642 | 19086 | AA892598 | | | General |
| 643 | 4725 | AA900290 | | Alpha-2-macroglobulin, ESTs, Moderately | d, General |
| | | | | similar to A2M2 MOUSE MURINOGLOBULIN 2 | |
| | | | | PRECURSOR [M. musculus], RIKEN cDNA | |
| | | | | 26137121 gene, alpha-2-macroglobulin, carbon | |
| | | | | catabolite repression 4 homolog (S. cerevisiae), | |
| | | | | expressed sequence AW456442, pregnancy- | |
| | | | | zone protein | |
| 644 | 6727 | AI233213 | | | a, g |
| 645 | 21816 | AI231217 | | EST, Highly similar to S61A RAT PROTEIN | h, General |
| | | | | TRANSPORT PROTEIN SEC61 ALPHA | |
| | | | | SUBUNIT [R. norvegicus], ESTs, Highly similar | |
| | | | | to S61A RAT PROTEIN TRANSPORT | |
| | | | | PROTEIN SEC61 ALPHA SUBUNIT | |
| | | | | [R. norvegicus], SEC61, alpha subunit (S. | |
| | | | | cerevisiae), SEC61, alpha subunit 2 (S. | |
| | | | | cerevisiae), Sec61 alpha form 2, protein | |
| | | | | transport protein SEC61 alpha subunit isoform 1 | |
| 646 | 2069 | AI103616 | | ESTs, Weakly similar to B Chain B, Crystal | g, i, |
| | | | | Structure Of Rac1 In Complex With The | General |
| | | | | Guanine Nucleotide Exchange Region Of | |
| | | | | Tiam1 [H. sapiens], ESTs, Weakly similar to | |
| | | | | RAS-RELATED C3 BOTULINUM TOXIN | |
| | | | | SUBSTRATE 1 [M. musculus], KIAA717 protein, | |
| | | | | KIAA74 gene product, Mus musculus mRNA for | |
| | | | | Rac3, complete cds, RAS-related C3 botulinum | |
| | | | | substrate 1, RIKEN cDNA 178H16 gene, | |
| | | | | aplysia ras-related homolog A2, plysia ras- | |
| | | | | related homolog A2, ras homolog gene family, | |
| | | | | member A, ras homolog gene family, member | |
| | | | | C, ras-related C3 botulinum toxin substrate 1 | |
| | | | | (rho family, small GTP binding protein Rac1) | |
| 647 | 22717 | AI235948 | | ESTs, Weakly similar to NIDOGEN | e, g |
| | | | | [R. norvegicus], RIKEN cDNA 2945C1 gene, | |
| | | | | nidogen (enactin), nidogen 1, | |
| | | | | sparc/osteonectin, cwcv and kazal-like domains | |
| | | | | proteoglycan 1 | |
| 648 | 11205 | AI172057 | | | General |
| 649 | 22498 | AA944806 | | | c |
| 650 | 7192 | AI178530 | | | c |
| 651 | 8058 | AA818475 | | | General |
| 652 | 18800 | AI178504 | | | General |
| 653 | 12376 | AI103939 | | | i |
| 654 | 12695 | AI170948 | | G protein-coupled receptor 4, G protein-coupled | i, General |
| | | | | receptor 65 | |
| 655 | 5227 | AA925924 | | | f, i, |
| | | | | | General |
| 656 | 7936 | AA875495 | | | i, General |
| 657 | 4491 | AA818798 | | cathepsin Z | General |
| 658 | 13647 | AA892367 | | EST, Weakly similar to 6S RIBOSOMAL | General |
| | | | | PROTEIN L3 [R. norvegicus], EST, Weakly | |
| | | | | similar to I8451 ribosomal protein L3 | |
| | | | | [H. sapiens], ESTs, Moderately similar to 6S | |
| | | | | RIBOSOMAL PROTEIN L3 [R. norvegicus], | |
| | | | | ESTs, Moderately similar to I8451 ribosomal | |
| | | | | protein L3 [H. sapiens], ESTs, Weakly similar to | |
| | | | | RL3 MOUSE 6S RIBOSOMAL PROTEIN L3 | |
| | | | | [M. musculus], RIKEN cDNA 11157H16 gene, | |
| | | | | ribosomal protein L3, ribosomal protein L3-like | |
| 659 | 25716 | X61295 | | | e |
| 660 | 10547 | AA800186 | | | General |
| 661 | 7760 | AI030806 | | | b, General |
| 662 | 16687 | AI013553 | | | a |
| 663 | 24040 | AA957422 | | CD3 antigen, zeta polypeptide, CD3Z antigen, | General |
| | | | | zeta polypeptide (TiT3 complex), Fc fragment | |
| | | | | of IgE, high affinity I, receptor for; gamma | |
| | | | | polypeptide, Fc receptor, IgE, high affinity I, | |
| | | | | gamma polypeptide, expressed sequence | |
| | | | | AI573376 | |
| 664 | 16154 | AI009661 | | ALL1-fused gene from chromosome 1q | i |
| 665 | 14390 | AI232385 | | | g |
| 666 | 14651 | AI235919 | | | General |
| 667 | 17289 | AA891785 | | ESTs, Highly similar to ISOCITRATE | b |
| | | | | DEHYDROGENASE [R. norvegicus], Isocitrate | |
| | | | | dehydrogenase 1, soluble, isocitrate | |
| | | | | dehydrogenase 1 (NADP+), soluble, isocitrate | |
| | | | | dehydrogenase 2 (NADP+), mitochondrial | |
| 668 | 12609 | AA997486 | | | d, i, |
| | | | | | General |
| 669 | 21657 | X61381 | | ESTs, Moderately similar to S17182 interferon- | b, i, |
| | | | | induced protein 1-8U [H. sapiens], ESTs, | General |
| | | | | Weakly similar to putative haemopoietic | |
| | | | | membrane protein [M. musculus], Human DNA | |
| | | | | sequence from clone RP4-781L3 on | |
| | | | | chromosome 1p34.3-36.11 Contains a | |
| | | | | pseudogene similar to IFITM3 (interferon | |
| | | | | inducedntransmembrane protein 3 (1-8U)), | |
| | | | | STSs and GSSs, RIKEN cDNA 1114C5 gene, | |
| | | | | interferon induced transmembrane protein 3 (1-8U), | |
| | | | | interferon induced transmembrane protein | |
| | | | | 3-like, interferon-inducible protein 16 | |
| 670 | 5384 | AA891041 | | Jun-B oncogene, jun B proto-oncogene | General |
| 671 | 5613 | AI044850 | | | b |
| 672 | 16136 | AI103983 | | RIKEN cDNA B43311C9 gene, caspase | General |
| | | | | recruitment domain family, member 6, nucleolar | |
| | | | | protein 3 (apoptosis repressor with CARD | |
| | | | | domain) | |
| 673 | 23270 | AI179601 | | | General |
| 674 | 14464 | AI104848 | | | General |
| 675 | 6053 | AA818655 | | | d |
| 676 | 19067 | AA859663 | | | General |
| 677 | 22877 | AI178819 | | adrenal gland protein AD-4 | General |
| 678 | 14257 | AI230460 | | MARCKS-like protein, Mus musculus 8 days | e |
| | | | | embryo cDNA, RIKEN full-length enriched | |
| | | | | library, clone: 573519L1, full insert sequence, | |
| | | | | Myristoylated alanine-rich protein kinase C | |
| | | | | substrate, macrophage myristoylated alanine- | |
| | | | | rich C kinase substrate, myristoylated alanine | |
| | | | | rich protein kinase C substrate, myristoylated | |
| | | | | alanine-rich protein kinase C substrate | |
| | | | | (MARCKS, 8K-L) | |
| 679 | 5132 | AA925342 | | myomesin 1, myomesin 1 (skelemin) (185 kD), | General |
| | | | | myomesin 2 | |
| 680 | 2706 | AA965083 | | | General |
| 681 | 22615 | AA945643 | | | f, General |
| 682 | 13460 | AI175375 | | | General |
| 683 | 8707 | AI059707 | | | i, General |
| 684 | 17948 | AA944581 | | HIV-1 Rev binding protein-like, Rev/Rex | General |
| | | | | activation domain binding protein-related | |
| 685 | 13082 | AI136848 | | | a, b |
| 686 | 16074 | AA874874 | | alcohol dehydrogenase 5, alcohol | f |
| | | | | dehydrogenase 5 (class III), chi polypeptide | |
| 687 | 8861 | AI070306 | | | General |
| 688 | 8594 | AI012932 | | | General |
| 689 | 22311 | AI176007 | | pM5 protein | General |
| 690 | 24268 | AI172281 | | | e |
| 691 | 9073 | AA892273 | | | a, b |
| 692 | 22423 | AA893164 | | | b |
| 693 | 5550 | AI044628 | | | General |
| 694 | 3895 | AA894029 | | | b |
| 695 | 19069 | AA943737 | endothelial differentiation | endothelial differentiation sphingolipid G-protein- | General |
| | | | sphingolipid G-protein- | coupled receptor 1, endothelial differentiation, | |
| | | | coupled receptor 1, | G-protein-coupled receptor 6, endothelial | |
| | | | endothelial differentiation, | differentiation, sphingolipid G-protein-coupled | |
| | | | sphingolipid G-protein- | receptor, 1, endothelial differentiation, | |
| | | | coupled receptor, 1 | sphingolipid G-protein-coupled receptor, 3, | |
| | | | | endothelial differentiation, sphingolipid G- | |
| | | | | protein-coupled receptor, 5, endothelial | |
| | | | | differentiation, sphingolipid G-protein-coupled | |
| | | | | receptor, 8, sphingosine 1-phosphate receptor | |
| 696 | 22666 | AA900974 | | | d, General |
| 697 | 2852 | AI177059 | | | e, General |
| 698 | 12108 | AA848963 | | | a |
| 699 | 23240 | AA893939 | | Deleted in split-hand/split-foot 1 region, split | i |
| | | | | hand/foot deleted gene 1 | |
| 700 | 4161 | AI112141 | | | General |
| 701 | 18272 | AA799294 | | | c, e, |
| | | | | | General |
| 702 | 23612 | AI170751 | | | General |
| 703 | 4790 | AA900875 | | Cbp/p3-interacting transactivator, with Glu/Asp- | General |
| | | | | rich carboxy-terminal domain, 1, Cbp/p3- | |
| | | | | interacting transactivator, with Glu/Asp-rich | |
| | | | | carboxy-terminal domain, 2, Cbp/p3-interacting | |
| | | | | transactivator, with Glu/Asp-rich carboxy- | |
| | | | | terminal domain, 4, expressed sequence | |
| | | | | AW742964 | |
| 704 | 16168 | AA893280 | | RIKEN cDNA 1312C15 gene, RIKEN cDNA | General |
| | | | | 23176L9 gene, adipose differentiation related | |
| | | | | protein, adipose differentiation-related protein | |
| 705 | 11644 | AI235282 | | EST, Moderately similar to S25111 alpha-2- | General |
| | | | | macroglobulin receptor precursor - mouse | |
| | | | | [M. musculus], ESTs, Highly similar to S25111 | |
| | | | | alpha-2-macroglobulin receptor precursor - | |
| | | | | mouse [M. musculus], low density lipoprotein | |
| | | | | receptor-related protein 1, low density | |
| | | | | lipoprotein-related protein 1 (alpha-2- | |
| | | | | macroglobulin receptor), low density lipoprotein- | |
| | | | | related protein 1B (deleted in tumors) | |
| 706 | 19057 | AI235094 | | Homo sapiens cDNA FLJ13271 fis, clone | General |
| | | | | OVARC11, Homo sapiens, Similar to cortactin | |
| | | | | isoform B, clone MGC: 3591 IMAGE: 3637586, | |
| | | | | mRNA, complete cds, Mus musculus, Similar to | |
| | | | | cortactin isoform B, clone MGC: 18474 | |
| | | | | IMAGE: 3981559, mRNA, complete cds, | |
| | | | | R.norvegicus myr3 mRNA for myosin I heavy | |
| | | | | chain, RIKEN cDNA 2312N23 gene, RIKEN | |
| | | | | cDNA 91323P14 gene, cortactin, ems1 | |
| | | | | sequence (mammary tumor and squamous cell | |
| | | | | carcinoma-associated (p8/85 src substrate), | |
| | | | | myosin IE | |
| 707 | 9501 | AI638949 | | | a |
| 708 | 24310 | AA924578 | | | e, General |
| 709 | 1283 | U61729 | | DNA segment, Chr 4, Brigham & Women's | General |
| | | | | Genetics 593 expressed, ESTs, Moderately | |
| | | | | similar to JC4899 proline rich protein - rat | |
| | | | | [R. norvegicus], RIKEN cDNA 6111E17 gene, | |
| | | | | hypothetical protein FLJ2312, proline-rich | |
| | | | | protein with nuclear targeting signal | |
| 710 | 7681 | AI030449 | | | General |
| 711 | 1439 | U57391 | | | General |
| 712 | 20872 | X51707 | ribosomal protein S19 | EST, Moderately similar to 4S RIBOSOMAL | f |
| | | | | PROTEIN S19 [R. norvegicus], EST, Weakly | |
| | | | | similar to RS19_HUMAN 4S RIBOSOMAL | |
| | | | | PROTEIN S19 [H. sapiens], ribosomal protein | |
| | | | | S19 | |
| 713 | 21660 | AI169751 | | ESTs, Moderately similar to S17182 interferon- | b, General |
| | | | | induced protein 1-8U [H. sapiens], ESTs, | |
| | | | | Weakly similar to putative haemopoietic | |
| | | | | membrane protein [M. musculus], Human DNA | |
| | | | | sequence from clone RP4-781L3 on | |
| | | | | chromosome 1p34.3-36.11 Contains a | |
| | | | | pseudogene similar to IFITM3 (interferon | |
| | | | | inducedntransmembrane protein 3 (1-8U)), | |
| | | | | STSs and GSSs, RIKEN cDNA 1114C5 gene, | |
| | | | | interferon induced transmembrane protein 3 (1-8U), | |
| | | | | interferon induced transmembrane protein | |
| | | | | 3-like, interferon-inducible protein 16 | |
| 714 | 19454 | AA998888 | | Mus musculus adult male stomach cDNA, | f |
| | | | | RIKEN full-length enriched library, | |
| | | | | clone: 22118M5, full insert sequence, expressed | |
| | | | | sequence AI8352, fibronectin type 3 and SPRY | |
| | | | | domain-containing protein, midline 1, midline 1 | |
| | | | | (Opitz/BBB syndrome), midline 2, ring finger | |
| | | | | protein 28 | |
| 715 | 12979 | AI169177 | | immediate early response 3 | General |
| 716 | 5675 | AI045026 | | | General |
| 717 | 17903 | AI231083 | | | d, General |
| 718 | 6743 | AI231219 | | | d, General |
| 719 | 23538 | AI102727 | | solute carrier family 2 (phosphate transporter), | General |
| | | | | member 1, solute carrier family 2 (phosphate | |
| | | | | transporter), member 2, solute carrier family 2, | |
| | | | | member 1, solute carrier family 2, member 2 | |
| 720 | 20617 | X53581 | | | e |
| 721 | 19416 | AA850244 | | | General |
| 722 | 16191 | AA874982 | | | c |
| 723 | 8745 | AI069939 | | | d |
| 724 | 14494 | AI234222 | | | g |
| 725 | 22681 | AA944413 | | | General |
| 726 | 21579 | AI103572 | | | General |
| 727 | 23978 | AA925352 | | | h, General |
| 728 | 25742 | X80130 | | | a, b, c |
| 729 | 14458 | AI145095 | | | General |
| 730 | 6172 | AA819646 | | | General |
| 731 | 1998 | AA817864 | | | b |
| 732 | 25545 | S77900 | | | c |
| 733 | 12999 | AI176276 | | Homo sapiens cDNA FLJ1257 fis, clone | d |
| | | | | NT2RM4895, UDP-N-acteylglucosamine | |
| | | | | pyrophosphorylase 1 | |
| 734 | 1159 | AA891949 | | | g, General |
| 735 | 18907 | AI178971 | | EST, Moderately similar to HART1 hemoglobin | a, b, |
| | | | | alpha-1 chain - rat [R. norvegicus], EST, Weakly | General |
| | | | | similar to A45964 hemoglobin alpha chain - | |
| | | | | mouse [M. musculus], ESTs, Moderately similar | |
| | | | | to HART1 hemoglobin alpha-1 chain - rat | |
| | | | | [R. norvegicus], Hemoglobin, alpha 1, | |
| | | | | hemoglobin alpha, adult chain 1, hemoglobin, | |
| | | | | alpha 1, hemoglobin, alpha 2 | |
| 736 | 25647 | U83119 | | | e |
| 737 | 17892 | AI228438 | | | a |
| 738 | 18447 | AA849939 | | | b, General |
| 401 | 18473 | AI168975 | | | General |
| 739 | 4222 | AA860024 | | EST, Moderately similar to EF1G_HUMAN | General |
| | | | | ELONGATION FACTOR 1-GAMMA | |
| | | | | [H. sapiens], ESTs, Highly similar to | |
| | | | | EF1G_HUMAN ELONGATION FACTOR 1- | |
| | | | | GAMMA [H. sapiens], Homo sapiens cDNA | |
| | | | | FLJ11216 fis, clone PLACE182, eukaryotic | |
| | | | | translation elongation factor 1 gamma | |
| 740 | 15197 | AI105444 | | | General |
| 741 | 22958 | AI171374 | | | d, General |
| 742 | 9111 | AI073176 | | makorin, ring finger protein, 1, makorin, ring | General |
| | | | | finger protein, 2, makorin, ring finger protein, 3, | |
| | | | | makorin, ring finger protein, 4 | |
| 743 | 23110 | AI169640 | | | d |
| 744 | 20998 | AA799803 | | EST, Moderately similar to MAS2_HUMAN | General |
| | | | | MANNAN-BINDING LECTIN SERINE | |
| | | | | PROTEASE 2 PRECURSOR [H. sapiens], | |
| | | | | Rattus norvegicus mRNA for serine protease, | |
| | | | | complete cds, complement C1r-like proteinase | |
| | | | | precursor, complement component 1, r | |
| | | | | subcomponent, complement component 1, s | |
| | | | | subcomponent, protein C | |
| 745 | 23184 | AI176554 | | | d |
| 746 | 14149 | AA998172 | | platelet-activating factor acetylhydrolase, | i |
| | | | | isoform 1b, alpha2 subunit, platelet-activating | |
| | | | | factor acetylhydrolase, isoform lb, beta subunit | |
| | | | | (3 kD) | |
| 747 | 23816 | AA956596 | | | e |
| 748 | 8494 | AI059968 | | | i, General |
| 749 | 21841 | AA996499 | | EST, Weakly similar to C1QA_HUMAN | b, General |
| | | | | COMPLEMENT C1Q SUBCOMPONENT, A | |
| | | | | CHAIN PRECURSO [H. sapiens], complement | |
| | | | | component 1, q subcomponent, alpha | |
| | | | | polypeptide, complement component 1, q | |
| | | | | subcomponent, beta polypeptide, expressed | |
| | | | | sequence AI255395, expressed sequence | |
| | | | | AI385742 | |
| 750 | 19765 | AI230945 | | | d |
| 751 | 6002 | AA818101 | | | b |
| 752 | 21945 | AI104132 | | | e |
| 753 | 4496 | AA892400 | | | i |
| 754 | 12921 | AI112636 | | Homo sapiens BAC clone RP11-335J18 from 2, | General |
| | | | | RIKEN cDNA 17124F2 gene, expressed | |
| | | | | sequence AI325217, uridine phosphorylase | |
| 755 | 14516 | AI013197 | | | e |
| 756 | 11504 | AI171652 | | | General |
| 757 | 3698 | AI172193 | | | e |
| 758 | 5886 | AI045807 | | | General |
| 759 | 4154 | AI009467 | | | General |
| 760 | 4330 | AA818747 | | | c, General |
| 390 | 16597 | AI102486 | | DNA segment, Chr 7, ERATO Doi 486, | General |
| | | | | expressed, EST, Weakly similar to S37583 | |
| | | | | RING finger protein rfp - mouse [M. musculus], | |
| | | | | expressed sequence AI894158, expressed | |
| | | | | sequence AW46487, hypothetical gene | |
| | | | | MGC1127 | |
| 761 | 15191 | AI176456 | | ESTs, Moderately similar to AF78844 1 hqp376 | General |
| | | | | protein [H. sapiens], expressed sequence | |
| | | | | AA49533 | |
| 762 | 10654 | AI235991 | | | f |
| 763 | 14341 | AI227596 | | | General |
| 764 | 22915 | AI228299 | | | a, c, |
| | | | | | General |
| 765 | 6638 | AI137579 | | | c, General |
| 766 | 25250 | D10706 | | | h |
| 767 | 24072 | AI231093 | | | e |
| 768 | 15628 | AI230338 | | EST AI31731, EST, Weakly similar to R3HU16 | General |
| | | | | ribosomal protein S16, cytosolic [H. sapiens], | |
| | | | | expressed sequence AA42385, ribosomal | |
| | | | | protein S16 | |
| 769 | 18831 | AI104357 | | ESTs, Weakly similar to A29861 actin gamma | c, h, |
| | | | | [H. sapiens], ESTs, Weakly similar to I39393 | General |
| | | | | alpha-actin [H. sapiens], ESTs, Weakly similar | |
| | | | | to S38782 actin beta' chain [H. sapiens], Homo | |
| | | | | sapiens mRNA; cDNA DKFZp434B2115 (from | |
| | | | | clone DKFZp434B2115), RIKEN cDNA | |
| | | | | 1752K15 gene, RIKEN cDNA 1761J2 gene, | |
| | | | | actin-like 7a, expressed sequence AL2324, | |
| | | | | expressed sequence AV259599, melanoma X- | |
| | | | | actin, uncharacterized hypothalamus protein | |
| | | | | HARP11 | |
| 770 | 8343 | AI059502 | | | i |
| 771 | 7749 | AA848804 | | ESTs, Moderately similar to 167338A | General |
| | | | | transcription factor BTF3a [H. sapiens], ESTs, | |
| | | | | Weakly similar to 167338A transcription factor | |
| | | | | BTF3a [H. sapiens], Homo sapiens cDNA | |
| | | | | FLJ14844 fis, clone PLACE1133, highly similar | |
| | | | | to TRANSCRIPTION FACTOR BTF3, RIKEN | |
| | | | | cDNA 1754E11 gene, RIKEN cDNA | |
| | | | | 4632412E9 gene, basic transcription factor 3, | |
| | | | | expressed sequence AI316863 | |
| 772 | 9984 | AA955536 | | ELL gene (11-19 lysine-rich leukemia gene), | General |
| | | | | ESTs, Weakly similar to ELL MOUSE RNA | |
| | | | | POLYMERASE II ELONGATION FACTOR ELL | |
| | | | | [M. musculus], Mus musculus, clone | |
| | | | | IMAGE: 358397, mRNA, partial cds, Mus | |
| | | | | musculus, clone MGC: 11987 IMAGE: 361737, | |
| | | | | mRNA, complete cds, eleven-nineteen lysine- | |
| | | | | rich leukemia gene, hypothetical protein | |
| | | | | FLJ2279 | |
| 773 | 17264 | D25233 | retinoblastoma 1, | ESTs, Highly similar to RB MOUSE | General |
| | | | retinoblastoma 1 | RETINOBLASTOMA-ASSOCIATED PROTEIN | |
| | | | (including osteosarcoma) | [M. musculus], retinoblastoma 1, retinoblastoma | |
| | | | | 1 (including osteosarcoma) | |
| 774 | 5356 | AI177813 | | modulator recognition factor I | General |
| 775 | 8626 | AI059449 | | | General |
| 776 | 2522 | AA964957 | | | a |
| 777 | 6291 | AI144797 | | | h |
| 642 | 19085 | AA892598 | | | General |
| 778 | 18366 | AA799442 | | | General |
| 779 | 1686 | AI179971 | hemoglobin alpha, adult | EST, Moderately similar to HART1 hemoglobin | b, General |
| | | | chain 2, hemoglobin, | alpha-1 chain - rat [R. norvegicus], ESTs, | |
| | | | alpha 1 | Moderately similar to HART1 hemoglobin alpha- | |
| | | | | 1 chain - rat [R. norvegicus], RIKEN cDNA | |
| | | | | 25142H12 gene, hemoglobin alpha, adult chain | |
| | | | | 1, hemoglobin, alpha 1, hemoglobin, alpha 2 | |
| 780 | 20004 | AI031036 | | DNA segment, Chr 3, ERATO Doi 555, | c |
| | | | | expressed, ESTs, Weakly similar to A3521 | |
| | | | | ferroxidase [R. norvegicus], ESTs, Weakly | |
| | | | | similar to CERU MOUSE CERULOPLASMIN | |
| | | | | PRECURSOR [M. musculus], ESTs, Weakly | |
| | | | | similar to KUHU ferroxidase [H. sapiens], | |
| | | | | ceruloplasmin, ceruloplasmin (ferroxidase), | |
| | | | | coagulation factor V, coagulation factor VIII, | |
| | | | | procoagulant component (hemophilia A), | |
| | | | | hephaestin | |
| 781 | 4969 | AI113008 | | | f, g, |
| | | | | | General |
| 782 | 6697 | AI045340 | | | General |
| 783 | 13420 | AA892042 | | | d, h, |
| | | | | | General |
| 784 | 21527 | AA851733 | | | f |
| 785 | 22017 | AA944209 | | ESTs, Highly similar to PROTO-ONCOGENE | General |
| | | | | SERINE/THREONINE-PROTEIN KINASE PIM- | |
| | | | | 1 [M. musculus], ESTs, Highly similar to S55333 | |
| | | | | protein kinase pim-2 [M. musculus], ESTs, | |
| | | | | Moderately similar to S55333 protein kinase | |
| | | | | pim-2 [M. musculus], Pim-1 oncogene, pim-1 | |
| | | | | oncogene, pim-2 oncogene, proviral integration | |
| | | | | site 1, serine threonine kinase pim3 | |
| 786 | 9388 | AA894173 | | | b |
| 787 | 23197 | AI172159 | | | General |
| 788 | 3518 | AA944584 | | | b, f |
| 422 | 18606 | X53504 | | EST, Moderately similar to S35531 ribosomal | e, General |
| | | | | protein L12, cytosolic [H. sapiens], EST, Weakly | |
| | | | | similar to 6S RIBOSOMAL PROTEIN L12 | |
| | | | | [R. norvegicus], hypothetical protein, ribosomal | |
| | | | | protein L12 | |
| 789 | 23123 | AA924794 | | | g, General |
| 790 | 3081 | AA999171 | signal transducer and | Signal transducer and activator of transcription | h |
| | | | activator of transcription | 3, expressed sequence AA48197, signal | |
| | | | 1, signal transducer and | transducer and activator of transcription 1, | |
| | | | activator of transcription | signal transducer and activator of transcription | |
| | | | 1, 91 kD | 1, 91 kD, signal transducer and activator of | |
| | | | | transcription 2, signal transducer and activator | |
| | | | | of transcription 3, signal transducer and | |
| | | | | activator of transcription 3 (acute-phase | |
| | | | | response factor), signal transducer and | |
| | | | | activator of transcription 4 | |
| 791 | 17358 | AI179147 | | electron-transfer-flavoprotein, beta polypeptide | e, h |
| 792 | 2435 | AI639411 | | hypothetical protein | h |
| 793 | 24651 | M83678 | | RAB13, member RAS oncogene family, RIKEN | b |
| | | | | cDNA 617N3 gene, expressed sequence | |
| | | | | AW17754 | |
| 794 | 17675 | AI137597 | | | f |
| 795 | 4407 | H33528 | | | General |
| 796 | 4874 | AA923850 | | | General |
| 797 | 16001 | AA866452 | actin, alpha, cardiac, | EST, Moderately similar to ATHUC actin, | a, b, c, g, |
| | | | actin, alpha, cardiac | cardiac muscle [H. sapiens], ESTs, Highly | General |
| | | | muscle | similar to ACTC_HUMAN ACTIN, ALPHA | |
| | | | | CARDIA [R. norvegicus], actin, alpha, cardiac, | |
| | | | | actin, alpha, cardiac muscle, smooth muscle | |
| | | | | alpha-actin | |
| 798 | 18883 | AA799992 | | | General |
| 799 | 10972 | AI233956 | | | General |
| 800 | 1114 | AI029917 | | | f |
| 801 | 15382 | AI172302 | | | General |
| 802 | 15568 | AI010257 | | | General |
| 803 | 10532 | AI009602 | | | g, General |
| 804 | 11873 | AI232326 | | | General |
| 805 | 13161 | AI138093 | | | e |
| 806 | 11485 | AI235348 | | | f |
| 807 | 25689 | X52815 | | | g, General |
| 808 | 1831 | AI071137 | | cell division cycle 25 homolog B (S. cerevisiae), | a, b, |
| | | | | cell division cycle 25 homolog C (S. cerevisiae), | General |
| | | | | cell division cycle 25B, cell division cycle 25C, | |
| | | | | expressed sequence AI64853 | |
| 809 | 25846 | AI638988 | | | General |
| 810 | 20086 | AI013260 | | | g |
| 811 | 20041 | AI112161 | | | i, General |
| 812 | 18867 | D88250 | | EST, Moderately similar to MAS2_HUMAN | a, b, |
| | | | | MANNAN-BINDING LECTIN SERINE | General |
| | | | | PROTEASE 2 PRECURSOR [H. sapiens], EST, | |
| | | | | Weakly similar to JC6554 probable serine | |
| | | | | proteinase [R. norvegicus], ESTs, Weakly | |
| | | | | similar to JC6554 probable serine proteinase | |
| | | | | [R. norvegicus], MASP-2 protein, complement | |
| | | | | component 1, r subcomponent, complement | |
| | | | | component 1, s subcomponent, mannan- | |
| | | | | binding lectin serine protease 1, mannan- | |
| | | | | binding lectin serine protease 2 | |
| 813 | 7615 | AI030163 | | | d |
| 814 | 23833 | AI113026 | | ESTs, Weakly similar to AGP2_RAT | General |
| | | | | ANGIOPOIETIN-2 (ANG-2) [R. norvegicus], | |
| | | | | angiopoietin 1, angiopoietin 2, angiopoietin-like | |
| | | | | 3, angiopoietin-like 4, angiopoietin-like factor | |
| 815 | 23731 | AA894004 | | ESTs, Highly similar to CAPG MOUSE | f, g, |
| | | | | MACROPHAGE CAPPING PROTEIN | General |
| | | | | [M. musculus], ESTs, Moderately similar to | |
| | | | | CAPG MOUSE MACROPHAGE CAPPING | |
| | | | | PROTEIN [M. musculus], RIKEN cDNA | |
| | | | | 573416F2 gene, capping protein (actin | |
| | | | | filament), gelsolin-like | |
| 816 | 21400 | M36410 | sepiapterin reductase, | | General |
| | | | sepiapterin reductase (7, 8 | | |
| | | | dihydrobiopterin: NADP+ | | |
| | | | oxidoreductase) | | |
| 817 | 20763 | AA925516 | | | b |
| 818 | 15218 | AI102495 | | RIKEN cDNA 1319I21 gene, expressed | General |
| | | | | sequence AL2431, nucleoside phosphorylase, | |
| | | | | purine-nucleoside phosphorylase | |
| 819 | 3088 | AI013369 | | | c |
| 820 | 10317 | AI172339 | | | General |
| 821 | 17468 | AA892545 | | | d |
| 822 | 23566 | AA955482 | | Mitochondrial Acyl-CoA Thioesterase, acyl- | General |
| | | | | Coenzyme A thioesterase 2, mitochondrial, acyl | |
| | | | | Coenzyme A thioesterase 3, mitochondrial | |
| 823 | 15510 | AA875428 | | | g, General |
| 824 | 17408 | AA818971 | | | h |
| 825 | 16608 | AA996840 | | | h |
| 826 | 15308 | AI072896 | | | h |
| 827 | 7397 | AI104244 | | ATP synthase, H+ transporting, mitochondrial | h |
| | | | | F1 complex, O subunit, ATP synthase, H+ | |
| | | | | transporting, mitochondrial F1 complex, O | |
| | | | | subunit (oligomycin sensitivity conferring | |
| | | | | protein), EST, Moderately similar to ATPO RAT | |
| | | | | ATP SYNTHASE OLIGOMYCIN SENSITIVITY | |
| | | | | CONFERRAL PROTEIN PRECURSOR, | |
| | | | | MITOCHONDRIAL [R. norvegicus], EST, | |
| | | | | Weakly similar to ATPO RAT ATP SYNTHASE | |
| | | | | OLIGOMYCIN SENSITIVITY CONFERRAL | |
| | | | | PROTEIN PRECURSOR, MITOCHONDRIAL | |
| | | | | [R. norvegicus], ESTs, Highly similar to | |
| | | | | ATPO_HUMAN ATP SYNTHASE | |
| | | | | OLIGOMYCIN SENSITIVITY CONFERRAL | |
| | | | | PROTEIN PRECURSOR, MITOCHONDRIAL | |
| | | | | [H. sapiens], ESTs, Moderately similar to | |
| | | | | ATPO_HUMAN ATP SYNTHASE | |
| | | | | OLIGOMYCIN SENSITIVITY CONFERRAL | |
| | | | | PROTEIN PRECURSOR, MITOCHONDRIAL | |
| | | | | [H. sapiens], Human DNA sequence from clone | |
| | | | | RP4-730D4 on chromosome 20q12 Contains a | |
| | | | | pseudogene similar to part of ATP50 (ATP | |
| | | | | synthase, H+ transporting, mitochondrial F1 | |
| | | | | complex, O subunit (oligomycin sensitivity | |
| | | | | conferring protein)), an EST, an STS and | |
| | | | | GSSs, Mus musculus adult male cerebellum | |
| | | | | cDNA, RIKEN full-length enriched library, | |
| | | | | clone: 1500031D10, full insert sequence | |
| 828 | 18542 | AA893493 | | ESTs, Highly similar to RL26_HUMAN 6S | e |
| | | | | RIBOSOMAL PROTEIN L26 [H. sapiens], | |
| | | | | ESTs, Highly similar to S33713 ribosomal | |
| | | | | protein L26, cytosolic [H. sapiens], ESTs, | |
| | | | | Weakly similar to 6S RIBOSOMAL PROTEIN | |
| | | | | L26 [R. norvegicus], ribosomal protein L26, | |
| | | | | ribosomal protein L26 pseudogene 1 | |
| 829 | 19094 | AI232021 | | ESTs, Highly similar to Human Translation | e |
| | | | | Initiation Factor Eif1, Nmr, 29 Structures | |
| | | | | [H. sapiens], RIKEN cDNA 151M16 gene, | |
| | | | | RIKEN cDNA 3111N14 gene, putative | |
| | | | | translation initiation factor, suppressor of | |
| | | | | initiator codon mutations, related sequence 1 | |
| | | | | (S. cerevisiae) | |
| 830 | 14449 | AI234152 | | | General |
| 831 | 12096 | AA819415 | | | i |
| 832 | 6135 | AA819065 | | | d |
| 833 | 21785 | AI177312 | | | g, General |
| 834 | 18454 | AI231776 | | | General |
| 835 | 21581 | AA944828 | | | c, General |
| 836 | 15383 | AA955358 | | | General |
| 837 | 16071 | AA997821 | | | General |
| 838 | 16533 | AA998174 | | | g, General |
| 839 | 5950 | AI169266 | | Homo sapiens cDNA FLJ13819 fis, clone | General |
| | | | | THYRO1452, actin related protein 2/3 complex, | |
| | | | | subunit 5 (16 kD), hypothetical protein similar to | |
| | | | | actin related protein 2/3 complex, subunit 5 | |
| 840 | 15004 | AI235224 | | EST, Moderately similar to TIM1 RAT | f, g, |
| | | | | METALLOPROTEINASE INHIBITOR 1 | General |
| | | | | PRECURSOR [R. norvegicus], EST, Weakly | |
| | | | | similar to TIM1 RAT METALLOPROTEINASE | |
| | | | | INHIBITOR 1 PRECURSOR [R. norvegicus], | |
| | | | | tissue inhibitor of metalloproteinase, tissue | |
| | | | | inhibitor of metalloproteinase 1 (erythroid | |
| | | | | potentiating activity, collagenase inhibitor) | |
| 798 | 18881 | AA799992 | | | a, General |
| 841 | 21185 | AI170056 | | | General |
| 842 | 5049 | D10655 | | ESTs, Weakly similar to ODP2 RAT | e |
| | | | | DIHYDROLIPOAMIDE | |
| | | | | ACETYLTRANSFERASE COMPONENT OF | |
| | | | | PYRUVATE DEHYDROGENASE COMPLEX | |
| | | | | [R. norvegicus], Mus musculus, clone | |
| | | | | IMAGE: 3586777, mRNA, partial cds, Pyruvate | |
| | | | | dehydrogenase complex, lipoyl-containing | |
| | | | | component X; E3-binding protein, RIKEN cDNA | |
| | | | | 1617E1 gene, RIKEN cDNA 493529O8 gene, | |
| | | | | dihydrolipoamide S-acetyltransferase (E2 | |
| | | | | component of pyruvate dehydrogenase | |
| | | | | complex), dihydrolipoamide branched chain | |
| | | | | transacylase (E2 component of branched chain | |
| | | | | keto acid dehydrogenase complex; maple syrup | |
| | | | | urine disease), dihydrolipoamide branched | |
| | | | | chain transacylase E2 | |
| 843 | 17810 | AA799778 | | ATP synthase, H+ transporting, mitochondrial | i |
| | | | | F complex, subunit b, isoform 1, ATP synthase, | |
| | | | | H+ transporting, mitochondrial F complex, | |
| | | | | subunit b, isoform 1, ESTs, Moderately similar | |
| | | | | to ATPF RAT ATP SYNTHASE B CHAIN, | |
| | | | | MITOCHONDRIAL PRECURSOR | |
| | | | | [R. norvegicus], expressed sequence C76477 | |
| 844 | 10071 | AI639058 | | chromosome 18 open reading frame 1, | i, General |
| | | | | transmembrane, prostate androgen induced | |
| | | | | RNA | |
| 845 | 2913 | AI232272 | | | General |
| 846 | 5703 | AI045136 | | | i, General |
| 847 | 3143 | AI232408 | | | General |
| 848 | 10780 | AI136555 | | DC6 protein, DKFZP564O823 protein, ESTs, | g, General |
| | | | | Weakly similar to S24169 mucin - rat | |
| | | | | [R. norvegicus], hypothetical protein FLJ13725, | |
| | | | | mucin 13, epithelial transmembrane | |
| 849 | 5357 | AI179507 | | | b, General |
| 850 | 18909 | AA849426 | | | General |
| 321 | 22696 | AA945996 | | | General |
| 851 | 9815 | AA848218 | | | General |
| 852 | 20761 | AA900331 | | | General |
| 853 | 18686 | D00729 | dodecenoyl-Coenzyme A | ESTs, Highly similar to D3D2 RAT 3,2-TRANS- | e, h |
| | | | delta isomerase (3,2 trans | ENOYL-COA ISOMERASE, MITOCHONDRIAL | |
| | | | enoyl-Coenyme A | PRECURSOR [R. norvegicus], Homo sapiens, | |
| | | | isomerase), dodecenoyl- | Similar to dodecenoyl-Coenzyme A delta | |
| | | | Coenzyme A delta | isomerase (3,2 trans-enoyl-Coenzyme A | |
| | | | isomerase (3,2 trans- | isomerase), clone MGC: 393 IMAGE: 363566, | |
| | | | enoyl-Coenzyme A | mRNA, complete cds, dodecenoyl-Coenzyme A | |
| | | | isomerase) | delta isomerase (3,2 trans.enoyl-Coenyme A | |
| | | | | isomerase), dodecenoyl-Coenzyme A delta | |
| | | | | isomerase (3,2 trans-enoyl-Coenzyme A | |
| | | | | isomerase) | |
| 854 | 2378 | AA964501 | | | d |
| 855 | 17307 | H32747 | | | h |
| 856 | 10837 | AI072144 | | | General |
| 857 | 4130 | AA800298 | | | e, f, |
| | | | | | General |
| 858 | 18529 | AI230716 | | | g, General |
| 859 | 7362 | AI029026 | | | General |
| 860 | 2730 | AI012962 | | CAAX box 1, DKFZP564B147 protein, EST, | General |
| | | | | Moderately similar to CXX1_HUMAN CAAX | |
| | | | | BOX PROTEIN 1 [H. sapiens] | |
| 861 | 21766 | AA850916 | | | General |
| 862 | 13240 | AI169311 | | | d, General |
| 863 | 12664 | AA999110 | | preferentially expressed antigen in melanoma | General |
| 864 | 24520 | L20869 | | ESTs, Highly similar to PAP1_HUMAN | General |
| | | | | PANCREATITIS-ASSOCIATED PROTEIN 1 | |
| | | | | PRECURSOR [H. sapiens], RIKEN cDNA | |
| | | | | 212L15 gene, expressed sequence AI449515, | |
| | | | | pancreatitis-associated protein, regenerating | |
| | | | | islet-derived 3 gamma | |
| 865 | 10549 | AA801255 | | | General |
| 866 | 8737 | AI071926 | | | h |
| 867 | 12674 | AI235358 | | ESTs, Moderately similar to UCR2 RAT | a, c |
| | | | | UBIQUINOL-CYTOCHROME C REDUCTASE | |
| | | | | COMPLEX CORE PROTEIN 2 PRECURSOR | |
| | | | | [R. norvegicus], RIKEN cDNA 15406 gene, | |
| | | | | ubiquinol-cytochrome c reductase core protein | |
| | | | | II | |
| 868 | 8032 | AI058398 | | | f |
| 869 | 2587 | AI232103 | | | General |
| 870 | 13547 | M63983 | hypoxanthine guanine | EST, Moderately similar to The Crystal | i |
| | | | phosphoribosyl | Structure Of Icam-2 {SUB 25-216 [H. sapiens], | |
| | | | transferase, hypoxanthine | ESTs, Weakly similar to HPRT RAT | |
| | | | phosphoribosyltransferas | HYPOXANTHINE-GUANINE | |
| | | | e 1 (Lesch-Nyhan | PHOSPHORIBOSYLTRANSFERASE | |
| | | | syndrome) | [R. norvegicus], HHGP protein, hypoxanthine | |
| | | | | guanine phosphoribosyl transferase, | |
| | | | | hypoxanthine phosphoribosyltransferase 1 | |
| | | | | (Lesch-Nyhan syndrome), intercellular | |
| | | | | adhesion molecule 2 | |
| 871 | 2135 | AI010195 | | | General |
| 872 | 6881 | AI010169 | | | General |
| 873 | 5494 | AI044425 | | | General |
| 874 | 16834 | AA963324 | | | d |
| 875 | 5699 | AI045128 | | ESTs, Highly similar to T5615 hypothetical | General |
| | | | | protein DKFZp761G1515.1 [H. sapiens], protein | |
| | | | | kinase, lysine deficient 3, serologically defined | |
| | | | | colon cancer antigen 43 | |
| 876 | 6282 | AA819523 | | | c |
| 877 | 18598 | AI070775 | | | General |
| 878 | 5297 | AI170379 | | | General |
| 879 | 7867 | AI043695 | | ESTs, Highly similar to PUR1_HUMAN | d |
| | | | | AMIDOPHOSPHORIBOSYLTRANSFERASE | |
| | | | | PRECURSOR [H. sapiens], ESTs, Moderately | |
| | | | | similar to PUR1_HUMAN | |
| | | | | AMIDOPHOSPHORIBOSYLTRANSFERASE | |
| | | | | PRECURSOR [H. sapiens], RIKEN cDNA | |
| | | | | 573454C12 gene, expressed sequence | |
| | | | | AA675351, expressed sequence C79945, | |
| | | | | glutamine fructose-6-phosphate transaminase | |
| | | | | 2, glutamine-fructose-6-phosphate | |
| | | | | transaminase 2, phosphoribosyl pyrophosphate | |
| | | | | amidotransferase | |
| 880 | 6097 | AA818929 | | | General |
| 881 | 10115 | AI058890 | | | d |
| 882 | 3738 | AA998256 | | | General |
| 883 | 9770 | AI060016 | | | General |
| 884 | 18830 | AA964496 | | ESTs, Weakly similar to A29861 actin gamma | g, h |
| | | | | [H. sapiens], ESTs, Weakly similar to I39393 | General |
| | | | | alpha-actin [H. sapiens], ESTs, Weakly similar | |
| | | | | to S38782 actin beta' chain [H. sapiens], Homo | |
| | | | | sapiens mRNA; cDNA DKFZp434B2115 (from | |
| | | | | clone DKFZp434B2115), RIKEN cDNA | |
| | | | | 1752K15 gene, RIKEN cDNA 1761J2 gene, | |
| | | | | actin-like 7a, expressed sequence AL2324, | |
| | | | | expressed sequence AV259599, melanoma X- | |
| | | | | actin, uncharacterized hypothalamus protein | |
| | | | | HARP11 | |
| 885 | 7161 | AI233407 | | | General |
| 886 | 19729 | AA818910 | | | General |
| 887 | 15524 | D13124 | | ATP synthase, H+ transporting, mitochondrial F | b |
| | | | | complex, subunit c (subunit 9) isoform 3, ATP | |
| | | | | synthase, H+ transporting, mitochondrial F | |
| | | | | complex, subunit c (subunit 9), isoform 2, | |
| | | | | ESTs, Moderately similar to AT93 MOUSE ATP | |
| | | | | SYNTHASE LIPID-BINDING PROTEIN P3 | |
| | | | | PRECURSOR [M. musculus], RIKEN cDNA | |
| | | | | 18141M8 gene | |
| 888 | 16679 | AI105268 | | | d |
| 889 | 19184 | AI178025 | | ESTs, Weakly similar to TGIF MOUSE 5′-TG-3′ | General |
| | | | | INTERACTING FACTOR [M. musculus], Homo | |
| | | | | sapiens TGF beta-induced transcription factor 2 | |
| | | | | like mRNA, partial sequence, RIKEN cDNA | |
| | | | | 54345H2 gene, RIKEN cDNA 573599O9 gene, | |
| | | | | TG interacting factor, TGFB-induced factor | |
| | | | | (TALE family homeobox), TGFB-induced factor | |
| | | | | 2 (TALE family homeobox) | |
| 890 | 6015 | AA818158 | | | f |
| 891 | 21659 | AI010584 | | ESTs, Moderately similar to S17182 interferon- | General |
| | | | | induced protein 1-8U [H. sapiens], ESTs, | |
| | | | | Weakly similar to putative haemopoietic | |
| | | | | membrane protein [M. musculus], Human DNA | |
| | | | | sequence from clone RP4-781L3 on | |
| | | | | chromosome 1p34.3-36.11 Contains a | |
| | | | | pseudogene similar to IFITM3 (interferon | |
| | | | | inducedntransmembrane protein 3 (1-8U)), | |
| | | | | STSs and GSSs, RIKEN cDNA 1114C5 gene, | |
| | | | | interferon induced transmembrane protein 3 (1- | |
| | | | | 8U), interferon induced transmembrane protein | |
| | | | | 3-like, interferon-inducible protein 16 | |
| 892 | 18612 | AI228624 | ribosomal protein L29 | EST, Moderately similar to 6S RIBOSOMAL | General |
| | | | | PROTEIN L29 [R. norvegicus], EST, Weakly | |
| | | | | similar to S65784 ribosomal protein L29, | |
| | | | | cytosolic [H. sapiens], ESTs, Highly similar to | |
| | | | | S65784 ribosomal protein L29, cytosolic | |
| | | | | [H. sapiens], ESTs, Weakly similar to 6S | |
| | | | | RIBOSOMAL PROTEIN L29 [M. musculus], | |
| | | | | Human DNA sequence from clone RP4-595K12 | |
| | | | | on chromosome 1p31.2-31.3 Contains a | |
| | | | | pseudogene similar to 6S RPL29 (ribosomal | |
| | | | | protein L29 (cell surface heparin binding protein | |
| | | | | HIP)), a chromosome 1 specific mRNA | |
| | | | | (KIAA499), a novel mRNA (KIAA433), ESTs, | |
| | | | | STSs, GSSs and a CpG Island, ribosomal | |
| | | | | protein L29 | |
| 893 | 19291 | AA963933 | | | General |
| 732 | 21583 | S77900 | | EST, Weakly similar to MOHULP myosin | c, General |
| | | | | regulatory light chain, placental [H. sapiens], | |
| | | | | ESTs, Moderately similar to MOHULP myosin | |
| | | | | regulatory light chain, placental [H. sapiens], | |
| | | | | myosin regulatory light chain, myosin, light | |
| | | | | polypeptide, regulatory, non-sarcomeric (2 kD) | |
| 894 | 7888 | AI233583 | | Homo sapiens, clone MGC: 14993 | h |
| | | | | IMAGE: 361346, mRNA, complete cds, arginyl- | |
| | | | | tRNA synthetase | |
| 895 | 21079 | AA800245 | | Mus musculus adult male stomach cDNA, | f |
| | | | | RIKEN full-length enriched library, | |
| | | | | clone: 22118M5, full insert sequence, expressed | |
| | | | | sequence AI8352, fibronectin type 3 and SPRY | |
| | | | | domain-containing protein, midline 1, midline 1 | |
| | | | | (Opitz/BBB syndrome), midline 2, ring finger | |
| | | | | protein 28 | |
| 896 | 17949 | AI011480 | | HIV-1 Rev binding protein-like, Rev/Rex | g, i |
| | | | | activation domain binding protein-related | |
| 897 | 4089 | AI180251 | | | d |
| 898 | 3823 | AI233147 | | DNA segment, Chr 17, human D6S81E 1, | General |
| | | | | ESTs, Highly similar to 533681 translation | |
| | | | | initiation factor elF-4A.I [H. sapiens], HLA-B | |
| | | | | associated transcript 1, Human clone 23933 | |
| | | | | mRNA sequence, eukaryotic translation | |
| | | | | initiation factor 4A, isoform 1, mitochondrial | |
| | | | | DEAD-box polypeptide 28, nuclear RNA | |
| | | | | helicase, DECD variant of DEAD box family | |
| 899 | 17506 | AI070068 | | growth arrest and DNA-damage-inducible 45 | General |
| | | | | beta, growth arrest and DNA-damage-inducible, | |
| | | | | alpha, growth arrest and DNA-damage- | |
| | | | | inducible, beta | |
| 900 | 21667 | AI013773 | | | General |
| 901 | 8919 | AI103388 | | ADP-ribosylation factor 6, ADP-ribosylation | g, i, |
| | | | | factor domain protein 1, 64 kD, ESTs, | General |
| | | | | Moderately similar to S39543 GTP-binding | |
| | | | | protein - mouse [M. musculus], RIKEN cDNA | |
| | | | | 23175M17 gene, RIKEN cDNA 2919I22 gene, | |
| | | | | hypothetical protein FLJ22595 | |
| 902 | 24064 | AA957480 | | | h |
| 903 | 5902 | AI045871 | | | i |
| 904 | 7913 | AI043849 | | ELL-RELATED RNA POLYMERASE II, | General |
| | | | | ELONGATION FACTOR, ESTs, Highly similar | |
| | | | | to ELL2_HUMAN RNA POLYMERASE II | |
| | | | | ELONGATION FACTOR ELL2 [H. sapiens], | |
| | | | | ESTs, Weakly similar to ELL MOUSE RNA | |
| | | | | POLYMERASE II ELONGATION FACTOR ELL | |
| | | | | [M. musculus], ESTs, Weakly similar to | |
| | | | | ELL2_HUMAN RNA POLYMERASE II | |
| | | | | ELONGATION FACTOR ELL2 [H. sapiens], | |
| | | | | Mus musculus, clone IMAGE: 358397, mRNA, | |
| | | | | partial cds, Mus musculus, clone MGC: 11987 | |
| | | | | IMAGE: 361737, mRNA, complete cds, eleven- | |
| | | | | nineteen lysine-rich leukemia gene, | |
| | | | | hypothetical protein FLJ22637 | |
| 905 | 2980 | AI177761 | | CD68 antigen, Lysosomal associated | b |
| | | | | membrane protein 1 (12 kDa), lysosomal | |
| | | | | membrane glycoprotein 1, similar to S6841 | |
| | | | | (cattle) glucose induced gene | |
| 906 | 8477 | AI179167 | | | General |
| 907 | 11203 | AA892554 | | | General |
| 908 | 22150 | AA893607 | | | General |
| 909 | 10185 | H33426 | | | g |
| 910 | 15626 | X17665 | | EST AI31731, EST, Weakly similar to R3HU16 | f, h, |
| | | | | ribosomal protein S16, cytosolic [H. sapiens], | General |
| | | | | expressed sequence AA42385, ribosomal | |
| | | | | protein S16 | |
| 911 | 2096 | AI233801 | | | h |
| 912 | 1809 | AA946503 | | | f |
| 913 | 23501 | AA955251 | | | General |
| 914 | 23927 | AA957007 | | RIKEN cDNA 615A7 gene, glutathione S- | e |
| | | | | transferase M2 (muscle), glutathione S- | |
| | | | | transferase M5, glutathione 5-transferase, mu | |
| | | | | 5, glutathione S-transferase, mu type 3 (Yb3) | |
| 915 | 6205 | AI104907 | | | e |
| 916 | 8822 | AI070158 | | | i |
| 917 | 5754 | AA850738 | | | General |
| 918 | 21822 | AI228642 | | | h |
| 919 | 19075 | AI236473 | | | General |
| 920 | 2361 | AA875512 | | ESTs, Moderately similar to E Chain E, Human | a |
| | | | | Embryonic Gower Ii Carbonmonoxy | |
| | | | | Hemoglobin {SUB 2-147 [H. sapiens], | |
| | | | | hemoglobin Z, beta-like embryonic chain, | |
| | | | | hemoglobin beta chain complex, hemoglobin, | |
| | | | | epsilon 1 | |
| 921 | 4420 | AI171916 | | | g, General |
| 922 | 3819 | AI176209 | | EST, Weakly similar to 143B_HUMAN 14-3-3 | General |
| | | | | PROTEIN BETA/ALPHA [H. sapiens], ESTs, | |
| | | | | Highly similar to A Chain A. 14-3-3 | |
| | | | | ZetaPHOSPHOPEPTIDE COMPLEX | |
| | | | | [H. sapiens], makorin, ring finger protein, 3, | |
| | | | | stratifin, tyrosine 3-monooxgenase/tryptophan 5 | |
| | | | | monooxgenase activation protein, beta | |
| | | | | polypeptide, tyrosine 3- | |
| | | | | monooxygenase/tryptophan 5-monooxygenase | |
| | | | | activation protein, zeta polypeptide | |
| 923 | 22042 | AA946476 | | | i, General |
| 924 | 3016 | AA818069 | | | a, f |
| 925 | 22487 | AI102578 | | EST, Highly similar to I49523 Mouse primary | General |
| | | | | response gene B94 mRNA, 3′end - mouse | |
| | | | | [M. musculus], Homo sapiens, clone | |
| | | | | MGC: 16332 IMAGE: 3842543, mRNA, complete | |
| | | | | cds, RIKEN cDNA 129I6 gene, RIKEN cDNA | |
| | | | | 1613K19 gene, similar to S. cerevisiae Sec6p | |
| | | | | and R. norvegicus rsec6, tumor necrosis factor, | |
| | | | | alpha-induced protein 2 | |
| 926 | 5507 | AI176584 | | | General |
| 927 | 22077 | AI177099 | | | General |
| 928 | 25386 | L37970 | | | b |
| 929 | 13930 | AA800613 | | zinc finger protein 36, zinc finger protein | General |
| | | | | homologous to Zfp-36 in mouse | |
| 930 | 11484 | AI179612 | | | General |
| 931 | 3417 | AI012337 | | ESTs, Weakly similar to NHPX RAT NHP2/RS6 | g, General |
| | | | | FAMILY PROTEIN YEL26W HOMOLOG | |
| | | | | [R. norvegicus], RIKEN cDNA 24113M7 gene, | |
| | | | | non-histone chromosome protein 2 (S. | |
| | | | | cerevisiae)-like 1, nucleolar protein family A, | |
| | | | | member 2 (H/ACA small nucleolar RNPs), | |
| | | | | sperm specific antigen 1 | |
| 932 | 14677 | AI234620 | | | c |
| 933 | 2134 | AA894212 | | | a |
| 586 | 17203 | AI171844 | | ATP synthase, H+ transporting, mitochondrial | b, e |
| | | | | F1 complex, epsilon subunit, RIKEN cDNA | |
| | | | | 24143G19 gene, expressed sequence AV645 | |
| 934 | 13291 | AI137286 | | | General |
| 935 | 22899 | AA956555 | | | General |
| 936 | 19913 | AI639155 | | | General |
| 937 | 1097 | AF016296 | | | General |
| 938 | 4594 | Y07704 | | | e, General |
| 939 | 22146 | AA893980 | | | General |
| 940 | 13382 | AI101527 | | signal recognition particle 19 kD | a, g |
| 941 | 2399 | AI030247 | | | General |
| 942 | 17644 | AA924036 | | | General |
| 943 | 895 | AI009614 | | | General |
| 944 | 1802 | AA817841 | | | c |
| 945 | 3003 | AA997330 | | | d, General |
| 946 | 9757 | AI170693 | | | c |
| 947 | 23583 | AI234819 | | | General |
| 948 | 8886 | AI233766 | | proteasome (prosome, macropain) activator | f, General |
| | | | | subunit 3 (PA28 gamma; Ki) | |
| 949 | 24185 | AA893708 | | KIAA56 gene product, KIAA144 protein | h |
| 950 | 22929 | AI071578 | | DNA segment, human D4S114, P311 protein | General |
| 951 | 18271 | AA925267 | | | e |
| 952 | 3773 | AA998356 | | B-cell CLL/lymphoma 3, molecule possessing | General |
| | | | | ankyrin repeats induced by lipopolysaccharide | |
| | | | | (MAIL), homolog of mouse, nuclear factor of | |
| | | | | kappa light polypeptide gene enhancer in B- | |
| | | | | cells inhibitor, epsilon | |
| 953 | 18108 | X62528 | | EST, Highly similar to 16527A RNase inhibitor | f |
| | | | | [H. sapiens], EST, Moderately similar to RINI | |
| | | | | RAT RIBONUCLEASE INHIBITOR | |
| | | | | [R. norvegicus], ESTs, Weakly similar to | |
| | | | | AF7418 1 MATER protein [M. musculus], | |
| | | | | expressed sequence AW546468, expressed | |
| | | | | sequence C835, ribonuclease/angiogenin | |
| | | | | inhibitor | |
| 954 | 6176 | AA819657 | | | a |
| 955 | 14250 | AA799729 | phosphodiesterase 4B, | endothelin receptor type A, phosphodiesterase | General |
| | | | cAMP specific, | 1B, Ca2+-calmodulin dependent, 63 kDa, | |
| | | | phosphodiesterase 4B, | phosphodiesterase 4B, cAMP specific, | |
| | | | cAMP-specific (dunce | phosphodiesterase 4B, cAMP-specific (dunce | |
| | | | (Drosophila)-homolog | (Drosophila)-homolog phosphodiesterase E4), | |
| | | | phosphodiesterase E4) | phosphodiesterase 8A, phosphodiesterase 8B, | |
| | | | | phosphodiesterase 9A | |
| 956 | 22948 | AI105421 | | | d |
| 957 | 10991 | AI104342 | | | b |
| 958 | 11563 | AI102560 | | | a |
| 959 | 25669 | X07314 | | | a, b |
| 960 | 9277 | AI072424 | | | i |
| 961 | 13467 | AI138034 | | UDP-glucose ceramide glucosyltransferase | General |
| 962 | 11516 | AI103962 | | | h |
| 963 | 20110 | AF071204 | | KIT ligand, kit ligand, stem cell factor KL-2 | b |
| | | | | precursor | |
| 964 | 19320 | AA891666 | melanoma antigen, family | Homo sapiens, Similar to neurofilament, heavy | i |
| | | | D, 1 | polypeptide (2 kD), clone MGC: 271 | |
| | | | | IMAGE: 463424, mRNA, complete cds, MAGE- | |
| | | | | E1 protein, MAGEF1 protein, Neurofilament, | |
| | | | | heavy polypeptide, RIKEN cDNA 2117K23 | |
| | | | | gene, RIKEN cDNA 383417A13 gene, General | |
| | | | | transcription factor IIH, polypeptide 1 (62 kD | |
| | | | | subunit), melanoma antigen, family D, 1, | |
| | | | | melanoma antigen, family D, 2, necdin, | |
| | | | | neurofilament, heavy polypeptide, | |
| | | | | neurofilament, heavy polypeptide (2 kD) | |
| 965 | 13029 | AI179391 | | | d, General |
| 966 | 10348 | AI069934 | | | e |
| 967 | 8495 | AI059971 | | Homo sapiens (clone NCD18) tumor necrosis | d, g, i, |
| | | | | factor receptor related protein mRNA, complete | General |
| | | | | exon and repeat region, lymphotoxin B | |
| | | | | receptor, lymphotoxin beta receptor (TNFR | |
| | | | | superfamily, member 3), tumor necrosis factor | |
| | | | | receptor superfamily, member 8 | |
| 968 | 14968 | K02815 | | ESTs, Moderately similar to S4363 class II | General |
| | | | | histocompatibility antigen RT1-B alpha chain | |
| | | | | precursor - rat [R. norvegicus], histocompatibility | |
| | | | | 2, O region alpha locus, major | |
| | | | | histocompatibility complex, class II, DO alpha | |
| 969 | 19358 | AI009675 | | | a, b, |
| | | | | | General |
| 970 | 4031 | AI176989 | | | a |
| 971 | 19005 | AI175875 | | | d |
| 972 | 21661 | AI176479 | | ESTs, Moderately similar to S17182 interferon- | b, f, |
| | | | | induced protein 1-8U [H. sapiens], ESTs, | General |
| | | | | Weakly similar to putative haemopoietic | |
| | | | | membrane protein [M. musculus], Human DNA | |
| | | | | sequence from clone RP4-781L3 on | |
| | | | | chromosome 1p34.3-36.11 Contains a | |
| | | | | pseudogene similar to IFITM3 (interferon | |
| | | | | inducedntransmembrane protein 3 (1-8U)), | |
| | | | | STSs and GSSs, RIKEN cDNA 1114C5 gene, | |
| | | | | interferon induced transmembrane protein 3 (1-8U), | |
| | | | | interferon induced transmembrane protein | |
| | | | | 3-like, interferon-inducible protein 16 | |
| 973 | 13596 | U90261 | | Arg/Abl-interacting protein ArgBP2, EST, | c, h |
| | | | | Weakly similar to JC5583 85K SH3 domain- | |
| | | | | containing proline-rich protein - mouse | |
| | | | | [M. musculus], ESTs, Moderately similar to | |
| | | | | T1418 SH3-containing protein p415 - rat | |
| | | | | [R. norvegicus], Homo sapiens, clone MGC: 212 | |
| | | | | IMAGE: 36777, mRNA, complete cds, Mus | |
| | | | | musculus betaPix-d mRNA, complete cds, | |
| | | | | Pak3 binding protein, RIKEN cDNA 1738J6 | |
| | | | | gene, RIKEN cDNA 2123O3 gene, SH3-domain | |
| | | | | kinase binding protein 1 | |
| 974 | 11724 | AI102812 | | | a, b, |
| | | | | | General |
| 975 | 2042 | AI101921 | | | General |
| 976 | 25632 | U75405 | | | e |
| 977 | 10227 | AI102248 | | | h, General |
| 978 | 7224 | AI009820 | | | h |
| 979 | 2131 | AI171091 | | | h |
| 980 | 20055 | AI230762 | | ESTs, Weakly similar to CALPONIN, ACIDIC | General |
| | | | | ISOFORM [R. norvegicus], Human DNA | |
| | | | | sequence from clone RP1-9E21 on | |
| | | | | chromosome 1q24-25 Contains a pseudogene | |
| | | | | similar to calponin, a novel mRNA (clone | |
| | | | | 25151), STSs, GSSs and a CpG Island, RIKEN | |
| | | | | cDNA 1614M3 gene, acidic calponin, calponin | |
| | | | | 2, calponin 3, acidic | |
| 981 | 18897 | AA875207 | hemoglobin beta chain | | d |
| | | | complex, hemoglobin, | | |
| | | | beta | | |
| 982 | 18468 | AA859966 | | Homo sapiens cDNA FLJ14666 fis, clone | General |
| | | | | NT2RP23, weakly similar to TUMOR | |
| | | | | NECROSIS FACTOR, ALPHA-INDUCED | |
| | | | | PROTEIN 1, Homo sapiens polymerase delta- | |
| | | | | interacting protein 1 mRNA, complete cds, | |
| | | | | MSTP28 protein, tumor necrosis factor, alpha- | |
| | | | | induced protein 1 (endothelial) | |
| 983 | 22820 | AA848315 | | GMPR2 for guanosine monophosphate | General |
| | | | | reductase isolog, IMP (inosine monophosphate) | |
| | | | | dehydrogenase 2, RIKEN cDNA 2314P21 | |
| | | | | gene, RIKEN cDNA 573544D12 gene, | |
| | | | | expressed sequence AA95985, guanosine | |
| | | | | monophosphate reductase, inosine 5′- | |
| | | | | phosphate dehydrogenase 2 | |
| 984 | 17901 | AA944355 | | | General |
| 985 | 8305 | AI111695 | | | a |
| 986 | 14842 | AI237724 | | | General |
| | 26167 | AI044866 | | | General |
| 987 | 10097 | AI639425 | | | f, General |
| 988 | 6861 | AI176970 | | | h |
| 989 | 18859 | AA799467 | | | g |
| 990 | 21510 | AA851620 | | | General |
| 991 | 3561 | AB020504 | | | General |
| 992 | 22898 | AA901107 | | | General |
| 993 | 14668 | AI012185 | | | c |
| 994 | 23035 | AA945712 | | | d |
| 995 | 6330 | AI170426 | | | b |
| 996 | 3808 | AI008643 | | DnaJ (Hsp4) homolog, subfamily B, member 1, | General |
| | | | | DnaJ (Hsp4) homolog, subfamily B, member | |
| | | | | 12, DnaJ (Hsp4) homolog, subfamily B, | |
| | | | | member 4, DnaJ (Hsp4) homolog, subfamily B, | |
| | | | | member 5, ESTs, Weakly similar to | |
| | | | | HS4L_HUMAN HEAT SHOCK 4 KDA | |
| | | | | PROTEIN 1 HOMOLOG [H. sapiens], RIKEN | |
| | | | | cDNA 1729A2 gene, RIKEN cDNA 2136G19 | |
| | | | | gene | |
| 997 | 10334 | AI060295 | | RAB5B, member RAS oncogene family, RIKEN | a |
| | | | | cDNA 3732413A17 gene, expressed sequence | |
| | | | | AI663973 | |
| 998 | 22762 | D89730 | | EGF-containing fibulin-like extracellular matrix | c, General |
| | | | | protein 1, EGF-containing fibulin-like | |
| | | | | extracellular matrix protein 2, EST, Weakly | |
| | | | | similar to FBL3_RAT EGF-CONTAINING | |
| | | | | FIBULIN-LIKE EXTRACELLULAR MATRIX | |
| | | | | PROTEIN 1 PRECURSOR (FIBULIN-3) (FIBL- | |
| | | | | 3) (T16 PROTEIN) [R. norvegicus], ESTs, | |
| | | | | Weakly similar to FBL3_RAT EGF- | |
| | | | | CONTAINING FIBULIN-LIKE | |
| | | | | EXTRACELLULAR MATRIX PROTEIN 1 | |
| | | | | PRECURSOR (FIBULIN-3) (FIBL-3) (T16 | |
| | | | | PROTEIN) [R. norvegicus], epidermal growth | |
| | | | | factor-containing fibulin-like extracellular matrix | |
| | | | | protein 1, epidermal growth factor-containing | |
| | | | | fibulin-like extracellular matrix protein 2 | |
| 999 | 15837 | AI010411 | | ESTs, Highly similar to 2819A set gene | g |
| | | | | [R. norvegicus], ESTs, Highly similar to I59377 | |
| | | | | template activating factor-I, splice form alpha | |
| | | | | [H. sapiens], ESTs, Highly similar to | |
| | | | | SET_HUMAN SET PROTEIN [H. sapiens], | |
| | | | | Human DNA sequence from PAC 3P2 on | |
| | | | | chromosome Xq21.1-Xq21.3. Contains set | |
| | | | | pseudogene, ESTs and STS, KIAA175 protein, | |
| | | | | NADH dehydrogenase (ubiquinone) 1, | |
| | | | | subcomplex unknown, 2 (14.5 kD, B14.5b), SET | |
| | | | | translocation, SET translocation (myeloid | |
| | | | | leukemia-associated) | |
| 1000 | 4169 | AI009669 | | RIKEN cDNA 229H22 gene, membrane- | General |
| | | | | spanning 4-domains, subfamily A, member 1, | |
| | | | | membrane-spanning 4-domains, subfamily A, | |
| | | | | member 11, membrane-spanning 4-domains, | |
| | | | | subfamily A, member 12 4-domains, subfamily | |
| | | | | A, member 7, membrane-spanning 4-domains, | |
| | | | | subfamily A, member 2 (Fc fragment of IgE, | |
| | | | | high affinity I, receptor for; beta polypeptide), | |
| | | | | membrane-spanning 4-domains, subfamily A, | |
| | | | | member 3 (hematopoietic cell-specific), | |
| | | | | membrane-spanning 4-domains, subfamily A, | |
| | | | | member 4A, membrane-spanning 4-domains, | |
| | | | | subfamily A, member 5, membrane-spanning 4- | |
| | | | | domains, subfamily A, member 6A, membrane- | |
| | | | | spanning 4-domains, subfamily A, member 8, | |
| | | | | testes development-related NYD-SP21 | |
| 1001 | 22596 | AA955298 | | | General |
| 1002 | 11483 | AF020618 | | | General |
| 1003 | 17540 | AA955914 | | EST, Highly similar to FBRL MOUSE | General |
| | | | | FIBRILLARIN [M. musculus], EST, Weakly | |
| | | | | similar to A38712 fibrillarin [H. sapiens], ESTs, | |
| | | | | Highly similar to A38712 fibrillarin [H. sapiens], | |
| | | | | ESTs, Weakly similar to FBRL MOUSE | |
| | | | | FIBRILLARIN [M. musculus], expressed | |
| | | | | sequence AL22665, fibrillarin | |
| 1004 | 26030 | M34331 | | | e |
| 1005 | 15569 | Z78279 | collagen, type I, alpha 1, | EST, Highly similar to CA1B_HUMAN | c, e |
| | | | procollagen, type I, alpha 1 | COLLAGEN ALPHA 1(XI) CHAIN | |
| | | | | PRECURSOR [H. sapiens], EST, Weakly | |
| | | | | similar to CA11_HUMAN COLLAGEN ALPHA | |
| | | | | 1(I) CHAIN PRECURSOR [H. sapiens], EST, | |
| | | | | Weakly similar to CA25_HUMAN COLLAGEN | |
| | | | | ALPHA 2(V) CHAIN PRECURSO [H. sapiens], | |
| | | | | EST, Weakly similar to CGHU1S collagen | |
| | | | | alpha 1(I) chain precursor [H. sapiens], ESTs, | |
| | | | | Weakly similar to CA11_HUMAN COLLAGEN | |
| | | | | ALPHA 1(I) CHAIN PRECURSOR [H. sapiens], | |
| | | | | Homo sapiens cDNA FLJ2845 fis, clone | |
| | | | | ADKA191, Homo sapiens mRNA for putative | |
| | | | | emu2 protein, collagen, type I, alpha 1 | |
| 1006 | 22378 | AA944212 | | | General |
| 1007 | 23155 | AA926016 | | DNA segment, Chr 7, ERATO Doi 486, | i |
| | | | | expressed, EST, Weakly similar to S37583 | |
| | | | | RING finger protein rfp - mouse [M. musculus], | |
| | | | | ESTs, Weakly similar to JE343 terf protein - rat | |
| | | | | [R. norvegicus], RING finger protein terf, | |
| | | | | expressed sequence AI894158, expressed | |
| | | | | sequence AW46487, hypothetical protein | |
| | | | | FLJ22638, hypothetical protein MGC16175, ret | |
| | | | | finger protein, ret finger protein-like 3 antisense, | |
| | | | | tripartite motif protein 11, tripartite motif protein | |
| | | | | 17 | |
| 1008 | 4832 | AA800190 | | ESTs, Moderately similar to 17149A glycogen | General |
| | | | | phosphorylase [H. sapiens], ESTs, Weakly | |
| | | | | similar to 17149A glycogen phosphorylase | |
| | | | | [H. sapiens], phosphorylase, glycogen; brain | |
| 1009 | 18774 | AI010279 | | | a, i |
| 1010 | 21798 | AA926365 | | CGI-69 protein, EST, Moderately similar to | b |
| | | | | T43493 hypothetical protein DKFZp434C119.1 | |
| | | | | [H. sapiens], mitochondrial carrier family protein, | |
| | | | | mitochondrial solute carrier | |
| 1011 | 3392 | AA998205 | | | d |
| 1012 | 9796 | AI071990 | | | General |
| 1013 | 16314 | AA859348 | | | General |
| 1014 | 2757 | AA997851 | | | c, General |
| 1015 | 4636 | AA899491 | | | i |
| 1016 | 21644 | AI179609 | | | General |
| 480 | 11727 | AA849518 | | | d |
| 1017 | 345 | AI059916 | | Signal transducer and activator of transcription | f |
| | | | | 1, signal transducer and activator of | |
| | | | | transcription 1, signal transducer and activator | |
| | | | | of transcription 1, 91 kD, signal transducer and | |
| | | | | activator of transcription 3, signal transducer | |
| | | | | and activator of transcription 3 (acute-phase | |
| | | | | response factor) | |
| 1018 | 22773 | AA859885 | | | c |
| 1019 | 2767 | AA963201 | | | General |
| 1020 | 22036 | AI070141 | | | General |
| 1021 | 8580 | AI060314 | | | c |
| 1022 | 9362 | AI102706 | | | General |
| 1023 | 10659 | AI103059 | | | General |
| 1024 | 16549 | AA892270 | | | a |
| 1025 | 2254 | AI235325 | | | h, General |
| 1026 | 12366 | AI232088 | | | General |
| 1027 | 20897 | AI175812 | | coatomer protein complex, subunit alpha, | General |
| | | | | embryonic ectoderm development | |
| 1028 | 13270 | AI228760 | | | d |
| 1029 | 12031 | AA893860 | | ESTs, Highly similar to YSHUT threonine- | General |
| | | | | tRNA ligase [H. sapiens], ESTs, Moderately | |
| | | | | similar to YSHUT threonine- tRNA ligase | |
| | | | | [H. sapiens], hypothetical protein FLJ12528, | |
| | | | | threonyl-tRNA synthetase | |
| 1030 | 16859 | AI236753 | | | General |
| 1031 | 6982 | AI170793 | | Down syndrome cell adhesion molecule, | General |
| | | | | RIKEN cDNA 212A2 gene, deleted in colorectal | |
| | | | | carcinoma, myopalladin, palladin, putative | |
| | | | | neuronal cell adhesion molecule, titin | |
| | | | | immunoglobulin domain protein (myotilin) | |
| 1032 | 15518 | AI072945 | | | General |
| 1033 | 8612 | AI058527 | | | a, b, |
| | | | | | General |
| 1034 | 14289 | AA891838 | | | b |
| 580 | 22248 | AA943537 | | ajuba, expressed sequence AI48116, | General |
| | | | | expressed sequence R75157, zyxin | |
| 1035 | 12303 | AA944786 | | | d |
| 1036 | 17632 | AI102472 | | | c |
| 1037 | 19722 | AI013508 | | | General |
| 1038 | 15051 | AI236332 | | ESTs, Weakly similar to ATDA_HUMAN | General |
| | | | | DIAMINE ACETYLTRANSFERASE | |
| | | | | [H. sapiens], RIKEN cDNA 26116A3 gene, | |
| | | | | RIKEN cDNA 49344K22 gene, | |
| | | | | spermidine/spermine N1-acetyl transferase, | |
| | | | | spermidine/spermine N1-acetyltransferase | |
| 1039 | 7273 | AI234712 | | | f |
| 1040 | 15879 | AI228313 | | | General |
| 1041 | 10020 | AI045632 | | | f, General |
| 1042 | 22432 | AI171263 | | EST, Highly similar to FBRL MOUSE | g, General |
| | | | | FIBRILLARIN [M. musculus], EST, Weakly | |
| | | | | similar to A38712 fibrillarin [H. sapiens], ESTs, | |
| | | | | Highly similar to A38712 fibrillarin [H. sapiens], | |
| | | | | ESTs, Weakly similar to FBRL MOUSE | |
| | | | | FIBRILLARIN [M. musculus], expressed | |
| | | | | sequence AL22665, fibrillarin | |
| 1043 | 25705 | X59375 | | | General |
| 1044 | 4696 | AI237009 | | | c |
| 1045 | 18673 | AA849028 | | EST, Weakly similar to SNHUC8 multicatalytic | d |
| | | | | endopeptidase complex [H. sapiens], ESTs, | |
| | | | | Highly similar to PRC8 MOUSE | |
| | | | | PROTEASOME COMPONENT C8 | |
| | | | | [M. musculus], ESTs, Weakly similar to | |
| | | | | SNHUC8 multicatalytic endopeptidase complex | |
| | | | | [H. sapiens], proteasome (prosome, macropain) | |
| | | | | subunit, alpha type 3, proteasome (prosome, | |
| | | | | macropain) subunit, alpha type, 3 | |
| 1046 | 13166 | AI178736 | | | b |
| 1047 | 24128 | AA849766 | | DKFZP586E1519 protein | c |
| 1048 | 24042 | AI230002 | | | General |
| 1049 | 9339 | AI101160 | | | General |
| 1050 | 15190 | AI102562 | | EST, Moderately similar to Cd-7 Metallothionein | General |
| | | | | 2 [H. sapiens], EST, Moderately similar to | |
| | | | | SMHU1E metallothionein 1E [H. sapiens] | General |
| 1051 | 3433 | AI030339 | | | General |
| 1052 | 4459 | AA891721 | | | General |
| 1053 | 19012 | AI172056 | | | General |
| 1054 | 2802 | D85435 | | | c |
| 1055 | 14763 | AA944481 | | ESTs, Highly similar to AGP2_RAT | General |
| | | | | ANGIOPOIETIN-2 (ANG-2) [R. norvegicus], | |
| | | | | angiopoietin 1, angiopoietin 2, angiopoietin-like | |
| | | | | 3, angiopoietin-like 4, angiopoietin-like factor | |
| 1056 | 9583 | AI071185 | | | d, General |
| 1057 | 10394 | AA943564 | | | General |
| 1058 | 16821 | AA999042 | | | General |
| 1059 | 3718 | AI229643 | | | General |
| 1060 | 8022 | AI058357 | | | General |
| 1061 | 19840 | AA899197 | | EST, Moderately similar to JH148 nucleolin - | g, General |
| | | | | rat [R. norvegicus], EST, Weakly similar to | |
| | | | | NUCL_HUMAN NUCLEOLIN [H. sapiens], | |
| | | | | ESTs, Highly similar to FUS_HUMAN RNA- | |
| | | | | BINDING PROTEIN FUS [H. sapiens], ESTs, | |
| | | | | Highly similar to PAB1_HUMAN | |
| | | | | POLYADENYLATE-BINDING PROTEIN 1 | |
| | | | | [H. sapiens], ESTs, Moderately similar to | |
| | | | | PAB1_HUMAN POLYADENYLATE-BINDING | |
| | | | | PROTEIN 1 [H. sapiens], ESTs, Moderately | |
| | | | | similar to POLYADENYLATE-BINDING | |
| | | | | PROTEIN 1 [M. musculus], Mus musculus adult | |
| | | | | male testis cDNA, RIKEN full-length enriched | |
| | | | | library, clone: 493347N23, full insert sequence, | |
| | | | | RIKEN cDNA 493272K14 gene, fusion, derived | |
| | | | | from t(12;16) malignant liposarcoma, poly A | |
| | | | | binding protein, cytoplasmic 1, poly(A)-binding | |
| | | | | protein, cytoplasmic 3, poly(A)-binding protein, | |
| | | | | cytoplasmic, pseudogene 2 | |
| 1062 | 15215 | AI060260 | | | d |
| 1063 | 8053 | AI175033 | | | General |
| 1064 | 12086 | AA801116 | | | c |
| 1065 | 21447 | AI009608 | | | d |
| 1066 | 4232 | AI012958 | | | General |
| 1067 | 12978 | X96437 | | immediate early response 3 | General |
| 1068 | 1031 | AA892863 | | | d |
| 1069 | 18682 | AI059499 | | Homo sapiens, Similar to chaperonin containing | h |
| | | | | TCP1, subunit 8 (theta), clone MGC: 1224 | |
| | | | | IMAGE: 393332, mRNA, complete cds, T- | |
| | | | | complex 1, chaperonin containing TCP1, | |
| | | | | subunit 8 (theta), chaperonin subunit 8 (theta), | |
| | | | | expressed sequence AI528772, potassium | |
| | | | | large conductance calcium-activated channel, | |
| | | | | subfamily M, beta member 3-like, t-complex 1, t- | |
| | | | | complex protein 1 | |
| 1070 | 25806 | E00898 | | | g |
| 1071 | 4432 | AI101851 | | | d |
| 1072 | 2997 | AI030545 | | | i, General |
| 1073 | 6936 | AI010593 | | | d |
| 1074 | 11684 | AI010917 | | | g, General |
| 1075 | 804 | AI169756 | | Gene 33/Mig-6, RIKEN cDNA 132F13 gene | General |
| 1076 | 15192 | AI101099 | | ESTs, Moderately similar to AF78844 1 hqp376 | General |
| | | | | protein [H. sapiens], expressed sequence | |
| | | | | AA49533 | |
| 1077 | 19145 | AA964613 | | | f |
| 1078 | 3887 | AA893307 | | | a |
| 1079 | 13539 | AI177280 | | | General |
| 1080 | 5258 | AA926089 | | DKFZP564B769 protein, ESTs, Weakly similar | d |
| | | | | to T12483 hypothetical protein | |
| | | | | DKFZp564B769.1 [H. sapiens], KIAA164 protein | |
| 1081 | 17380 | AA799612 | | ESTs, Highly similar to S17516 hypothetical | h |
| | | | | protein [H. sapiens], ESTs, Highly similar to | |
| | | | | UBIQUITIN-CONJUGATING ENZYME E2-17 | |
| | | | | KD [R. norvegicus], ESTs, Moderately similar to | |
| | | | | UBC2_HUMAN UBIQUITIN-CONJUGATING | |
| | | | | ENZYME E2-17 KD [H. sapiens], ESTs, Weakly | |
| | | | | similar to UBIQUITIN-CONJUGATING | |
| | | | | ENZYME E2-17 KD [R. norvegicus], RIKEN | |
| | | | | cDNA 26131N2 gene, ubiquitin-conjugating | |
| | | | | enzyme E2A (RAD6 homolog), ubiquitin- | |
| | | | | conjugating enzyme E2B (RAD6 homolog), | |
| | | | | ubiquitin-conjugating enzyme E2B (RAD6 | |
| | | | | homology), ubiquitin-conjugating enzyme E2C, | |
| | | | | ubiquitin-conjugating enzyme E2I, ubiquitin- | |
| | | | | conjugating enzyme E2I (homologous to yeast | |
| | | | | UBC9) | |
| 544 | 11530 | AA799773 | | EST, Moderately similar to ABP2_HUMAN | i |
| | | | | ENDOTHELIAL ACTIN-BINDING PROTEIN | |
| | | | | [H. sapiens], ESTs, Moderately similar to | |
| | | | | ABP2_HUMAN ENDOTHELIAL ACTIN- | |
| | | | | BINDING PROTEIN [H. sapiens], filamin A, | |
| | | | | alpha (actin-binding protein-28), filamin B, beta | |
| | | | | (actin-binding protein-278) | |
| 1082 | 8541 | AI112310 | | DKFZP564O92 protein, disrupter of silencing 1 | d |
| 1083 | 15876 | AA892582 | | EST, Highly similar to 6S RIBOSOMAL | e, h |
| | | | | PROTEIN L8 [R. norvegicus], EST, Moderately | |
| | | | | similar to 6S RIBOSOMAL PROTEIN L8 | |
| | | | | [R. norvegicus], EST, Weakly similar to JN923 | |
| | | | | ribosomal protein L8, cytosolic [H. sapiens], | |
| | | | | ESTs, Highly similar to 6S RIBOSOMAL | |
| | | | | PROTEIN L8 [R. norvegicus], ESTs, Highly | |
| | | | | similar to RL8_HUMAN 6S RIBOSOMAL | |
| | | | | PROTEIN L [M. musculus], ESTs, Moderately | |
| | | | | similar to RL8_HUMAN 6S RIBOSOMAL | |
| | | | | PROTEIN L [M. musculus], expressed | |
| | | | | sequence AL2498, ribosomal protein L8 | |
| 1084 | 7806 | AA818421 | | | General |
| 1085 | 19011 | AI102618 | | | General |
| 1086 | 8938 | AI070590 | | | General |
| 1087 | 13266 | AI172326 | | | General |
| 1088 | 22765 | AI176265 | | | General |
| 1089 | 22196 | U21719 | | | General |
| 1090 | 6789 | AA998207 | | | i, General |
| 1091 | 1287 | AI172299 | | | f, General |
| 1092 | 13985 | AA819429 | | | e |
| 1093 | 5887 | AI179099 | | biotinidase, vanin 1, vanin 2, vanin 3 | b |
| 1094 | 15189 | M11794 | | | General |
| 1095 | 657 | AI010262 | | colony stimulating factor 2 receptor, beta 1, low- | General |
| | | | | affinity (granulocyte-macrophage), interleukin 4 | |
| | | | | receptor, interleukin 4 receptor, alpha | |
| 1096 | 14554 | U48828 | | | f, i, |
| | | | | | General |
| 1097 | 23096 | AA924352 | | ESTs, Moderately similar to acid phosphatase | a, b |
| | | | | [H. sapiens], LPAP for lysophosphatidic acid | |
| | | | | phosphatase, acid phosphatase 6, | |
| | | | | lysophosphatidic, expressed sequence | |
| | | | | AU22842 | |
| 1098 | 7120 | AI012393 | | ESTs, Weakly similar to JE343 terf protein - rat | a, b |
| | | | | [R. norvegicus], RIKEN cDNA 4921519C19 | |
| | | | | gene, RING finger protein terf, butyrophilin, | |
| | | | | subfamily 1, member A1, expressed sequence | |
| | | | | AA41499, expressed sequence AI894158, | |
| | | | | hypothetical protein FLJ22638, hypothetical | |
| | | | | protein MGC16175, ret finger protein, ret finger | |
| | | | | protein-like 3 antisense, tripartite motif protein | |
| | | | | 11, tripartite motif protein 17 | |
| 1099 | 13033 | AI113076 | | | c |
| 1100 | 12745 | AI011799 | | | a, General |
| 1101 | 8759 | AI237646 | | | General |
| 950 | 22930 | AI071578 | | DNA segment, human D4S114, P311 protein | e |
| 1102 | 4490 | AA851184 | | cathepsin Z | General |
| 1103 | 600 | AA800222 | | | c, e |
| 779 | 1687 | AI179971 | hemoglobin alpha, adult | EST, Moderately similar to HART1 hemoglobin | a, b, |
| | | | chain 2, hemoglobin, | alpha-1 chain - rat [R. norvegicus], ESTs, | General |
| | | | alpha 1 | Moderately similar to HART1 hemoglobin alpha- | |
| | | | | 1 chain - rat [R. norvegicus], RIKEN cDNA | |
| | | | | 25142H12 gene, hemoglobin alpha, adult chain | |
| | | | | 1, hemoglobin, alpha 1, hemoglobin, alpha 2 | |
| 1104 | 23139 | AI180040 | | | d, f |
| 1105 | 11876 | AI009321 | | | General |
| 1106 | 13568 | AA800169 | | | General |
| 1107 | 25671 | X07686 | | | e |
| 1108 | 2410 | AA964589 | | | General |
| 1109 | 23230 | AI236146 | | | d |
| 766 | 15134 | D10706 | | EST, Moderately similar to I3859 hypothetical | c |
| | | | | protein [H. sapiens], EST, Weakly similar to | |
| | | | | I3859 hypothetical protein [H. sapiens], ESTs, | |
| | | | | Weakly similar to ORNITHINE | |
| | | | | DECARBOXYLASE ANTIZYME [R. norvegicus], | |
| | | | | ornithine decarboxylase antizyme, ornithine | |
| | | | | decarboxylase antizyme 1, ornithine | |
| | | | | decarboxylase antizyme 2 | |
| 1110 | 6166 | AI136516 | | | General |
| 1111 | 12066 | AA800566 | | | c |
| 684 | 17947 | AA944581 | | HIV-1 Rev binding protein-like, Rev/Rex | i |
| | | | | activation domain binding protein-related | |
| 371 | 19256 | M15562 | | expressed sequence AI323765, | f, General |
| | | | | histocompatibility 2, class II antigen E alpha, | |
| | | | | major histocompatibility complex, class II, DP | |
| | | | | alpha 2 (pseudogene), major histocompatibility | |
| | | | | complex, class II, DR alpha | |
| 1112 | 23210 | H31420 | | | h |
| 1113 | 5844 | AA944367 | | N-acetylglucosamine-phosphate mutase | General |
| 1114 | 2992 | AI171776 | | | General |
| 1115 | 8515 | AA849917 | | | a, b |
| 1116 | 21974 | AA945769 | | ESTs, Weakly similar to T43483 translation | a |
| | | | | initiation factor IF-2 homolog [H. sapiens], | |
| | | | | hypothetical protein MGC536, novel putative | |
| | | | | protein similar to YIL91C yeast hypothetical 84 | |
| | | | | kD protein from SGA1-KTR7, translation | |
| | | | | initiation factor IF2 | |
| 1117 | 11896 | AA997845 | | dual-specificity tyrosine-(Y)-phosphorylation | a |
| | | | | regulated kinase 1B, dual-specificity tyrosine- | |
| | | | | (Y)-phosphorylation regulated kinase 3, kinase | |
| | | | | interacting with leukemia-associated gene | |
| | | | | (stathmin) | |
| 1118 | 10820 | AI009411 | | EST, Moderately similar to R3RT3 ribosomal | e, General |
| | | | | protein S3 - rat [R. norvegicus], EST, Weakly | |
| | | | | similar to R3RT3 ribosomal protein S3 - rat | |
| | | | | [R. norvegicus], hypothetical protein FLJ2359, | |
| | | | | ribosomal protein S3 | |
| 1119 | 6532 | AI234105 | | | General |
| 1120 | 6274 | AA819206 | | | General |
| 1121 | 23099 | AI112365 | | RIKEN cDNA 2112C16 gene, hypothetical | General |
| | | | | protein FLJ1292, mago-nashi (Drosophila) | |
| | | | | homolog, proliferation-associated, mago-nashi | |
| | | | | homolog, proliferation-associated (Drosophila) | |
| 1122 | 12898 | AI105049 | | | General |
| 1123 | 22011 | AI176212 | | | General |
| 1124 | 14693 | AI234830 | | | General |
| 1125 | 12107 | AI172460 | | | d |
| 1126 | 3799 | AF002281 | | ESTs, Weakly similar to LIM PROTEIN CLP36 | f, General |
| | | | | [R. norvegicus], PDZ and LIM domain 1 (elfin), | |
| | | | | PDZ and LIM domain 3, PDZ-LIM protein | |
| | | | | mystique, RIKEN cDNA 1113B1 gene, Rattus | |
| | | | | norvegicus LIM-domain protein LMP-1 mRNA, | |
| | | | | complete cds, Z-band alternatively spliced PDZ- | |
| | | | | motif, alpha-actinin-2-associated LIM protein, | |
| | | | | reversion induced LIM gene | |
| 1127 | 14955 | AA945750 | | | d |
| 1128 | 14862 | AI236048 | | | f |
| 1129 | 9317 | AI029174 | | | h |
| 1130 | 24028 | AI180239 | | | General |
| 1131 | 16081 | AI179610 | heme oxygenase | heme oxygenase (decycling) 1 | General |
| | | | (decycling) 1 | | |
| 1132 | 25918 | AI639204 | | | e |
| 1133 | 15042 | AI179422 | | | General |
| 1134 | 11242 | AI179260 | | | General |
| 1135 | 18018 | D12771 | solute carrier family 25 | EST, Moderately similar to S3894 ADP, ATP | i |
| | | | (mitochondrial carrier; | carrier protein T3 [H. sapiens], EST, Weakly | |
| | | | adenine nucleotide | similar to A29132 ADP, ATP carrier protein T2 | |
| | | | translocator), member 5 | [H. sapiens], ESTs, Moderately similar to | |
| | | | | ADT2_HUMAN ADP, ATP CARRIER PROTEIN, | |
| | | | | FIBROBLAST ISOFORM [H. sapiens], Mus | |
| | | | | musculus, Similar to CG4995 gene product, | |
| | | | | clone MGC: 7958 IMAGE: 358457, mRNA, | |
| | | | | complete cds, RIKEN cDNA 136L1 gene, | |
| | | | | RIKEN cDNA 493443G12 gene, folate | |
| | | | | transporter/carrier, solute carrier family 25 | |
| | | | | (mitochondrial carrier; adenine nucleotide | |
| | | | | translocator), member 5, solute carrier family | |
| | | | | 25 (mitochondrial carrier; adenine nucleotide | |
| | | | | translocator), member 6 | |
| 1136 | 6655 | AA850688 | | | f |
| 1137 | 10182 | AI176185 | FBJ osteosarcoma | | General |
| | | | oncogene, v-fos FBJ | | |
| | | | murine osteosarcoma | | |
| | | | viral oncogene homolog | | |
| 1138 | 20990 | AI176509 | | protein kinase C, nu | i |
| 1139 | 12118 | AA892775 | lysozyme, lysozyme | EST, Weakly similar to A Chain A, Mutant | g |
| | | | (renal amyloidosis) | Human Lysozyme With Foreign N-Terminal | |
| | | | | Residues {SUB 18-148 [H. sapiens], EST, | |
| | | | | Weakly similar to LYC1 RAT LYSOZYME C, | |
| | | | | TYPE 1 PRECURSOR [R. norvegicus], ESTs, | |
| | | | | Weakly similar to A Chain A, Mutant Human | |
| | | | | Lysozyme With Foreign N-Terminal Residues | |
| | | | | {SUB 18-148 [H. sapiens], RIKEN cDNA | |
| | | | | 1738F2 gene, RIKEN cDNA 9533J23 gene, | |
| | | | | lysozyme, lysozyme (renal amyloidosis), | |
| | | | | lysozyme homolog | |
| 1140 | 4355 | AI103410 | | | General |

TABLE 4

| Compound | Time (hrs) | Model Code |
|---|---|---|
| Ifosfamide | 48, 144 | a |
| Cyclophosphamide | 48, 192 | b |
| Minoxidil | 24, 360 | c |
| Hydralazine | 6 | d |
| BI-QT | 168, 336 | e |
| Clenbuterol | 6, 24 | f |
| Isoproterenol | 3, 6, 24 | g |
| Norepinephrine | 3, 6, 24 | h |
| Epinephrine | 3, 6, 24 | I |
| General | | General |

TABLE 5
GENERAL Attorney Docket No. 44921-5090US
Document No. 1828250.1

| Identifier | NonToxMean | NonToxSD | ToxMean | ToxSD | LDA score |
|---|---|---|---|---|---|
| 8938 | 72.49846847 | 35.616941 | 32.58413333 | 33.33534129 | 70.53453453 |
| 19696 | 61.17825826 | 20.87718965 | 41.4072 | 17.99470437 | 70.3003003 |
| 16448 | 316.0752853 | 106.4032634 | 196.5678667 | 148.6244308 | 72.45645646 |
| 1638 | 170.6204204 | 45.98409061 | 125.5168 | 50.68216788 | 67.8018018 |
| 17832 | 6223.652042 | 2633.02861 | 3682.600133 | 3280.902039 | 68.69069069 |
| 5596 | 129.2716817 | 48.34165983 | 86.29226667 | 48.08313596 | 65.53153153 |
| 15056 | 26.02435435 | 16.4477125 | 12.2056 | 15.84966299 | 65.66366366 |
| 1689 | 11552.76066 | 4830.350287 | 7248.0156 | 6306.417948 | 66.13813814 |
| 14464 | 151.2832132 | 63.47873283 | 100.9436 | 65.22443435 | 66.06606607 |
| 19913 | 50.12765766 | 19.66490555 | 34.7712 | 22.36177941 | 65.66966967 |
| 7362 | 185.6274775 | 46.43226766 | 150.2716 | 47.46886886 | 67.3993994 |
| 19067 | 51.95087087 | 25.98485092 | 31.64626667 | 22.82603751 | 65.44144144 |
| 7936 | 80.7251952 | 20.87755258 | 64.16906667 | 25.68788494 | 68.57057057 |
| 21400 | 122.3365165 | 36.82117511 | 93.13653333 | 43.86216043 | 66.1021021 |
| 14299 | 53.59687688 | 14.79619568 | 43.20906667 | 14.058604 | 67.74774775 |
| 18907 | 483.0658559 | 247.056126 | 297.2048 | 251.5669094 | 65.83183183 |
| 25517 | 188.9551051 | 94.03518084 | 115.5454667 | 105.9832198 | 67.26726727 |
| 1288 | 67.87501502 | 23.54663557 | 51.23253333 | 24.83954694 | 65.03303303 |
| 23822 | 337.497988 | 140.9213721 | 236.9002667 | 143.0727342 | 67.46546547 |
| 12613 | 89.39780781 | 31.97196903 | 94.58813333 | 53.76216973 | 65.85585586 |
| 22995 | 346.6827628 | 122.2171839 | 263.3754667 | 129.50731 | 66.15015015 |
| 18366 | 97.31693694 | 24.44041591 | 81.6344 | 24.11334744 | 66.94894895 |
| 4874 | 129.3038438 | 50.3843815 | 96.86506667 | 57.67563452 | 65.73573574 |
| 10547 | 26.3566967 | 11.08758866 | 19.384 | 11.36075476 | 65.81381381 |
| 11663 | 109.3640841 | 39.98991383 | 83.52146667 | 48.6847472 | 65.15315315 |
| 23099 | 113.1338438 | 62.46411954 | 156.8176 | 48.00433316 | 68.78078078 |
| 17285 | 246.9660661 | 68.23201281 | 208.7148 | 75.2358579 | 67.2012012 |
| 20975 | 157.1995195 | 37.21790724 | 135.5798667 | 44.41549326 | 66.33633634 |
| 3615 | 296.4857958 | 139.717057 | 216.0168 | 157.8900781 | 65.73573574 |
| 25469 | 3875.094264 | 1933.847909 | 3281.065067 | 2846.942639 | 66.13813814 |
| 18400 | 37.38276276 | 13.28625502 | 32.34986667 | 16.79435664 | 65.73573574 |
| 3561 | 62.44954955 | 35.76106582 | 45.69293333 | 34.03616518 | 65.30930931 |
| 6509 | 1.827387387 | 29.44622307 | 28.80293333 | 31.81768541 | 70.87087087 |
| 9111 | 110.7656757 | 44.35117669 | 68.14826667 | 53.68408666 | 68.28828829 |
| 12462 | 135.381021 | 42.97715274 | 97.76493333 | 48.98317153 | 68.5045045 |
| 1845 | 72.01876877 | 75.85335582 | 2.699866667 | 86.41553255 | 67.43543544 |
| 8594 | 243.5170571 | 68.45132346 | 302.068 | 77.87824043 | 68.25225225 |
| 4652 | 236.917958 | 75.80952172 | 175.702 | 90.61425001 | 65.36936937 |
| 2730 | 1383.565465 | 317.1048745 | 1138.2416 | 336.7685506 | 65.63363363 |
| 8919 | 219.293994 | 50.3691595 | 258.7578667 | 67.28264037 | 67.12312312 |
| 21185 | 595.4060961 | 186.9327029 | 474.0896 | 194.2886934 | 67.73573574 |
| 1831 | 212.2755556 | 76.52515314 | 152.4144 | 105.3498365 | 69.27327327 |
| 2997 | 80.00204204 | 28.71912254 | 59.6192 | 35.59476247 | 67.40540541 |
| 18909 | 1671.732853 | 392.4116077 | 1384.6024 | 451.8576469 | 66.53453453 |
| 26167 | 134.7165165 | 47.59463132 | 126.3729333 | 81.03451486 | 65.57357357 |
| 11865 | 65.93768769 | 16.95174622 | 61.7544 | 27.42951484 | 67.33933934 |
| 1283 | 107.8844745 | 35.04454514 | 97.00293333 | 52.32959867 | 65.40540541 |
| 14842 | 111.2535736 | 38.17357051 | 101.8753333 | 56.75867104 | 68.69069069 |
| 17264 | 30.31930931 | 10.9046027 | 24.5472 | 11.79646747 | 66.9009009 |
| 16092 | 369.1881381 | 133.2415014 | 383.0384 | 197.2260892 | 65.20720721 |
| 16001 | 6956.653874 | 4429.730358 | 7700.967467 | 5339.923013 | 65.97597598 |
| 385 | 53.32657658 | 20.34513196 | 24.41373333 | 19.32565245 | 81.59159159 |
| 6166 | 352.6468168 | 103.7327299 | 247.1990667 | 126.8492915 | 65.62162162 |
| 5132 | 1110.373213 | 230.1370785 | 886.4596 | 241.8634169 | 69.63363363 |
| 15727 | 49.35342342 | 15.34697312 | 35.6312 | 15.48039719 | 68.6006006 |
| 11403 | −4.132762763 | 101.6886382 | 84.9084 | 109.9803583 | 66.55255255 |
| 4861 | 171.4952252 | 48.73056765 | 124.3810667 | 61.74762146 | 71.03903904 |
| 19282 | −42.65402402 | 87.74398555 | 28.23106667 | 110.6660663 | 67.53753754 |
| 15383 | 79.91810811 | 30.652448 | 55.4604 | 38.95519784 | 65.003003 |
| 16071 | 140.2272372 | 49.35971496 | 109.4429333 | 72.91664278 | 65.33933934 |
| 17848 | 194.3432132 | 58.06606118 | 212.2409333 | 95.04139603 | 65.14114114 |
| 15628 | 106.193964 | 53.66420537 | 72.38413333 | 59.12261967 | 66.30630631 |
| 24886 | 1398.435015 | 383.9561012 | 1510.2572 | 582.9668179 | 66.00600601 |
| 3417 | 444.3617417 | 120.8043364 | 467.0837333 | 177.560328 | 65.20720721 |
| 18456 | 237.4204805 | 102.3981852 | 224.7922667 | 142.9449073 | 65.12312312 |
| 22711 | 219.0362462 | 49.22663825 | 165.8392 | 51.01685608 | 67.65165165 |
| 23773 | 252.4870571 | 83.55121918 | 334.7781333 | 82.8740413 | 69.58558559 |
| 22697 | 464.0506607 | 145.4066285 | 294.1353333 | 193.0841828 | 69.83783784 |
| 20776 | 69.56924925 | 16.97614522 | 55.06893333 | 19.69728055 | 66.12012012 |
| 22696 | 319.6695495 | 111.2920825 | 215.6545333 | 149.7528706 | 67.55555556 |
| 11684 | 180.4317718 | 53.62509097 | 134.8041333 | 81.03253826 | 67.63963964 |
| 4154 | 192.7478979 | 68.26975549 | 218.1085333 | 125.7269822 | 66.55855856 |
| 19291 | 26.68984985 | 16.60798861 | 34.80786667 | 29.24204855 | 67.95795796 |
| 3214 | 33.65321321 | 14.5901984 | 44.16 | 15.92048791 | 65.53753754 |
| 19781 | 55.21522523 | 26.60262402 | 59.48026667 | 47.15136707 | 65.35735736 |
| 13568 | 117.4451952 | 31.74744654 | 98.47613333 | 43.88527614 | 65.95795796 |
| 24107 | 111.7821622 | 27.76698092 | 105.1286667 | 43.0198754 | 69.44144144 |
| 2399 | 54.42369369 | 18.87054417 | 60.1316 | 30.20835042 | 65.48948949 |
| 12988 | 19.73492492 | 13.43661964 | 26.04933333 | 19.45953385 | 66.54054054 |
| 8612 | 44.25837838 | 26.76734752 | 43.31693333 | 38.46574605 | 67.50750751 |
| 20073 | 46.95612613 | 18.37152689 | 24.33906667 | 21.00683603 | 69.77177177 |
| 23409 | 784.9057357 | 234.5443921 | 514.8028 | 285.4738576 | 70.12012012 |
| 23566 | 202.5740541 | 51.57262868 | 247.5364 | 59.10443706 | 69.40540541 |
| 23833 | 3.16003003 | 19.1547679 | 20.5952 | 22.24789892 | 68.13813814 |
| 1349 | 63.36246246 | 18.54507329 | 46.76426667 | 23.6663677 | 66.82282282 |
| 1571 | 171.1223724 | 44.14656407 | 192.9042667 | 83.37646024 | 70.34234234 |
| 4459 | 51.14861862 | 18.05307097 | 35.30733333 | 21.06236097 | 67.38738739 |
| 14554 | 108.7731832 | 30.90475217 | 92.1288 | 53.21609427 | 70.19219219 |
| 19255 | 249.8988288 | 94.46639357 | 172.4934667 | 136.1692967 | 68.02402402 |
| 4355 | 116.2907207 | 41.25938907 | 132.4930667 | 69.32780921 | 65.42342342 |
| 14380 | 369.6702402 | 106.6646804 | 314.9093333 | 190.0506953 | 67.95795796 |
| 18447 | 689.994955 | 154.965744 | 665.2398667 | 273.6720581 | 65.59159159 |
| 9658 | 91.44114114 | 56.37854523 | 108.8808 | 80.81962342 | 66.74474474 |
| 10394 | 127.6029429 | 33.74101819 | 144.1212 | 59.26738732 | 69.95795796 |
| 2984 | 820.9295796 | 296.6266834 | 642.7485333 | 398.8418716 | 66.55855856 |
| 23187 | 382.4496697 | 84.05252115 | 338.7626667 | 132.5344805 | 65.12312312 |
| 14651 | 22.97741742 | 11.88799972 | 35.14666667 | 13.31225358 | 68.24024024 |
| 15556 | 773.6088288 | 170.1184709 | 580.3448 | 207.3705142 | 68.13813814 |
| 15929 | 226.1643844 | 73.67163957 | 298.5917333 | 109.9870393 | 65.95795796 |
| 22378 | 258.03 | 61.21775049 | 199.0550667 | 82.91016084 | 67.20720721 |
| 14184 | 219.4502703 | 100.3089978 | 265.0389333 | 189.0891682 | 66.24024024 |
| 6568 | 113.7420721 | 37.12194787 | 112.3276 | 67.20321817 | 67.22522523 |
| 26148 | 46.32885886 | 31.16904645 | 24.33973333 | 47.28917355 | 65.20720721 |
| 3143 | 327.6772973 | 85.34914028 | 265.438 | 130.1184031 | 65.27327327 |
| 3405 | 255.8100601 | 72.91699692 | 279.1788 | 114.8346427 | 65.89189189 |
| 2135 | 113.2241441 | 48.6776077 | 128.3670667 | 102.1457422 | 68.42642643 |
| 4594 | 101.8178679 | 36.76081853 | 64.6076 | 43.38325041 | 69.15315315 |
| 17320 | 193.7527027 | 56.48179636 | 216.2656 | 109.0445704 | 67.24324324 |
| 12946 | 214.3218919 | 48.7516373 | 169.7182667 | 64.40548158 | 67.63963964 |
| 5005 | 64.97573574 | 17.07035037 | 50.6384 | 24.06437257 | 66.24024024 |
| 2587 | 128.6519219 | 49.02276069 | 138.8965333 | 86.68669157 | 65.22522523 |
| 22036 | 232.5344745 | 70.13135612 | 288.1818667 | 90.96748966 | 69.42342342 |
| 18468 | 103.6571471 | 26.34536999 | 84.50693333 | 31.75542899 | 71.20720721 |
| 22247 | 547.1983483 | 167.2471262 | 600.6785333 | 306.5840909 | 65.50750751 |
| 20204 | 48.7542042 | 13.12382882 | 38.2412 | 18.85018108 | 67.80780781 |
| 18611 | 1866.745556 | 561.2759596 | 1985.5172 | 989.2441283 | 66.94294294 |
| 6262 | 833.3602703 | 209.3539434 | 667.2974667 | 262.0166407 | 66.35435435 |
| 24310 | 300.4368769 | 92.5559206 | 292.298 | 172.1727988 | 66.39039039 |
| 9970 | 41.8290991 | 16.71326192 | 53.35293333 | 22.98860668 | 68.10810811 |
| 3391 | 65.42762763 | 23.17844683 | 73.84173333 | 38.98158947 | 65.95795796 |
| 1867 | 563.8847447 | 144.4354691 | 617.586 | 252.7746934 | 67.44144144 |
| 22762 | 43.69891892 | 18.7980068 | 38.3012 | 30.06706931 | 66.10810811 |
| 21659 | 398.0121021 | 119.1107764 | 515.552 | 146.6284508 | 68.05405405 |
| 18406 | 103.7865465 | 38.59483955 | 126.8301333 | 74.69609428 | 66.55855856 |
| 956 | 248.7113213 | 78.1677381 | 165.5565333 | 101.821794 | 70.07207207 |
| 5950 | 310.0055856 | 91.83088416 | 333.1805333 | 170.7325037 | 66.42642643 |
| 2069 | 325.0245946 | 80.5912804 | 387.7393333 | 143.8666882 | 74.49249249 |
| 23166 | 106.8232132 | 27.84324275 | 111.3249333 | 53.83331056 | 67.80780781 |
| 6059 | 133.9323724 | 37.17657273 | 119.3445333 | 63.46730525 | 68.12612613 |
| 9339 | 169.3109309 | 44.91784911 | 188.1286667 | 84.28460894 | 67.05705706 |
| 1894 | 153.4639339 | 62.86198889 | 182.2790667 | 111.1631361 | 66.69069069 |
| 6430 | 155.9801201 | 68.97035674 | 183.3576 | 110.0622628 | 67.97597598 |
| 6044 | 228.932042 | 67.60066509 | 267.0017333 | 99.42927714 | 66.64264264 |
| 2042 | 157.1421021 | 70.75267 | 179.5984 | 120.4462597 | 65.44144144 |
| 20897 | 236.3516517 | 51.74161557 | 253.342 | 85.02425018 | 65.07507508 |
| 20461 | 93.10510511 | 26.50362991 | 60.50453333 | 31.08815397 | 72.00600601 |
| 24867 | 48.31201201 | 15.52563294 | 32.08106667 | 25.64892767 | 66.32432432 |
| 17361 | 97.88654655 | 33.24848768 | 63.50213333 | 42.27341108 | 66.95495495 |
| 13023 | 39.76930931 | 17.59981107 | 55.8428 | 25.74507088 | 65.87387387 |
| 2936 | 77.56312312 | 26.66715017 | 55.54626667 | 46.81207412 | 68.40840841 |
| 12195 | 40.29054054 | 28.19399017 | 67.17093333 | 34.30477164 | 66.5045045 |
| 7308 | 173.0977477 | 52.3843028 | 186.2728 | 105.4884207 | 65.97597598 |
| 7941 | 143.4922222 | 59.61799581 | 178.7176 | 114.5027757 | 72.84084084 |
| 3965 | 1082.078468 | 215.0078492 | 885.6898667 | 264.1772007 | 65.9039039 |
| 16613 | 104.2170571 | 30.30979396 | 75.58706667 | 43.71236237 | 68.09009009 |
| 20055 | 203.1345946 | 98.95872235 | 239.5048 | 162.7233699 | 66.42642643 |
| 9984 | 53.41801802 | 18.57642971 | 60.2156 | 33.93376956 | 65.74174174 |
| 19416 | 51.55012012 | 23.65535112 | 61.37493333 | 42.65959448 | 68.66066066 |
| 23501 | 570.5681381 | 188.6893698 | 600.8397333 | 313.8877745 | 65.39339339 |
| 22873 | 218.6465165 | 69.42724004 | 242.9489333 | 126.1457279 | 66.25825826 |
| 20392 | 108.1697898 | 36.74729252 | 91.06426667 | 58.95886771 | 66.12612613 |
| 22175 | 122.7006607 | 60.73210182 | 146.3976 | 102.685777 | 65.82582583 |
| 21644 | 42.73414414 | 20.02239798 | 27.44893333 | 27.65488167 | 66.02402402 |
| 12609 | 233.4871171 | 60.64644968 | 251.8496 | 101.0000546 | 65.97597598 |
| 11561 | 64.27129129 | 26.06807716 | 74.09653333 | 42.81586396 | 67.07507508 |
| 12364 | 167.1453453 | 34.88732627 | 180.3305333 | 101.9126453 | 67.11111111 |
| 6684 | 46.39993994 | 17.61944269 | 23.29093333 | 18.45816242 | 73.88588589 |
| 15088 | 234.1487087 | 50.98531943 | 298.5472 | 66.90284801 | 67.78978979 |
| 22042 | 80.95408408 | 64.01668998 | 134.0318667 | 71.90333572 | 70.96096096 |
| 10780 | 170.215015 | 52.15528635 | 216.1777333 | 103.2157802 | 70.06006006 |
| 13310 | 263.6483183 | 84.94409905 | 335.0121333 | 163.1743769 | 71.37537538 |
| 5867 | 125.3911411 | 28.71252609 | 153.1288 | 49.53867263 | 67.89189189 |
| 895 | 52.09792793 | 31.27119755 | 61.64933333 | 63.07948599 | 66.64264264 |
| 25765 | 69.91843844 | 15.25861986 | 53.73533333 | 20.65170364 | 70.30630631 |
| 8626 | 50.23366366 | 26.43575146 | 73.9184 | 40.96692697 | 65.37537538 |
| 20684 | 151.0114114 | 31.00777899 | 119.2346667 | 43.21294705 | 65.63963964 |
| 16546 | 274.2533333 | 69.74767882 | 201.9084 | 88.68051698 | 69.47147147 |
| 17903 | 136.4756156 | 43.38108814 | 154.49 | 81.74623287 | 65.60960961 |
| 20229 | 77.35189189 | 23.10267263 | 54.49306667 | 32.50505615 | 65.35735736 |
| 1687 | 5403.358649 | 1841.285364 | 3390.540933 | 2737.843207 | 69.29129129 |
| 9362 | 196.7336637 | 42.60975263 | 155.3865333 | 61.28247745 | 67.65765766 |
| 1097 | 291.0685886 | 93.99259964 | 205.6634667 | 145.7332274 | 69.30930931 |
| 22820 | 418.5933934 | 90.02872862 | 482.8748 | 142.1782873 | 65.22522523 |
| 22212 | 34.41504505 | 15.89632599 | 43.12893333 | 27.91798454 | 65.90990991 |
| 22146 | 78.36675676 | 27.69105018 | 53.51706667 | 38.33774334 | 67.63963964 |
| 22077 | 143.718018 | 57.69355684 | 155.6457333 | 99.80830983 | 65.05705706 |
| 8707 | 196.0183784 | 48.34247102 | 207.6557333 | 89.02444141 | 66.09009009 |
| 7681 | 78.76585586 | 32.16112279 | 91.39706667 | 50.62460284 | 66.12612613 |
| 8888 | 61.45741742 | 24.54918629 | 31.1132 | 35.73105445 | 70.54054054 |
| 18109 | 156.0742042 | 55.81778773 | 218.1410667 | 77.47029568 | 66.69069069 |
| 3982 | 268.0332132 | 75.24003617 | 183.6764 | 89.5029389 | 69.40540541 |
| 14737 | 216.4417718 | 57.2904164 | 276.9370667 | 85.0585165 | 65.07507508 |
| 20041 | 162.7117117 | 66.1931698 | 204.4074667 | 136.2028514 | 67.30930931 |
| 23963 | 50.19213213 | 27.25661616 | 72.6608 | 34.91346924 | 66.94294294 |
| 23015 | 76.28105105 | 21.35348484 | 84.84493333 | 42.91649851 | 66.06006006 |
| 12695 | 3.236666667 | 31.83601273 | 33.17786667 | 47.92099541 | 66.55855856 |
| 6697 | 72.20354354 | 25.83488711 | 97.43946667 | 34.27944486 | 66.09009009 |
| 8886 | 149.2603003 | 27.11847144 | 162.3432 | 53.10939425 | 69.07507508 |
| 21391 | 182.8798198 | 62.3648526 | 232.0578667 | 106.4582716 | 67.82582583 |
| 22544 | 96.31168168 | 45.91951934 | 99.76533333 | 82.65850379 | 65.24324324 |
| 5886 | 105.5642342 | 43.68595668 | 123.5329333 | 80.96381669 | 68.10810811 |
| 4093 | 761.2472372 | 171.2305171 | 753.6394667 | 320.9534796 | 65.87387387 |
| 19722 | 105.7899099 | 43.47888541 | 102.1081333 | 76.9549177 | 66.12612613 |
| 13772 | 831.1903303 | 186.4790627 | 832.472 | 347.4316136 | 65.07507508 |
| 6638 | 170.9305405 | 46.16586712 | 153.616 | 81.14338217 | 65.74174174 |
| 8230 | 64.91228228 | 22.10574191 | 64.138 | 40.92817014 | 65.14114114 |
| 16130 | 4786.742523 | 2836.481592 | 5484.7164 | 3926.112296 | 65.69369369 |
| 4790 | 173.9938438 | 84.09043096 | 428.1037333 | 790.0992435 | 65.0990991 |
| 15840 | 44.64630631 | 14.56553354 | 49.0236 | 55.86485158 | 65.42942943 |
| 21671 | 40.08372372 | 19.31257365 | 57.3588 | 54.19357641 | 65.1951952 |
| 10227 | 887.740991 | 218.0449747 | 580.4258667 | 314.355664 | 71.57357357 |
| 24028 | 400.1148048 | 104.0176495 | 256.5913333 | 152.1790505 | 66.77477477 |
| 19050 | 131.9762763 | 27.51290769 | 100.1389333 | 53.45738077 | 65.45945946 |
| 22487 | 18.75924925 | 11.45640602 | 31.10826667 | 18.85726968 | 66.79279279 |
| 3773 | 25.65585586 | 25.17539957 | 46.98506667 | 37.82222125 | 66.37837838 |
| 1159 | 114.0870871 | 32.96098549 | 75.41066667 | 40.31298622 | 70.95495495 |
| 5507 | 1321.152042 | 406.4159467 | 862.4322667 | 485.470829 | 68.13813814 |
| 15299 | 94.47942943 | 57.73406292 | 114.3178667 | 92.88693964 | 67.14714715 |
| 18569 | 1244.146697 | 488.8296588 | 1342.794267 | 1047.069388 | 68.44444444 |
| 23197 | 242.457027 | 65.24136019 | 168.2028 | 83.99489168 | 68.88888889 |
| 18272 | 88.60597598 | 50.87240463 | 110.8405333 | 93.53148608 | 66.29429429 |
| 20891 | 112.2740841 | 43.6111356 | 163.6264 | 66.8717327 | 68.54054054 |
| 20743 | 156.6083483 | 23.13382173 | 127.0229333 | 38.72581832 | 73.89189189 |
| 18887 | 193.9248348 | 38.32142815 | 153.4470667 | 56.84990586 | 71.50750751 |
| 4098 | 118.7078979 | 56.06679909 | 151.8290667 | 105.6281798 | 72.81081081 |
| 17644 | 473.8572072 | 88.11026526 | 382.0854667 | 151.7742492 | 65.89189189 |
| 2852 | 468.4916216 | 156.8990485 | 296.9326667 | 204.4010076 | 66.73873874 |
| 5351 | 805.1087087 | 183.5458093 | 780.6014667 | 374.2614004 | 65.39339339 |
| 5754 | 311.9932132 | 90.10901524 | 379.9041333 | 150.3765953 | 67.24324324 |
| 15218 | 446.0465766 | 118.9354806 | 469.6905333 | 233.6035158 | 65.30930931 |
| 17226 | 193.6157658 | 41.20808929 | 184.2581333 | 77.60555747 | 65.45945946 |
| 11387 | 65.62636637 | 19.53063089 | 48.0472 | 29.83941582 | 68.39039039 |
| 12898 | 1155.093604 | 326.9983162 | 920.6853333 | 556.14818 | 71.80780781 |
| 13266 | 88.83081081 | 34.14338586 | 125.1286667 | 96.18356271 | 67.77777778 |
| 22415 | 169.1916517 | 71.9805775 | 208.9908 | 154.7452282 | 65.4954955 |
| 1714 | 190.2463664 | 42.14693723 | 128.7704 | 61.18700552 | 76.12612613 |
| 7760 | 332.0363363 | 68.75841865 | 237.5545333 | 123.2838632 | 68.70870871 |
| 12664 | 100.1887087 | 31.55464398 | 58.20906667 | 49.64866227 | 66.54054054 |
| 17541 | 75.40276276 | 35.37305084 | 92.49773333 | 79.29429139 | 67.09309309 |
| 11714 | 688.3123724 | 141.9171508 | 511.8988 | 252.9565028 | 70.57657658 |
| 12752 | 18.40444444 | 9.926800286 | 29.5148 | 17.2954324 | 66.64264264 |
| 1218 | 70.99513514 | 23.00948946 | 48.79626667 | 44.46786686 | 65.30930931 |
| 4420 | 459.2146847 | 102.7069783 | 336.7681333 | 147.9624508 | 69.29129129 |
| 1287 | 642.0570571 | 156.8171393 | 458.5782667 | 226.0114284 | 70.17417417 |
| 4832 | 820.3085586 | 203.9557534 | 592.4816 | 268.1038956 | 66.97297297 |
| 1324 | 540.7645045 | 102.5052955 | 431.7101333 | 168.4053971 | 65.29129129 |
| 21660 | 1799.216336 | 582.415201 | 2101.294 | 1096.999967 | 70.52852853 |
| 16756 | 124.3146246 | 31.85423172 | 158.2822667 | 42.44045487 | 72.92492492 |
| 13539 | 74.31735736 | 27.19784127 | 88.31293333 | 53.87132795 | 66.57657658 |
| 3959 | 386.8933934 | 90.70995601 | 439.0906667 | 188.144252 | 68.94294294 |
| 13647 | 546.5458559 | 171.8763562 | 657.4113333 | 297.5909747 | 67.47747748 |
| 21510 | 412.0168468 | 117.3257767 | 392.8604 | 249.6312061 | 66.25825826 |
| 11724 | 117.5545045 | 49.09806535 | 64.2108 | 77.37112397 | 68.32432432 |
| 13336 | 363.4029129 | 111.7914019 | 247.1030667 | 179.801567 | 69.14114114 |
| 18278 | 3944.232192 | 998.8950354 | 2938.5136 | 1271.568221 | 65.85585586 |
| 7580 | 252.0656456 | 66.83673414 | 269.3414667 | 139.1313757 | 67.82582583 |
| 12274 | 118.6513213 | 43.99173734 | 76.60813333 | 69.01666727 | 65.50750751 |
| 4161 | 50.6696997 | 14.21036004 | 54.3908 | 26.64655319 | 67.09309309 |
| 11375 | 99.21636637 | 32.90484725 | 72.73453333 | 50.20147194 | 66.24024024 |
| 20038 | 196.8626126 | 54.36882244 | 161.6828 | 92.5331668 | 66.32432432 |
| 11205 | 384.3533333 | 106.3089306 | 318.15 | 166.5292475 | 65.35735736 |
| 23217 | 808.4991592 | 239.5294339 | 640.1121333 | 363.4106742 | 65.20720721 |
| 5844 | 33.20618619 | 18.54011387 | 42.9916 | 53.24256923 | 65.11111111 |
| 9770 | 46.44246246 | 19.92613955 | 59.5272 | 50.94862942 | 67.26126126 |
| 3207 | 253.0190991 | 79.66059119 | 133.1012 | 106.1880748 | 73.65765766 |
| 23033 | 536.1412613 | 107.4224838 | 393.7129333 | 195.3626471 | 67.99399399 |
| 15935 | 81.22621622 | 16.48918158 | 57.27653333 | 22.94097229 | 69.57357357 |
| 18581 | 215.9942042 | 55.20408872 | 269.8570667 | 123.1063629 | 67.90990991 |
| 14337 | 322.3194294 | 53.66143193 | 257.1765333 | 104.3393465 | 66.72672673 |
| 22691 | 1075.303183 | 228.9954085 | 784.5778667 | 424.8754518 | 69.17717718 |
| 10878 | 1385.995916 | 414.8758472 | 1866.4344 | 799.5945004 | 66.42642643 |
| 5356 | −5.118978979 | 21.00136767 | 22.42866667 | 35.86063261 | 66.85885886 |
| 8861 | 29.17072072 | 22.80576141 | 51.44973333 | 49.9321422 | 65.45945946 |
| 10097 | 124.147988 | 26.54127344 | 93.0952 | 47.52408514 | 67.07507508 |
| 3822 | 673.761982 | 186.9080741 | 872.1389333 | 350.816601 | 65.00900901 |
| 15568 | 246.6630631 | 83.93584731 | 297.0686667 | 191.9541491 | 69.84384384 |
| 16597 | 658.9440541 | 130.1613616 | 494.0310667 | 195.673027 | 68.54054054 |
| 21785 | 90.4812012 | 32.50010122 | 110.3524 | 67.21918687 | 66.51051051 |
| 14458 | 41.40780781 | 24.65869776 | 63.26986667 | 47.78168971 | 68.64264264 |
| 14397 | 66.74069069 | 45.47819109 | 118.4668 | 67.8647496 | 65.20720721 |
| 11504 | 544.7894294 | 138.227504 | 406.5510667 | 256.3281666 | 68.04204204 |
| 20582 | 473.1648348 | 82.13508879 | 386.0477333 | 141.7973162 | 67.24324324 |
| 25846 | 78.24933934 | 23.01624996 | 51.4856 | 35.68920123 | 65.14114114 |
| 15786 | 92.55792793 | 39.70677047 | 133.7984 | 64.20978712 | 69.82582583 |
| 20915 | 361.9305706 | 78.9856249 | 280.1098667 | 131.5644921 | 65.30930931 |
| 4710 | 138.5577477 | 40.89817673 | 145.0194667 | 87.66842411 | 65.45945946 |
| 18726 | 117.1451351 | 35.4755811 | 91.67053333 | 63.49358782 | 66.42642643 |
| 9353 | 179.7312012 | 43.05211274 | 133.356 | 62.74557773 | 66.02402402 |
| 18454 | 77.9424024 | 21.87075514 | 84.30893333 | 45.96557092 | 67.82582583 |
| 7749 | 1094.882583 | 214.8318019 | 1330.865067 | 358.0804114 | 73.09309309 |
| 19379 | 369.4203303 | 68.83430484 | 388.168 | 141.7341057 | 66.12612613 |
| 6632 | 118.8854655 | 42.29574415 | 139.8264 | 86.79784998 | 65.30930931 |
| 23596 | 319.7261261 | 76.98365859 | 257.4506667 | 138.4931514 | 65.00900901 |
| 17101 | 402.5251952 | 84.47841158 | 358.5670667 | 168.1087789 | 66.04204204 |
| 17901 | 306.0611712 | 61.0008545 | 354.7029333 | 111.765356 | 69.15915916 |
| 5458 | 152.2901201 | 57.22131483 | 202.6198667 | 101.1782236 | 69.89189189 |
| 11218 | 123.8714715 | 39.88322913 | 85.18106667 | 66.39136006 | 66.62462462 |
| 5703 | 186.4057357 | 57.53155348 | 215.8356 | 110.3789575 | 66.12612613 |
| 5969 | 447.1866967 | 131.1009133 | 443.3448 | 238.403989 | 65.90990991 |
| 14256 | 281.3109309 | 55.06653776 | 309.9438667 | 100.9660491 | 65.30930931 |
| 21583 | 378.1696697 | 104.8760736 | 296.7974667 | 186.0371966 | 69.59159159 |
| 13966 | 95.99645646 | 29.92445819 | 103.2432 | 57.73805872 | 65.59159159 |
| 15281 | 121.7892492 | 31.12274069 | 127.6637333 | 61.30898267 | 66.92492492 |
| 15879 | 203.2204805 | 53.26954563 | 193.0226667 | 97.94002102 | 66.85885886 |
| 20839 | 1313.201411 | 381.4093254 | 1380.0756 | 720.1580309 | 66.21021021 |
| 958 | 116.6435736 | 35.88540631 | 90.49386667 | 51.75431641 | 65.20720721 |
| 16847 | 968.6696396 | 266.3889987 | 1007.937333 | 502.0256491 | 65.00900901 |
| 6562 | 83.82294294 | 22.0288454 | 69.6552 | 35.19942849 | 66.51051051 |
| 23854 | 639.7981982 | 186.0579446 | 1106.001067 | 835.6522508 | 66.78078078 |
| 6431 | 85.04474474 | 33.9730999 | 112.5750667 | 88.16570698 | 66.22822823 |
| 1439 | 263.1485586 | 44.01928161 | 206.8229333 | 97.34968891 | 65.62762763 |
| 8495 | 119.0903604 | 32.55673471 | 165.8949333 | 64.95183826 | 70.66066066 |
| 1754 | 185.4703604 | 46.72545297 | 120.4281333 | 56.61101972 | 73.85585586 |
| 19298 | 311.8326126 | 101.6730563 | 432.0438667 | 159.034197 | 68.22822823 |
| 20983 | 430.2093994 | 86.98985623 | 324.0536 | 160.9977404 | 66.57657658 |
| 15708 | −2.214954955 | 17.56851948 | 21.55906667 | 29.04976049 | 68.12612613 |
| 11738 | 309.9483183 | 60.96372833 | 254.6993333 | 128.5746206 | 65.39339339 |
| 25730 | 216.6359159 | 52.68303932 | 220.2433333 | 124.4837884 | 68.87687688 |
| 16721 | 416.0622222 | 85.01057014 | 336.1954667 | 146.7998256 | 66.14414414 |
| 4222 | 862.0381682 | 176.5955454 | 841.7009333 | 394.791692 | 65.11111111 |
| 23978 | 161.4975075 | 32.00206436 | 124.3706667 | 60.97444 | 69.22522523 |
| 22248 | 377.2372372 | 120.7632366 | 484.9410667 | 239.3602421 | 65.90990991 |
| 17948 | 104.0551652 | 43.81607287 | 158.1704 | 73.64095512 | 68.70870871 |
| 1760 | 188.8481081 | 39.96338126 | 143.2396 | 56.35055556 | 66.77477477 |
| 22011 | 73.38075075 | 21.79957488 | 95.94973333 | 34.24838903 | 65.39339339 |
| 18800 | 50.7418018 | 28.55545096 | 75.78813333 | 54.26756691 | 65.60960961 |
| 16596 | 304.4108408 | 85.19455004 | 207.3538667 | 135.8143346 | 67.22522523 |
| 22596 | 61.19957958 | 21.32527899 | 69.88026667 | 43.86778542 | 66.52852853 |
| 21661 | 1069.555405 | 352.3216968 | 1334.385067 | 717.4373056 | 74.14414414 |
| 23270 | 157.5835135 | 49.86066962 | 189.7304 | 110.3524169 | 67.90990991 |
| 18612 | 101.4829429 | 29.32672384 | 110.9098667 | 60.75506965 | 65.39339339 |
| 14968 | 266.143003 | 78.1768716 | 182.1364 | 125.6208288 | 67.45945946 |
| 17329 | 309.0369369 | 106.691949 | 348.3042667 | 228.0234129 | 66.79279279 |
| 15558 | 262.0998198 | 56.66623661 | 250.9046667 | 117.1831148 | 66.79279279 |
| 23464 | 160.2329129 | 44.51031583 | 195.8149333 | 81.59754346 | 67.67567568 |
| 16918 | 1413.133213 | 460.8671011 | 1629.528533 | 964.4253086 | 67.59159159 |
| 17394 | 317.0911712 | 90.98244545 | 362.8506667 | 173.2022245 | 70.57657658 |
| 18313 | 2157.747387 | 789.8817749 | 2653.856667 | 1515.939988 | 74.81081081 |
| 11039 | 503.3321021 | 157.1580278 | 481.8897333 | 308.5116227 | 65.74174174 |
| 22150 | 156.7123123 | 42.50597598 | 161.4842667 | 85.18488354 | 68.77477477 |
| 12639 | 1460.773393 | 444.0930486 | 1532.136267 | 827.6481876 | 65.84384384 |
| 22552 | 452.1078979 | 128.6560655 | 397.0625333 | 223.6136537 | 65.72372372 |
| 18713 | 213.5248048 | 45.86269132 | 316.8322667 | 181.996688 | 66.0960961 |
| 6217 | 49.97828829 | 17.40052541 | 70.66093333 | 60.18859693 | 68.22822823 |
| 11484 | 43.83453453 | 33.19740784 | 98.62786667 | 90.02330089 | 65.57957958 |
| 22898 | 81.10201201 | 32.79572472 | 122.5690667 | 102.3056384 | 68.82882883 |
| 6789 | 472.1047748 | 73.2765577 | 396.8628 | 231.7026096 | 67.26126126 |
| 19086 | 76.89486486 | 21.62493249 | 94.3248 | 62.04318693 | 68.52852853 |
| 4232 | 177.7873273 | 83.58675137 | 201.4142667 | 182.0039652 | 65.44744745 |
| 24520 | 56.30951952 | 29.47547907 | 75.4008 | 68.456624 | 66.46246246 |
| 13029 | 196.834024 | 70.15560213 | 267.1646667 | 203.5623949 | 73.04504505 |
| 24237 | 217.5224324 | 82.75251167 | 302.9657333 | 199.4963587 | 65.47747748 |
| 17847 | 1113.351321 | 201.1117315 | 812.0026667 | 380.90747 | 68.51051051 |
| 25567 | 77.7403003 | 27.1627134 | 93.72613333 | 72.91970052 | 67.56156156 |
| 15510 | 272.4407808 | 55.22298509 | 200.1338667 | 99.88071575 | 67.17717718 |
| 11876 | 62.22171171 | 19.88087401 | 72.2732 | 47.79009971 | 66.21021021 |
| 13467 | 51.40810811 | 25.57745758 | 81.73026667 | 41.97561251 | 67.45945946 |
| 4130 | 249.0733634 | 69.7014771 | 158.9117333 | 109.0187202 | 67.95795796 |
| 7414 | 283.2390991 | 52.04591174 | 327.2016 | 105.3800878 | 67.09309309 |
| 2555 | 54.72588589 | 21.23393369 | 64.0644 | 46.62585561 | 70.74474474 |
| 10020 | 100.8382583 | 47.24575942 | 135.8046667 | 70.65937512 | 66.0960961 |
| 14185 | 436.7817417 | 182.2642932 | 549.93 | 410.4846216 | 68.62462462 |
| 22877 | 64.27474474 | 20.36443332 | 86.88 | 33.84910454 | 66.10810811 |
| 6532 | 474.2814414 | 113.5953258 | 580.0912 | 242.3604877 | 69.45945946 |
| 20313 | 35.65138138 | 14.1217306 | 20.53386667 | 19.15284637 | 67.97597598 |
| 1435 | 846.9484985 | 157.1005438 | 908.9862667 | 348.3857446 | 67.02702703 |
| 18744 | 101.0285285 | 27.59011635 | 108.8517333 | 62.28678176 | 68.55855856 |
| 8212 | 461.3372673 | 118.7607732 | 517.1966667 | 237.3930463 | 67.69369369 |
| 2913 | 723.989039 | 143.8207512 | 640.2332 | 269.3334688 | 65.00900901 |
| 21311 | 173.7901201 | 38.25513248 | 175.5097333 | 80.92387205 | 68.19219219 |
| 3278 | 83.38897898 | 34.32457211 | 172.1812 | 217.7508072 | 67.44744745 |
| 23276 | 864.8205706 | 125.0984636 | 866.9262667 | 382.9781801 | 66.44444444 |
| 19710 | 50.87462462 | 21.10533047 | 74.98946667 | 44.87698519 | 67.34534535 |
| 10532 | 57.90630631 | 19.90646746 | 87.9208 | 39.20180911 | 69.11111111 |
| 18797 | 95.17507508 | 48.29428804 | 156.7506667 | 96.37476132 | 65.90990991 |
| 20735 | 109.1551952 | 37.54872752 | 142.3949333 | 84.04939678 | 68.21021021 |
| 15277 | 757.2586486 | 119.9808792 | 772.7332 | 288.8291533 | 65.75975976 |
| 1686 | 115.4446847 | 148.2121685 | 126.2946667 | 331.9836149 | 67.32732733 |
| 20056 | 160.4448949 | 27.21506574 | 126.5577333 | 51.13005332 | 67.14114114 |
| 8597 | 157.2614114 | 34.24367592 | 183.6321333 | 76.66422264 | 69.00900901 |
| 24375 | 100.4056456 | 31.10670046 | 119.3706667 | 65.09911537 | 70.89489489 |
| 19069 | 402.5671772 | 120.7772574 | 499.0681333 | 256.026845 | 67.90990991 |
| 9815 | 78.68213213 | 21.46012066 | 74.7772 | 45.70091798 | 66.06006006 |
| 15365 | 1052.608979 | 246.8184515 | 1075.352133 | 560.3357167 | 67.60960961 |
| 6097 | 10.70741742 | 19.10787732 | 30.47386667 | 31.40837855 | 65.89189189 |
| 3819 | 184.0441742 | 53.0645961 | 189.8553333 | 116.8066017 | 67.54354354 |
| 4330 | 499.2403604 | 146.4246876 | 445.0722667 | 325.0491692 | 70.34234234 |
| 22796 | 40.27414414 | 17.36701142 | 58.12186667 | 29.99494889 | 67.37537538 |
| 19256 | 418.5910511 | 122.8042598 | 305.5228 | 209.8678472 | 72.66066066 |
| 23523 | 185.5400901 | 50.98658976 | 199.4593333 | 101.4936327 | 68.72672673 |
| 25689 | 1086.230781 | 330.8253231 | 1171.6916 | 684.1791479 | 69.37537538 |
| 8665 | 372.092973 | 137.4462761 | 624.3146667 | 937.9525293 | 67.38138138 |
| 23538 | 61.26486486 | 25.00630833 | 138.8268 | 167.9882378 | 65.08108108 |
| 15867 | 39.75294294 | 27.36145508 | 78.18026667 | 129.9322765 | 68.4984985 |
| 2153 | 211.6812312 | 59.63630688 | 333.5897333 | 329.2563808 | 65.5975976 |
| 21351 | 15.94681682 | 17.28550647 | 52.00893333 | 75.78061318 | 65.36336336 |
| 5357 | 23.81036036 | 13.3662107 | 45.72693333 | 55.65476952 | 66.54654655 |
| 4626 | 30.00354354 | 26.99813854 | 76.3696 | 88.61559315 | 67.51351351 |
| 3905 | 99.72459459 | 59.91890871 | 203.3474667 | 170.4065695 | 66.97897898 |
| 3808 | 492.6752553 | 142.0629658 | 603.0476 | 412.3249868 | 65.72972973 |
| 20193 | 11.44021021 | 14.63339473 | 31.99226667 | 27.42250706 | 66.56456456 |
| 19031 | 226.5542042 | 80.71764495 | 316.426 | 261.0315285 | 69.64564565 |
| 11203 | 48.56756757 | 15.08008286 | 70.0172 | 38.56319302 | 67.11111111 |
| 4490 | 72.03528529 | 74.96309352 | 158.0234667 | 146.7958664 | 65.72972973 |
| 16859 | 82.11129129 | 31.38637404 | 115.0214667 | 85.19712603 | 66.89489489 |
| 15518 | 363.7106907 | 72.93920813 | 316.3657333 | 200.8114001 | 69.34534535 |
| 5550 | 20.18411411 | 12.63553326 | 38.39026667 | 27.0406112 | 66.66066066 |
| 14989 | 637.2671471 | 135.4981746 | 654.208 | 329.2529091 | 68.82882883 |
| 22899 | 37.78258258 | 29.93899336 | 56.64346667 | 65.64725464 | 69.72972973 |
| 1514 | 219.8367568 | 52.39364439 | 221.7862667 | 127.4380644 | 65.84384384 |
| 3437 | 100.6198198 | 26.50625625 | 129.4196 | 63.31158428 | 72.14414414 |
| 22017 | 170.6417417 | 151.2467146 | 328.8046667 | 203.913878 | 67.71171171 |
| 8494 | 468.2971772 | 92.6200257 | 451.1066667 | 237.8252614 | 68.29429429 |
| 6743 | 1114.827808 | 214.5651039 | 921.2714667 | 440.2561661 | 65.11111111 |
| 5227 | 134.3546547 | 45.77664557 | 174.9353333 | 104.6009944 | 76.96096096 |
| 2767 | 32.35042042 | 36.14976138 | 45.7332 | 81.02978982 | 67.62762763 |
| 24783 | 147.3642342 | 35.36879761 | 112.6877333 | 56.06313294 | 65.7957958 |
| 14693 | 17.43990991 | 13.55657195 | 29.88053333 | 29.68700712 | 70.87687688 |
| 22915 | 128.6723123 | 32.54275983 | 143.9506667 | 69.03744709 | 66.51051051 |
| 2254 | 191.3588288 | 40.96615757 | 191.9765333 | 95.34089695 | 65.39339339 |
| 22689 | 17.02327327 | 10.95011976 | 26.6544 | 21.30223001 | 65.39339339 |
| 3014 | 224.8988288 | 56.86046275 | 155.4037333 | 104.1163236 | 73.45945946 |
| 2242 | 175.0172673 | 84.92653403 | 219.9044 | 174.5829997 | 69.26126126 |
| 11152 | 189.5948649 | 59.08901035 | 224.5426667 | 128.3708172 | 70.51051051 |
| 14117 | 1323.556096 | 231.1844794 | 1069.146933 | 350.1422127 | 66.40840841 |
| 8829 | 256.9392192 | 64.59833933 | 273.9869333 | 136.6482686 | 65.67567568 |
| 24042 | 110.1422523 | 35.33224916 | 122.708 | 74.49565322 | 69.60960961 |
| 19840 | 1389.296787 | 364.2648838 | 1418.3772 | 705.7096386 | 68.66066066 |
| 17107 | 3085.335195 | 786.4229829 | 3162.925067 | 1656.771851 | 65.90990991 |
| 3431 | 521.6373874 | 133.7542256 | 587.462 | 279.2035761 | 69.37537538 |
| 6100 | 1733.87003 | 500.8862283 | 1813.900533 | 971.1778472 | 68.04204204 |
| 4169 | 15.23216216 | 13.79962699 | 65.66466667 | 101.0717607 | 65.53153153 |
| 20761 | 40.35207207 | 22.88404336 | 74.42493333 | 75.27113246 | 65.42942943 |
| 8053 | 158.3908408 | 52.38168912 | 147.4453333 | 176.0430097 | 67.42942943 |
| 8314 | 37.74684685 | 20.62061151 | 65.18866667 | 60.69449107 | 70.91291291 |
| 13251 | 24.79555556 | 21.39480435 | 56.8832 | 48.20654526 | 67.71171171 |
| 19057 | 37.59798799 | 26.6987731 | 82.2904 | 45.74180362 | 71.84384384 |
| 18598 | 2.265165165 | 31.0457142 | 46.36 | 62.19549621 | 66.96096096 |
| 22681 | 290.1493994 | 74.02188774 | 341.2553333 | 196.8692173 | 67.90990991 |
| 5699 | 474.8620721 | 82.83755107 | 392.5314667 | 205.7781669 | 69.69369369 |
| 4944 | 97.85765766 | 33.05505292 | 129.9089333 | 73.44437126 | 70.07807808 |
| 18654 | 225.9345946 | 55.31875281 | 143.4742667 | 91.96906131 | 71.60960961 |
| 1748 | 13.15117117 | 11.26802396 | 24.1972 | 19.76149618 | 66.31231231 |
| 19723 | 110.8291592 | 44.46587867 | 143.9182667 | 89.66412878 | 66.74474474 |
| 11644 | 240.3689189 | 68.04134468 | 292.4717333 | 164.2458978 | 71.62762763 |
| 17100 | 1169.730811 | 269.2963014 | 1181.04 | 640.0103416 | 65.54354354 |
| 15642 | 244.0573273 | 64.24334959 | 314.7402667 | 102.3420021 | 65.15915916 |
| 8085 | 90.8796997 | 37.21612484 | 111.6589333 | 73.75941522 | 66.42642643 |
| 2341 | 214.7996096 | 62.77279195 | 228.1268 | 132.5745965 | 67.22522523 |
| 19544 | 877.0103003 | 207.0008993 | 914.8142667 | 413.8674842 | 65.59159159 |
| 24246 | 259.5653754 | 78.38332337 | 280.1245333 | 151.6943832 | 69.24324324 |
| 7471 | 174.3034535 | 51.22798503 | 433.0494667 | 531.6293255 | 67.91591592 |
| 24634 | 16.77540541 | 9.943729554 | 50.93453333 | 70.34485769 | 65.23123123 |
| 355 | 6.891771772 | 22.87156702 | 42.36933333 | 53.61521026 | 66.58258258 |
| 23165 | 143.0078679 | 41.53384915 | 207.1412 | 158.8961956 | 65.06306306 |
| 14341 | 13.8921021 | 17.44846007 | 26.80346667 | 67.66345777 | 65.57957958 |
| 18715 | 141.1687688 | 34.79869736 | 205.4153333 | 113.6473079 | 66.24624625 |
| 12366 | 41.18138138 | 16.18910839 | 70.5228 | 48.94326441 | 66.91291291 |
| 13646 | 539.8878378 | 151.4244238 | 779.5981333 | 361.1897058 | 66.46246246 |
| 21592 | 124.7615015 | 30.49860974 | 130.6713333 | 83.57220176 | 68.16216216 |
| 23964 | 78.86081081 | 26.59131706 | 124.9181333 | 53.47287058 | 72.81081081 |
| 18472 | 62.04138138 | 37.28023736 | 117.0882667 | 75.31753547 | 68.81081081 |
| 18507 | 526.0994595 | 129.9778234 | 680.3916 | 353.5398787 | 73.86186186 |
| 3433 | 847.1170571 | 407.7572204 | 1110.959467 | 924.250964 | 66.76276276 |
| 20944 | 395.7508108 | 65.89399347 | 402.8101333 | 182.7794632 | 65.47747748 |
| 14449 | 61.24468468 | 23.25839512 | 61.46186667 | 62.81005197 | 65.11111111 |
| 13929 | 104.2424925 | 41.66084094 | 145.694 | 84.57514859 | 71.06306306 |
| 18172 | 49.80393393 | 24.91031155 | 87.37426667 | 36.14079316 | 75.37537538 |
| 3669 | 26.48882883 | 18.05107279 | 29.7056 | 42.38183504 | 72.84684685 |
| 21975 | 171.0662462 | 55.4224825 | 227.5046667 | 130.6607994 | 76.01201201 |
| 3191 | 158.8891892 | 42.30405855 | 215.4716 | 68.50071702 | 67.52552553 |
| 15197 | 339.3753453 | 66.52029366 | 250.0181333 | 118.6375288 | 67.90990991 |
| 18217 | 124.8142042 | 42.23932068 | 138.7856 | 104.7169781 | 65.47747748 |
| 6172 | 21.8966967 | 10.84468475 | 18.30253333 | 25.61298552 | 65.47747748 |
| 9059 | 261.9566066 | 69.08379794 | 360.6361333 | 155.8157674 | 79.75975976 |
| 5193 | 1921.493814 | 375.9558746 | 1838.5492 | 938.8969967 | 66.87687688 |
| 19012 | 415.4101201 | 102.7400568 | 488.3222667 | 259.8339431 | 69.90990991 |
| 10972 | 179.3564865 | 58.78370918 | 255.3004 | 95.32119822 | 66.94294294 |
| 6274 | 356.3527327 | 70.55438269 | 394.8405333 | 170.0040819 | 67.54354354 |
| 20998 | 284.8785586 | 61.87358479 | 297.0229333 | 145.2585479 | 65.30930931 |
| 21816 | 256.1336937 | 58.5825438 | 269.8497333 | 132.1272928 | 66.21021021 |
| 2093 | 329.9577477 | 84.7105984 | 295.9622667 | 192.1529976 | 69.24324324 |
| 5622 | 201.8865465 | 54.07644043 | 215.5912 | 115.800727 | 65.17717718 |
| 18529 | 298.7122222 | 72.59347086 | 330.1838667 | 167.9640031 | 70.19219219 |
| 815 | 1294.80985 | 372.9112118 | 1445.012 | 797.6685248 | 67.54354354 |
| 12031 | 81.85009009 | 17.57488942 | 83.30773333 | 37.36427512 | 66.49249249 |
| 10549 | 20.88075075 | 9.268948244 | 51.72626667 | 49.07283376 | 67.2972973 |
| 19949 | 84.83585586 | 19.03876357 | 124.9334667 | 86.01204607 | 66.76276276 |
| 19729 | 98.26867868 | 41.10355435 | 141.7825333 | 164.3693855 | 66.46246246 |
| 22958 | 222.3516216 | 61.89665476 | 332.0626667 | 184.4991397 | 67.21321321 |
| 22196 | 53.36306306 | 18.57336843 | 48.3064 | 63.37479253 | 67.42942943 |
| 16080 | 11.52768769 | 28.05513739 | 48.91493333 | 54.81379034 | 66.93093093 |
| 22675 | 34.77126126 | 17.78391381 | 66.09426667 | 39.38159792 | 71.86186186 |
| 3817 | 170.7467267 | 54.41675831 | 253.9452 | 129.9122613 | 66.52852853 |
| 20868 | 56.04309309 | 26.6401069 | 97.7992 | 50.53188746 | 65.77777778 |
| 21632 | 199.0260961 | 70.73366374 | 277.3632 | 210.302496 | 72.52852853 |
| 5228 | 13.29966967 | 35.9860809 | 68.88893333 | 73.81005446 | 66.29429429 |
| 18695 | 33.05660661 | 35.62163487 | 87.28466667 | 75.91128948 | 65.90990991 |
| 23998 | 33.52453453 | 15.11942669 | 51.97706667 | 30.27439391 | 65.17717718 |
| 16394 | 799.7536336 | 252.3122848 | 919.6201333 | 622.9247718 | 68.96096096 |
| 2539 | 16.97189189 | 24.0247802 | 44.98853333 | 40.59096733 | 68.14414414 |
| 4407 | 78.10762763 | 19.73747997 | 79.0836 | 49.63701438 | 68.64264264 |
| 18473 | 214.0645946 | 63.96883025 | 268.3337333 | 138.6580163 | 69.77777778 |
| 17563 | 1512.565075 | 336.0712844 | 1552.276933 | 788.9080025 | 67.99399399 |
| 3430 | 113.1247748 | 24.63319195 | 120.2838667 | 55.86774387 | 70.51051051 |
| 15626 | 1559.982192 | 392.9104244 | 1587.424667 | 857.0575602 | 65.00900901 |
| 12921 | 193.4437838 | 64.18669085 | 210.2042667 | 123.8728695 | 67.17717718 |
| 18883 | 65.9106006 | 19.09173712 | 346.6489333 | 638.9246984 | 65.0990991 |
| 19075 | 112.8835736 | 29.19877566 | 190.2618667 | 134.2096951 | 65.12912913 |
| 18881 | 19.10243243 | 7.86572788 | 38.22826667 | 36.0712046 | 67.51351351 |
| 24272 | 9.686756757 | 22.75875573 | 53.3452 | 100.2345994 | 66.54654655 |
| 1893 | 331.2993994 | 138.696175 | 619.7997333 | 519.3306369 | 65.51351351 |
| 6438 | 27.83207207 | 12.16784973 | 49.74013333 | 41.92384442 | 65.27927928 |
| 7168 | −1.737027027 | 19.39544478 | 31.3912 | 55.21211843 | 66.0960961 |
| 15192 | 60.8587988 | 29.37339287 | 111.5484 | 79.91181268 | 67.94594595 |
| 5186 | 58.5721021 | 24.67471847 | 105.746 | 64.30219504 | 69.7957958 |
| 15841 | 45.80051051 | 15.16607716 | 71.3828 | 34.35511629 | 69.71171171 |
| 10659 | 119.6529129 | 40.63333371 | 180.052 | 95.89034622 | 65.11111111 |
| 8012 | 23.92666667 | 12.31341219 | 43.68653333 | 30.44973938 | 71.71171171 |
| 23123 | 291.002042 | 75.85347746 | 415.2902667 | 174.8884898 | 68.44444444 |
| 3823 | 367.4383483 | 77.89555445 | 473.9110667 | 191.4850628 | 65.56156156 |
| 5461 | 107.45003 | 35.26438695 | 152.7890667 | 85.66813767 | 67.04504505 |
| 13005 | 20.45213213 | 9.025723211 | 32.71026667 | 21.01691423 | 65.11111111 |
| 9796 | 3.506786787 | 12.46437314 | 24.92586667 | 22.49725167 | 70.96096096 |
| 18867 | 175.3500601 | 45.30841723 | 206.2506667 | 124.9503735 | 66.31231231 |
| 3941 | 162.8698198 | 50.68034573 | 252.2224 | 109.5806157 | 73.1951952 |
| 6585 | 671.153994 | 331.4712674 | 993.8865333 | 751.7989804 | 69.12912913 |
| 18389 | 686.5324925 | 327.0174426 | 1031.984133 | 716.273277 | 65.4954955 |
| 3799 | 347.6641742 | 84.61977436 | 421.5834667 | 235.3294566 | 73.71171171 |
| 17 | 102.115015 | 51.55145585 | 118.8796 | 142.6167557 | 68.37837838 |
| 23732 | 283.160991 | 120.5760896 | 363.3473333 | 280.470972 | 67.87987988 |
| 8022 | 11.01855856 | 20.97671253 | 36.92493333 | 41.91849093 | 66.82882883 |
| 3434 | 782.4078679 | 306.6851584 | 974.8838667 | 741.4375723 | 66.16216216 |
| 18831 | 2653.003964 | 594.2303789 | 2892.579333 | 1600.98895 | 69.84384384 |
| 15729 | 29.23543544 | 20.71589737 | 1.493333333 | 32.22650862 | 65.02702703 |
| 8058 | 313.8185285 | 63.47764541 | 320.8578667 | 155.8802739 | 66.14414414 |
| 18606 | 755.4990991 | 177.0235611 | 787.5954667 | 439.0702126 | 70.29429429 |
| 23612 | 135.0208408 | 35.35749654 | 148.7529333 | 85.93640724 | 66.06006006 |
| 5482 | 298.6487688 | 69.44419707 | 207.0368 | 124.5076928 | 70.06006006 |
| 23645 | 85.75402402 | 23.20623619 | 102.6801333 | 50.55266546 | 70.51051051 |
| 7913 | 38.34870871 | 24.32239025 | 87.43133333 | 106.9607276 | 65.98198198 |
| 12978 | 74.58318318 | 19.9642456 | 118.052 | 122.2691474 | 67.96396396 |
| 3718 | 27.41858859 | 10.36874312 | 61.1336 | 47.50227834 | 71.36336336 |
| 10837 | 72.48654655 | 25.13861801 | 110.2788 | 107.4329066 | 66.46246246 |
| 21682 | −19.73867868 | 23.16892194 | 26.7004 | 68.44642538 | 68.82882883 |
| 19658 | 8.038738739 | 13.08811047 | 28.9032 | 36.72423098 | 65.94594595 |
| 2310 | 34.66915916 | 34.32976119 | 84.04733333 | 108.9209867 | 65.34534535 |
| 12583 | 25.53441441 | 10.87954672 | 43.09533333 | 25.2762214 | 65.12912913 |
| 16168 | 183.7095495 | 39.71588486 | 223.3278667 | 116.8093091 | 67.42942943 |
| 17950 | 39.89375375 | 17.03772996 | 62.28813333 | 44.1986732 | 71.06306306 |
| 21667 | 25.55798799 | 16.28610574 | 54.18893333 | 38.98081703 | 71.84384384 |
| 11483 | 105.6160961 | 33.39285637 | 140.626 | 92.63212292 | 69.1951952 |
| 21766 | 102.3873874 | 30.78764276 | 154.6985333 | 60.50836756 | 68.51051051 |
| 17485 | 37.34957958 | 18.51871628 | 41.9596 | 55.13918699 | 66.74474474 |
| 6548 | 223.351982 | 64.71562964 | 309.3073333 | 182.737113 | 71.34534535 |
| 16136 | 111.0614114 | 38.38598665 | 168.1841333 | 92.55572117 | 67.62762763 |
| 23013 | 312.8023423 | 67.22989834 | 314.1472 | 191.3850765 | 68.74474474 |
| 11708 | 656.2660961 | 100.9917709 | 672.7158667 | 295.123462 | 66.29429429 |
| 23583 | 55.02069069 | 15.34138636 | 67.3188 | 41.45032967 | 68.66066066 |
| 21581 | 574.5948649 | 100.9941053 | 585.4786667 | 247.833259 | 65.71171171 |
| 21709 | 251.0371772 | 46.08494762 | 304.7734667 | 105.6013574 | 72.37837838 |
| 23731 | 156.557988 | 34.90185251 | 178.8264 | 93.8952906 | 74.59459459 |
| 21443 | 211.1505105 | 55.24450794 | 252.3273333 | 130.5721587 | 69.47747748 |
| 10820 | 2016.868679 | 394.8495465 | 2134.5488 | 916.5400649 | 66.42642643 |
| 6226 | 180.9392192 | 56.11190149 | 208.4389333 | 126.2274074 | 67.69369369 |
| 6881 | 187.4048048 | 36.38697325 | 541.9369333 | 770.4809513 | 67.6996997 |
| 15540 | 50.16471471 | 19.80763612 | 125.0138667 | 146.3339 | 67.81381381 |
| 16053 | 104.7786486 | 40.36047993 | 203.0978667 | 222.8617765 | 65.38138138 |
| 11258 | 9.338348348 | 12.8288048 | 46.81333333 | 66.06550364 | 65.21321321 |
| 6102 | 34.6881982 | 14.54131359 | 67.62853333 | 59.94280266 | 65.06306306 |
| 2992 | 32.3545045 | 14.84118742 | 72.47946667 | 60.57864843 | 69.57957958 |
| 16312 | 62.09135135 | 18.03624356 | 105.7618667 | 84.27319098 | 73.72972973 |
| 21579 | 89.67822823 | 30.10965619 | 156.6705333 | 114.2049362 | 66.46246246 |
| 21514 | 51.22918919 | 17.14684899 | 86.27786667 | 58.87843485 | 66.6966967 |
| 13619 | 126.2540541 | 35.20172348 | 165.5262667 | 129.0628828 | 66.97897898 |
| 7161 | 37.29990991 | 14.33737399 | 64.94226667 | 34.29983416 | 66.61261261 |
| 2410 | 17.10996997 | 9.41125795 | 24.29386667 | 32.68288621 | 70.18018018 |
| 7806 | 32.89921922 | 12.13507758 | 51.75133333 | 33.42676074 | 66.31231231 |
| 20035 | 160.8397898 | 51.06549667 | 231.2181333 | 153.5927743 | 71.94594595 |
| 16 | 1215.400871 | 271.7923445 | 1738.301333 | 669.9464909 | 71.1951952 |
| 18095 | 851.0671171 | 114.9507612 | 826.31 | 360.5139865 | 69.27927928 |
| 21165 | 26.14231231 | 19.68375564 | 62.034 | 45.32615008 | 69.11111111 |
| 23632 | 117.9095495 | 35.38523686 | 145.4554667 | 107.2681995 | 68.46246246 |
| 20869 | 65.51135135 | 31.29957449 | 121.7910667 | 62.82875419 | 70.37837838 |
| 24040 | 367.1426426 | 108.9136658 | 462.4882667 | 299.6696857 | 70.31231231 |
| 2706 | 445.8896396 | 93.42872356 | 418.4548 | 256.4528316 | 68.82882883 |
| 13460 | 339.8782282 | 58.70581576 | 244.3704 | 90.92216048 | 71.30930931 |
| 17540 | 338.986006 | 101.9061709 | 406.9857333 | 274.3575698 | 71.04504505 |
| 2757 | 855.6433033 | 177.028321 | 1118.036 | 404.6706488 | 70.14414414 |
| 2742 | 77.8221021 | 60.84443291 | −2.269733333 | 113.8540968 | 69.24324324 |
| 16982 | 1.248288288 | 13.68589859 | 124.3068 | 417.5962153 | 67.76576577 |
| 24716 | −9.734984985 | 7.656839278 | 29.73813333 | 81.36529343 | 65.68168168 |
| 9452 | 222.9467267 | 74.4629772 | 544.6782667 | 684.875271 | 68.28228228 |
| 356 | 32.46015015 | 31.50629355 | 134.3224 | 195.0399764 | 69.5975976 |
| 17506 | 37.83588589 | 22.00037604 | 118.3630667 | 122.8476271 | 70.41441441 |
| 22626 | 68.76153153 | 29.2925403 | 144.0328 | 125.3132555 | 66.1981982 |
| 23005 | 78.15855856 | 30.11948205 | 158.4333333 | 155.1551272 | 69.2972973 |
| 15300 | 219.4616517 | 88.68064613 | 311.8266667 | 287.0270087 | 69.68168168 |
| 17734 | 820.2270571 | 276.7461649 | 1276.288267 | 898.5903168 | 70.91291291 |
| 24235 | 184.918018 | 49.36407201 | 273.1097333 | 144.2502354 | 66.46246246 |
| 2741 | 28.07504505 | 16.06701889 | 47.6888 | 39.7256073 | 65.66366366 |
| 16314 | 47.75924925 | 46.46160475 | 119.6102667 | 83.53206694 | 72.03003003 |
| 5494 | 47.25780781 | 24.4794228 | 55.16266667 | 76.43192798 | 65.34534535 |
| 7299 | 224.0332733 | 96.06234119 | 400.7057333 | 225.7313284 | 68.59459459 |
| 23044 | 224.0903604 | 40.57434413 | 241.4468 | 121.4169255 | 65.86186186 |
| 16821 | 737.4866967 | 122.45802 | 776.4358667 | 364.0221538 | 66.66066066 |
| 15185 | 836.8953754 | 182.6575292 | 876.6856 | 449.6124882 | 66.96096096 |
| 15283 | 304.6465165 | 59.01860559 | 335.8565333 | 134.6477232 | 71.30930931 |
| 17104 | 533.3456156 | 140.8351573 | 579.2497333 | 305.4651559 | 66.29429429 |
| 5675 | 126.4663664 | 44.91054543 | 313.3089333 | 357.5454003 | 67.68168168 |
| 11873 | 136.426997 | 52.38531024 | 295.0414667 | 330.1990618 | 66.1981982 |
| 22765 | 19.95795796 | 9.841589035 | 49.7652 | 48.36528961 | 66.54654655 |
| 17161 | 196.9924625 | 63.30941186 | 344.6728 | 305.35076 | 69.5975976 |
| 2629 | 21.25555556 | 9.303175262 | 35.06306667 | 45.76494903 | 68.03003003 |
| 11531 | 347.5362763 | 125.6397047 | 546.3824 | 512.2328472 | 68.41441441 |
| 10182 | 14.42633634 | 45.39196964 | 99.80266667 | 141.6511343 | 65.44744745 |
| 5297 | 208.922012 | 59.24907417 | 321.2946667 | 201.7556369 | 68.18018018 |
| 22667 | 49.03594595 | 33.93458508 | 123.0734667 | 105.981439 | 66.24624625 |
| 21239 | 106.1072072 | 43.32188757 | 180.5274667 | 121.9250473 | 68.61261261 |
| 18349 | 171.9368769 | 41.29067474 | 215.8301333 | 142.6442584 | 68.82882883 |
| 3003 | 49.46108108 | 48.66360051 | 113.7446667 | 117.8949655 | 65.64564565 |
| 18830 | 3900.264985 | 802.5740391 | 4205.326133 | 2430.213531 | 67.26126126 |
| 14250 | 150.0712012 | 40.36395039 | 164.3112 | 114.6934595 | 66.52852853 |
| 16215 | 215.1935736 | 46.06168644 | 201.6325333 | 109.0627132 | 65.1951952 |
| 23578 | 302.91997 | 69.08516771 | 321.9876 | 155.9602644 | 67.77777778 |
| 3738 | 207.3542943 | 57.37567983 | 226.4605333 | 130.4036913 | 65.02702703 |
| 16081 | 81.31096096 | 37.49730539 | 417.8996 | 776.3912064 | 68.58258258 |
| 567 | 8.786756757 | 19.6043637 | 119.8282667 | 194.0106893 | 66.43243243 |
| 6982 | 9.056966967 | 12.04326158 | 84.65053333 | 136.9829322 | 68.1981982 |
| 8759 | 29.31588589 | 30.75732946 | 117.6049333 | 149.3627949 | 67.01501502 |
| 15382 | 40.64624625 | 27.64454786 | 130.1093333 | 138.4893887 | 68.18018018 |
| 15212 | 48.23750751 | 21.91906398 | 102.0592 | 92.55491027 | 66.26426426 |
| 12581 | 40.43765766 | 14.53283175 | 76.5044 | 64.68039423 | 69.21321321 |
| 15301 | 86.80627628 | 47.38871887 | 143.158 | 166.8604371 | 73.31531532 |
| 11242 | 29.36057057 | 16.46344227 | 59.1448 | 57.52839734 | 69.42942943 |
| 17735 | 872.2572072 | 315.0601774 | 1495.168667 | 967.1088715 | 68.01201201 |
| 20433 | 63.74864865 | 21.11187056 | 93.78546667 | 72.70130138 | 74.84684685 |
| 9424 | 294.5164565 | 73.42587321 | 375.1704 | 261.9582482 | 69.27927928 |
| 1609 | 752.904024 | 207.8298617 | 910.3266667 | 706.5647674 | 65.12912913 |
| 21841 | 363.3037237 | 98.73789477 | 431.2704 | 274.1229162 | 66.51051051 |
| 19011 | 558.6072072 | 112.2070055 | 604.9225333 | 312.0176449 | 69.1951952 |
| 13420 | 394.5314114 | 82.81294506 | 420.2732 | 237.3869261 | 66.89489489 |
| 19085 | 54.89846847 | 12.86081694 | 60.8328 | 34.85106767 | 69.11111111 |
| 11849 | 839.1966967 | 180.5114352 | 877.4672 | 435.6222224 | 66.31231231 |
| 25705 | 330.1517117 | 76.02852844 | 373.9126667 | 179.3556642 | 71.71171171 |
| 23471 | 68.07546547 | 27.22670055 | 350.3384 | 553.0776103 | 69.3993994 |
| 20449 | 77.32024024 | 56.32117764 | 261.8505333 | 376.7640297 | 67.23123123 |
| 23626 | 52.57429429 | 23.49905084 | 131.8013333 | 133.2193841 | 68.11411411 |
| 11114 | 31.76129129 | 15.33633558 | 81.476 | 83.14401664 | 70.26426426 |
| 5384 | 39.72459459 | 30.39866325 | 108.9032 | 122.9407092 | 67.23123123 |
| 357 | 23.45963964 | 10.14080555 | 42.20293333 | 47.2347585 | 67.57957958 |
| 21683 | 47.01618619 | 17.95548224 | 87.94773333 | 74.53349783 | 68.91291291 |
| 4491 | 41.94915916 | 25.39221348 | 46.314 | 95.16841592 | 67.66366366 |
| 22197 | 120.2223123 | 36.70029871 | 189.3146667 | 92.40831407 | 70.21021021 |
| 11153 | 416.2181682 | 95.50995272 | 466.0797333 | 254.846382 | 68.96096096 |
| 11208 | 330.6584685 | 103.4859949 | 400.4674667 | 249.141563 | 70.24624625 |
| 25379 | 185.6321021 | 40.4422624 | 201.9674667 | 102.7164965 | 66.14414414 |
| 19358 | 2787.216847 | 870.0544471 | 1645.817733 | 1445.279135 | 70.64264264 |
| 1221 | 4.623993994 | 6.543957295 | 162.8805333 | 351.0642505 | 69.0990991 |
| 244 | 58.45195195 | 21.55924157 | 230.3276 | 403.0327754 | 68.13213213 |
| 657 | 213.6305105 | 44.46268652 | 438.9708 | 365.3515242 | 69.44744745 |
| 18597 | 94.84132132 | 24.63570978 | 165.3626667 | 174.1846481 | 74.04804805 |
| 13930 | 97.47840841 | 25.86326313 | 169.7642667 | 155.6754159 | 73.68168168 |
| 3050 | 70.99321321 | 30.97427774 | 178.296 | 152.017052 | 71.96396396 |
| 14840 | 16.23504505 | 9.495416923 | 29.5148 | 45.54277471 | 73.2972973 |
| 15892 | 46.68723724 | 28.17584274 | 113.8484 | 106.7604541 | 70.33033033 |
| 19184 | 16.72972973 | 15.38497159 | 42.66706667 | 53.94375038 | 67.94594595 |
| 13291 | 158.8336937 | 36.43992152 | 190.7336 | 125.3794767 | 68.46246246 |
| 21653 | 108.6768168 | 33.8784464 | 180.1490667 | 85.84586661 | 71.86186186 |
| 21740 | 254.009009 | 65.86437227 | 345.4734667 | 223.7874086 | 72.82882883 |
| 21796 | 253.457958 | 76.45052978 | 344.1602667 | 201.6813064 | 77.71171171 |
| 4048 | 6.934114114 | 28.88749489 | 109.7854667 | 203.1293868 | 69.6996997 |
| 4725 | 67.20492492 | 45.50835024 | 196.1262667 | 241.2131264 | 66.26426426 |
| 12979 | 283.0270571 | 69.70681028 | 474.0812 | 466.1164226 | 70.33033033 |
| 10317 | 168.6707207 | 98.09793834 | 398.7746667 | 372.3880631 | 67.81381381 |
| 4969 | −27.81138138 | 29.49309846 | 39.90693333 | 109.5442595 | 66.78078078 |
| 22666 | 64.99294294 | 29.95874228 | 132.2769333 | 77.22446481 | 72.46246246 |
| 24219 | 270.5740541 | 56.99366137 | 310.9173333 | 195.1191918 | 70.03003003 |
| 9166 | 58.24063063 | 36.37848468 | 115.5628 | 87.83244861 | 67.41141141 |
| 16533 | 297.6689489 | 80.00007371 | 353.3222667 | 204.2514635 | 74.44444444 |
| 13240 | 36.84933934 | 24.2983443 | 217.4728 | 295.2240047 | 71.01501502 |
| 15191 | 73.55387387 | 147.0000176 | 835.7512 | 1240.522261 | 68.64864865 |
| 22615 | 38.06543544 | 38.14715918 | 179.3072 | 213.8526855 | 71.98198198 |
| 20589 | 141.2745946 | 40.41159864 | 220.17 | 222.0598445 | 66.86486486 |
| 21284 | 11.28018018 | 11.32357446 | 42.2628 | 45.75836649 | 68.03003003 |
| 7700 | 38.38453453 | 15.97951121 | 74.8852 | 63.58685582 | 68.11411411 |
| 24209 | −0.938468468 | 32.11077523 | 98.36706667 | 131.0425901 | 70.84684685 |
| 12745 | 171.2609309 | 45.01220927 | 256.3192 | 127.4189463 | 70.31231231 |
| 10071 | 255.8928829 | 73.65188401 | 333.818 | 244.1968914 | 74.0960961 |
| 20740 | 416.8621321 | 104.9626061 | 504.2676 | 311.2743053 | 72.37837838 |
| 15004 | 272.1264565 | 96.15890067 | 711.5694667 | 654.1443082 | 69.98198198 |
| 21663 | 329.2990991 | 103.8992814 | 468.7245333 | 414.0000322 | 68.04804805 |
| 13634 | 865.2964264 | 132.9344871 | 1009.422133 | 569.5991098 | 70.97897898 |
| 22311 | 129.7924324 | 36.03268841 | 167.8010667 | 130.8693873 | 69.57957958 |
| 15644 | 1108.512883 | 193.4400937 | 1168.529067 | 588.9984693 | 71.71171171 |
| 21657 | 810.1552553 | 149.7486347 | 950.3389333 | 452.1725091 | 77.34534535 |
| 15051 | 100.5486787 | 36.5306203 | 423.4646667 | 539.2944453 | 71.83183183 |
| 804 | 48.453003 | 17.3476378 | 136.0454667 | 252.5131317 | 70.4984985 |
| 17477 | 48.03099099 | 18.31845744 | 130.196 | 122.9390777 | 73.53153153 |
| 9583 | 63.32945946 | 23.97049962 | 156.3501333 | 138.8519633 | 70.56456456 |
| 806 | 104.0790991 | 38.57735781 | 262.0394667 | 206.8242271 | 75.38138138 |
| 17736 | 495.3388889 | 193.1228301 | 840.7049333 | 673.1283815 | 74.3963964 |
| 8477 | 633.9702102 | 137.901978 | 773.1670667 | 441.5078833 | 71.27927928 |
| 13633 | 305.4563664 | 90.26020694 | 409.7552 | 276.7145158 | 70.67867868 |
| 11404 | 95.40525526 | 25.45018912 | 124.0573333 | 81.37672486 | 75.06306306 |
| 23038 | 12.70459459 | 31.90466814 | 123.0434667 | 144.4113666 | 75.2972973 |
| 22432 | 117.0051652 | 32.59966127 | 143.2058667 | 83.64197807 | 68.89489489 |
| 21654 | 195.4411111 | 36.28835456 | 269.9418667 | 198.8356806 | 71.8978979 |
| 15042 | 39.07891892 | 31.16281275 | 133.2384 | 112.6081482 | 73.96396396 |
| 9423 | 161.4653153 | 43.39107869 | 234.2049333 | 186.0967937 | 69.64564565 |
| 23869 | 37.38426426 | 23.1410014 | 146.1506667 | 169.2856008 | 69.44744745 |
| 15189 | 263.7455856 | 123.0099327 | 829.2574667 | 845.5430688 | 72.18018018 |
| 22677 | 130.0316817 | 78.66102337 | 291.6418667 | 164.9890655 | 74.37837838 |
| 15190 | 192.534955 | 113.543054 | 768.8886667 | 843.1346494 | 68.48048048 |
| 14763 | 15.69915916 | 55.29195278 | 160.1976 | 217.6567383 | 68.03003003 |
| 24200 | 589.2201201 | 119.7479072 | 757.8837333 | 476.5973886 | 72.46246246 |
| 574 | 557.0646847 | 127.5518084 | 682.7218667 | 404.188059 | 76.0960961 |
| 1892 | 982.935015 | 274.4452657 | 1255.822533 | 798.7510893 | 75.72972973 |
| 15003 | 57.02492492 | 43.85939442 | 361.8852 | 467.3514167 | 70.43243243 |
| 17401 | 519.3374474 | 129.24869 | 1091.634533 | 771.6544413 | 72.93093093 |
| 3049 | 237.4652252 | 63.18596703 | 414.9229333 | 350.7621453 | 76.48048048 |
| 5292 | 80.4015015 | 36.86776863 | 190.9689333 | 128.0774443 | 73.23123123 |
| 22321 | 153.3996697 | 50.22365877 | 299.7406667 | 179.4179144 | 75.74774775 |
| 6606 | 1714.972342 | 475.267636 | 2441.403733 | 1648.142289 | 75.8978979 |
| 15002 | 155.3522823 | 52.61286866 | 957.8806667 | 1185.872821 | 77.91591592 |

TABLE 5A
IFOSFAMIDE Attorney Docket No. 44921-5090US
Timepoint(s): 48, 144 hrs Document No. 1828250.1

| Identifier | NonToxMean | NonToxSD | ToxMean | ToxSD | LDA score |
|---|---|---|---|---|---|
| 16448 | 295.3424587 | 122.9466968 | 75.04222222 | 17.90922442 | 96.9924812 |
| 26150 | 3548.292872 | 2396.6288611 | 261.2477778 | 231.8709164 | 96.74185464 |
| 24012 | 893.9981198 | 297.5327424 | 1533.29 | 247.1554579 | 96.49122807 |
| 19358 | 2632.632831 | 1141.272779 | 398.9844444 | 344.5671647 | 96.11528822 |
| 1687 | 5198.764855 | 2223.536206 | 734.3333333 | 690.6095483 | 95.98997494 |
| 18907 | 460.9429339 | 259.0969563 | 59.24111111 | 41.11022058 | 95.73934837 |
| 23096 | 86.6802686 | 114.0827577 | 325.1088889 | 55.08510812 | 95.36340852 |
| 1689 | 11121.36928 | 5507.347687 | 1809.905556 | 1809.714704 | 94.98746867 |
| 4031 | 62.98836777 | 33.23422192 | 131.4044444 | 16.31989439 | 94.61152882 |
| 12745 | 188.7965909 | 81.83675846 | 279.2366667 | 29.31450665 | 94.61152882 |
| 7120 | 52.7047314 | 30.78982343 | 4.4 | 8.386788718 | 94.48621554 |
| 9073 | 23.56423554 | 15.1644237 | 60.74222222 | 12.81258442 | 94.36090226 |
| 18881 | 24.01061983 | 21.70350112 | 36.94333333 | 4.767224035 | 94.36090226 |
| 17832 | 5911.541281 | 2981.707879 | 745.6577778 | 722.4025935 | 94.11027569 |
| 17883 | 4401.742872 | 2443.409704 | 2660.705556 | 301.8011314 | 94.11027569 |
| 25517 | 175.8206612 | 106.8012091 | 69.60333333 | 9.200444283 | 93.98496241 |
| 25742 | 8347.828905 | 5377.253327 | 4622.804444 | 568.0292832 | 93.98496241 |
| 20859 | 5611.207438 | 3111.792163 | 3360.028889 | 326.5182489 | 93.85964912 |
| 17687 | 82.56880165 | 27.62361939 | 135.0622222 | 16.95063625 | 93.85964912 |
| 14981 | 5912.081136 | 3450.077362 | 3177.887778 | 363.279144 | 93.60902256 |
| 13382 | 130.2001033 | 65.68493084 | 271.6855556 | 61.41857478 | 93.60902256 |
| 1845 | 60.05626033 | 81.65511417 | 66.3544444444 | 23.27943788 | 93.35839599 |
| 8515 | 473.0626033 | 167.3302597 | 211.4588889 | 47.70883551 | 93.23308271 |
| 19518 | 37.11066116 | 37.86481857 | 63.35444444 | 14.86912751 | 93.23308271 |
| 19013 | 19.1711157 | 11.63088946 | 37.75666667 | 5.040974608 | 93.10776942 |
| 6727 | 142.0419628 | 37.81757964 | 224.84 | 31.88848969 | 93.10776942 |
| 8612 | 48.80396694 | 32.68228712 | 1.941111111 | 8.541677886 | 93.10776942 |
| 13336 | 342.3178926 | 143.4507323 | 164.2811111 | 43.96638757 | 92.85714286 |
| 1831 | 201.9417769 | 86.41174095 | 73.95777778 | 27.44454581 | 92.85714286 |
| 21152 | 6146.16719 | 3971.973661 | 3550.796667 | 411.3731839 | 92.85714286 |
| 17917 | 156.96514462809 | 154.714297 | 108.8144444 | 67.88608493 | 92.85714286 |
| 16001 | 7525.862996 | 4857.843227 | 3916.177778 | 472.7264354 | 92.0651629 |
| 460 | 3364.606777 | 1604.578237 | 2261.226667 | 182.9600171 | 92.60651629 |
| 16687 | 198.8393802 | 57.67831435 | 275.5644444 | 18.73961053 | 92.48120301 |
| 23157 | 285.4082645 | 617.3022225 | 224.7822222 | 10.76623167 | 92.35588972 |
| 25669 | 6222.719731 | 3227.407969 | 3764.182222 | 347.3161204 | 92.23057644 |
| 1169 | 50.48876033 | 27.83295948 | 74.21777778 | 8.167539069 | 92.10526316 |
| 3016 | 3592.913471 | 1785.617911 | 2301.943333 | 216.9545399 | 92.10526316 |
| 16274 | 55.744462809917 | 47.41495515 | 141.867777777 | 25.78003965 | 91.85463659 |
| 24632 | 52.85609504 | 18.69125368 | 82.03666667 | 14.81684008 | 91.85463659 |
| 11896 | 55.327334710743 | 42.34783266 | 18.16222222 | 28.27681284 | 91.85463659 |
| 13082 | 10.859938016528 | 26.2467805 | 23.61222222 | 9.427467552 | 91.72932331 |
| 2522 | 184.46438016528 | 111.8533887 | 46.68888888888 | 51.38384776 | 91.72932331 |
| 3887 | 46.36555785 | 57.74475168 | 73.69222222 | 17.93160393 | 91.72932331 |
| 12108 | 73.58801653 | 52.40913753 | 163.39 | 29.09819754 | 91.47869674 |
| 17541 | 76.55303719 | 44.33328287 | 201.26 | 71.45394531 | 91.31161236 |
| 20484 | 5409.051632 | 3145.685654 | 3026.711111 | 370.2778782 | 91.22807018 |
| 2134 | 65.17946281 | 18.86782948 | 95.17666667 | 11.55191326 | 91.22807018 |
| 15084 | 176.612562 | 89.38204501 | 122.1722222 | 13.03599227 | 91.22807018 |
| 2361 | 0.3254958677685 | 19.51740492 | 26.99444444444 | 7.107026648 | 91.10275689 |
| 16549 | 79.11066116 | 40.95106431 | 98.56888889 | 8.069004654 | 90.97744361 |
| 25469 | 3999.101715 | 2264.177054 | 702.9644444 | 600.79311881 | 90.97744361 |
| 20735 | 112.5160124 | 52.71330364 | 175.523333 | 22.133977271 | 90.85213033 |
| 25468 | 5394.421612 | 3205.5442951 | 824.2277778 | 807.14183881 | 90.85213033 |
| 25439 | 4434.09407 | 2841.5371851 | 2727.964444 | 324.22106681 | 90.85213033 |
| 449 | 3119.018574 | 1522.513541 | 2215.582222 | 195.3333825 | 90.85213033 |
| 12674 | 1727.625393 | 573.63472451 | 1266.653333 | 82.76753817 | 90.85213033 |
| 11563 | 174.858657 | 303.62734 | 214.8022222 | 29.01315339 | 90.72681704 |
| 18774 | 66.48446281 | 31.75449128 | 114.2411111 | 16.06167912 | 90.60150376 |
| 6044 | 231.0816736 | 76.35066102 | 348.8511111 | 57.568058951 | 90.60150376 |
| 1748 | 14.56117769 | 12.82988335 | 49.91333333 | 22.47527031 | 90.55973266 |
| 14983 | 6793.828554 | 4617.087648 | 3724.592222 | 430.8225521 | 90.47619048 |
| 22266 | 284.4200826 | 89.54957173 | 384.9488889 | 72.4625021 | 90.47619048 |
| 3582 | 107.9265289 | 49.73139083 | 139.1611111 | 15.562306581 | 89.97493734 |
| 9501 | 122.450186 | 39.23734565 | 174.8344444 | 17.04399242 | 89.84962406 |
| 8305 | 188.2241322 | 80.74238021 | 299.8333333 | 35.697739421 | 89.84962406 |
| 1321 | 257.13541322314 | 246.8321581 | 336.54 | 27.19207605 | 89.72431078 |
| 6073 | 77.30741736 | 45.53543396 | 2.547777778 | 15.49715762 | 89.47368421 |
| 17214 | 105.1832231 | 37.14938326 | 153.9688889 | 17.60818378 | 89.47368421 |
| 16130 | 5153.919545 | 3243.176778 | 2936.187778 | 393.2186706 | 89.34837093 |
| 17781 | 483.679938 | 99.18061461 | 610.55 | 47.74043151 | 89.22305764 |
| 1685 | 20026.96275 | 13736.02987 | 3216.13 | 2935.386281 | 89.22305764 |
| 11724 | 106.343719 | 62.42593547 | 41.25111111 | 21.447198211 | 89.22305764 |
| 25051 | 4810.628574 | 2521.049129 | 2890.385556 | 362.7703017 | 89.09774436 |
| 14982 | 4946.49657 | 2696.923427 | 2752.296667 | 286.3110091 | 89.09774436 |
| 21974 | 240.1690289 | 61.55796238 | 316.4166667 | 27.59858828 | 89.09774436 |
| 4011 | 19.46816116 | 27.20707326 | 43.91444444 | 8.420742413 | 89.05597327 |
| 17728 | 3421.904587 | 1622.29565 | 2250.813333 | 252.529158 | 88.97243108 |
| 1684 | 6011.583285 | 3680.358832 | 1062.024444 | 953.8925876 | 88.97243108 |
| 16132 | 5183.351736 | 3083.300296 | 3153.822222 | 430.5672499 | 88.97243108 |
| 956 | 234.1724587 | 94.16687929 | 134.0722222 | 31.39964521 | 88.84711779 |
| 16818 | 161.801095 | 135.1141994 | 0.75888888888 | 18.92479752 | 88.84711779 |
| 21985 | 53.52345041 | 19.80522822 | 93.46555556 | 21.81963514 | 88.8053467 |
| 22915 | 127.3294008 | 42.33833705 | 209.1655556 | 47.93496091 | 88.8053467 |
| 18400 | 34.77673554 | 14.19749027 | 54.33 | 8.082788813 | 88.59649123 |
| 15666 | 4342.886095 | 2307.299639 | 2738.857778 | 401.0623884 | 88.59649123 |
| 2341 | 211.188719 | 84.11933769 | 328.2733333 | 52.26430426 | 88.59649123 |
| 25319 | 7042.756198 | 4653.236324 | 3585.176667 | 527.9172732 | 88.59649123 |
| 1830 | 90.31886364 | 49.04982802 | 26.87222222 | 15.87381317 | 88.47117794 |
| 19774 | 88.07989669 | 142.4449065 | 182.3022222 | 57.69461322 | 88.34586466 |
| 18899 | 55.8539876 | 45.8862251 | 69.30555556 | 7.4076432 | 88.34586466 |
| 17892 | 480.7777273 | 340.3353069 | 90.3 | 47.72109282 | 88.34586466 |
| 17829 | 3872.9525 | 2217.680538 | 1132.295556 | 803.6216543 | 88.34586466 |
| 15892 | 59.0617562 | 57.20439404 | 114.7355556 | 29.46817483 | 88.17878028 |
| 10334 | 16.015123966942 | 42.1503708 | 39.90777778 | 24.16863814 | 88.0952381 |
| 19421 | 5009.232851 | 2685.195976 | 2845.693333 | 357.5013906 | 87.96992481 |
| 1688 | 11130.52589 | 7196.98987 | 2484.48 | 2360.420294 | 87.96992481 |
| 18867 | 176.6817149 | 66.61303833 | 284.2722222 | 52.36278229 | 87.92815372 |
| 6176 | 154.8005992 | 169.3703287 | 44.49444444 | 18.01095855 | 87.84461153 |

TABLE 5B
CYCLOPHOSPHAMIDE Attorney Docket No. 44921-5090US
Timepoint(s): 48, 192 hrs Document No. 1828250.1

| Identifier | NonToxMean | NonToxSD | ToxMean | ToxSD | LDA score |
|---|---|---|---|---|---|
| 26150 | 3550.217066 | 2394.035515 | 157.7688889 | 162.0784058 | 98.37092732 |
| 25386 | 24.9310124 | 36.32254298 | −68.57555556 | 15.60065312 | 97.86967419 |
| 16448 | 295.9027893 | 121.8653206 | 44.90888889 | 18.08804954 | 97.7443609 |
| 19358 | 2636.427252 | 1134.146396 | 194.9288889 | 156.5233457 | 97.11779449 |
| 24228 | 168.861095 | 40.48597179 | 333.42 | 69.01608979 | 96.86716792 |
| 21661 | 1086.248058 | 455.9224318 | 2415.018889 | 423.7342941 | 96.86716792 |
| 1687 | 5202.480537 | 2216.464369 | 534.5122222 | 565.4217475 | 96.49122807 |
| 18907 | 461.2086777 | 258.6905152 | 44.95 | 35.28762283 | 96.11528822 |
| 1686 | 148.1554959 | 218.0270589 | −201.9277778 | 67.46000644 | 96.11528822 |
| 1998 | 40.01597107 | 38.7933197 | 71.79555556 | 9.409961625 | 95.98997494 |
| 1689 | 11129.27421 | 5494.666153 | 1384.795556 | 1607.871884 | 95.61403509 |
| 20073 | 43.11921488 | 19.54197757 | −0.895555556 | 7.625319848 | 95.23809524 |
| 13336 | 342.4632025 | 143.3142244 | 156.4666667 | 33.10822368 | 94.98746867 |
| 7120 | 52.70555785 | 30.79313655 | 4.355555556 | 7.29294884 | 94.98746867 |
| 1831 | 201.9578306 | 86.42452713 | 73.09444444 | 19.2436081 | 94.61152882 |
| 8612 | 48.78669421 | 32.71563104 | 2.87 | 6.185450671 | 94.61152882 |
| 11724 | 106.6169008 | 62.1452118 | 26.56 | 13.12789968 | 94.36090226 |
| 1845 | 60.04539256 | 81.69007625 | −65.77 | 19.051931 | 94.36090226 |
| 17832 | 5916.115806 | 2973.775003 | 499.65 | 667.4359424 | 94.23558897 |
| 25468 | 5399.580888 | 3198.650731 | 546.7733333 | 612.8920215 | 94.11027569 |
| 13082 | −10.82754132 | 26.30571767 | 21.87 | 5.790556968 | 93.98496241 |
| 1830 | 90.50177686 | 48.82027778 | 17.03555556 | 9.656973013 | 93.98496241 |
| 3251 | −61.64512397 | 81.86963205 | 81.07444444 | 30.20336451 | 93.73433584 |
| 17917 | −156.6332438 | 155.4144821 | 90.96555556 | 42.50735351 | 93.73433584 |
| 4238 | 318.5010744 | 99.31434354 | 473.2188889 | 45.50499738 | 93.35839599 |
| 16924 | 284.114814 | 236.6961766 | 57.18888889 | 48.89563898 | 93.35839599 |
| 25742 | 8356.250227 | 5370.596207 | 4169.924444 | 782.1241441 | 93.35839599 |
| 19255 | 231.8945248 | 108.3436072 | 79.43888889 | 49.01015238 | 93.35839599 |
| 25517 | 176.3818595 | 106.1264971 | 39.42333333 | 22.40504017 | 93.23308271 |
| 25469 | 4003.064938 | 2258.520949 | 489.8311111 | 522.1870591 | 93.10776942 |
| 21841 | 360.488595 | 139.5071375 | 870.68 | 189.0930325 | 92.94068505 |
| 22697 | 420.5272727 | 166.7676437 | 144.0044444 | 72.63931375 | 92.73182957 |
| 18695 | 40.07559917 | 49.03434584 | 114.08 | 27.53621888 | 92.60651629 |
| 18867 | 173.8182025 | 55.92279476 | 438.2655556 | 131.8203047 | 92.5647452 |
| 6330 | 160.3402479 | 54.52644832 | 240.7866667 | 22.77017128 | 92.48120301 |
| 1174 | −48.14640496 | 47.57161759 | 27.14555556 | 18.43397414 | 92.43943191 |
| 20763 | 258.8693595 | 130.0566692 | 151.8366667 | 22.57655144 | 92.35588972 |
| 20110 | −2.24018595 | 28.2945376 | 42.64444444 | 16.35918863 | 92.31411863 |
| 3518 | 404.4912397 | 138.5004293 | 215.8088889 | 56.08156971 | 92.10526316 |
| 23033 | 502.3732438 | 155.7958596 | 359.8811111 | 43.27514975 | 91.97994987 |
| 2980 | 171.7519215 | 89.4989369 | 518.63 | 180.6041871 | 91.93817878 |
| 17203 | 2171.309979 | 878.7260956 | 1319.43 | 172.0204484 | 91.85463659 |
| 20859 | 5610.85407 | 3111.772817 | 3379.032222 | 457.9566721 | 91.72932331 |
| 15966 | 249.7821694 | 74.82839403 | 152.0977778 | 32.29464343 | 91.60401003 |
| 8515 | 473.117624 | 167.1169592 | 208.5 | 69.50892694 | 91.60401003 |
| 17883 | 4402.743079 | 2442.456899 | 2606.916667 | 397.6454441 | 91.35338346 |
| 21211 | 1538.095062 | 538.7278117 | 901.0088889 | 200.9820645 | 91.22807018 |
| 13310 | 271.6145661 | 101.156491 | 558.1344444 | 173.5313029 | 91.0609858 |
| 17728 | 3422.378161 | 1622.03739 | 2225.345556 | 215.6580097 | 90.97744361 |
| 21657 | 832.9477893 | 275.7890614 | 1094.251111 | 90.77742181 | 90.85213033 |
| 15174 | 161.2747521 | 57.93907132 | 279.3077778 | 31.29556829 | 90.81035923 |
| 12988 | 20.65045455 | 15.46554859 | 38.92444444 | 4.434817108 | 90.72681704 |
| 11375 | 94.3567562 | 40.54539428 | 48.32444444 | 13.41039999 | 90.72681704 |
| 15524 | 1465.04093 | 489.2772934 | 1043.641111 | 119.2313393 | 90.60150376 |
| 24651 | 65.80146694 | 22.37305955 | 98.52555556 | 7.582849252 | 90.60150376 |
| 3895 | 20.64446281 | 14.16436528 | 71.39333333 | 29.04984725 | 90.55973266 |
| 5887 | 130.3261777 | 57.18441991 | 283.29 | 98.99798344 | 90.55973266 |
| 449 | 3127.170289 | 1516.361114 | 1777.201111 | 235.8423585 | 90.47619048 |
| 21575 | 144.471095 | 44.31940873 | 212.9177778 | 21.65509454 | 90.35087719 |
| 21798 | 1159.905062 | 287.1257193 | 702.1577778 | 174.5795168 | 90.3091061 |
| 25669 | 6228.35157 | 3222.530309 | 3461.314444 | 489.4610866 | 90.22556391 |
| 6263 | 206.163843 | 69.44186843 | 116.5922222 | 29.10138518 | 90.22556391 |
| 21660 | 1806.16876 | 731.2076053 | 3494.71 | 712.7416667 | 90.18379282 |
| 445 | 44.98719008 | 18.89067489 | 67.78777778 | 8.422898518 | 90.10025063 |
| 13166 | 3.887975207 | 35.11781292 | 42.94333333 | 16.84001633 | 90.10025063 |
| 25676 | 210.0928926 | 171.2011894 | 50.54333333 | 45.36792094 | 89.97493734 |
| 1688 | 11144.47357 | 7181.274687 | 1734.404444 | 1994.331749 | 89.97493734 |
| 17305 | 3940.808802 | 1953.453496 | 2324.277778 | 192.1924222 | 89.97493734 |
| 14289 | 34.21475207 | 17.22421328 | 60.96444444 | 13.73651076 | 89.97493734 |
| 1684 | 6017.427397 | 3672.839597 | 747.74111111 | 796.7840408 | 89.84962406 |
| 6059 | 132.4696901 | 43.4127263 | 60.36111111 | 13.91325128 | 89.80785297 |
| 23096 | 86.53258264 | 113.7166287 | 333.0511111 | 61.13002647 | 89.80785297 |
| 16001 | 7528.734483 | 4855.212997 | 3761 .755556 | 695.9839265 | 89.72431078 |
| 22 | 116.6855992 | 93.01095806 | 193.5 | 61.61612228 | 89.47368421 |
| 22749 | 38.73283058 | 42.66388367 | −65.43333333 | 63.23661657 | 89.43191312 |
| 5357 | 26.56760331 | 27.40335928 | 55.77555556 | 8.202799996 | 89.43191312 |
| 8597 | 156.9250207 | 47.15359875 | 264.1766667 | 46.35992558 | 89.43191312 |
| 7308 | 167.7827273 | 64.37333882 | 330.7888889 | 73.19458458 | 89.43191312 |
| 6002 | 126.4257851 | 130.9476267 | 174.8411111 | 33.83913579 | 89.34837093 |
| 18447 | 672.8122521 | 185.9974265 | 1110.67 | 260.403038 | 89.30659983 |
| 9073 | 23.56169421 | 15.18098129 | 60.87888889 | 11.06225389 | 89.30659983 |
| 5613 | 164.2440289 | 58.38802972 | 92.37111111 | 18.86019581 | 89.22305764 |
| 20647 | 17.54665289 | 13.04689843 | 49.17444444 | 14.26841977 | 88.93065998 |
| 22423 | 93.0628719 | 51.79439474 | 203.6655556 | 63.38365781 | 88.93065998 |
| 17289 | 2775.794938 | 1111.145258 | 1625.054444 | 320.3537481 | 88.84711779 |
| 10991 | 460.4735331 | 153.3327258 | 812.2366667 | 192.550642 | 88.68003342 |
| 20433 | 66.49049587 | 38.21838922 | 122.35 | 37.76470939 | 88.68003342 |
| 5307 | 42.15944215 | 100.4921326 | 51.52222222 | 5.932277762 | 88.59649123 |
| 21152 | 6144.862479 | 3972.39901 | 3620.961111 | 607.7025155 | 88.47117794 |
| 344 | 1165.158781 | 479.1208035 | 2218.087778 | 646.4923876 | 88.42940685 |
| 7760 | 308.1499587 | 98.82016401 | 251.4311111 | 19.11712874 | 88.34586466 |
| 19257 | −19.99838843 | 37.95188675 | 33.55777778 | 22.04014563 | 88.30409357 |
| 16925 | 861.1141116 | 256.1801506 | 441.9555556 | 152.8097041 | 88.30409357 |

TABLE 5C
MINOXIDIL Attorney Docket No. 44921-5090US
Timepoint(s): 24, 360 hrs Document No. 1828250.1

| Identifier | NonToxMean | NonToxSD | ToxMean | ToxSD | LDA score |
|---|---|---|---|---|---|
| 22773 | 437.8067556 | 113.9173578 | 770.9766667 | 186.8354675 | 96.76616915 |
| 14668 | 52.87129363 | 22.73990709 | 112.885 | 12.59923768 | 96.64179104 |
| 16426 | 1350.468049 | 590.2190351 | 2794.431667 | 568.1514554 | 95.89552239 |
| 15185 | 833.5899795 | 276.3338857 | 1365.58 | 220.1438492 | 94.7761194 |
| 21239 | 120.463306 | 69.30761948 | 22.36166667 | 26.2895199 | 94.52736318 |
| 20740 | 421.6917864 | 176.0108843 | 658.555 | 67.87724295 | 93.53233831 |
| 1514 | 218.1905339 | 120.2409921 | 350.3033333 | 76.61980049 | 93.53233831 |
| 22300 | 807.8076386 | 277.8860352 | 1278.253333 | 336.4332626 | 93.28358209 |
| 5969 | 440.1926078 | 161.104246 | 289.7616667 | 31.34946342 | 92.66169154 |
| 8580 | 51.76552361 | 27.86640474 | 56.36666667 | 3.172429143 | 92.1641791 |
| 24128 | 236.1605133 | 238.7952892 | 298.9316667 | 9.054462804 | 91.91542289 |
| 25094 | 129.5336345 | 54.38900928 | 180.97 | 12.31225893 | 91.41791045 |
| 15134 | 1062.092444 | 298.8329959 | 1215 | 76.86737279 | 91.29353234 |
| 22762 | 41.85238193 | 21.96300286 | 16.695 | 4.829284626 | 91.16915423 |
| 22915 | 128.4721971 | 43.93624616 | 157.3266667 | 6.991568255 | 90.7960199 |
| 15098 | 238.0394867 | 751.9793033 | 40.31166667 | 6.580618259 | 90.67164179 |
| 20848 | 1347.288172 | 440.8872106 | 1885.28 | 179.2055575 | 90.29850746 |
| 15292 | 68.32913758 | 25.701704461 | 38.195 | 6.108986004 | 90.29850746 |
| 6638 | 168.8499384 | 59.642012651 | 100.725 | 19.79344715 | 90.29850746 |
| 19480 | 1688.225893 | 545.93845291 | 2041.73 | 65.26765661 | 90.17412935 |
| 18725 | 42.04147844 | 24.15465745 | 25.46666667 | 2.55266658 | 89.30348259 |
| 24106 | 41.35211499 | 56.74408849 | 16.57166667 | 2.102678451 | 89.17910448 |
| 16322 | 112.6205544 | 33.29871385 | 157.76 | 12.297368821 | 88.93034826 |
| 18047 | 182.3833881 | 65.23717287 | 156.8233333 | 3.930957475 | 88.93034826 |
| 3088 | 42.74778234 | 32.65876885 | 36.32166667 | 2.583597621 | 88.93034826 |
| 24536 | 856.056037 | 267.7588433 | 1092.88 | 78.40703846 | 88.80597015 |
| 13596 | 1759.77768 | 617.566612 | 2637.788333 | 450.2181273 | 88.68159204 |
| 10829 | 53.38402464 | 32.19043564 | 91.23166667 | 25.31275285 | 88.1840796 |
| 4749 | 217.9386653 | 220.4647071 | 118.0333333 | 10.68132326 | 88.1840796 |
| 23596 | 301.3894251 | 101.2974417 | 216.15 | 32.28049442 | 88.05970149 |
| 23109 | 722.1644969 | 230.6924095 | 993.4616667 | 57.08507665 | 87.81094527 |
| 21818 | 39.564846 | 21.39138776 | 67.12666667 | 17.1317304 | 87.81094527 |
| 16312 | 70.97347023 | 42.54349354 | 36.005 | 8.413006003 | 87.68656716 |
| 18500 | 257.9291786 | 65.24252149 | 417.76 | 156.6989757 | 87.68656716 |
| 16124 | 224.9221561 | 101.6943359 | 131.69 | 19.99114204 | 87.68656716 |
| 7063 | 2777.985873 | 1129.206637 | 3441.838333 | 176.2347945 | 87.56218905 |
| 15569 | 401.426694 | 190.4056947 | 515.0683333 | 33.01171151 | 87.43781095 |
| 6282 | 185.8444559 | 66.66380694 | 222.7066667 | 17.68440631 | 87.43781095 |
| 3014 | 207.0456263 | 79.85192971 | 167.0216667 | 17.48674746 | 87.31343284 |
| 2005 | 37.20353183 | 19.95236328 | 15.27 | 9.920762068 | 87.31343284 |
| 1609 | 794.7036756 | 398.5283242 | 975.035 | 83.35706059 | 87.18905473 |
| 17224 | 1521.834374 | 458.5879475 | 1286.183333 | 91.95915412 | 87.06467662 |
| 7299 | 268.1671047 | 158.3330032 | 287.415 | 29.82061351 | 86.8159204 |
| 21583 | 357.890924 | 134.8922926 | 649.1816667 | 148.858798 | 86.8159204 |
| 12674 | 1706.457515 | 558.4805731 | 2754.293333 | 745.0542962 | 86.69154229 |
| 21581 | 571.0711704 | 161.1591653 | 712.2466667 | 73.11932649 | 86.69154229 |
| 14983 | 6631.179507 | 4479.972588 | 15391.655 | 5866.928943 | 86.69154229 |
| 19728 | 38.61924025 | 25.9328192 | 50.46333333 | 6.484111864 | 86.56716418 |
| 15048 | 1611.983429 | 522.4000395 | 2280.323333 | 234.5551045 | 86.44278607 |
| 16130 | 5031.751396 | 3133.431058 | 11743.30333 | 4153.659745 | 86.44278607 |
| 15886 | 399.6893224 | 123.6458295 | 489.11 | 29.43699373 | 86.31840796 |
| 20038 | 184.9781314 | 66.60638817 | 135.2616667 | 21.11623017 | 86.31840796 |
| 22498 | 73.93030801 | 77.50115096 | 98.645 | 15.11319258 | 86.31840796 |
| 17541 | 78.86632444 | 48.15834116 | 75.85166667 | 8.561437769 | 86.31840796 |
| 25050 | 4339.285318 | 2419.508026 | 9783.871667 | 2938.951862 | 86.19402985 |
| 16191 | 20.2310883 | 14.26758646 | 4.41 | 4.338188562 | 86.19402985 |
| 25319 | 6869.121766 | 4515.612917 | 15949.715 | 5786.841993 | 86.19402985 |
| 17908 | 47.90513347 | 39.02335634 | 14.825 | 7.884016109 | 86.19402985 |
| 18826 | 935.1048255 | 304.215653 | 683.405 | 108.4606395 | 86.06965174 |
| 2757 | 903.1013347 | 270.6404596 | 1366.795 | 213.6819286 | 86.06965174 |
| 4696 | 238.4688296 | 90.50570088 | 214.46 | 18.47564018 | 85.94527363 |
| 17632 | 89.20907598 | 33.46363831 | 125.705 | 17.0903935 | 85.94527363 |
| 12086 | 51.39412731 | 23.122172 | 54.58333333 | 4.50703746 | 85.94527363 |
| 3252 | 258.1934702 | 91.63035437 | 348.4483333 | 45.97108675 | 85.69651741 |
| 14384 | 513.7437577 | 159.8243939 | 625.8883333 | 35.06961501 | 85.69651741 |
| 19421 | 4905.447166 | 2612.813526 | 10187.86167 | 2889.206221 | 85.69651741 |
| 21152 | 6009.901417 | 3871.75871 | 13313.35 | 3985.438002 | 85.69651741 |
| 15741 | 278.6177002 | 91.60974085 | 198.5683333 | 40.29901359 | 85.69651741 |
| 14677 | 40.8373306 | 19.03190667 | 33.94833333 | 2.932817189 | 85.44776119 |
| 25439 | 4340.748193 | 2767.496686 | 9451.473333 | 3142.022674 | 85.44776119 |
| 25051 | 4710.8946 | 2442.685332 | 10025.33833 | 2691.144779 | 85.44776119 |
| 18272 | 93.70318275 | 63.79624646 | 56.58 | 5.891994569 | 85.32338308 |
| 17829 | 3780.245647 | 2195.997395 | 7286.673333 | 2466.488402 | 85.19900498 |
| 12135 | 41.10051335 | 18.68621179 | 30.72833333 | 5.003268598 | 85.19900498 |
| 7192 | 37.9426694 | 19.2370438 | 19.48166667 | 7.447752457 | 85.07462687 |
| 18021 | 141.865154 | 49.71003269 | 104.6766667 | 14.10675252 | 85.07462687 |
| 13033 | 81.59747433 | 28.86269731 | 101.9116667 | 14.09055062 | 84.95024876 |
| 8641 | 286.4467351 | 108.7810528 | 206.685 | 28.50822109 | 84.95024876 |
| 211 | 324.7765092 | 106.4010454 | 351.405 | 22.32212064 | 84.95024876 |
| 16132 | 5081.47 | 3013.732273 | 10408.45833 | 3076.016463 | 84.95024876 |
| 25742 | 8155.523142 | 5230.123357 | 18369.11 | 5977.202401 | 84.82587065 |
| 3682 | 162.7474127 | 76.60995297 | 186.9 | 12.30250056 | 84.82587065 |
| 20484 | 5303.545113 | 3085.010121 | 10399.15333 | 3206.107941 | 84.82587065 |
| 16886 | 1122.859877 | 543.2204479 | 1799.311667 | 394.2385372 | 84.82587065 |
| 18644 | 3361.113121 | 1749.187247 | 6525.706667 | 2192.432727 | 84.82587065 |
| 25545 | 386.3327515 | 254.1459957 | 516.5216667 | 112.7519602 | 84.82587065 |
| 9757 | 562.985462 | 133.7021905 | 565.03 | 25.3630495 | 84.82587065 |
| 18831 | 2631.251478 | 977.9807206 | 3436.33 | 405.701451 | 84.82587065 |
| 14981 | 5794.853676 | 3386.181972 | 11325.75333 | 3532.04975 | 84.70149254 |
| 16001 | 7355.064107 | 4741.387194 | 15974.51167 | 5460.203589 | 84.57711443 |
| 2802 | 398.1116632 | 105.2482808 | 550.6433333 | 97.96724303 | 84.57711443 |
| 16224 | 1413.696817 | 705.4146787 | 2619.071667 | 453.1790805 | 84.57711443 |
| 12066 | 379.866961 | 109.3099973 | 512.685 | 57.09029681 | 84.45273632 |
| 600 | 302.0152567 | 62.04587159 | 263.125 | 18.47615084 | 84.45273632 |
| 6133 | 625.038501 | 196.2520832 | 823.2183333 | 78.76218215 | 84.45273632 |
| 20004 | 39.32911704 | 20.17920335 | 71.94833333 | 20.30710951 | 84.45273632 |

TABLE 5D
HYDRALAZINE Attorney Docket No. 44921-5090US
Timepoint(s): 6 hrs Document No. 1828250.1

| Identifier | NonToxMean | NonToxSD | ToxMean | ToxSD | LDA score |
|---|---|---|---|---|---|
| 21351 | 25.28877049 | 50.01340774 | 80.7 | 6.247287411 | 99.50372208 |
| 11728 | 334.3587705 | 101.5532665 | 753.134 | 68.66121125 | 99.37965261 |
| 13270 | 149.1222746 | 73.25022364 | 476.398 | 65.59619936 | 99.25558313 |
| 15885 | 123.8732172 | 45.22333596 | 280.744 | 25.11900934 | 99.13151365 |
| 2416 | 304.2884631 | 93.70017094 | 501.23 | 5.625997689 | 99.00744417 |
| 6548 | 229.8347541 | 94.58230898 | 694.764 | 100.4268218 | 98.75930521 |
| 23110 | 186.4067623 | 60.6339517 | 344.77 | 15.43075176 | 98.75930521 |
| 25567 | 76.14856557 | 37.53074352 | 230.18 | 33.0816505 | 98.75930521 |
| 11727 | 291 .7623975 | 103.4853208 | 604.39 | 48.07210054 | 98.75930521 |
| 5258 | 369.7012705 | 94.61155347 | 664.15 | 45.82124889 | 98.63523573 |
| 3050 | 90.40352459 | 81.2674084 | 228.84 | 38.57028455 | 98.63523573 |
| 11726 | 252.4103279 | 113.6082746 | 533.508 | 40.55234297 | 98.38709677 |
| 3601 | 79.51719262 | 113.1260376 | 143.32 | 14.31897343 | 98.38709677 |
| 22464 | 108.1432172 | 46.05567267 | 278.934 | 40.19916765 | 98.38709677 |
| 19765 | 307.5082582 | 99.80835218 | 198.27 | 13.5588071 | 98.38709677 |
| 5129 | 78.23938525 | 35.29730289 | 169.976 | 12.30108654 | 98.2630273 |
| 7867 | 65.29440574 | 53.54378614 | 248.112 | 37.43640367 | 98.2630273 |
| 6936 | 75.82836066 | 36.41221995 | 198.006 | 27.93613484 | 98.13895782 |
| 20735 | 112.1542623 | 51.03917009 | 261.236 | 24.8571364 | 98.01488834 |
| 2378 | 53.8614959 | 22.53663678 | 129.844 | 19.37597043 | 98.01488834 |
| 19894 | 191.3700205 | 73.43627719 | 446.052 | 124.8858075 | 98.01488834 |
| 7299 | 263.2301025 | 149.2293008 | 773.116 | 120.8479132 | 98.01488834 |
| 7615 | 24.77413934 | 27.20961181 | 91.918 | 11.35597508 | 97.89081886 |
| 6562 | 79.63844262 | 27.10251 284 | 47.556 | 3.120437469 | 97.76674938 |
| 6053 | 7.246311475 | 11.37850051 | 36.264 | 5.106792535 | 97.76674938 |
| 8745 | 100.6684631 | 32.79596914 | 215.862 | 38.97077007 | 97.76674938 |
| 3003 | 59.87430328 | 63.74773452 | 417.552 | 106.1544362 | 97.76674938 |
| 23419 | 209.1499385 | 112.264608 | 347.558 | 37.75746774 | 97.76674938 |
| 6094 | 94.49360656 | 45.97738444 | 223.658 | 29.8297464 | 97.76674938 |
| 15841 | 52.45456967 | 26.02317729 | 85.158 | 5.254756893 | 97.6426799 |
| 6743 | 1056.460881 | 304.7263469 | 691.138 | 71.24019245 | 97.6426799 |
| 6560 | 261.8389139 | 76.83317084 | 432.406 | 30.29522537 | 97.6426799 |
| 5531 | 91.45577869 | 33.33677889 | 172.85 | 10.81836864 | 97.51861042 |
| 22614 | 39.96616803 | 26.33375601 | 156.954 | 73.5844544 | 97.51861042 |
| 12999 | 358.3752664 | 406.08666 | 639.448 | 135.479653 | 97.51861042 |
| 15212 | 58.32362705 | 48.5372259 | 116.17 | 20.97174409 | 97.51861042 |
| 20734 | 99.62717213 | 46.30310971 | 247.582 | 38.20457198 | 97.51861042 |
| 13083 | 12.19112705 | 11.35329523 | 49.166 | 8.230603259 | 97.51861042 |
| 23139 | 889.4179303 | 280.7060842 | 624.278 | 26.8757199 | 97.51861042 |
| 23035 | 125.0278893 | 44.18525457 | 245.704 | 21.65331915 | 97.39454094 |
| 12303 | 24.63354508 | 39.92502514 | 112.714 | 15.48603338 | 97.39454094 |
| 20901 | 126.0247131 | 49.181078 | 192.442 | 12.25019877 | 97.39454094 |
| 12609 | 229.6128689 | 70.58678004 | 395.284 | 29.79925553 | 97.39454094 |
| 17468 | 63.40719262 | 14.11477671 | 41.45 | 2.517806585 | 97.39454094 |
| 3430 | 110.9984016 | 34.42005532 | 189.106 | 13.11816794 | 97.27047146 |
| 3615 | 273.272418 | 142.0209262 | 575.834 | 43.26622967 | 97.27047146 |
| 2729 | 526.5145902 | 211.8606837 | 276.05 | 17.51608546 | 97.27047146 |
| 3941 | 178.5560656 | 73.37835308 | 380.742 | 47.43371923 | 97.27047146 |
| 4749 | 216.1424795 | 220.4400788 | 273.36 | 7.217873648 | 97.27047146 |
| 13240 | 82.07870902 | 170.9465173 | 177.364 | 78.48562945 | 97.27047146 |
| 22958 | 242.8463934 | 113.1769807 | 375.692 | 31.51711075 | 97.27047146 |
| 3988 | 103.0042828 | 44.78536688 | 201.586 | 16.73593828 | 97.14640199 |
| 23230 | 263.4350205 | 77.37219894 | 162.82 | 9.76721813 | 97.14640199 |
| 18897 | 143.05782791 | 58.80227535 | 66.48 | 4.752252098 | 97.14640199 |
| 13966 | 94.300655741 | 36.7385077 | 194.022 | 33.47051419 | 97.02233251 |
| 8495 | 127.5997131 | 44.48435803 | 192.222 | 8.326732252 | 97.02233251 |
| 23632 | 120.1993648 | 57.71823343 | 219.914 | 21.75806126 | 96.89826303 |
| 22411 | 89.94428279 | 39.81299229 | 183.18 | 16.21797614 | 96.89826303 |
| 22666 | 78.62934426 | 49.73954055 | 125.038 | 7.350821043 | 96.77419355 |
| 18673 | 133.5581967 | 39.76929661 | 237.244 | 27.73314137 | 96.77419355 |
| 5292 | 103.9889959 | 77.71665696 | 376.76 | 113.5779994 | 96.77419355 |
| 3049 | 270.0245287 | 176.9428259 | 498.444 | 86.81850022 | 96.77419355 |
| 12098 | −4.470143443 | 38.06130214 | 90.994 | 33.14980513 | 96.65012407 |
| 4725 | 89.900799181 | 121.3694137 | 239.676 | 67.29365074 | 96.65012407 |
| 25379 | 182.44686481 | 58.70865091 | 314.97 | 26.02254695 | 96.52605459 |
| 23184 | 51.544364751 | 20.45905428 | 99.778 | 11.46841183 | 96.52605459 |
| 4432 | 44.89356557 | 28.7463111 | 116.542 | 21.41826253 | 96.52605459 |
| 11331 | 84.0029918 | 27.00256457 | 140.22 | 14.35317212 | 96.52605459 |
| 2242 | 179.3839549 | 109.2501927 | 449.872 | 63.97026981 | 96.40198511 |
| 22948 | 226.7368238 | 88.46303286 | 406.712 | 35.71772767 | 96.27791563 |
| 21474 | 26.06686475 | 37.62057819 | 58.854 | 16.86412405 | 96.27791563 |
| 13420 | 392.077377 | 136.1010405 | 643.844 | 55.5189907 | 96.27791563 |
| 9658 | 89.48319672 | 60.98883523 | 244.732 | 62.7517846 | 96.15384615 |
| 8786 | 227.0502869 | 57.31974738 | 354.85 | 38.90896812 | 96.15384615 |
| 10019 | 135.9156148 | 51.50047551 | 215.752 | 15.05196067 | 96.15384615 |
| 21447 | 84.93477459 | 72.3054854 | 134.306 | 15.02143402 | 96.15384615 |
| 1031 | 70.47788934 | 23.78814851 | 110.658 | 6.08208188 | 96.15384615 |
| 4089 | 86.77180328 | 50.36619051 | 152.214 | 18.13651786 | 96.15384615 |
| 7220 | 76.98762295 | 52.95135462 | 51 .862 | 0.787953044 | 96.02977667 |
| 14955 | −144.6063525 | 185.8863263 | 181.094 | 80.56877081 | 96.02977667 |
| 15215 | 46.76629098 | 71.69519515 | 96.74 | 4.716312119 | 96.02977667 |
| 101151 | 72.56655738 | 39.87979807 | 176.686 | 32.01721303 | 96.02977667 |
| 51861 | 68.7789959 | 43.12884438 | 140.366 | 31.8155682 | 95.9057072 |
| 6135 | 96.06844262 | 60.215514 | 18.306 | 5.138353822 | 95.9057072 |
| 3392 | 18.26368852 | 19.32174615 | 37.24 | 5.439172731 | 95.9057072 |
| 19005 | 23.93286885 | 27.2110811 | 58.162 | 3.216227604 | 95.78163772 |
| 16834 | 82.12 | 36.37427009 | 148.728 | 10.54378111 | 95.78163772 |
| 12107 | 214.0981148 | 48.85349382 | 306.29 | 27.59810954 | 95.78163772 |
| 20823 | 4789.302008 | 1460.120187 | 3761.908 | 72.57799164 | 95.78163772 |
| 1571 | 172.7995902 | 52.57153601 | 290.112 | 33.25647711 | 95.78163772 |
| 16679 | 85.16483607 | 55.49961919 | 114.73 | 3.186847973 | 95.78163772 |
| 20740 | 420.9239754 | 173.9236038 | 780.866 | 98.83567059 | 95.65756824 |
| 13029 | 203.5453893 | 109.8932269 | 435.558 | 85.38083315 | 95.65756824 |
| 17903 | 134.3879918 | 52.4369357 | 244.54 | 29.13306197 | 95.65756824 |
| 8541 | 52.66313525 | 43.41680816 | 155.044 | 32.98623092 | 95.65756824 |
| 9583 | 81.29731557 | 72.74693735 | 147.982 | 35.59071396 | 95.65756824 |
| 18172 | 56.73938525 | 31.97006523 | 114.12 | 11.09620881 | 95.5756824 |
| 17342 | 96.22192623 | 40.72937854 | 186.694 | 24.4790059 | 95.53349876 |

TABLE 5E
BI-QT Attorney Docket No. 44921-5090US
Timepoint(s): 168, 336 hrs Document No. 1828250.1

| Identifier | NonToxMean | NonToxSD | ToxMean | ToxSD | LDA score |
|---|---|---|---|---|---|
| 15571 | 1146.880144 | 395.2301612 | 344.0114286 | 78.5393484 | 99.50124688 |
| 4130 | 224.4683539 | 90.2743589 | 61.24857143 | 21.27678346 | 99.25187032 |
| 13985 | 192.7222428 | 107.6912308 | 940.0885714 | 548.037149 | 99.12718204 |
| 6155 | 2926.942387 | 1119.661491 | 839.7942857 | 183.1872543 | 99.12718204 |
| 6153 | 545.0967695 | 216.1557262 | 150.66 | 30.14385012 | 99.00249377 |
| 22385 | 157.9224074 | 76.66175396 | 26.55 | 7.325947038 | 98.87780549 |
| 24072 | 21.71541152 | 30.2751282 | 58.57571429 | 11.67921067 | 98.87780549 |
| 25632 | 1153.660144 | 384.1645161 | 332.5 | 83.94025951 | 98.62842893 |
| 16909 | 282.7741975 | 106.2392905 | 497.3228571 | 47.77587387 | 98.62842893 |
| 17739 | 96.96164609 | 61.17559301 | 15.43 | 9.458525255 | 98.62842893 |
| 22386 | 377.1867078 | 156.9790178 | 80.55714286 | 28.64955364 | 98.37905237 |
| 3780 | 265.3458642 | 204.4183726 | 1800.691429 | 455.8843974 | 98.37905237 |
| 18541 | 1459.720021 | 519.9366207 | 2922.245714 | 346.1402564 | 98.25436409 |
| 25647 | 188.3438683 | 104.7747009 | 650.29 | 186.492508 | 98.25436409 |
| 2852 | 428.362037 | 187.9366738 | 135.16 | 25.3881685 | 98.25436409 |
| 23927 | 105.5380864 | 89.82305587 | 472.3742857 | 134.9741641 | 98.12967581 |
| 20925 | 1087.997305 | 385.8777079 | 2342.994286 | 432.2368176 | 98.12967581 |
| 14516 | 127.7729835 | 70.60924102 | 518.35 | 279.8149362 | 98.00498753 |
| 16426 | 1381.330185 | 604.334374 | 445.4371429 | 127.9430389 | 98.00498753 |
| 17358 | 1801.238971 | 591.0796197 | 3310.938571 | 322.5233568 | 97.75561097 |
| 6156 | 612.5829218 | 252.0319969 | 178.0885714 | 39.95518424 | 97.75561097 |
| 15438 | 225.8336214 | 74.28338074 | 78.97571429 | 17.44229425 | 97.75561097 |
| 2103 | 2781.732942 | 832.9661434 | 4941.147143 | 817.8200858 | 97.63092269 |
| 25663 | 398.4210082 | 286.1815128 | 2127.534286 | 860.1651723 | 97.63092269 |
| 20617 | 225.4005761 | 146.6464791 | 637.2714286 | 207.0700452 | 97.63092269 |
| 24521 | 5871.713724 | 1743.189805 | 11366.89286 | 2042.009624 | 97.50623441 |
| 15569 | 407.4651646 | 186.980155 | 79.59142857 | 23.82907847 | 97.50623441 |
| 26030 | 1019.45144 | 416.7005322 | 2307.601429 | 329.0417094 | 97.50623441 |
| 25716 | 629.3167284 | 321.6678121 | 2028.478571 | 531.6434546 | 97.50623441 |
| 25671 | 96.61096708 | 54.5217446 | 324.9414286 | 104.2805727 | 97.50623441 |
| 25918 | 40.67644033 | 24.17785017 | 151.8957143 | 52.53468056 | 97.38154613 |
| 4468 | 238.3986831 | 90.76454198 | 87.61 | 25.76276836 | 97.25685786 |
| 10820 | 2016.776502 | 528.6935889 | 3615.775714 | 669.0374831 | 97.25685786 |
| 25691 | 910.3856584 | 238.3647268 | 1740.794286 | 305.2156721 | 97.25685786 |
| 14928 | 875.0561111 | 272.4300315 | 1524.474286 | 184.1657844 | 97.25685786 |
| 5596 | 118.6190741 | 52.69410791 | 48.98142857 | 5.334752192 | 97.13216958 |
| 17203 | 2126.510741 | 848.1329048 | 4186.382857 | 491.1376271 | 97.13216958 |
| 19094 | 1802.569053 | 511.7092442 | 3011.387143 | 403.2550496 | 97.13216958 |
| 26133 | 187.2550412 | 152.2951324 | 651.9285714 | 291.6253559 | 97.13216958 |
| 16953 | 1149.682613 | 395.8125977 | 2207.674286 | 341.0510964 | 97.0074813 |
| 17687 | 84.04351852 | 28.20993149 | 47.67285714 | 4.369506892 | 97.0074813 |
| 24264 | 226.0653704 | 60.74150388 | 104.7857143 | 21.85609058 | 96.88279302 |
| 24268 | 160.5231687 | 93.51216047 | 48.98571429 | 15.7582157 | 96.75810474 |
| 18542 | 1410.127613 | 591.2972027 | 3140.035714 | 530.3397695 | 96.75810474 |
| 25702 | 669.0991152 | 161.3180834 | 1261.007143 | 209.6897601 | 96.75810474 |
| 14696 | 1412.849074 | 542.7044511 | 2881.84 | 376.1605253 | 96.63341646 |
| 6205 | 418.8476543 | 103.1580387 | 277.4428571 | 24.53683742 | 96.63341646 |
| 22930 | 325.4591564 | 178.4676831 | 65.04428571 | 24.94755423 | 96.63341646 |
| 18606 | 747.3085185 | 251.2163681 | 1476.484286 | 266.8125197 | 96.50872818 |
| 24310 | 294.4701029 | 113.7572958 | 86.83714286 | 30.73016689 | 96.50872818 |
| 3324 | 22.2987037 | 20.44562524 | 74.09714286 | 16.49790766 | 96.3840399 |
| 10498 | 1222.585761 | 451.0798756 | 2408.967143 | 311.0807358 | 96.3840399 |
| 5049 | 619.2253909 | 166.158467 | 363.0071429 | 70.05085812 | 96.25935162 |
| 8137 | 271.8144444 | 88.72552217 | 168.51 | 14.26964844 | 96.25935162 |
| 10109 | 1183.328992 | 282.8203183 | 2069.12 | 324.8440536 | 96.25935162 |
| 12639 | 1454.544053 | 544.733031 | 2790.017143 | 354.1095492 | 96.25935162 |
| 16938 | 1732.038395 | 603.1174343 | 3439.441429 | 525.2777621 | 96.25935162 |
| 18686 | 797.3768313 | 271.7240004 | 1443.837143 | 186.2989828 | 96.25935162 |
| 17066 | 98.15664609 | 33.72052363 | 215.0885714 | 53.79859336 | 96.25935162 |
| 23687 | 3345.324444 | 1382.257243 | 7377.175714 | 1444.234973 | 96.25935162 |
| 20812 | 1511.57249 | 561.539999 | 2893.697143 | 338.0689935 | 96.13466334 |
| 815 | 1320.18214 | 490.833371 | 2569.515714 | 392.7361894 | 96.00997506 |
| 15876 | 1311.420782 | 462.6508596 | 2416.09 | 321.7240623 | 96.00997506 |
| 16918 | 1453.174733 | 600.1195123 | 3050.62 | 559.0751988 | 96.00997506 |
| 10971 | 41.39814815 | 20.52713419 | 89.83857143 | 19.14333951 | 95.88528678 |
| 4594 | 96.26329218 | 56.87162938 | 34.98285714 | 8.56469831 | 95.88528678 |
| 18611 | 1884.292037 | 683.8558856 | 3369.444286 | 364.8119431 | 95.63591022 |
| 10684 | 634.5722634 | 194.941 2802 | 243.7285714 | 63.49565351 | 95.63591022 |
| 14944 | 58.02588477 | 31.88395661 | 147.6442857 | 46.06569253 | 95.51122195 |
| 17204 | 3058.224198 | 1371.907148 | 6358.744286 | 945.0767043 | 95.51122195 |
| 13161 | 121.0037654 | 66.35605552 | 5.641428571 | 12.79682569 | 95.51122195 |
| 600 | 302.8107202 | 61.35003616 | 213.4528571 | 13.31856188 | 95.26184539 |
| 2413 | 550.3817078 | 157.9279858 | 393.6171429 | 25.89780539 | 95.26184539 |
| 20484 | 5276.654918 | 3057.15583 | 11538.15714 | 2130.598132 | 95.26184539 |
| 2418 | 132.1191358 | 59.23141538 | 253.6085714 | 33.20308943 | 95.26184539 |
| 961 | 106.1441975 | 72.02636277 | 198.4485714 | 29.59039114 | 95.26184539 |
| 22717 | 660.8265226 | 261.2636841 | 213.93 | 65.08793052 | 95.26184539 |
| 19452 | 209.6715638 | 157.5847402 | 375.5571429 | 58.84777983 | 95.13715711 |
| 3271 | 449.0984979 | 108.7471593 | 281.7471429 | 23.07254625 | 95.13715711 |
| 21945 | 218.1630041 | 109.3266587 | 448.4914286 | 101.8108389 | 95.13715711 |
| 4499 | 719.5167901 | 247.3565527 | 1216.784286 | 136.5163842 | 95.13715711 |
| 1218 | 63.7545679 | 32.64389831 | 29.65 | 5.581594754 | 95.13715711 |
| 10821 | 28.84495885 | 92.35105778 | 195.0385714 | 76.96438774 | 95.13715711 |
| 16613 | 96.69055556 | 35.6771092 | 39.28285714 | 12.23417306 | 95.01246883 |
| 17305 | 3852.408107 | 1893.75281 | 7999.945714 | 1186.256538 | 94.88778055 |
| 25354 | −8.257839506 | 17.0880336 | 20.40571429 | 4.437070569 | 94.88778055 |
| 3698 | 33.28786008 | 27.43589635 | 80.73 | 13.43121737 | 94.76309227 |
| 14345 | 77.20395062 | 46.0865573 | 126.7142857 | 13.59995693 | 94.63840399 |
| 52 | 73.23440329 | 32.77127365 | 41.34571429 | 6.458371459 | 94.63840399 |
| 18272 | 91.36792181 | 61.71129822 | 224.0171429 | 54.62458016 | 94.51371571 |
| 17155 | 550.6687243 | 206.3019369 | 1013.405714 | 154.9823162 | 94.51371571 |
| 10878 | 1540.94323 | 698.826885 | 2431.565714 | 292.1649588 | 94.38902743 |
| 23816 | 25.7423251 | 31.57965814 | 91.71714286 | 17.98398242 | 94.38902743 |
| 14257 | 278.4844239 | 118.5763469 | 95.20571429 | 43.19293031 | 94.26433915 |
| 18271 | 366.7478189 | 234.4263298 | 758.8957143 | 120.4147542 | 94.13965087 |
| 10348 | 42.71962963 | 22.74256127 | 90.58 | 27.06055309 | 94.13965087 |

TABLE 5F
CLENBUTEROL Attorney Docket No. 44921-5090US
Timepoint(s): 6, 24 hrs Document No. 1828250.1

| Identifier | NonToxMean | NonToxSD | ToxMean | ToxSD | LDA score |
|---|---|---|---|---|---|
| 15002 | 370.7732305 | 762.3101051 | 857.3685714 | 201.3203655 | 98.12967581 |
| 15003 | 111.9262757 | 226.4263127 | 803.4357143 | 204.423891 | 97.75561097 |
| 15004 | 352.0210082 | 330.3718172 | 1485.817143 | 313.2749957 | 97.63092269 |
| 1114 | 289.5106996 | 128.09997 | 616.3842857 | 115.4233377 | 96.63341646 |
| 10654 | 167.5170576 | 183.1920641 | 342.4728571 | 116.8456235 | 96.13466334 |
| 23015 | 76.27308642 | 26.56987258 | 149.2642857 | 33.70650227 | 96.13466334 |
| 3799 | 363.2015226 | 137.4649001 | 592.75 | 88.40371862 | 94.76309227 |
| 22445 | 187.4601852 | 96.30841724 | 346.9 | 73.06204008 | 93.89027431 |
| 8598 | 92.98133745 | 30.67544775 | 122.1057143 | 4.580188705 | 93.76558603 |
| 6353 | 1105.17216 | 361.2250369 | 1556.151 | 84.84804712 | 93.51620948 |
| 3518 | 397.6903909 | 137.6434541 | 634.0728571 | 71.35343666 | 93.3915212 |
| 19048 | 63.7594856 | 27.04252873 | 26.51285714 | 6.722062467 | 93.3915212 |
| 23731 | 158.5925309 | 56.30608791 | 242.5957143 | 29.07243417 | 93.26683292 |
| 19074 | 424.6941152 | 99.82039291 | 593.8114286 | 57.41912208 | 93.26683292 |
| 19452 | 209.7605761 | 157.5451603 | 369.3771429 | 82.57209975 | 93.26683292 |
| 20743 | 149.177284 | 28.99798959 | 96.58142857 | 21.32392352 | 92.89216808 |
| 21996 | 261.8768519 | 104.143056 | 112.9628571 | 43.96131205 | 92.64339152 |
| 14982 | 4860.389568 | 2678.032152 | 8103.668571 | 940.6427668 | 92.39401496 |
| 8032 | 75.87524691 | 53.81786981 | 141.9471429 | 32.61585081 | 92.39401496 |
| 14989 | 629.8680453 | 200.2881357 | 891.6857143 | 101.9140749 | 92.26932668 |
| 1287 | 594.0475926 | 196.3334958 | 353.5457143 | 71.27292236 | 92.1446384 |
| 1809 | 32.34382716 | 58.37065997 | 293.4257143 | 129.594233 | 92.10901318 |
| 22043 | 102.109177 | 96.83781169 | 148.4242857 | 16.99378598 | 91.89526185 |
| 21661 | 1101.633868 | 485.7460291 | 1726.452857 | 226.0415466 | 91.64588529 |
| 25319 | 6920.877058 | 4634.32659 | 11059.19143 | 2293.2798 | 91.64588529 |
| 345 | 112.8551852 | 266.5500429 | 224.4 | 42.67381008 | 91.64588529 |
| 25050 | 4374.355391 | 2497.20802 | 6571.208571 | 1012.44378 | 91.64588529 |
| 23601 | 384.3886008 | 116.1146338 | 554.39 | 50.0575622 | 91.52119701 |
| 14981 | 5813.459815 | 3435.434206 | 9243.827143 | 1354.118299 | 91.39650873 |
| 15434 | 195.1638477 | 97.45664823 | 100.8371429 | 16.67855281 | 91.39650873 |
| 21527 | 75.16168724 | 45.20590689 | 106.3257143 | 15.41491362 | 91.39650873 |
| 11114 | 40.55353909 | 38.70912826 | 237.0042857 | 146.076882 | 91.36088351 |
| 4770 | 180.5046091 | 60.88092848 | 267.2942857 | 27.91134468 | 91.14713217 |
| 6015 | 120.4807407 | 40.49804833 | 65.17285714 | 17.13646669 | 91.14713217 |
| 13480 | 185.4077984 | 139.8113369 | 107.6842857 | 9.097274055 | 91.14713217 |
| 20582 | 450.8917901 | 111.5774689 | 336.2228571 | 39.54263678 | 91.14713217 |
| 19454 | 332.32 | 191.2144958 | 589.4842857 | 109.3836124 | 91.02244389 |
| 6100 | 1768.235638 | 676.3611509 | 2666.937143 | 328.8918907 | 90.89775561 |
| 23139 | 888.9618724 | 281.9240049 | 731.6957143 | 30.00259925 | 90.89775561 |
| 4969 | −15.96524691 | 53.65377688 | 190.3014286 | 104.7143313 | 90.86213039 |
| 14983 | 6685.244938 | 4598.068119 | 10386.47286 | 2329.410815 | 90.64837905 |
| 25439 | 4368.391687 | 2826.996699 | 6802.121429 | 1259.273538 | 90.64837905 |
| 20859 | 5537.508374 | 3107.487037 | 7833.655714 | 691.3458646 | 90.52369077 |
| 18313 | 2294.341728 | 1076.241882 | 3669.408571 | 557.9740799 | 90.39900249 |
| 21152 | 6045.10216 | 3947.773763 | 9826.062857 | 1984.203876 | 90.39900249 |
| 15666 | 4273.786008 | 2282.850009 | 7078.084286 | 1502.015073 | 90.39900249 |
| 21079 | 621.3493621 | 190.0544448 | 903.9914286 | 181.3617693 | 90.39900249 |
| 10829 | 53.25578189 | 32.17850139 | 94.72857143 | 14.33844765 | 90.39900249 |
| 14862 | 173.56107 | 103.8304052 | 313.7571429 | 41.23265684 | 90.27431421 |
| 8886 | 150.7763374 | 36.02695349 | 197.3885714 | 13.45373359 | 90.27431421 |
| 25650 | 65.36985597 | 81.73459012 | 27.37285714 | 3.678630698 | 90.14962594 |
| 10097 | 115.1417284 | 36.54092976 | 80.26428571 | 11.00299029 | 90.14962594 |
| 18400 | 35.27985597 | 14.3952905 | 24.98571429 | 1.97574338 | 90.02493766 |
| 4242 | 223.0401029 | 79.47610213 | 141.5642857 | 28.33963588 | 89.90024938 |
| 23632 | 119.0809053 | 55.29789649 | 269.0771429 | 76.56900172 | 89.86462415 |
| 10684 | 633.1000412 | 197.1977869 | 345.9428571 | 115.571451 | 89.7755611 |
| 10020 | 107.3964403 | 54.06388278 | 234.8757143 | 59.68441082 | 89.73993587 |
| 20484 | 5335.127654 | 3144.056813 | 7478.478571 | 887.0682476 | 89.65087282 |
| 9084 | 47.74730453 | 20.27875282 | 23.58428571 | 5.660526646 | 89.65087282 |
| 19071 | 65.65384774 | 40.19826257 | 166.9 | 26.36572586 | 89.49055932 |
| 19145 | 352.1509259 | 86.22845731 | 430.0128571 | 13.94929235 | 89.40149626 |
| 16074 | 179.5921605 | 44.12121825 | 149.5614286 | 9.893212199 | 89.40149626 |
| 6655 | 47.39563786 | 45.76462342 | 75.70285714 | 63.85409768 | 89.36587104 |
| 11485 | 147.5652469 | 63.95343317 | 94.19 | 10.3080163 | 89.27680798 |
| 16130 | 5073.927263 | 3229.765392 | 7856.3 | 1434.059483 | 89.27680798 |
| 19256 | 388.9738889 | 155.386905 | 263.9557143 | 32.81897919 | 89.1521197 |
| 16613 | 96.35598765 | 36.11198381 | 62.51142857 | 9.952628511 | 89.1521197 |
| 20872 | 1267.149588 | 381.2549453 | 1749.421429 | 210.9154919 | 89.02743142 |
| 6843 | 227.6846296 | 126.3785005 | 323.74 | 37.41478407 | 89.02743142 |
| 3016 | 3546.519774 | 1779.472329 | 5154.142857 | 502.8494973 | 89.02743142 |
| 4130 | 223.36393 | 91.7292491 | 137.9271429 | 35.95580422 | 88.90274314 |
| 20944 | 391.7425514 | 111.4665412 | 519.79 | 57.30979817 | 88.90274314 |
| 12542 | 193.5654938 | 67.02161167 | 277.7957143 | 32.18979075 | 88.90274314 |
| 6606 | 1847.296523 | 927.5598068 | 3657.978571 | 955.8918461 | 88.86711792 |
| 5227 | 142.4888889 | 66.57666343 | 322.36 | 96.11474549 | 88.86711792 |
| 10498 | 1231.989856 | 469.1475752 | 1756.054286 | 236.0194512 | 88.77805486 |
| 21705 | 132.8482305 | 71.83874577 | 48.9 | 41.86501563 | 88.77805486 |
| 17736 | 557.2155556 | 368.3172294 | 1245.955714 | 344.7801629 | 88.74242964 |
| 23109 | 724.9338272 | 232.8308439 | 762.4342857 | 50.51037644 | 88.65336658 |
| 14554 | 103.3044444 | 36.04972964 | 69.87142857 | 8.746093278 | 88.5286783 |
| 17675 | 276.5611317 | 83.82010501 | 230.74 | 18.6991408 | 88.5286783 |
| 15626 | 1554.498724 | 554.6532886 | 2088.282857 | 269.7848761 | 88.5286783 |
| 18108 | 161.9191358 | 27.31024814 | 189.0414286 | 11.60434894 | 88.40399002 |
| 20975 | 151.1588477 | 43.90183399 | 101.5428571 | 15.55203064 | 88.40399002 |
| 22615 | 71.49800412 | 120.413319 | 168.23 | 77.57154418 | 88.3683648 |
| 15886 | 399.2487654 | 123.4960866 | 506.9228571 | 28.74756495 | 88.27930175 |
| 7273 | 43.3237037 | 16.16773292 | 35.15285714 | 2.460641612 | 88.27930175 |

TABLE 5G
ISOPROTERENOL Attorney Docket No. 44921-5090US
Timepoint(s): 3, 6, 24 hrs Document No. 1828250.1

| Identifier | NonToxMean | NonToxSD | ToxMean | ToxSD | LDA score |
|---|---|---|---|---|---|
| 6606 | 1800.953354 | 841.8089691 | 4533.411538 | 976.4774713 | 97.34177215 |
| 15002 | 362.1182917 | 761.5650645 | 952.3530769 | 353.3322857 | 94.38169426 |
| 2069 | 330.4443333 | 99.06165286 | 528.5230769 | 63.4645261 | 94.30379747 |
| 15004 | 340.5709583 | 304.91227 | 1385.297692 | 580.7340909 | 94.25511198 |
| 15003 | 101.0504792 | 194.3109826 | 885.8453846 | 459.6024724 | 94.25511198 |
| 22321 | 175.5143542 | 95.44895753 | 480.2076923 | 181.5534099 | 92.86270691 |
| 1892 | 1014.269417 | 431.5795254 | 2036.402308 | 441.2482924 | 92.3563778 |
| 23964 | 87.10035417 | 36.01308076 | 146.31 | 28.23942693 | 92.27848101 |
| 24246 | 258.3080625 | 103.8022036 | 420.2730769 | 76.96344569 | 91.89873418 |
| 3959 | 385.6767292 | 112.3892003 | 643.9315385 | 134.1636163 | 91.85004869 |
| 18313 | 2264.4315 | 1049.245188 | 4139.14 | 636.8504939 | 90.83739046 |
| 3669 | 24.24370833 | 25.41941681 | 61.18692308 | 13.12603735 | 90.07789679 |
| 4969 | −15.42985417 | 58.24869563 | 75.33307692 | 46.63097672 | 90.0292113 |
| 23709 | 1016.678979 | 330.0606558 | 1487.833846 | 307.0387944 | 89.57156767 |
| 19840 | 1354.720813 | 457.9859582 | 2096.473846 | 395.7357092 | 89.31840312 |
| 20743 | 149.6088542 | 28.98896918 | 104.9215385 | 12.37454029 | 89.06523856 |
| 17736 | 547.4900833 | 358.0131362 | 1287.17 | 348.8565629 | 88.88997079 |
| 1159 | 105.747 | 38.69650897 | 63.84769231 | 15.54218729 | 88.60759494 |
| 14737 | 224.8601458 | 64.13600274 | 339.1892308 | 59.31126628 | 88.55890944 |
| 574 | 564.377375 | 209.6574783 | 1093.738462 | 264.717403 | 88.51022395 |
| 17734 | 894.7706667 | 475.7469159 | 1926.886923 | 478.4763098 | 88.51022395 |
| 22432 | 117.4335417 | 48.45540046 | 220.7423077 | 46.81354792 | 88.51022395 |
| 4420 | 432.5164792 | 132.8366231 | 282.9146154 | 47.80957394 | 88.17916261 |
| 25050 | 4367.979354 | 2513.148279 | 5792.699231 | 1037.709475 | 88.10126582 |
| 7414 | 286.8900208 | 63.9356574 | 435.0638462 | 95.41052689 | 87.87731256 |
| 15510 | 258.4175833 | 78.34841326 | 171.8730769 | 39.83706825 | 87.79941577 |
| 23123 | 313.5087083 | 106.6805904 | 539.3753846 | 168.5141659 | 87.624148 |
| 18172 | 56.3719375 | 32.08588383 | 92.37615385 | 20.41992635 | 87.34177215 |
| 15277 | 746.0483958 | 180.8008198 | 991.1115385 | 163.9308576 | 87.29308666 |
| 2742 | 62.1860625 | 78.24081855 | −112.1046154 | 118.687108 | 87.24440117 |
| 15840 | 45.2428125 | 31.12066696 | 21.56230769 | 4.773264351 | 87.16650438 |
| 3417 | 436.0942917 | 135.1741359 | 646.2084615 | 94.274477 | 87.0399221 |
| 17161 | 210.4053333 | 114.7786226 | 702.9215385 | 445.4059396 | 86.81596884 |
| 10185 | 36.474875 | 19.37632107 | 17.75615385 | 5.00987448 | 86.58227848 |
| 11531 | 367.8715625 | 247.9511191 | 984.9476923 | 415.6048795 | 86.43622201 |
| 12118 | 525.9024375 | 286.5326737 | 1465.937692 | 830.3963866 | 86.43622201 |
| 6727 | 144.8295625 | 38.96930285 | 96.43692308 | 14.21936847 | 86.40701011 |
| 16001 | 7345.807333 | 4838.698104 | 11675.05923 | 2291.390292 | 86.28042843 |
| 25689 | 1079.736667 | 440.6276535 | 1835.08 | 326.588564 | 86.28042843 |
| 1435 | 850.562875 | 227.4687537 | 1162.532308 | 75.79278167 | 85.97857838 |
| 20086 | 339.539 | 144.1424378 | 609.0123077 | 127.8668778 | 85.85199611 |
| 17477 | 68.31060417 | 75.88876388 | 109.0323077 | 48.77181976 | 85.72541383 |
| 6477 | 5.359333333 | 15.35545886 | 22.17461 538 | 16.11649653 | 85.67672833 |
| 17735 | 989.8021875 | 537.5096938 | 2383 | 662.4920784 | 85.55014606 |
| 1714 | 174.2624792 | 56.10541512 | 124.7115385 | 23.63710496 | 85.52093476 |
| 16092 | 360.0260417 | 151.5217582 | 591.7107692 | 142.5327169 | 85.52093476 |
| 21624 | 2033.794917 | 921.0432897 | 3697.166154 | 1089.50728 | 85.47224927 |
| 8495 | 127.1643333 | 44.65774149 | 168.53 | 25.3563848 | 85.44303797 |
| 18859 | 95.7876875 | 32.60736678 | 61.36307692 | 16.69836339 | 85.39435248 |
| 16533 | 300.4727917 | 119.4261477 | 496.9438462 | 129.0346388 | 85.34566699 |
| 17949 | 237.7122292 | 81.85675264 | 366.7838462 | 47.06676739 | 85.34566699 |
| 15089 | 120.1443333 | 52.12069944 | 203.1538462 | 65.88051654 | 85.34566699 |
| 11684 | 172.3714167 | 64.35917746 | 93.21230769 | 32.84176943 | 85.2677702 |
| 5458 | 156.57825 | 68.43444463 | 290.8907692 | 49.90688137 | 85.09250243 |
| 15365 | 1018.750083 | 343.2321479 | 1480.175385 | 235.1792331 | 85.01460565 |
| 1606 | 40.3269375 | 19.28859363 | 32.19923077 | 4.513729909 | 84.93670886 |
| 14397 | 75.95272917 | 52.19745226 | 171.3492308 | 54.78632029 | 84.83933788 |
| 14126 | −12.87691667 | 41.57314317 | 52.57692308 | 43.59800079 | 84.83933788 |
| 11404 | 98.37770833 | 44.48352439 | 188.2038462 | 46.46541878 | 84.79065239 |
| 4944 | 101.6122083 | 43.04690565 | 202.1107692 | 61.72363492 | 84.79065239 |
| 18403 | 2207.970875 | 927.6718268 | 2522.709231 | 214.4526646 | 84.43037975 |
| 17401 | 659.922375 | 472.3825853 | 1060.563846 | 329.8673005 | 84.03115871 |
| 20057 | 25.6423125 | 28.07360317 | 39.74769231 | 9.578609984 | 84.00194742 |
| 25439 | 4352.672708 | 2836.742218 | 6259.254615 | 1468.846717 | 83.87536514 |
| 4074 | 66.60789583 | 51.21037266 | 1.167692308 | 21.81347602 | 83.87536514 |
| 18529 | 295.4269792 | 102.4645218 | 459.8169231 | 84.03101524 | 83.82667965 |
| 9720 | 58.4780625 | 27.10112016 | 114.7053846 | 55.47800504 | 83.77799416 |
| 21152 | 6031.195688 | 3970.47468 | 8594.473846 | 1968.859692 | 83.74878286 |
| 22717 | 656.7182708 | 267.8686827 | 571.8792308 | 63.22450085 | 83.74878286 |
| 20448 | 80.24770833 | 108.8299206 | 515.7284615 | 404.6230235 | 83.72930867 |
| 6043 | 46.586875 | 26.67707328 | 82.86538462 | 18.27293336 | 83.70009737 |
| 15032 | 40.13775 | 16.13252205 | 30.22692308 | 7.604412518 | 83.67088608 |
| 15741 | 279.19375 | 92.18165366 | 220.4023077 | 29.92799112 | 83.67088608 |
| 20848 | 1339.717271 | 436.8491685 | 1875.133077 | 329.9010208 | 83.62220058 |
| 22578 | 233.7812083 | 97.26518768 | 366.1269231 | 104.8709029 | 83.57351509 |
| 18830 | 3838.6645 | 1335.735088 | 6680.843077 | 2106.098773 | 83.5248296 |
| 17305 | 3870.303771 | 1954.445915 | 5424.934615 | 718.797816 | 83.49561831 |
| 17204 | 3073.473479 | 1424.8566621 | 4272.376923 | 474.0101731 | 83.49561831 |
| 3822 | 697.3917292 | 234.9086936 | 1196.067692 | 338.7275933 | 83.39824732 |
| 8919 | 223.3019167 | 55.93447508 | 314.5523077 | 34.97798025 | 83.32035054 |
| 9929 | 222.790625 | 86.93019568 | 157.6746154 | 23.94974273 | 83.29113924 |
| 21785 | 91.6991875 | 41.01657742 | 167.7553846 | 45.87078675 | 83.27166504 |
| 15837 | 513.2281875 | 149.8040944 | 723.5046154 | 109.1594744 | 83.24245375 |
| 20449 | 103.2453958 | 167.2783907 | 701.81 | 549.4084078 | 83.22297955 |
| 13382 | 134.4617708 | 68.16933521 | 70.797692311 | 33.81142069 | 83.16455696 |
| 11137 | 1708.081146 | 672.1168288 | 2312.163846 | 292.5725507 | 83.16455696 |
| 7941 | 146.1314375 | 73.11194489 | 310.5623077 | 103.0019034 | 83.14508277 |
| 14390 | 88.9756875 | 35.05866141 | 73.76153846 | 8.114264031 | 83.11587147 |
| 14494 | 220.109125 | 57.14414432 | 174.4023077 | 25.75250478 | 83.11587147 |
| 9059 | 279.2145 | 101.1511552 | 428.9946154 | 88.0439283 | 83.06718598 |
| 15841 | 52.17541667 | 26.06232737 | 75.34 | 15.9500559 | 83.06718598 |
| 17195 | 572.3617292 | 139.5046295 | 734.0430769 | 72.94451903 | 82.98928919 |
| 21101 | 48.02770833 | 47.96669222 | 76.54307692 | 24.39873371 | 82.98928919 |
| 23731 | 157.1883958 | 54.41178011 | 255.67 | 64.86708654 | 82.89191821 |
| 25806 | 49.2876875 | 11.70393941 | 35.52153846 | 7.93145725 | 82.86270691 |
| 16847 | 970.4330833 | 347.2918939 | 1382.567692 | 209.2795291 | 82.86270691 |
| 10532 | 63.6660625 | 28.75410722 | 97.56846154 | 22.81496725 | 82.81402142 |
| 10780 | 177.7290208 | 67.53442396 | 268.5346154 | 45.48876887 | 82.81402142 |
| 17563 | 1505.677375 | 501.166256 | 2170.345385 | 344.9394213 | 82.81402142 |

TABLE 5H
NOREPINEPHRINE Attorney Docket No. 44921-5090US
Timepoint(s): 3, 6, 24 hrs Document No. 1828250.1

| Identifier | NonToxMean | NonToxSD | ToxMean | ToxSD | LDA score |
|---|---|---|---|---|---|
| 20826 | 258.7765145 | 73.83046558 | 8.665454545 | 5.441719147 | 100 |
| 18831 | 2701.02971 | 902.4997428 | 12.83 | 17.86234867 | 100 |
| 168 | 2667.635685 | 650.4891795 | 367.9990909 | 88.51448655 | 100 |
| 15876 | 1357.014232 | 440.9642379 | 16.57 | 3.755039281 | 100 |
| 16953 | 11191.077344 | 379.7935463 | 9.108181818 | 4.834130363 | 100 |
| 11849 | 1865.7830705 | 248.1407998 | 20.31 | 8.51322853 | 100 |
| 15492 | 1292.0097095 | 82.35511783 | 13.51 | 11.36765323 | 100 |
| 7888 | 324.7854979 | 75.98150734 | 40.00181818 | 20.22509472 | 100 |
| 15626 | 1597.37305 | 509.1606259 | 15.50454545 | 7.333530342 | 100 |
| 18682 | 514.2239004 | 145.6935245 | 31.97 | 7.347626828 | 100 |
| 15843 | 380.979917 | 98.04058409 | 41.20727273 | 15.37995129 | 100 |
| 18686 | 824.6674481 | 257.1839854 | 12.93545455 | 4.370193048 | 100 |
| 15345 | 172.306805 | 58.27631348 | −35.76818182 | 9.988857611 | 100 |
| 17358 | 1863.2689 | 558.8049123 | 43.91818182 | 15.50249452 | 100 |
| 3203 | 313.5804772 | 81.62083102 | 41.95272727 | 10.88050283 | 100 |
| 18318 | 1165.68083 | 348.3546489 | 2.997272727 | 9.69866495 | 100 |
| 2096 | 2451.416369 | 695.361932 | 32.81909091 | 39.101207 | 100 |
| 10227 | 823.3695228 | 258.3265781 | −72.06363636 | 25.16414206 | 100 |
| 23978 | 153.6884232 | 40.89784476 | 15.28727273 | 8.150722779 | 100 |
| 1580 | 636.5954772 | 179.4959326 | 16.11 | 14.35383085 | 100 |
| 20984 | 546.2710996 | 155.5949645 | −31.04 | 14.24214029 | 100 |
| 968 | 121 .9038382 | 30.64176184 | −0.500909091 | 6.679936309 | 100 |
| 20385 | 770.4556224 | 203.3023468 | 45.79090909 | 22.17806099 | 100 |
| 13596 | 1810.789336 | 569.1435287 | 3.454545455 | 6.840249065 | 100 |
| 20243 | 129.0574689 | 37.6968512 | −2.9 | 5.883240604 | 100 |
| 21816 | 260.0651037 | 74.61300111 | −1.751818182 | 7.742645308 | 100 |
| 16038 | 2615.561763 | 767.9946959 | 24.85 | 14.65344874 | 100 |
| 23300 | 880.673278 | 226.537123 | 50.66 | 38.86598822 | 100 |
| 8789 | 506.2074896 | 143.295597 | 14.34090909 | 15.86950563 | 100 |
| 9317 | 654.9811826 | 184.0629493 | 17.41454545 | 19.26111075 | 100 |
| 13420 | 403.804917 | 125.1092091 | −7.362727273 | 2.670884089 | 100 |
| 18160 | 1953.342905 | 669.0636466 | 2.142727273 | 3.021476761 | 100 |
| 730 | 146.8325934 | 41.118501 | −3.327272727 | 15.87339478 | 100 |
| 11531 | 1392.2085477 | 269.4537066 | 30.74090909 | 35.84799449 | 100 |
| 21211 | 1560.984502 | 496.0263201 | 13.86909091 | 11.47315428 | 100 |
| 10019 | 1140.5473237 | 45.77309846 | −30.74818182 | 8.831346237 | 100 |
| 8737 | 829.5111826 | 270.0174124 | 0.809090909 | 6.715150712 | 100 |
| 1514 | 225.5738174 | 115.6570504 | −33.27 | 21.5859042 | 100 |
| 5283 | 68.47738589 | 21.21791889 | −3.467272727 | 2.931996217 | 100 |
| 4670 | 983.1897303 | 268.442193 | 63.66727273 | 37.3271644 | 100 |
| 24765 | 8698.1611 | 2736.932551 | −107.0854545 | 118.6060407 | 100 |
| 6550 | 432.3638382 | 131.5445574 | −10.41636364 | 13.92439677 | 100 |
| 3202 | 386.2796888 | 107.8897806 | 29.91363636 | 17.24658649 | 100 |
| 3081 | 386.0928838 | 116.5134935 | −18.26181818 | 11.95994216 | 100 |
| 15957 | 337.8810581 | 103.8628968 | 2.243636364 | 6.556252394 | 100 |
| 15239 | 838.2483817 | 252.8998782 | 7.542727273 | 10.01967973 | 100 |
| 8829 | 262.205083 | 83.13534615 | −4.712727273 | 6.07799653 | 100 |
| 6291 | 102.256722 | 42.41222875 | −51.31636364 | 18.26697472 | 100 |
| 17100 | 1195.278278 | 362.7244946 | 27.93181818 | 13.41732374 | 100 |
| 4948 | 319.2764938 | 84.0755349 | 33.645454551 | 7.327518494 | 100 |
| 692 | 141.6327801 | 45.02646423 | −1.7272727271 | 3.0384736 | 100 |
| 16608 | 886.6689834 | 258.0808715 | 7.421 | 40.76969733 | 100 |
| 17380 | 546.1136515 | 142.0524293 | −50.93363636 | 78.15540177 | 100 |
| 17234 | 288.0742946 | 76.62794552 | 14.30363636 | 9.793244889 | 100 |
| 24185 | 79.67609959 | 21.07355559 | 4.180909091 | 3.012754403 | 100 |
| 6861 | 179.5580498 | 49.98628922 | 5.142727273 | 8.269084703 | 100 |
| 11066 | 291.9389627 | 89.63060929 | −11.11818182 | 6.621064594 | 100 |
| 18830 | 4002.810249 | 1320.092723 | 5.034545455 | 21.76197986 | 100 |
| 18215 | −13.02128631 | 27.8157068 | 116.2727273 | 18.53949411 | 100 |
| 7224 | 468.9245851 | 121.384228 | 45.83272727 | 22.3816653 | 100 |
| 17408 | 3033.101079 | 948.9602239 | −25.32181818 | 36.9366403 | 100 |
| 24783 | 139.1163693 | 39.73307083 | 5.153636364 | 2.656310497 | 100 |
| 15571 | 1161.553154 | 369.1839332 | −6.980909091 | 12.89713957 | 100 |
| 6927 | 550.1127178 | 154.4853899 | 24.69636364 | 25.61807498 | 100 |
| 24596 | 116.1997303 | 35.60561059 | −15.29363636 | 8.096082105 | 100 |
| 3549 | 102.1588797 | 27.48608017 | 4.924545455 | 4.636889828 | 100 |
| 15365 | 1054.916888 | 313.7007295 | −20.69272727 | 34.80709384 | 100 |
| 15308 | 264.0688382 | 78.16576449 | 17.53454545 | 16.16938735 | 100 |
| 24064 | 438.5977801 | 121.1468558 | 6.629090909 | 32.59995597 | 100 |
| 23705 | 257.5565145 | 72.23324097 | 12.20909091 | 7.991269554 | 100 |
| 23372 | 292.9917842 | 103.874663 | −86.64727273 | 33.45484392 | 100 |
| 2250 | 755.4497718 | 209.0912074 | 24.20636364 | 14.2096814 | 100 |
| 19408 | 1230.174647 | 382.1879294 | 4.725454545 | 14.95432136 | 100 |
| 18663 | 598.094668 | 164.4549192 | 3.831818182 | 8.740671391 | 100 |
| 23109 | 742.8126971 | 202.9740661 | −34.62181818 | 6.237901599 | 100 |
| 3254 | 200.6041079 | 137.1574 | 1086.558182 | 93.09191155 | 100 |
| 24528 | 37.71585062 | 168.6386969 | 1093.480909 | 136.4743304 | 100 |
| 25250 | 1227.876784 | 368.4067726 | −15.41181818 | 7.229391148 | 100 |
| 3046 | −91.81815353 | 101.5951868 | 683.6272727 | 290.0463678 | 100 |
| 17535 | 235.9384855 | 61.09067701 | 0.272727273 | 5.405567668 | 100 |
| 2131 | 152.2524896 | 47.30103826 | −23.71909091 | 8.362459512 | 100 |
| 18541 | 1514.294979 | 503.4161297 | −0.957272727 | 2.214023897 | 100 |
| 15103 | 330.838361 | 71.95446887 | 5.620909091 | 6.117156945 | 100 |
| 18358 | 389.3404772 | 84.80796568 | 56.13818182 | 21.38603087 | 100 |
| 23210 | 207.9242946 | 50.957339 | 12.97454545 | 8.530906591 | 100 |
| 2435 | 123.8113693 | 34.12545346 | 1.357272727 | 2.768819571 | 100 |
| 2254 | 190.9323651 | 53.97349834 | 1.659090909 | 3.713471299 | 100 |
| 21822 | 542.4963485 | 104.8273279 | 109.2954545 | 23.83776767 | 100 |
| 11936 | 533.6882988 | 146.0354132 | −29.16727273 | 32.27326265 | 100 |
| 22205 | 695.3240664 | 170.2714155 | 87.88818182 | 18.07752739 | 100 |
| 2103 | 2876.211846 | 769.3677303 | 16.01181818 | 16.50655313 | 100 |
| 17307 | 254.348527 | 66.81528147 | −33.49090909 | 6.670976622 | 100 |
| 11516 | 117.570083 | 49.71968898 | −26.68454545 | 15.52986244 | 100 |
| 2413 | 560.0561618 | 138.3695518 | 26.70545455 | 12.44173972 | 100 |
| 16538 | 285.3856224 | 74.85853067 | −12.47818182 | 6.74247257 | 100 |
| 15511 | 487.8557676 | 135.2102351 | 12.42090909 | 2.812619614 | 100 |
| 14928 | 904.0945021 | 252.3238249 | 15.91272727 | 12.98053242 | 100 |
| 20750 | 221.4425311 | 45.74314584 | 11.22636364 | 5.453324257 | 100 |
| 7427 | 408.6870332 | 93.45830495 | 13.85727273 | 9.079045204 | 100 |

TABLE 5I
EPINEPHRINE Attorney Docket No. 44921-5090US
Timepoint(s): 3, 6, 24 hrs Document No. 1828250.1

| Identifier | NonToxMean | NonToxSD | ToxMean | ToxSD | LDA score |
|---|---|---|---|---|---|
| 17734 | 888.2450722 | 418.3316328 | 2967.57375 | 919.3804659 | 97.875 |
| 11531 | 366.5253814 | 225.3556456 | 1452.2325 | 595.7111333 | 97.875 |
| 17736 | 542.5831959 | 312.8985462 | 2046.95 | 809.1560881 | 97.25 |
| 6585 | 711.781732 | 439.0153319 | 2196.87375 | 557.9498605 | 96.875 |
| 4098 | 118.7445979 | 63.79468172 | 309.755 | 107.3850764 | 96.5 |
| 385 | 47.90284536 | 22.42491611 | −1.535 | 7.489754907 | 95.375 |
| 21657 | 830.2778144 | 270.4242843 | 1288.78125 | 225.8066116 | 93.875 |
| 5458 | 157.4439588 | 68.27623153 | 322.3525 | 66.81148527 | 93.5 |
| 5227 | 143.2841443 | 69.29449686 | 251.66375 | 41.31225136 | 93.375 |
| 20041 | 164.6427629 | 82.38204257 | 313.96 | 49.87761737 | 93.25 |
| 2069 | 332.6504948 | 100.9144709 | 518.57375 | 79.72361649 | 92.625 |
| 15277 | 748.0543299 | 182.1555459 | 1022.66625 | 111.9218696 | 92.375 |
| 16017 | 117.0040206 | 26.56818581 | 79.4475 | 8.153501877 | 92.125 |
| 18654 | 207.2880825 | 76.29506097 | 104.18 | 38.72291422 | 91.625 |
| 22937 | 18.95884536 | 18.38238104 | 50.77875 | 11.76095166 | 91.625 |
| 15283 | 306.3364124 | 85.1441326 | 436.77125 | 57.95379032 | 91.375 |
| 17735 | 995.2225773 | 522.782963 | 2925.1375 | 973.5225995 | 91.25 |
| 6789 | 449.154701 | 138.2297816 | 359.835 | 27.13126241 | 91.125 |
| 2655 | 163.7682887 | 72.87378638 | 356.14 | 113.777997 | 91.125 |
| 22247 | 553.3174433 | 208.9330121 | 883.6025 | 221.399766 | 90.875 |
| 25717 | 50.10529897 | 34.58913722 | 19.7575 | 3.491445669 | 90.375 |
| 12376 | 1960.584433 | 624.6888448 | 1264.64 | 263.3373639 | 90.125 |
| 1892 | 1019.932247 | 428.2951523 | 2331.92625 | 591.4267078 | 90.125 |
| 14989 | 627.0824536 | 195.6606757 | 1027.835 | 168.3073491 | 90 |
| 17810 | 1493.767773 | 569.0764586 | 1603.45875 | 88.72371038 | 90 |
| 24209 | 21.31257732 | 81.64677329 | 122.2275 | 56.00067136 | 89.875 |
| 22042 | 96.09375258 | 70.77738592 | 131.2975 | 22.11521826 | 89.75 |
| 19710 | 53.04616495 | 26.30615046 | 122.3625 | 31.83640852 | 89.625 |
| 574 | 569.978701 | 216.8213901 | 1085.00875 | 293.1615851 | 89.5 |
| 8919 | 224.3806804 | 56.72687008 | 306.18375 | 34.96332648 | 89.375 |
| 9166 | 68.21876289 | 52.54762518 | 200.9225 | 94.02400662 | 89.375 |
| 17949 | 238.0037113 | 79.79173057 | 429.7825 | 104.2303763 | 89.375 |
| 17361 | 91.87804124 | 39.57467877 | 53.98125 | 9.356979729 | 89.375 |
| 20461 | 86.35030928 | 31.05618459 | 48.105 | 12.54410732 | 89.375 |
| 18389 | 775.2413196 | 506.6075311 | 1720.30875 | 416.281741 | 89.375 |
| 18313 | 2294.41367 | 1079.245371 | 3493.16375 | 482.7251869 | 89.25 |
| 12695 | 5.350185567 | 36.75666009 | 82.9525 | 38.93520139 | 89.25 |
| 12609 | 229.6410515 | 71 .4952706 | 331 .44875 | 38.34897147 | 88.875 |
| 1714 | 173.6201443 | 56.23925673 | 132.68375 | 15.0060054 | 88.625 |
| 4636 | 119.3749691 | 72.97797428 | 180.37625 | 24.15894152 | 88.5 |
| 15048 | 1617.959423 | 528.6993884 | 1750.94375 | 133.3480796 | 88.375 |
| 10071 | 265.9264742 | 134.3201197 | 479.4825 | 124.2355416 | 88.375 |
| 14149 | 131.0243711 | 89.55041357 | 221.705 | 45.87699828 | 88.375 |
| 15032 | 40.0965567 | 16.05370374 | 26.53 | 7.701617641 | 88.25 |
| 1483 | 27.79618557 | 22.28955541 | −3.34875 | 14.07984825 | 88.25 |
| 18018 | 764.9609072 | 193.4526605 | 979.63875 | 69.04549213 | 88.125 |
| 26164 | 80.68028866 | 33.33373002 | 111.5275 | 9.02472279 | 88.125 |
| 2742 | 60.12063918 | 82.15513864 | −95.82 | 62.86507934 | 88 |
| 14554 | 103.4118557 | 36.00705678 | 67.53875 | 10.90265818 | 87.875 |
| 8707 | 193.2597113 | 63.01577128 | 271.675 | 29.02663358 | 87.75 |
| 23240 | 442.1703918 | 137.4123052 | 518.92125 | 18.93327942 | 87.75 |
| 15949 | 281.7723505 | 72.62837121 | 352.95875 | 23.64593438 | 87.75 |
| 8494 | 451.5067423 | 171.6423091 | 607.5525 | 45.40360425 | 87.75 |
| 15181 | 160.2542268 | 47.84356903 | 150.69625 | 9.379378501 | 87.625 |
| 15603 | 45.94983505 | 37.69467445 | 42.9275 | 3.258236815 | 87.625 |
| 8495 | 127.0749278 | 43.9921576 | 199.80375 | 30.07248003 | 87.5 |
| 17947 | 140.6838763 | 54.78480727 | 245.63625 | 64.99176738 | 87.375 |
| 3405 | 251.0247835 | 79.63950605 | 421.23375 | 70.31434744 | 87.375 |
| 16154 | 122.7517732 | 309.7565389 | 168.96625 | 45.83850345 | 87.375 |
| 3669 | 24.59857732 | 25.52578984 | 62.7625 | 17.01112472 | 87.25 |
| 7941 | 149.1416082 | 78.32922502 | 230.84 | 33.51091038 | 87.25 |
| 12581 | 52.69251546 | 57.30569446 | 81.97875 | 29.11044606 | 87.125 |
| 20990 | 118.4963918 | 51.60603594 | 199.04125 | 46.08950219 | 87.125 |
| 22873 | 215.6249278 | 84.79180636 | 374.4175 | 84.61650291 | 87.125 |
| 13547 | 173.9781443 | 55.77956063 | 229.9025 | 19.9192095 | 87 |
| 5902 | 51.03164948 | 33.30889814 | 85.59875 | 17.2955068 | 86.875 |
| 18095 | 826.025134 | 210.4110436 | 1122.11875 | 133.4793588 | 86.875 |
| 16092 | 363.112268 | 154.8973929 | 549.41125 | 70.32665649 | 86.875 |
| 9277 | 49.07348454 | 18.38794729 | 83.37375 | 23.5619772 | 86.875 |
| 8822 | 131.5994227 | 84.01472082 | 198.895 | 48.51834322 | 86.75 |
| 4496 | 60.76754639 | 22.7136402 | 43.95875 | 7.690518165 | 86.75 |
| 24012 | 909.5962887 | 309.4378079 | 667.5625 | 86.87059337 | 86.5 |
| 20204 | 45.97138144 | 14.86260572 | 18.33625 | 13.96489674 | 86.5 |
| 7936 | 75.96758763 | 23.26395527 | 53.92625 | 8.149094494 | 86.5 |
| 19768 | 684.5306186 | 188.7390939 | 915.77 | 92.35212674 | 86.375 |
| 23155 | 219.8035258 | 63.23831524 | 303.14125 | 39.03249724 | 86.375 |
| 5703 | 189.27259791 | 73.01735577 | 255.63375 | 30.86881271 | 86.375 |
| 18774 | 67.780082471 | 32.25676833 | 41 .66375 | 6.299668784 | 86.25 |
| 3941 | 178.642268 | 74.52946804 | 299.69625 | 65.18136389 | 86.25 |
| 19320 | 108.0233196 | 34.25470953 | 97.82 | 5.847399911 | 86.125 |
| 22727 | 7269.390247 | 2432.77062 | 6112.755 | 594.1530463 | 86 |
| 2997 | 74.51882474 | 33.02943782 | 56.02875 | 8.847379656 | 86 |
| 12096 | 131.6709278 | 53.17077259 | 235.45125 | 57.54323255 | 86 |
| 8343 | 55.92898969 | 19.9088009 | 59.54125 | 3.826958649 | 86 |
| 10016 | 257.6524536 | 332.4004743 | 355.33625 | 79.38333001 | 86 |
| 1521 | 75.17245361 | 42.17580483 | 156.41125 | 41.7286354 | 85.875 |
| 2459 | 115.9692371 | 265.1998591 | 1327.6475 | 966.8365364 | 85.875 |
| 82 | 41.9002268 | 19.59839295 | 21.44375 | 6.478637285 | 85.75 |
| 17145 | 186.5703299 | 91.1146045 | 110.36875 | 29.37672813 | 85.75 |
| 22605 | 45.56035052 | 41.38235837 | 223.62375 | 129.4260965 | 85.625 |
| 19392 | 596.7033402 | 175.621725 | 834.42 | 135.9808925 | 85.625 |
| 21654 | 203.556433 | 94.47999788 | 448.57875 | 177.9926862 | 85.625 |
| 353 | 99.58771134 | 42.81116446 | 357.2475 | 264.3302602 | 85.625 |

We claim:

1. A method of predicting at least one toxic effect of a compound, comprising:
(a) preparing a gene expression profile of a tissue or cell sample exposed to the compound; and
(b) comparing the gene expression profile to a database comprising at least part of the data or information of Tables 5-5I.

2. A method of claim 1, wherein the gene expression profile prepared from the tissue or cell sample comprises the level of expression for at least one gene.

3. A method of claim 2, wherein the level of expression is compared to a Tox Mean and/or NonTox Mean value in Tables 5-5I.

4. A method of claim 3, wherein the level of expression is normalized prior to comparison.

5. A method of claim 1, wherein the database comprises substantially all of the data or information in Tables 5-5I.

6. A method of predicting at least one toxic effect of a compound, comprising:
(a) detecting the level of expression in a tissue or cell sample exposed to the compound of two or more genes from Tables 5-5I; wherein differential expression of the genes in Tables 5-5I is indicative of at least one toxic effect.

7. A method of predicting the progression of a toxic effect of a compound, comprising:
(a) detecting the level of expression in a tissue or cell sample exposed to the compound of two or more genes from Tables 5-5I; wherein differential expression of the genes in Tables 5-5I is indicative of toxicity progression.

8. A method of predicting the cardiotoxicity of a compound, comprising:
(a) detecting the level of expression in a tissue or cell sample exposed to the compound of two or more genes from Tables 5-5I; wherein differential expression of the genes in Tables 5-5I is indicative of cardiotoxicity.

9. A method of identifying an agent that modulates the onset or progression of a toxic response, comprising:
(a) exposing a cell to the agent and a known toxin; and
(b) detecting the expression level of two or more genes from Tables 5-5I; wherein differential expression of the genes in Tables 5-5I is indicative of toxicity.

10. A method of predicting the cellular pathways that a compound modulates in a cell, comprising:
(a) detecting the level of expression in a tissue or cell sample exposed to the compound of two or more genes from Tables 5-5I; wherein differential expression of the genes in Tables 5-5I is associated the modulation of at least one cellular pathway.

11. The method of any one of claims 6-10, wherein the expression levels of at least 3 genes are detected.

12. The method of any one of claims 6-10, wherein the expression levels of at least 4 genes are detected.

13. The method of any one of claims 6-10, wherein the expression levels of at least 5 genes are detected.

14. The method of any one of claims 6-10, wherein the expression levels of at least 6 genes are detected.

15. The method of any one of claims 6-10, wherein the expression levels of at least 7 genes are detected.

16. The method of any one of claims 6-10, wherein the expression levels of at least 8 genes are detected.

17. The method of any one of claims 6-10, wherein the expression levels of at least 9 genes are detected.

18. The method of any one of claims 6-10, wherein the expression levels of at least 10 genes are detected.

19. A method of claim 6 or 7, wherein the effect is selected from the group consisting of myocarditis, arrhythmias, tachycardia, myocardial ischemia, angina, hypertension, hypotension, dyspnea, and cardiogenic shock.

20. A method of claim 8, wherein the cardiotoxicity is associated with at least one heart disease pathology selected from the group consisting of myocarditis, arrhythmias, tachycardia, myocardial ischemia, angina, hypertension, hypotension, dyspnea, and cardiogenic shock.

21. A method of claim 10, wherein the cellular pathway is modulated by a toxin selected from the group consisting of cyclophosphamide, ifosfamide, minoxidil, hydralazine, BI-QT, clenbuterol, isoproterenol, norepinephrine, and epinephrine.

22. A set of at least two probes, wherein each of the probes comprises a sequence that specifically hybridizes to a gene in Tables 5-5I.

23. A set of probes according to claim 22, wherein the set comprises probes that hybridize to at least 3 genes.

24. A set of probes according to claim 22, wherein the set comprises probes that hybridize to at least 5 genes.

25. A set of probes according to claim 22, wherein the set comprises probes that hybridize to at least 7 genes.

26. A set of probes according to claim 22, wherein the set comprises probes that hybridize to at least 10 genes.

27. A set of probes according to any one of claims 22-26, wherein the probes are attached to a solid support.

28. A set of probes according to claim 27, wherein the solid support is selected from the group consisting of a membrane, a glass support and a silicon support.

29. A solid support comprising at least two probes, wherein each of the probes comprises a sequence that specifically hybridizes to a gene in Tables 5-5I.

30. A solid support of claim 29, wherein the solid support is an array comprising at least 10 different oligonucleotides in discrete locations per square centimeter.

31. A solid support of claim 29, wherein the array comprises at least about 100 different oligonucleotides in discrete locations per square centimeter.

32. A solid support of claim 29, wherein the array comprises at least about 1000 different oligonucleotides in discrete locations per square centimeter.

33. A solid support of claim 29, wherein the array comprises at least about 10,000 different oligonucleotides in discrete locations per square centimeter.

34. A computer system comprising:
(a) a database containing information identifying the expression level in a tissue or cell sample exposed to a cardiotoxin of a set of genes comprising at least two genes in Tables 5-5I; and
(b) a user interface to view the information.

35. A computer system of claim 34, wherein the database further comprises sequence information for the genes.

36. A computer system of claim 34, wherein the database further comprises information identifying the expression level for the set of genes in the tissue or cell sample before exposure to a cardiotoxin.

37. A computer system of claim 34, wherein the database further comprises information identifying the expression level of the set of genes in a tissue or cell sample exposed to at least a second cardiotoxin.

38. A computer system of any of claims 34-37, further comprising records including descriptive information from an external database, which information correlates said genes to records in the external database.

39. A computer system of claim 38, wherein the external database is GenBank.

40. A method of using a computer system of any one of claims 34-37 to present information identifying the expression level in a tissue or cell of at least one gene in Tables 5-5I, comprising:
comparing the expression level of at least one gene in Tables 5-5I in a tissue or cell exposed to a test agent to the level of expression of the gene in the database.

41. A method of claim 40, wherein the expression levels of at least two genes are compared.

42. A method of claim 40, wherein the expression levels of at least five genes are compared.

43. A method of claim 40, wherein the expression levels of at least ten genes are compared.

44. A method of claim 40, further comprising the step of displaying the level of expression of at least one gene in the tissue or cell sample compared to the expression level when exposed to a toxin.

45. A method of claim 9, wherein the known toxin is a cardiotoxin.

46. A method of claim 42, wherein the cardiotoxin is selected from the group consisting of cyclophosphamide, ifosfamide, minoxidil, hydralazine, BI-QT, clenbuterol, isoproterenol, norepinephrine, and epinephrine.

47. A method of any one of claims 6-10, wherein nearly all of the genes in Tables 5-5I are detected.

48. A method of claim 47, wherein all of the genes in at least one of Tables 5-5I are detected.

49. A kit comprising at least one solid support of any one of claims 29-33 packaged with gene expression information for said genes.

50. A kit of claim 49, wherein the gene expression information comprises gene expression levels in a tissue or cell sample exposed to a cardiotoxin.

51. A kit of claim 50, wherein the gene expression information is in an electronic format.

52. A method of any one of claims 6-10, wherein the compound exposure is in vivo or in vitro.

53. A method of any one of claims 6-10, wherein the level of expression is detected by an amplification or hybridization assay.

54. A method of claim 53, wherein the amplification assay is quantitative or semi-quantitative PCR.

55. A method of claim 53, wherein the hybridization assay is selected from the group consisting of Northern blot, dot or slot blot, nuclease protection and microarray assays.

56. A method of identifying an agent that modulates at least one activity of a protein encoded by a gene in Tables 5-5I comprising:
(a) exposing the protein to the agent; and
(b) assaying at least one activity of said protein.

57. A method of claim 56, wherein the agent is exposed to a cell expressing the protein.

58. A method of claim 57, wherein the cell is exposed to a known toxin.

59. A method of claim 58 wherein the toxin modulates the expression of the protein.
